(19) United States
(12) Patent Application Publication
(10) Pub. No.: US 2024/0093208 A1
(43) Pub. Date: Mar. 21, 2024

(54) DE NOVO ENGINEERING OF A BACTERIAL LIFESTYLE PROGRAM

(71) Applicant: The Board of Trustees of the University of Illinois, Urbana, IL (US)

(72) Inventors: Ting Lu, Savoy, IL (US); Wentao Kong, Champaign, IL (US)

(73) Assignee: The Board of Trustees of the University of Illinois, Urbana, IL (US)

(21) Appl. No.: 18/463,837

(22) Filed: Sep. 8, 2023

Related U.S. Application Data
(60) Provisional application No. 63/404,971, filed on Sep. 9, 2022.

(51) Int. Cl. C12N 15/63; C12N 1/20; C12N 9/02; C12N 9/22; C12N 9/28; C12N 9/38; C12N 15/11; C12N 15/74

(52) U.S. Cl. CPC C12N 15/635; C12N 1/20; C12N 9/0071; C12N 9/22; C12N 9/2417; C12N 9/2471; C12N 15/11; C12N 15/74; C12Y 114/14; C12Y 302/01001; C12Y 302/01023; C12N 2310/20; C12N 2509/00; C12N 2800/101; C12N 2800/80; C12N 2830/002; C12N 2830/005

(57) ABSTRACT

Provided herein are systems that provide a genetic program to control bacterial life cycle and function execution, thereby conferring programmable microbial transition between planktonic and biofilm states and facilitating the development of cellular functions across physiological domains.

### PRIORITY

This application claims the benefit of 63/404,971, filed on Sep. 9, 2022, which is incorporated by reference herein in its entirety.

### GOVERNMENT SUPPORT

This invention was made with government support under N000141612525 awarded by the Office of Naval Research, under 1553649 awarded by the National Science Foundation, and under GM133579 awarded by the National Institute of Health. The government has certain rights in the invention.

### SEQUENCE LISTING

The instant application contains a Sequence Listing which has been submitted electronically in XML format and is hereby incorporated by reference in its entirety. Said XML copy, created on Oct. 30, 2023, is named 745307_UIUC-041_SL.xml and is 146,377 bytes in size.

### BACKGROUND

Synthetic biology has shown remarkable potential to program living microorganisms for applications. However, a significant discrepancy exists between the current engineering practice-which focuses predominantly on planktonic cells—and the ubiquitous observation of microbes in nature that constantly alternate their lifestyles upon environmental variations. Methods are needed in the art for regulation of the bacterial life cycle and that enables phase-specific gene expression.

### SUMMARY

Provided herein are methods of controlling transition between planktonic growth phase and biofilm growth phase in a bacterial host cell. The methods comprise growing a bacterial host cell in a medium, wherein the bacterial host cell comprises:
(i) a recombinant polynucleotide encoding one or more biofilm assembly proteins operably linked to a first repressible promoter; and
(ii) a recombinant polynucleotide encoding a protease capable of breaking down the one or more biofilm assembly proteins operably linked to a second repressible promoter.

The addition of a repressor for the first repressible promoter to the medium results in suppression of the expression of the recombinant polynucleotide encoding one or more biofilm assembly proteins and expression of the recombinant polynucleotide encoding a protease such that the bacterial host cell exhibits planktonic growth phase. In the absence of the repressor for the first repressible promoter and the presence of repressor for the second repressible promoter in the medium results in expression of the recombinant polynucleotide encoding one or more biofilm assembly proteins and suppression of the expression of the recombinant polynucleotide encoding a protease such that the bacterial host cell exhibits biofilm growth phase.

In some aspects the bacterial host cell additionally comprises a recombinant polynucleotide encoding a protein operably linked to an inducible promoter for orthogonal expression in both biofilm growth phase and planktonic growth phase, wherein when an inducer is added to the medium, the bacterial host cell expresses the protein in both biofilm growth phase and planktonic growth phase. The bacterial host can cell additionally comprise a recombinant polynucleotide encoding a protein operably linked to the second repressible promoter for protein expression in planktonic growth phase. A second repressible promoter can be P_{sczD}, wherein the host cell additionally comprises a polynucleotide encoding a sczA operably linked to a P_{sczA} promoter. The first repressible promoter can be P_{zitR}, wherein the bacterial host cell additionally comprises a polynucleotide encoding zitR operably linked to the P_{zitR} promoter. The repressor can be zinc. The one or more biofilm assembly genes can encode P1, P2, P3, P4, P5, P6, P7, P8, P9, P10, P11, P12, P13, P14, P15, P16, P17, P18, P19, P20, P21, P22, P23, P24, P25, P26, P27, P28, P29, P30, P31, P32, P33, P34, P35, P36, P37, P38, P39, P40, P41, P42, P43, P44, P45, P45IS1, P45IS2, P45IS3, P45IS4, or P45IS5. The protease can be Neutral protease B, Bacillolysin, or Subtilisin E. The inducible promoter can be P_{nisA}. The inducer can be nisin.

An aspect provides expression cassettes, vectors, and recombinant bacterial host cells comprising a recombinant polynucleotide encoding one or more biofilm assembly proteins operably linked to a first repressible promoter; and a recombinant polynucleotide encoding a protease capable of breaking down the one or more biofilm assembly proteins operably linked to a second repressible promoter. The expression cassettes, vectors, and recombinant bacterial host cells can further comprise a recombinant polynucleotide encoding a protein operably linked to an inducible promoter. The expression cassettes, vectors, and recombinant bacterial host cells can additionally comprise a recombinant polynucleotide encoding a protein operably linked to the second repressible promoter. The expression cassettes, vectors, and recombinant bacterial host cells can further comprise a recombinant polynucleotide encoding a protein operably linked to an inducible promoter and a recombinant polynucleotide encoding a protein operably linked to the second repressible promoter.

Other aspects provide expression cassettes comprising a polynucleotide encoding one or more biofilm assembly genes operably linked to an inducible or repressible promoter. The inducible promoter can be P_{nisA} and the expression cassette can further comprise a polynucleotide encoding nisK/nisR operably linked to a constitutive promoter. The expression cassettes can be present in a vector or a population of host cells. The population of host cells can be used to express one or more biofilm assembly genes such that the host cells form a biofilm in culture. Nisin can be added to the population of host cells in culture such that the population of host cells expresses the one or more biofilm assembly genes and forms a biofilm.

In some aspects, the repressible promoter of an expression cassette can be P_{sczD}, and the expression cassette can further comprise a polynucleotide encoding sczA operably linked to a P_{sczA} promoter. These expression cassettes can be present in a vector or a population of host cells. The population of host cells can be used to express one or more biofilm assembly genes such that the population of host cells form a biofilm in culture. Zinc can be added to the population of host cells in culture such that the population of host cells express the one or more biofilm assembly genes and forms a biofilm.

In some aspects, the repressible promoter of an expression cassette is P_{zitR}, and further comprises a polynucleotide encoding zitR that is also operably linked to the repressible promoter P_{zitR}. The expression cassette can be present in a vector or a population of host cells. The population of host cells can be used to control expression of one or more biofilm assembly genes in a population of host cells in culture. Zinc can be added to the population of host cells in culture such that the population of host cells does not express the one or more biofilm assembly genes. Optionally the zinc can be removed such that the population of host cells expresses the one or more biofilm assembly genes and forms a biofilm.

Another aspect provides an expression cassette comprising one or more biofilm assembly genes operably linked to a constitutive promoter, a gRNA having specificity for the constitutive promoter, and a polynucleotide encoding a dCas, wherein the gRNA having specificity for the constitutive promoter and the polynucleotide encoding dCas are operably linked to an inducible promoter. The inducible promoter can be P_{nisA} and the expression cassette can further comprise a polynucleotide encoding nisK/nisR operably linked to a constitutive promoter. The expression cassette can be present in a vector or a population of host cells. The population of host cells can be used in a method of controlling expression one or more biofilm assembly genes in a population of host cells in culture. Nisin can be added to the population of host cells in culture such that the population of host cells express the gRNA having specificity for the constitutive promoter and the dCas such that expression of the one or more biofilm assembly genes is prevented. Optionally, nisin can be removed such that the population of host cells express the one or more biofilm assembly genes and forms a biofilm.

Even another aspect comprises an expression cassette comprising:
(a) a polynucleotide encoding a protease operably linked to repressible promoter P_{sczD},
(b) a polynucleotide encoding sczA operably linked to a P_{sczA} promoter
(c) a polynucleotide encoding one or more biofilm assembly genes and zitR operably linked repressible promoter P_{zitR}.

The polynucleotide encoding a protease can be operably linked to repressible promoter P_{sczD}, and can further comprise one or more functional genes or marker genes also operably linked to the repressible promoter P_{sczD}. The expression cassette can further comprise a polynucleotide encoding one or more functional genes or marker genes operably linked to a P_{nisA} promoter. The expression cassette can be present in a vector or a population of host cells. The population of host cells can be used in a method of controlling expression of one or more biofilm assembly genes in a population of host cells in culture in the absence of zinc such that the population of host cells form a biofilm. Optionally, zinc can be added to the population of host cells such that the population of host cells switches to planktonic growth.

The population of host cells can comprise a polynucleotide encoding one or more functional genes or marker genes operably linked to a P_{nisA} promoter. Nisin can be added to the population of host cells such that the polynucleotide encoding the one or more functional genes or marker genes is expressed.

In an aspect, the one or more biofilm assembly genes can encode P1, P2, P3, P4, P5, P6, P7, P8, P9, P10, P11, P12, P13, P14, P15, P16, P17, P18, P19, P20, P21, P22, P23, P24, P25, P26, P27, P28, P29, P30, P31, P32, P33, P34, P35, P36, P37, P38, P39, P40, P41, P42, P43, P44, P45, P45IS1, P45IS2, P45IS3, P45IS4, or P45IS5. The protease can be Neutral protease B, Bacillolysin, or Subtilisin E. The zitR transcriptional repressor protein and P_{zitR} can be derived from Lactococcus. The P_{sczD} promoter, sczA, and P_{sczA} promoter can be derived from Lactococcus Iactis. The P_{nisA} and nisK/nisR can be derived from Streptococcus.

Another aspect provides a biofilm assembly protein comprising P45IS5 (SEQ ID NO:51). Even another aspect comprises a biofilm assembly protein comprising P1, P2, P3, P4, P5, P6, P7, P8, P9, P10, P11, P12, P13, P14, P15, P16, P17, P18, P19, P20, P21, P22, P23, P24, P25, P26, P27, P28, P29, P30, P31, P32, P33, P34, P35, P36, P37, P38, P39, P40, P41, P42, P43, P44, P45, and SEQ ID NO:49, wherein SEQ ID NO:49 is present in the biofilm assembly protein such that the protein is biologically functional and is capable of being cleaved by one or more proteases.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Panels a-g. Characterization of matrix scaffold proteins. a, Conceptual design of a lifestyle controlling program. Responding to environmental signals, the program directs the cell to transit between the single-celled planktonic state and the sessile biofilm state. b, Characterization of biofilms formed on glass coverslips by a library of 45 L. lactis strains that express predicted surface proteins (P1 to P45). c, Characterization of biofilms formed on treated plastic surfaces by the 45 protein-producing strains. d, SEM images of the biofilms formed on glass cover slips by the control and the P45-producing strains. e, Images of auto-aggregation observed in test tubes containing the cultures of selected strains. f, Quantification of the auto-aggregation ability of the strain library at pH 7.4 and 5.0. g, Temporal auto-aggregation kinetics of the P41- and P45-expressing strains. Data are presented as mean±s.d. from 3 independent experiments, and representative pictures from different samples are shown.

FIG. 2 Panels a-d. Controllable biofilm assembly with engineered gene circuits. a, Nisin-induced formation of synthetic biofilms. In this design, NisR/K forms a two-component system that is induced by nisin to drive the genes encoding matrix proteins P6, P25, P40 and P45. b, Nisin-triggered repression of synthetic biofilms. Nisin induces the expression of dcas9 and gRNA, which together form a complex that binds to the promoter P_{con} and blocks the expression of the downstream scaffold genes. c, Zinc-induced formation of synthetic biofilms. Upon the binding by zinc, the transcriptional repressor SczA releases itself from the promoter P_{sczA}, leading to the expression of the matrix genes. d, Zinc-triggered repression of synthetic biofilms. In the presence of zinc, the transcriptional repressor ZitR shuts down the expression of itself and the downstream matrix genes. Experimental data are presented as mean±s.d. from 3 independent experiments.

FIG. 3 Panels a-f. Directed biofilm decomposition through rational protein design. a, Protease-based dispersal of the synthetic biofilms made of P6, P25, P40 and P45. The biofilms on glass cover slips were first treated by PBS (control), Proteinase K (10 μg ml⁻¹) and Trypsin (10 μg ml⁻¹) for 2 hours at room temperature and then quantified by crystal violet staining. b, SEM images of intact, untreated biofilms and Proteinase K-treated biofilms on glass cover slips. c, Images of the cultures of the untreated and Proteinase K-treated biofilm-forming strains in test tubes at pH 7.4. d, The predicted structure of the matrix scaffold protein P45, the five insertion sites and the designed peptide linker sequence. The linker sequence contains multiple protease cutting sites. Introducing the linker to the insertion sites results in five P45 variants, namely IS1, IS2, IS3, IS4 and IS5. GLFGKLYFEG is SEQ ID NO:50. e, Quantification of the protease-based dispersal of the biofilms of the P45 variants. f, SEM images of the IS2, IS4 and IS5 biofilms with or without Proteinase K treatment. The variant IS5 allows the cell to form a dense biofilm that can be effectively decomposed by Proteinase K, which serves as the best matrix building block for lifestyle programming. Data are presented as mean±s.d. from 3 independent experiments, and representative images from experiments are shown.

FIG. 4 Panels a-h. Autonomous transition between the planktonic and biofilm phases. a, Circuit design for zinc-responsive cellular phase transition. In the absence of zinc, the matrix protein (IS5) is actively expressed but the synthesis of GFP and IS5-degrading protease X is suppressed, driving the cells to form biofilm. In the presence of zinc, IS5 synthesis is sequestered while the protease X and GFP are actively encoded, directing the cells to be planktonic along with GFP production. b, In vivo validation of biofilm decomposition by Protease B and C. P45-Zn-gfp: a strain carrying a protease-deficient version of the circuit. P45-Zn-gfp-prob and P45-Zn-gfp-probc: the two circuit-loaded strains utilizing Protease B and Protease C respectively. c-h, State transitions under different temporal patterns of zinc availability. Here, the biofilm state is characterized by biofilm accumulation while the planktonic state is characterized by GFP production. The cell remained in the planktonic (panel c) or biofilm (panel d) states in the constant presence or absence of zinc respectively; however, it alternated between the two states in concert with the change of the zinc availability (panels e-h). In all cases, GFP level is anticorrelated with biofilm thickness. In panels c-h, gray and blue background colors correspond to the presence and absence of zinc, respectively. Experimental data are presented as mean±s.d. from 3 independent experiments.

FIG. 5 Panels a-g. Applications of the lifestyle program for phase-specific biomolecule production. a, Design of a modular, generic gene circuit. Sensing zinc availability, the circuit enables both responsive, autonomous phase transition and exclusive synthesis of the functional molecule (X) in the planktonic state. b, Schematic illustration of the function of amylase, which converts the polymeric carbohydrate, starch, into the simple sugars glucose and maltose. c-d, Quantification of the biofilm thickness and amylase activity of the amylase-encoding strain in zinc-changing environments. e, Schematic diagram of the function of mHO-1 characterized by ELISA. f-g, Quantification of the biofilm thickness and bioactivity of the mHO-1-encoding strain in changing environments. In panels c, d, f, and g, gray and blue background colors correspond to the presence and absence of zinc, respectively. Data are presented as mean±s.d. from 3 independent experiments.

FIG. 6 Panels a-j. Engineered function realization decoupled to phase transition. a, Schematic diagram of the gene circuit that confers zinc-responsive phase transition and nisin-inducible production of beta-galactosidase (Bga). b-e, Biofilm thickness and hydrolytic activity of the circuit (panel a)-loaded strain in the presence of zinc but absence of nisin (b), in the absence of zinc and nisin (c), in the presence of zinc and changing nisin (d) and in the absence of zinc but presence of varying nisin (e). Each microcentrifuge tube contains X-gal and the supernatant of the culture at the corresponding condition and time; its color (yellow or blue) indicates the level of Bga in the culture. f, Schematic diagram of the gene circuit that enables zinc-responsive phase transition and nisin-inducible production of the bacteriocin Pediocin (Ped). g-j, Biofilm thickness and antimicrobial activity of the circuit (panel f)-loaded strain in the presence of zinc but absence of nisin (g), in the absence of zinc and nisin (h), in the presence of zinc and varying nisin (i) and in the absence of zinc but presence of varying nisin (j). Each image shows the inhibition zone caused by the supernatant of the culture at the corresponding condition and time; the size of the zone reflects the concentration of bioactive Pediocin in the culture. In panels b-e and g-j, gray, green and blue background colors correspond to the presence of zinc only, presence of both zinc and nisin (d and i), absence of zinc and presence of nisin (e and j), and absence of both zinc and nisin, respectively. Data are presented as mean±s.d. from 3 independent experiments and representative images from experiments are shown.

FIG. 7 Panels a-b. Additional characterization of biofilm matrix proteins. a, Plasmid for constitutive expression of the biofilm matrix proteins. b, Thickness of the biofilms formed on the surface of non-tissue culture treated 96-well plate by the library of 45 L. lactis strains that express predicted surface proteins (P1 to P45). c, SEM images of the biofilms formed by the strains encoding the proteins P6, P13, P25 and P40. Data are presented as mean±s.d. from 3 independent experiments, and representative pictures from different samples are shown.

FIG. 8 Panels a-b. Dispersal of synthetic biofilms from plastic surfaces. a, Protease-based dispersal of the biofilms made of P6, P25, P40 and P45. The biofilms on a polystyrene cell culture treated 96 well plate were directly quantified by crystal violet staining without any treatment, or treated by PBS or Proteinase K (10 μg ml⁻¹) for 2 hours at room temperature before being quantified. b, SEM images of intact, untreated biofilms and Proteinase K-treated biofilms on polystyrene plastic sheets.

FIG. 9 Panels a-b. Additional characterizations of the P45 variants. a, Quantification of biofilms formed on the polystyrene cell culture treated 96 well plate for the variants IS1-IS5. b, Images of test tubes containing the cultures of the variants at pH 7.4 and pH 5.0. c, Quantification of the aggregation ability of the variants at pH 7.4 and pH 5.0. For all panels, the strain P45 was used as a control. Data are presented as mean±s.d. from 3 independent experiments. Representative pictures from different samples are shown.

FIG. 10 Panels a-b. Protease secretion and in vitro biofilm dispersal. a, Protease secretion by L. lactis NZ9000 upon nisin induction. Lane 1, protein ladder. Lane 2, control without protease secretion. Lane 3 and 4, Protease A. Lane 5 and 6, Protease B. Lane 7 and 8, Protease C. Black arrow indicates the band of Usp45. Red arrow indicates Protease A. Green arrow indicates Protease B. Blue arrow indicates Protease C. The absence of the Usp45 band in Lane 5-9 suggests that Proteases B and C both exhibit proteolytic activity to digest Usp45. b, Inhibition of the IS5 biofilm by the supernatants of the protease-secreting strains. Overnight culture of IS5 was diluted with fresh medium to the OD₆₀₀ of 0.04, then 120 μl of the diluted culture was added to a cell culture treated 96-well plate. 30 μl of L. lactis NZ9000 supernatants containing different proteases were added into the IS5 culture. Biofilm thickness was measured after growth for 24 hours. Data are presented as mean±s.d. from 3 independent experiments.

FIG. 11 Panels a-i. Plasmid maps and control experiments for planktonic-biofilm transition. a, Map of the plasmid IS5-Zn-gfp-prob. b, Map of the plasmid P45-Zn-gfp. c, Gene circuit of the plasmid P45-Zn-gfp. d-i, State transition experiments for the strain carrying the plasmid P45-Zn-gfp under different temporal patterns of zinc availability. Compared to the case of the strain carrying the plasmid IS5-Zn-gfp-prob (FIG. 4), the biofilm of the P45-Zn-gfp loaded strain cannot be decomposed once it forms. Experimental data are presented as mean±s.d. from 3 independent experiments.

FIG. 12 Panels a-f. Increased antibiotic resistance coupled with biofilm formation. a, Design of the gene circuit IS5-orf29-P7-Erm-Zn-gfp-prob. Building on the circuit IS5-Zn-gfp-prob, this system was established by introducing the transcriptional activator gene Orf29 at the downstream of IS5 and using the cognate promoter P7 to drive the expression of the erythromycin (Erm) resistance gene. b, Validations of the biofilm-coupled Erm resistance with colony forming unit counting. Cells containing the circuit IS5-orf29-P7-Erm-Zn-gfp-prob or the circuit IS5-Zn-gfp-prob were pre-cultured in the GM17/Cm/Zn media to be induced to the planktonic state or in the GM17/Cm/EDTA media to be induced to the biofilm state for 36 h with inoculations to fresh medium occurring every 12 h. Then, cell cultures with OD600 of 1.0 were serially diluted by 10⁰-10⁶ folds, and 0.5 μl of diluted cultures were added onto the agar plate supplemented with Cm to select all cells and the agar plate with Erm to select cells with the Erm resistance. c,d, State transitions of the strain carrying the circuit IS5-orf29-P7-Erm-Zn-gfp-prob under different temporal patterns of zinc availability. The Erm resistance was coupled with biofilm formation. e,f, State transition experiments for the control strain carrying IS5-Zn-gfp-prob under different temporal patterns of zinc availability. The Erm resistance remained low regardless of the life cycle. Data are presented as mean±s.d. from 3 independent experiments.

FIG. 13 Panels a-f. Control experiments for coordinated lifestyle transition and amylase synthesis. a-b, Quantification of the biofilm thickness and amylase activity of the amylase-encoding strain, which carries the plasmid IS5-Zn-amy-prob in the constant presence (a) and absence (b) of zinc. c-f, Quantification of the biofilm thickness and amylase activity of the strain carrying the plasmid P45-Zn-amy in four different zinc-changing environments. Experimental data are presented as mean±s.d. from 3 independent experiments.

FIG. 14 Panels a-f. Control experiments for coordinated lifestyle transition and mHO-1 synthesis. a-b, Quantification of the biofilm thickness and mHO-1 concentration of the mHO-1-encoding strain, which carries the plasmid IS5-Zn-mHO-1-prob in the constant presence (a) and absence (b) of zinc. c-f, Quantification of the biofilm thickness and mHO-1 concentration of the strain carrying the plasmid P45-Zn-mHO-1 in four different zinc-changing environments. Experimental data are presented as mean±s.d. from 3 independent experiments.

FIG. 15 Panels a-j. Application of the lifestyle program for phase-specific, intracellular enzyme production. a, Design of a gene circuit (P45-Zn-gusA-prob) for GusA production by leveraging the modular structure in FIG. 5a. Here, the functional gene is gusA, which encodes beta-glucuronidase that converts p-nitrophenyl-s-D-glucopyranoside (PNPG) into the products, glucuronic acid and para-nitrophenol (PNP). PNP can be quantitatively measured by spectrometry at 420 nm. Compared to the functional molecules demonstrated in FIG. 5, one key difference here is that GusA remains intracellular and is not secreted to extracellular milieu. b-e, Quantification of the biofilm thickness and GusA activity of the strain carrying the plasmid P45-Zn-gusA-prob in different zinc-changing environments. Notably, in response to zinc variations, cellular phase transitioned between the planktonic and biofilm states owing to the coordinated expression of IS5 and Protease B. However, there was no obvious reduction of GusA activity due to its high stability in the cell. f, Gene circuit for the plasmid P45-Zn-gusA. g-j, Quantification of the biofilm thickness and GusA activity of the strain carrying the plasmid P45-Zn-gusA in different zinc-changing environments. Neither biofilm decomposition nor GusA reduction was observed for this construct due to the lack of active degradation of IS5 and GusA. Experimental data are presented as mean±s.d. from 3 independent experiments.

FIG. 16 Panels a-e. Optimization of phase-specific control of intracellular GusA via engineered fast degradation. a, Gene circuit for the optimized system, IS5-Zn-gusA-tag-prob-Pcst-lon, which contains an orthogonal protein degradation system (mf-lon) and a degradation tag for GusA (gusA/tag). When zinc is present, IS5 expression is suppressed but Protease B is actively produced and secreted to disperse existing IS5 biofilm. Meanwhile, gusA is actively expressed with a fast degradation tag that can be recognized by the protease Mf-lon. In this case, the cell is in the planktonic state with a high level of tagged GusA. When zinc is absent, IS5 expression is turned on while the synthesis of Protease B is shut off, leading to biofilm formation. Meanwhile, the production of new GusA molecules is suppressed but the protease Mf-lon continues to actively digest existing tagged GusA, resulting in reduction of intracellular GusA concentration. The gene mf-lon is under the control of the low pH inducible promoter P_{cst} which is only active in the stationary phase, which reduces metabolic load and avoids excessive digestion of GusA when zinc is present. b-e, Quantification of the biofilm thickness and GusA activity of the strain carrying the plasmid IS5-Zn-gusA-tag-prob-Pcst-lon in different zinc-changing environments. With the optimized system, both cellular phase and GusA bioactivity showed clear transitions in response to environmental zinc availability. Experimental data are presented as mean±s.d. from 3 independent experiments.

FIG. 17 Panels a-d. Growth of strains at induced or uninduced state. a, Growth of cells with the nisin induced biofilm formation circuit in FIG. 2a. Cells form biofilms when nisin is added for induction at time 2 h (Nisin+). b, Growth of cells with the nisin triggered repression of biofilm formation in FIG. 2b. Cells form biofilms when nisin is absent (Nisin−). c, Growth of cells with the zinc induced biofilm formation circuit in FIG. 2c. Cells form biofilms when zinc is present in the culture (Zn+). d, Growth of cells with the zinc triggered repression of biofilm formation in FIG. 2d. Biofilm formation is induced when EDTA is present (Zn−). Cells that can form biofilm or aggregate were vortexed vigorously to keep them well mixed in the culture for measurement of OD600. L. lactis NZ9000 containing the corresponding empty inducible plasmid was used as blank. Data are presented as mean±s.d. from 3 independent experiments.

FIG. 18 Detailed protocol for the state transition experiment. For the Zn+/Zn−/Zn+ transition, overnight cultures grown in GM17/Cm medium are diluted 1:50 with fresh GM17/Cm/Zn medium. Then, 1 ml of the dilution is inoculated into three 12-well plates with glass cover slips on the bottom and grown for 12 hours. The supernatants are carefully removed by pipette and 1 ml of fresh GM17/Cm/Zn is added to grow for another 12 hours. After 24 hours, the process is repeated. At hour 36, one 12-well plate is used to measure the enzyme in the supernatant and quantify the biofilm on the glass cover slip for the Zn+ condition. The remaining two 12-well plates are used for transition to the Zn− condition. First, the supernatants are removed, and the wells are washed once with 1 ml of M17 medium to remove remaining zinc in the well. Then, 1 ml of fresh GM17/Cm/EDTA medium is added and the culture is grown for 12 hours. Every 12 hours, the supernatant is removed and fresh GM17/Cm/EDTA is added. At hour 72, one plate is used to measure enzymes and biofilm for the Zn− condition and the remaining one is washed by M17 medium and then goes on to the next Zn+ condition. At hour 108, the last plate is measured. For other transitions such as Zn+/Zn+/Zn+, the procedure is same as above except that GM17/Cm/Zn medium is used in all conditions.

FIG. 19 Panels a-b. Quantification of pediocin production by agar diffusion assay. a, Inhibition zones with different units of pediocin (left) and the corresponding standard curve (right). b, Control experiment for the nisin inducer. The amount of nisin used for induction does not cause the formation of inhibition zone.

### DETAILED DESCRIPTION

Biofilms are important for bacterial ecology and evolution and have implications in the human gut microbiome where they enables bacteria to persist through variations in nutrient availability and can be used in wastewater treatment and environmental cleanup. Methods of controlling a switch between planktonic and biofilm life phases can be useful in manipulating host cells. Provided herein are gene circuits that can control the transition between planktonic and biofilm states. Gene circuit designs can include biofilm assembly genes to program a biofilm state, which can be reversed by a protease that degrades the biofilm Expression of these components in response to an inducer and/or repressor can lead to reversible transition between two phases. Despite the conceptual simplicity of this strategy, achieving effective transition is non-trivial. Both rational protein design and screening can be required to optimize these components. Additional components provide the ability to enable both coupled and orthogonal gene expression. For the coupled function, cells in the planktonic life phase can express a recombinant protein the in the presence of a repressor or inducer. For the orthogonal function, which can be controlled independently of life phase by a second external input, cells could be induced to express another recombinant protein.

The designs presented herein have modularity, such that components behave similarly in isolation to the way they do in combination. In addition to demonstrating the modular control of biofilm formation by multiple inputs, control of life phase (e.g., biofilm or planktonic) can be coupled with a secondary function. This coupling can enable engineered biological devices to capitalize on the benefits of each phase for optimal performance.

Many applications can be envisioned. For example, methods and compositions can be used for smart drug delivery. Bacteria entering a planktonic phase can form a biofilm in response to signals detected upon reaching their final desired location. On-demand transitioning of bacterial states can be also useful for biomanufacturing, where the planktonic state can enable more effective production of biomolecules, while the biofilm state can enable long-term survival in harsh environments.

Provided herein are synthetic genetic programs that regulate the bacterial life cycle and enables phase-specific gene expression. The program is orthogonal and harnesses engineered proteins as biofilm matrix building blocks. It is also highly controllable, allowing directed biofilm assembly and decomposition as well as responsive autonomous planktonic-biofilm phase transition. Coupled to synthesis modules, it is further programmable for various functional realizations that conjugate phase-specific biomolecular production with lifestyle alteration. This provides a versatile platform for microbial engineering across physiological regimes, thereby shedding light on a promising path for gene circuit applications in complex contexts.

Engineered organisms harboring gene circuits can be developed to encode novel cellular behaviors and functions¹⁻¹⁵. Gene circuits can be used in chemical synthesis^{16,17}, material fabrication^{18,19}, environmental remediation^{20,21} and disease treatment²²⁻²⁴. To date, the vast majority of these synthetic systems are designed, constructed and demonstrated in well controlled settings whereby cells remain exclusively planktonic and programmed functions are executed in exponential growth phase. By contrast, microorganisms in natural habitats often live in and switch between two distinctive lifestyles, a single-celled, planktonic form and a sessile, community form called biofilm²⁵⁻²⁸. The former allows cells to rapidly utilize substrate and thrive in nutrient-rich conditions; the latter provides microbes protection against disturbances and enhancement in substrate consumption under stress²⁹. Such a lifestyle alternation enables cells to cope with environmental variations between limited resource supply and transient nutrient pulse such as the cases of deep oceans with marine snow^{30,31} and the human gut with daily food intake^{32,33}. As a result, there exists a remarkable mismatch between engineered microbial plankton prevalent in the current synthetic biology practice and the ubiquitous observation of lifestyle switching microbes in natural contexts.

Provided herein is a platform with the traits of orthogonality, modularity and programmability. Adopting Lactococcus lactis (L. lactis) as the cellular chassis, 45 putative surface-associated proteins were expressed and characterized from which orthogonal building blocks for biofilm organization were identified. Gene circuit engineering was combined with protein design to establish externally controllable biofilm assembly and decomposition as well as autonomous planktonic-biofilm phase transition in response to zinc availability. The utility of the platform is demonstrated with different modes of synthesis of functional biomolecules. These systems provide a genetic program to control bacterial life cycle and function execution, thereby conferring programmable microbial transition between planktonic and biofilm states and facilitating the development of cellular functions across physiological domains.

Polynucleotides

Polynucleotides are polymers of nucleotides e.g., linked nucleosides. A polynucleotide can be, for example, a ribonucleic acid (RNA), a deoxyribonucleic acid (DNA), a threose nucleic acids (TNA), a glycol nucleic acid (GNA), a peptide nucleic acid (PNA), a locked nucleic acid (LNA), cDNA, genomic DNA, chemically synthesized RNA or DNA, or combinations or hybrids thereof. Polynucleotides of can be recombinant polynucleotides. A recombinant polynucleotide is a polynucleotide that is not in its native state, e.g., the polynucleotide comprises a nucleotide sequence not found in nature, or the polynucleotide is in a non-naturally occurring context, for example, separated from nucleotide sequences with which it typically is in proximity in nature, or adjacent (or contiguous with) nucleotide sequences with which it typically is not in proximity. For example, a recombinant polynucleotide can be cloned into a vector, or otherwise recombined with one or more additional nucleic acid.

Polynucleotides can be modified by, for example, chemical modification with respect to A, G, U (T in DNA) or C nucleotides. Modifications can be on the nucleoside base and/or sugar portion of the nucleosides which comprise the polynucleotide. In some embodiments, multiple modifications can be included in the modified nucleic acid or in one or more individual nucleoside or nucleotide. For example, modifications to a nucleoside can include one or more modifications to the nucleobase and the sugar. Polynucleotides contain less than an entire microbial genome and can be single- or double-stranded nucleic acids. Polynucleotides can be purified free of other components, such as proteins, lipids, and other polynucleotides. Polynucleotides can be isolated from nucleic acid sequences present in, for example, a bacterial or yeast culture. Polynucleotides can be synthesized in the laboratory, for example, using an automatic synthesizer. An amplification method such as PCR can be used to amplify polynucleotides from either genomic DNA or cDNA encoding the polypeptides.

A polypeptide can be produced recombinantly. A polynucleotide encoding a polypeptide can be introduced into a recombinant expression vector, which can be expressed in a suitable expression host cell system. A variety of bacterial, yeast, plant, mammalian, and insect expression systems are available in the art and any such expression system can be used. Polynucleotides can comprise coding sequences for naturally occurring polypeptides or can encode altered sequences that do not occur in nature.

“Operably linked” refers to the expression of a gene that is under the control of a promoter with which it is spatially connected. A promoter can be positioned 5′ (upstream) or 3′ (downstream) of a gene under its control. A promoter can be positioned 5′ (upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

Polynucleotides can encode full-length polypeptides, polypeptide fragments, and variant or fusion polypeptides. A polynucleotide can encode a polypeptide, which can be an enzyme or protein that has biological activity. A polynucleotide can encode any polypeptide (e.g., a recombinant non-naturally occurring polypeptide or a naturally occurring polypeptide).

A polypeptide expressed by a polynucleotide can react substantially the same as a wild-type polypeptide in an assay of biological activity, e.g., has 80-120% of the activity of the wild-type polypeptide. A wild-type polypeptide is a polypeptide that is not genetically altered and that has an average biological activity in a natural population of the organism from which it is derived.

Expression Cassettes

Expression cassettes or constructs comprise two or more polynucleotide sequences and can comprise one or more promoters or other expression control sequences (e.g., enhancers, transcriptional terminator sequences, etc.), one or more coding polynucleotides, one or more non-coding polynucleotides. Expression cassettes or constructs can be inserted into a vector, transformed into a host cell, e.g., a bacterial host cell. The expression cassettes can be linear or circular. A linear or circular expression cassette can be integrated into a vector, host bacterial genome, or expression plasmid within the host cell.

The terms “derived from” or “from” when used in reference to a polynucleotide or polypeptide indicate that its sequence is identical or substantially identical to that of the organism of interest. For example a Mucus binding Mub polynucleotide derived from Lactobacillus acidophilus refers to a Mucus binding Mub polynucleotide from Lactobacillus acidophilus having a sequence identical or substantially identical (e.g., about 85, 90, 95, 97, 98, 99%, or more identical) to a native Mucus binding Mub polynucleotide from Lactobacillus acidophilus.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NO:1-66) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence (e.g., SEQ ID NO:1-66).

Polypeptides and polynucleotides that are sufficiently similar to polypeptides and polynucleotides described herein (e.g., SEQ ID NO:1-66) can be used herein. Polypeptides and polynucleotides that are about 90, 91, 92, 93, 94 95, 96, 97, 98, 99 99.5% or more identical to the polypeptides and polynucleotides described herein can also be used.

For example, a polypeptide of polynucleotide can have 80% 85%, 90%, 91%, 92%, 93%, 94%, 95%, 96%, 97%, 98%, 99% or more identity to SEQ ID NO:1-66.

Vectors

A vector is a polynucleotide that can be used to introduce polynucleotides or expression cassettes into one or more host cells. Vectors include cloning vectors, expression vectors, shuttle vectors, plasmids, cassettes, and the like. Any suitable vector can be used to deliver polynucleotides or expression cassettes to a population of host cells.

A plasmid is a circular double-stranded DNA construct used as a cloning and/or expression vector. Some plasmids can take the form of an extrachromosomal self-replicating genetic element (episomal plasmid) when introduced into a host cell. Other plasmids integrate into a host cell chromosome when introduced into a host cell. Expression vectors can direct the expression of polynucleotides to which they are operatively linked. Expression vectors can cause host cells to express polynucleotides and/or polypeptides other than those native to the host cells, or in a non-naturally occurring manner in the host cells. Some vectors may result in the integration of one or more polynucleotides (e.g., recombinant polynucleotides) into the genome of a host cell.

Polynucleotides or expression cassettes can be cloned into an expression vector optionally comprising expression control elements, including for example, origins of replication, promoters, enhancers, or other regulatory elements that drive expression of the polynucleotides or expression cassettes in host cells. One or more polynucleotides or expression cassettes can be present in the same vector. Alternatively, each polynucleotide or expression cassette can be present in a different vector.

Host Cells

A host cell or population of host cells can be any suitable host cell, for example, a bacterial cell such as Enterococcus sp., Streptococcus sp., Leuconostoc sp., Lactobacillus sp., and Pediococcus sp., Bacillus sp., Escherichia sp. Other examples include Streptococcus pyogenes, Streptococcus agalactiae, Streptococcus pneumoniae, Streptococcus zooepidemicus, Enterococcus faecalis, E. coli, Bacillus subtilis, Bacillus amyloliquefaciens, Bacillus licheniformis, Bacillus cereus, Lactobacillus helveticus, Lactobacillus casei, Lactobacillus plantarum, Lactobacillus paraplantarum, Lactobacillus keid, Lactobacillus gassei, Lactobacillus salivarius, Lactobacillus casei, Lactobacillus paracasei, Lactobacillus brevis, Lactobacillus acidophilus, Lactobacillus delbrueckii, Lactobacillus rhamnosus, and Lactobacillus reuter.

Promoters

A polynucleotide described herein can be operably linked to a promoter. An expression cassette can comprise one or more promoters operably linked to one or more polynucleotides. A promoter can be a constitutive promoter. A constitutive promoter can drive the expression of polynucleotides continuously and without interruption in response to internal or external cues. Constitutive promoters can provide robust polynucleotide expression. Bacterial constitutive promoters include, for example, promoter of an IcnA gene in gene cluster of lactococcin A from Lactococcus, E. coli promoters Pspc, Pbla, PRNAI, PRNAII, P1 and P2 from rrnB, and the lambda phage promoter PL. Constitutive promoters can be functional in a wide range of host cells.

A promoter can be an inducible promoter. An inducible promoter can drive expression of polynucleotides selectively and reliably in response to a specific stimulus. In some embodiments an inducible promoter will drive no polynucleotide expression in the absence of its specific stimulus, but drive robust polynucleotide expression upon exposure to its specific stimulus. Additionally, some inducible promoters can induce a graded level of expression that is tightly correlated with the amount of stimulus received. Stimuli for inducible promoters include, for example, heat shock, exogenous compounds or a lack thereof (e.g., a sugar, metal, drug, or phosphate), salts or osmotic shock, oxygen, and biological stimuli (e.g., a growth factor or pheromone).

Inducible promoters can be regulated by positive and negative control. A positively inducible promoter is inactive in an off state such that an activator cannot bind to the promoter. Once an inducer binds to the activator, then the activator protein can bind to the promoter, turning it on such that transcription occurs.

A negatively inducible promoter is inactive when bound to a repressor protein, such that the transcription does not occur. Once an inducer binds the repressor, the repressor is removed from the promoter and transcription is turned on.

In a Tet-On system the activator rtTA (reverse tetracycline-controlled transactivator) is inactive and cannot bind tetracycline response elements (TRE) in a promoter. Tetracycline and its derivatives are inducing agents that allow promoter activation such that transcription occurs.

A negative inducible pLac promoter requires removal of the lac repressor (lacI protein) for transcription to be activated. In the presence of lactose or lactose analog IPTG, the lac repressor undergoes a conformational change that removes the repressor from lacO sites within the promoter and such that transcription occurs.

In the absence of arabinose regulatory protein AraC binds O and I1 sites upstream of pBad, a negative inducible, thereby blocking transcription. The addition of arabinose causes AraC to bind I1 and I2 sites, allowing transcription to begin. In addition to arabinose, cAMP complexed with cAMP activator protein (CAP) can also stimulate AraC binding to I1 and I2 sites. Supplementing cell growth media with glucose decreases cAMP and represses pBad, decreasing promoter leakiness.

Another example of an inducible promoter is a positive inducible alcohol regulated promoters (AlcA promoter with AlcR activator).

Inducible promoters can be used to limit the expression of polynucleotides in desired circumstances. For example, since high levels of recombinant protein expression may sometimes slow the growth of a host cell, the host cell may be grown in the absence of recombinant polynucleotide expression, and then the promoter can be induced when the host cells have reached a desired density. Exemplary bacterial inducible promoters include for example promoters P_{nisA}, P_{nisF}, P_{zitR}, P_{sczD}, P_{cst}, P_{lac}, Pₜᵣₚ, P_{lac}, P_{T7}, P_{BAD}, and P_{lacUV5}. An inducible promoter can function in a wide range of host cells, e.g., bacterial cells.

A repressible promoter can be a positive repressible promoter or a negative repressible promoter. A positive repressible promoter works with an activator. When an activator is bound to the promoter transcription is turned on. When a repressor binds the activator protein, the activator cannot bind the promoter and transcription is turned off. A negative repressible promoter works by a co-repressor binding to a repressor protein, such that the repressor protein can bind to the promoter. The bound repressor then prevents transcription from occurring, such that transcription is turned off. Where a repressor is present, but no co-repressor, the repressor cannot bind to the promoter and transcription is turned on.

Tet-off systems can be used herein. Tetracycline repressor (TetR) can bind to tetracycline operator sequences (TetO), preventing transcription. In the presence of tetracycline (Tet), TetR preferentially binds Tet over the TetO elements, allowing transcription to proceed. This inducible system can also act as a repressible system using a tetracycline-controlled transactivator (tTA). TetR can be fused with the transcriptional activation domain VP16 from herpes simplex virus. tTA binds to promoters containing TetO elements (often linked in groups of seven as a Tet Response Element (TRE)), allowing transcription to proceed. When tetracycline or one of its derivatives is added, it binds tTA, resulting in a confirmation change that prevents binding to the promoter and turning transcription off.

Cumate-inducible gene expression systems can be used herein. Chimeric transactivator, cTA, which is a fusion of CymR and activation domain VP16, binds to promoters containing putative operator sequences (CuO) (linked in groups of 6), allowing transcription to proceed. When cumate is added, it binds cTA, resulting in a confirmation change that prevents binding to the promoter and such that transcription is turned off.

Biofilm Assembly Genes

A biofilm is any syntrophic consortium of microbial cells where the cells stick to each other and optionally, also to a living or non-living surface. The cells can become embedded within an extracellular matrix comprising extracellular polymeric substances (EPSs). Microbial cells within the biofilm can express EPS components, such extracellular polysaccharides, proteins, lipids and DNA. A biofilm can comprise a three-dimensional structure. Microbial cells growing in biofilms are distinct from planktonic cells, which are single cells that “float” in a liquid medium.

Polynucleotides as described herein can encode cell surface proteins that are involved in biofilm assembly. An expression cassette, vector, or population of host cells can comprise one or more polynucleotides encoding biofilm assembly proteins (e.g., 1, 2, 3, 4, 5, or more). A biofilm assembly protein can be, for example, cell surface proteins such as mucus-binding proteins with an LPXTG-motif (SEQ ID NO: 67) cell wall anchor, mannose-specific adhesin with an LPXTG-motif (SEQ ID NO: 67) cell wall anchor, or a Mucus binding protein Mub, adhesion proteins, cell surface protein CscC, outer membrane proteins, and K×YK×GK×W signal domain proteins. Biofilm assembly proteins, such as cell surface proteins, can be derived from Lactobacillus sp., such as Lactobacillus helveticus, Lactobacillus casei, Lactobacillus plantarum, Lactobacillus paraplantarum, Lactobacillus kenri, Lactobacillus gasseri, Lactobacillus paracasei, Lactobacillus brevis, Lactobacillus acidophilus, Lactobacillus delbrueckii, Lactobacillus rhamnosus, and Lactobacillus reuteri. Examples of cell surface proteins that can be used in the compositions and methods here include those listed in Table 1, and include, for example, P6, P12, P13, P23, P25, P32, P39, P40, P41, and P45. In an aspect a biofilm gene encodes P1-P45 (SEQ ID NO:1-45) or P1-P45 with one or more insertion sequences (e.g., P45IS1, P45IS2, P45IS3, P45IS4, P45IS5).

TABLE 1

| Biofilm | | |
|---|---|---|
| assembly | | |
| Gene/UniProt | | |
| number | Organism | Sequence |
| P1 | Lactobacillus | Adhesion exoprotein. |
| Q046R7 (SEQ | gasseri | MTDAGLTKIQ NAVGDNYSVS LADTTGTLVI NKAKASAVFS GDPSYTYTGT PVSANDYLGK |
| ID NO: 1) | ATCC33323 | YSIKLTEPNN PTYNLVAGDI EFKFNGNWTT QAPVKVGQYE VRLSQQGWNH IKAINSDNVE |
| | | WSATASAGTG TYTINQAKVT ADLSGSNSMT YTGSAVTTND LYSQDSTIKV VINGTDITNL |
| | | PQTFELKDGD YVWQTTAGQA PKDVGNYQIK LTAAGISHIQ KQINDALGAG NVALTTTADN |
| | | AGTANFEIKQ AVAENVQLYG DEQSTYDGDT VTFDPTNLDV KNNFGFHNVE GLTIPNFTSA |
| | | DFDWYDANGE NRIAAPKNAG HYTLKLNDQG KQVLADANKN YTFVDQNGKS TISGQITYVV |
| | | TPAELVVKVT GKASKVYNNQ NAKITQDQIN QGDIKLVWGN STTEPTDLGE FTLTPDDLEV |
| | | VDASGQPAIH ANYVDGQQTG DTYYVRLTAD ALAKIKQLSG AANYNISQAT DTATYQIYAH |
| | | KAELTLTGNQ TTAYGTELPF NESKYTLDFT NWVNTNIPKP VITWQNGEML INGQQPEDGY |
| | | SYHTGDLYVE GYSDGGVPTN AGSYKVKISA NLTKELQKIF PDYDFSGNID SSTLNSNKTV |
| | | NNDPVEASHE PASYVITPAE ATITINGAQH VKYGESTAIA GDQYTASVTA PVSGNETNVV |
| | | TDVALTSDDL TTVPSNAGVG SYTIKLTPAG LAKIQAAIIG HGDVTKNYGW TQAGNATANF |
| | | FVDQMPVTIT VSGGRTVTYG TQAWLRAIKA NPAGYTLTVT TENGTNLSYT ANDGDLVENQ |
| | | TPGNVGEYQV ELSAQGLTNI GKALGTNYAY PQIAADVTAK GTFTVNRGAV TITLKGSDGK |
| | | PYNAQQTLPS GLNLSKYGLD YSATVYSADG KAQMLNLTAN DLQIIGNATN VGTYQVELSQ |
| | | AGQEKIEQLT GNNGANYKWT FKTNADYVVK AATASAELSG SNQKTEDGSA VTTTEVNSNG |
| | | QILVHLTYPG SNVQSTYTLQ DGDYTWETED GQTISAPTNA GTYTIKLNKQ AILAHLQVAL |
| | | NQQAGLGDND QPNVTVSADK LSGQASFKIN PQALTDVTIS SPDQSKTYDA QVADLDVNGI |
| | | TITANGIVAN NPLVNPGISA SDFIWYDETG NKLESAPADV GTYQARLNAS TLAELQNANP |
| | | NYQFSSVTGL INYTINPAPA TATISGSATR DYNAQTTSVS DVMNNIKWDA TGLVTDQDLN |
| | | LTGLTANSYA WYSKDADGNY VAMTGNPVNA GTYYLHLTKS AIEQVKADNS NYDFTSVNGE |
| | | FTYTINAVNG IATLSGSSSK TYDGQAVTTA EVNSINGDII VNFTFPGSSA QSTYVLQTGD |
| | | YTWENKDGQV ITAPTSAGTY TIKLSADGIT NLQNAINQYA GQGNVTLDVQ DLLGAAVYTI |
| | | KQKALDVILG NNSTGTDGKT YDGQAGVINT QAVNFGVFTT SGLVNGETLN AANLTSDDYE |
| | | WVDVSGNAIT APINAGTYYI ALTANGLKKL QADNPNYVVS ESGQFTYVIS PAEENVTVSG |
| | | SQESTSTSID SANFTVHAPA GVTVPAGMTY EFATGVPSES GVYVIKLTPE SITTLEKANP |
| | | NYKLDISSDA KFILDAILNI EFEDTQDGNK QVGKTITKTG VANSTINDLK LVVPENYELA |
| | | PDQELPTSYT FGKTLNQNMY IKLVHKLNEL NPTDPSTNPD PTNKNWFREN GLVKDITRTI |
| | | NYKGLSDDQF AQIPEAQKVQ TVEFTRTAKY DLITGKIVAN SEGSWTAVDG KDTFAGFTPF |
| | | TFAGYTAAPA RVEQVKVTGD DKNSQITVAY TANTQTGKIS YVDSDGKEVG QTALTGKTDQ |
| | | SVEVNPEAPT GWQIVSGQDI PKTVIATPTG VPTVVVKVEH STITVTPGTP EKDIPTGPVP |
| | | GDPSKNYEKL ASLMSTPTRT IVVTDPSGKQ TRVTQTVNFT RTATFDEVTG EITYSDWKNS |
| | | EPAEWQAYAA PEVAGYTATS SVSAKSVTAE TKNETVNISY TANTQTGKIT YVDSDGKEVG |
| | | QTAISGKTGE TVKVTPEVPS GWRIVLGQDI PETVTMGANG GPTVVVKVTH STITVTPETP |
| | | EKDIPTGPVP GDPSKNYEKL GSLTSTPTRT IVVTDPSGKQ TKVTQTVNFT RTATFDEVTG |
| | | EITYSDWTSS EPAEWSEYTA PEVAGYTATS NVSVKPVTAE TKNETVNISY TANTQTGKIT |
| | | YVDGDGKEVG QTTISGKTGE TVKVTPEVPS GWRIVPGQDI PETITATATG VPTVVVKVER |
| | | STITVTPETP EKDIPTGPVP GDPSKNYEKL GSLTSTPTRT IVVTDPSGKQ TTVTQTVNFT |
| | | RTATFDEVTG EITYSDWTSS EPAEWQAYTA PEVAGYTATS NVSAKPVTAE TKNETVNISY |
| | | TANTQTGKIT YVDGDGKEVG QTTISGKTGE TVKVTPEVPS GWRIVPGQDI PETITATATG |
| | | VPTVVVKVER STITVTPETP EKDIPTGSVP GDPSKNYEKL ASLTSTPTRT IVVTDPSGKQ |
| | | TRVTQTVNFT RTATFDEVTG GITYSDWKLQ KSNAASHVAQ WDSYTPQVIT HYVPSVAEVP |
| | | AKVVNAHTAN SQVEITYAPA SESQVIRYVD QNGKEISTQI VPGKYGVDTT FTPKLPNNWQ |
| | | AANTIPTSIK IGENGGLTTI VVEAKTEKVQ QAKTVTETIH YHTANGKQLF ADKEMEVNFF |
| | | RTGVKNLVTG EITWNNWNKD KESFNEVPSP KVSGYMASPT KVAVQTVTPN SEDLVENVIY |
| | | TKNSQTHPTI PENKPNKPQE ENVSKQETKT QDKLIHEYGY KKRADGRLVD HTGHVYPASS |
| | | KVKENGAIYS EKGELLSVGS RRKHELPQTG LHDNSLIAAI GSLLAGISIF GLLGGRKKKD |
| | | DDK |
| P2 | Lactobacillus | Lactiplantibacillus plantarum subsp. plantarum ATCC |
| D7VB22 (SEQ | plantarum | MSFLDRLKGMLQALNSTEAATSATEAPRSIAAQTAAAPTVNQTEALVLVHHLDQDGNELQ |
| ID NO: 2) | ATCC14917 | AADMIAGTIGEEIHLPAVSITGYHLVHIEGLTRWFTTPQASITLTYERQAGQPVWMYAYD |
| | | IDRRELIGRPTMYRGKLGTPYEVSAPTVAGFKLLRSVGDVTGEYTTTSKTVLFFYRNQNW |
| | | QQTDLSTGFVQVNKLTAVYPYPGATTTNYLTKLQPGSTYKTYMRVRLVTHETWYAIGDDQ |
| | | WIPETHLQLTTGDTLLLKLPAGYRVQNKRPVRQTGVVSFVPGKQVHTYIEPYGRYLTTVT |
| | | HGDTVNLIERMADDNGVVWYRLQDQGYLPGRYLTKLDPPFA |
| P3 | Lactobacillus | Mucus-binding protein, LPXTG-motif (SEQ ID NO: 67) cell wall anchor |
| F9UR18 (SEQ | plantarum | OS = Lactiplantibacillus plantarum (strain ATCC BAA-793/NCIMB 8826/ |
| ID NO: 3) | WCFS1 | WCFS1) |
| | | MSKDNQKMTGDSVYRVKMYKDGKRWVYAGATTLALAAGLVFANVNASADTAASSDATTEQ |
| | | VSSAASSAATSSTATSSAATDASSASSTATSTSSTASSTATTSSSAASSTASSAVTSTTS |
| | | AASSSAETVSATTPASSDATSTSTATVAATAAKASVVTPASAAATATTTATTTAATTAPT |
| | | VTAPASEAANQTAAGSVDAGTLTSATQSGGSGNLQDQAQYIQENVDGTNIKVTAGHTYAV |
| | | AIRLTKSQALDWANASGQVSIAPNGSNSNGTWTAVEYATESGKEYSYAAGASTATVDITK |
| | | LTDADSYVTVLYTFKANDDATTGSRAAYLEFTGTTSVNKLSTNTNNTDANQQIEAWSYAT |
| | | QVMDTSVAAGTVVVHYVDENGNKIADDTTVQGDVDNTYTVTPATFSNYTLDTTKSSALTG |
| | | TVAADTTDSDGNVTAAGTELTLVYSQNTEASNLTVNYVDADGNTILPSKTYTEGADGTAA |
| | | EVGGAYSVNAASIDGYTLTGDATQTGTFVSGGNTVTFTYTKDAAPVEQSTVTVNYVDADG |
| | | NTIKAATTQTLDNGSTYTVETPTIDGYTYKSADAALTGTVDGNKTITLTYTKNATPVEQS |
| | | TVTVNYVDADGNTIKAATTQTLDNGSTYTVETPTIDGYTYKSADAALTGTVDGNKTITLT |
| | | YTKDSTTPVENKANLTINYVDADGNTIKASSVTEYIVGQAYTVGQPEIAGYSYNHSTGDA |
| | | IAGTIGYNGNTVTLVYTKNGGTTTAPTTAPTVAPTTAPTVAPTTAPTTAPTVAPTTAPTT |
| | | APTTAPTVAPTTAPGTGDNVNGGGTGTTTTAPVTTPSDDTVDNGNGSSNNGSSTTTSTAP |
| | | ATTVSDDEVTPTTTATTNNGTSGVVPASASLKPVVTTKTTTSDAKTLPQTDEDENGTALA |
| | | VLGLSTLLMGSALYFGVSRRKHEA |
| P4 | Lactobacillus | Cell surface protein, CscC family OS = Lactiplantibacillus plantarum |
| F9USN0 (SEQ | plantarum | (strain ATCC BAA-793/NCIMB 8826/WCFS1) |
| ID NO: 4) | WCFS1 | MRRICKVLMVIISIILGSGAPLNMAIPPLLALAAPDTSSSSTMSSSAISKVTDTNVMAVS |
| | | ADVTSTTDTSDTSSSDSTSATSTTTGNDTTETADTAVESGTVGTVAWTIDDAGVLTLSGG |
| | | SFADLTGKRSPWYDYASSITNIKITDEITVTTASNYGYLFASLANVATVTGLNKLSMSGV |
| | | TSTQSIFYRDSKLTSVDFGQTDFSTVTTMESMFEGCSVLTKVNTTNWNVSHVKSFKRTFY |
| | | MCGKLTMLDVSNWDVTQVTNLDSTFSGCSSLPELDVSRWNTANVTTLASTFYSCSSVKII |
| | | NASGWDTARVTDMTATFMNCTLATELNVSGWDTAKVTSMSRMFFYCENVIQLDVSGWITS |
| | | QVTSLGSMFQNCSKVVTLDVGTWDTSKVTDMSFLFGGCSSLTTLNLEKWDTGSVTTLYST |
| | | FYNCSGLTSLLVDTWDTSKVTNCFWTFGGCSSLTTLNLRSWDLQSATASYGNFENGSKKL |
| | | QHLTLGPNFTFHNDKTMYLPEPSKQLPYNGTWQRNNDDPTYTSAELMTNYDGATMAGTYN |
| | | WVKTSGTVLVKYVDGDGVEIADEETSSGTSGDAYQTTAKTIDGYTLHATPTNATGTYDAS |
| | | TITVTYVYDGNLFFNSSPTMLDFGSHTISGTTETYAPTLDKTLAVQNNGQISSTWNLTAE |
| | | LDSSGFVGADTGKMLLATLYYQTDDGKMTLSPGVAVQVYSQTTTDHKSVDISEHWSSNLG |
| | | LLLEVPNGAAMADTYQGTISWRLNNTVANN |
| P5 | Lactobacillus | Cell surface protein. MAVQPATLGQ ELNLNNQQTI NADSPTSSNE VVVKCVDDAG |
| C8UWM1 (SEQ | rhamnosus | NTLVKDTVLQ GEVGKPYTIK PATIANYQYA KLANGSAPIN GTFSKGTLTV TLVYTKVPVT |
| ID NO: 5) | GG | QRTVNVKYVD EHGNEIAPAT TLTGTVGGSY TAVPANVKNY EYAHLAANSA PEKGSFTANP |
| | | QTVTFVYTEK PAAQGSVTER FVDEAGKRIA PDKTLTGQVG DLYEARPIEI SDYAFSRVAQ |
| | | GSAPAGNTFI NGNVIVTFVY KQVPATQGSV TVRYVDENGN ELAPNRVLAG QSGSAYTTGP |
| | | ITINGYRYVR LAADSAAASG TFPKDTGLVV SFVYTKPAIP VTPTTPETST VPSTSSQSAT |
| | | TEVITPSAQR RLPNTNEKHE YGIAAVGLAL LSLMGLGSTL LFRKAKRQ |
| P6 | Lactobacillus | Cell surface protein OS = Lactiplantibacillus plantarum |
| D7V8E8 (SEQ | plantarum | subsp. plantarum ATCC 14917 |
| ID NO: 6) | ATCC14917 | MYTENTGKHHRNGLPVWLLPLLVVISFWGVSQNIMVVDASSSVTVLPGNGGTLPLVNQLV |
| | | IKQNDTALQGITNNAGDRGSLTPKNGAQRVLIHKVKDSDTITSTYGTVGTFHGQEVTAKV |
| | | TISHIKVHDDSHKAPSGMKQTDGAFQIGPGFSSDTTMSNVAQFNVSYEFYYADTHAAVNI |
| | | QNAFITLSSLDGPVAGTSTGFEYTAYLGAGKIYTVENSIVKQIANPLGGGQLVMAGQTAR |
| | | DASWPYTSSTAATFGVSGTKLEFIYGTTRVNSGNSWLQPVYNVSTITLGTPAIATPTLSA |
| | | TQSATDKQNRTLTYDLQQKVNVLDQDLMTKYKDWSENITIPANAKYAKGEVVNDAGQALP |
| | | STAYQVSYDEKTHQVKWHLTDAGIKSLPFKGETYHFKAQVQFSDDVDDQAKVTATGQTAI |
| | | DKQIKTSNTVTNTIDNQATITVHHYMTDSTDKVAPDETVKVGYGKAYDVTKQVKTITGYK |
| | | RNATLDEHTRGTASKTTKEAVMYYDPLPYNIHVNYLLTDGQKLDELDVTGLYGDTYTTEA |
| | | TDFEDLYTVDTDRLPTNAQGTVTEKPTTVNYYYQPTTGQWVDVGNQSSVLVRQDTKHNVR |
| | | SVSQIYANDSGFTVKYNQDAAQVAIAASDTNGTQDNSLVFDYNSKYTFELSKNETVTFKV |
| | | DDQGQVTATRVLGAEQTVTTFDKSGQLKTVTTVTNANGTKSQQTNTVDGLKSMVTGEQYD |
| | | LGLLNGLKVTAQKEINPSQAATTESKTTTDTSQSGSNQSTSTTATDQTGTNESTAGSSTN |
| | | ATNASSSVDASSANSQGDTEATSQSGTSASADSKTDSSVASSTSQTTDGETTNTGDTTTG |
| | | TTTGSGLGFKSPFTEDQNTSSALGSAQTSSSLNSDTSAAVQALIAEPNSTPVVLDEDASF |
| | | EEGVPVNDPVFSNDEGVSPNNNPSSAATPLAQATNTRARLTQNGKLLYEGTLKADQGEQN |
| | | LYVSPDTTVEVDGGADGDGFYLDTYDGDKGMAYTLGSGYAWAAENNDVTAAPASSATTSS |
| | | ESAASEPSVNSSDSSRTASSAVDHSTSSASTSDASQSSHSTSSGESSHPESSSGSSTTSD |
| | | SADVDKQAAARSSQTQSNSVNGSSQAVSSSTVTSQSSVPTKANTKQASSTPTTKANRATV |
| | | AAATSSTAPRQSRATTASASVPSVTSASAAAASRDKQRSAFKKQHPILNQILPKTNSAVA |
| | | TWLVWLGVGLLLLTVAITMVIKKRGRD |
| P7 | Lactobacillus | Mucus-binding protein, LPXTG-motif (SEQ ID NO: 67) cell wall anchor |
| F9USN7 (SEQ | plantarum | OS = Lactiplantibacillus plantarum (strain ATCC BAA-793/NCIMB 8826/ |
| ID NO: 7) | WCFS1 | WCFS1) |
| | | MNKRKIITNNPPKWHLITGIAATILASIILTNQDAFAATDSTTAPTTTAPTVQQTAPTNP |
| | | LSGSQVTLTSTTGSSATGSTTTSSPVATSTAAMPVKSTATSGSLMSAMASTSATSGHAAE |
| | | PSSSVTEAASTNNLIPTSAAMASSATTKYPTDTTATPNASSSLTSAESSTPNKAMSTSQQ |
| | | TVSSGVIHSTTPASSASMPVPTSVAETASAAAPSVTNSTAANSTAPTSVMTTDSAAESVP |
| | | LSTSSETSSEKLAAASTTSTSQISDGSEVIHPMTSAISSSSSAPTSGAKMAASAASAASA |
| | | SVITSAVNSIAASTYSADASAASVESAATPDTSHATVPASTATSAATTFQITSVINSLAS |
| | | STYSEYAEQANAEAASAATTAEKPATSVGTVVPTAATTPTESIDTWMPNKHLQEAVLREL |
| | | QALKLPDHQFKSVNDITKDDMQLLTQFYGENTYIDGHTPYSLEGLQYATNLKTIWLNNGL |
| | | NALGGYYNGDVTDISPLAGLTKLTVLNIQHNRVSDLSPIAHLTNLQELDVAYNHIADLSV |
| | | FKDLPNLKTTTYLGQTILEPLVYVDQDTTSATLKNRFYLPNGQQAVLKSQAAILKPVQLT |
| | | PNGQFYYRFYFNGAGKAVNGDLSNVVPDGQGGLTFNQLVPQIPGFTGDANGQFVTNGVSI |
| | | NVVPNDKNFYLVAQGSDGSSPVFHVFQPYVLAAKAAPVTIHHIDRNGAALRDSEELTGLV |
| | | GEDYQSTPADITNYTHVETQGAPQGTFSAEPQAVTYVYDKTAGAPVTVSYQDEQGKTLQP |
| | | DTTCNGLAGDPYTTKPLEIAGYDLTKTPDNAAGTFTAEPQHVIYIYTKQVPQPVTASYQD |
| | | EDGKALQPDITHTGEIGAAYETKALEIPDYDLVKTIGNATGTFTKEPQQVTYIYTKQIPQ |
| | | PVTASYQDEDGKTLQPDITHTGEIGAAYETKALEIPGYQLIKTPTNATGSFTKEPQHVLY |
| | | VYEKQAVLPVTVSYQDADGKPLHADIVLSGDFGQNYQTEQLSIPGYVENKVVGPTIGTFG |
| | | TTAQHVVYTYTPEPSGPEQPTPGPEPEPVPEPQPTPAPQPEPTPQPSPTPQPSPAPQPNP |
| | | APQPSPVPQPNPAPQPGSSLLAKAPVSQGTTTSQSSPTTSPQPTPIAPVSALAQPGKQQA |
| | | PATVATHNSGQLPQTSEQSEHGATLGGILAALFTGLGWLGLAAKFKKRE |
| P8 | i | Adherence-associated mucus-binding protein, LPXTG-motif (SEQ ID NO: |
| F9USH8 | plantarum | 67) cell wall anchor OS = Lactiplantibacillus plantarum (strain ATCC |
| (SEQ ID | WCFS1 | BAA-793/NCIMB 8826/WCFS1) |
| NO: 8) | | MRYTRGKWRVTNPKVWLFSSVLILGWRIVPTVAQASEAETVTMSSHSVQLETDNQDQLTE |
| | | VARISKTAVTRDSHSVTAQSSKSADRTSSEQPATTGTVEAVSPTTSEAQQRSTQQDKTAV |
| | | DQQASDSTAASAGASTNQASAATSSDQAPAANSTGTHHAIDMASSASALGADSGAHSESL |
| | | SEAQHSGGQGKTIDSDLSGTVHSQSSVSTVTTATPVNSNSSLESDKFTSTRSRAVAATDQ |
| | | MSSRVEKRALNKTNVTKSINIPVATKQPSKQRTVTASSFLTTAKNLADKNYLDQYAKQHG |
| | | QAALIALIQDWLSTYRIIALTGITIVNSSFDGSVATISGGLHVINTGATIRSGQDDEWET |
| | | IINGGLSVTNNTITFTTTNGLVDRPVANQDMDFTKPRPTGNGAIKGLPSVTVDSSLINAQ |
| | | EFSQAQINISDFYDQLVTAGTILSATNGGTLSKMLIGESGTADLGSYQGHHYYAVNIDLN |
| | | DWHSGIRTTGFNNDDVVIYNVVTAAPALTIGGGFSSSTPNLVWNFNHAMRIQNTTMITGK |
| | | IVAPHAVFTTNQNVDSAAVLQYGYGDVDSAIRETITSQNEHNYGFGQVVTDDPLDYLIAV |
| | | IKSDGTSIDTLAGFRHLLATGQLKITITDAAGTRLSGLNAVDTHIAGQHCYLITYQFGDQ |
| | | TATTWLNVQPSHEPIIPISRIPEYSAITRTINYQDERTGAVLAGPVIQNVRVVRFAIFNA |
| | | KTHELLGYDTNGDGIVDTSDGTIAWLLVPPTDQDWVQVVSPDLSAQGYQAPDIPVVAGQT |
| | | VIINGGDRTMNTNVIVKYQQQTHIATTQRTVTRTINYIDGGTLQPIASLHAVVQTVKYQL |
| | | LAVVAHDGTILGYDTNGDGQIETQLADEAWLIVGSGPWFGAVKSPDLSHEGYAAPDLKVV |
| | | PEQMVAGVDDKDVTINVYYRLATQAVTVYQNKRRVISYIDRQTHQSIATTVQQLVIYQRT |
| | | AIIEKKTGKCLGYDLNGDGLVDTSQADYAWILVGSGQFAAVTSPTLVVQGYTDPDIRTVA |
| | | AQTVAITDPDLMTTIVTYDHRIITVTPGNPARPGQPVDPDNPNILFPDEGGDTDLTHTVT |
| | | RIIHYVYEDGTTAAASVLQTVQFQRNAMIDLVTGEVTYQEWVPVSVTEMAGVISPIVAGA |
| | | TTTLTEVAAQQVSVTTADQVVVVTYKKSAIKPEEPGQPEQPSQPEEPGQPEQPSQPEEPG |
| | | QPEQPSQPEEPGHPEQPSQPEEPGQPEQPSQPEEPGQSEKPGELQKPSQPADSEQPDGLS |
| | | DQANLSRNQAEQSRTSQPSQAESDQSVVQTNQQKTAASVSGIGWVSGPAVSKRTTKHHRM |
| | | TTLPQTDEQNTQLSLLGMIGLALSSILGWLKIKSRD |
| P9 | Lactobacillus | Adhesion exoprotein OS= Lacticaseibacillus paracasei (strain ATCC |
| Q033L8 | casei | 334/BCRC 17002/CCUG 31169/CIP 107868/KCTC 3260/NRRL B-441) |
| (SEQ ID | ATCC334 | MTAIGAKAFNANLIPEVAIAGTPTIDQEAFSNNRITVLHAATAVPTTPDALNQNADAYTD |
| NO: 9) | | SAHVSLRDLFSVAISGVSQDQIVVSNIQGTGVAFNTATKSFTMPAGTEQFSFNWSLKAAD |
| | | GTTYTGLYKVHLNDPVIHAHDINLFTGQVWKPELNFGGAVKKNGTEIIEIPLSDLTWTVT |
| | | DQNGAVIASKDRDGVVTGSVPSDQVIWYTVTYAYGAESGSAKIFYNQRLAASYSLTGTQT |
| | | ATATGQPITVDLTAFSLSLGDGFNAGALQLSDLNFFDASGNQIAADALTKTGVYRVELSK |
| | | AAWARIAELTNDAGESAANYNFTGTSTAQLIIGRTATGQLNNSGFTYDGTTLASQAPKLV |
| | | LNVTLSDGSQQAIDLTSTDISLVEADSPDVGTYRYLLNGSGLTRIQAILGDEVTIDQTDI |
| | | NTHPGVITITPATATATVNGTQFVYDGKTTASQASGLQLTLTAGSGTTVVDLSSTDIVVG |
| | | SDSVNVGDYQYQLSQNGVAKVEQALNANYQLPSDLLGSLTGTITIAPAQGTAELRDDSFI |
| | | YDGQTEASQVQGLTGDVTIGNVTVPVILTSVDFVVGNDGVNVGSYQYTLTATGIAKLQQA |
| | | VGSNYQLTVSELAKLTGNINITPATTTADSNDGSFMYDGQTKASQAQGLTAVVELGDDTT |
| | | SIKLDASDIVVADDGVNVGSYHYRLSTDAITKLQQVAGPNYQLKADDLAALMGIITITPA |
| | | EGTATVNDTTFVYDGRTKASEASGLNGVVYLSRGTARLTVALTTQDIVVDGDNTTTGTYH |
| | | YHLSHSGIAKLKAAAGTNYALNETDLNALTGTITITPLTVVATVNNGHFQYDGVTRASQA |
| | | SGLLVTVQLPTGAQTVALTNADIDVANDSATVGTYTYRLSASGIAKVMVALGPNYQINDT |
| | | TMNGTITITPAVLSGQLSGMQQKIYDGQPGELNAQHFELIFTDGSHIILEDSDLAFADGI |
| | | APIVVGRYAVTLSAGGLKRIQALLPNYLLENVDTQQAVFEIVAKSGPLPDTGTGTDTGTG |
| | | TNTGTSTGHETGKVPSVTGRPSQSINQQTPVKTTHQLPQTGDRSANDLSIVGLILTSIAS |
| | | LFGLAGVRNKKRSE |
| P10 | Lactobacillus | LPXTG-motif (SEQ ID NO: 67) cell wall anchor domain protein |
| D7VAH4 | plantarum | OS = Lactiplantibacillus plantarum subsp. plantarum ATCC 14917 |
| (SEQ ID | ATCC14917 | MTMLPLNCQRHYISILKEWGSLKPNNVNNQNKRHQSRWVITSATAMILTTLTIASQAAAA |
| NO: 10) | | DDTVTTTTNEPTNSQLNTNTQVNATQVNLKADTSTSVSTIKSDQSAVAATSPTTSTGSPS |
| | | EHSSSVNTNPQQQSANPASQSQATTTSESTPTTDIKHPTQTAPAQTTSASTTEPTTESNT |
| | | ESATDSQAKATTTDNQASKQPSQQAAPAPSNSTTTEVNTQSATSSASTDDKIVTNVNQEK |
| | | LVLKTNQPVVRAISRTASENINDWMPNTLLQQEVLSQLRKQNPDRTWNSAADITKADMLL |
| | | LTTYYGKDTYIDGKTSYSLEGLQYATNLTTVWLNNNLNAPSGSYYSDVTDISPLANLQKL |
| | | QVVNIQQNRITDISPLANLKNLTEVDAAYNHISDFSPLKGFKNLKGTFSNQFITLPPAYI |
| | | SADNNIATLAIDCYLPDGSKVQLKPNNGVGETVFYKNGQLYVRWYFNGAGGGNYDSNGHI |
| | | YYTNMKPQQPGLTGPTFNGTTVIPMDDYYFMTAASDGNNFVVVRPYVLAATAAPITVKYV |
| | | DALTGESLVTADLTLNGIVGQPYTTQRIDDELPNYDFTNIVGNASGVFTADAQTVTYYYT |
| | | RKDAGDITIHMVDANGNLVYEPQILPGKHNLGNAYNLDAPTFDHFKLQQTIGNAAGVFTT |
| | | DPQSITFVYVRLDAGNITVKYQDKQGKQLKPDKTISGSQSLGQAYTTEPLDIENYTLTTT |
| | | PTNATGTFTDQEQTVIYVYVRRDAGQIVVKYQDSAGNPLAPDKLLDGKEQLGAAYQTEAI |
| | | SIPNFYLVATPANATGTFSTDAQTVIYQYTRSNAGHITVKYQDANGTTLAPDDVLTGDGQ |
| | | LGRPYQTSAKTIENYRLIQTPANATGQFSDQAQTVIYVYTREDAGDITVQYLDENGQQLA |
| | | ADSVLSGQGQLGRPYETSPLNINGYTVKSTQGNTTGTYTVQPQRVVYIYDRTAGQPVTAK |
| | | YQDQDGKSIHPDVVHSGYLGDNYSTEQLVIDGYTFKAVQGDVSGTFGTSAKTVTYVYERT |
| | | AGLPVTAKYLDEHGKSIHPDVVHSGYLGDSYSTEQLVIDGYTFKAVQGDVSGTFGTTAKT |
| | | VTYVYTVNTPTIPDTQGTVTVHYMTKDGIKLNEPTVLSGKTGTTYQTVPLTFTDHELVGQ |
| | | PENAMGLFTADNVDVTYVYQATDTTGTDDIIDPEEPEQPTKPIKPVEPTTPETPNEPGTT |
| | | VTQPDRIKPTQPAVAVKPAATVKPTLKPAAAQASLVKTTSPVTEHSAQLPQTNEQTGKLA |
| | | VILGLLLSIVTFGFYGKHRQS |
| P11 | Lactobacillus | LPXTG-motif (SEQ ID NO: 67) cell wall anchor domain protein |
| D7VFA8 | plantarum | OS = Lactiplantibacillus plantarum subsp. plantarum ATCC 14917 |
| (SEQ ID | ATCC14917 | MTKSIIKRSMIILNKRKIITNNPPKWHLITGIAATILASIILTNQDAFAATDSTTAPTTT |
| NO: 11) | | APTVQQTAPTNPLSGSQVTLTSTTGSSATGSTTTSSPVATSTAAMPVKSTATSGSLMSAM |
| | | ASTSATSGHAAEPSSSVTEAASTNNLIPTSAAMASSATTKYPTDTTATPNASSSLTSAES |
| | | STPNKAMSTSQQTVSSGVIHSTTPASSASMPVPTSVAETASAAAPSVTNSTAANSTAPTS |
| | | VMTTDSAAESVPLSTSSETSSEKLAAASTTSTSQISDGSEVIHPMTSAISSSSSAPTSGA |
| | | KMAASAASAASASVITSAVNSIAASTYSADASAASVESAATPDTSHATVPASTATSAATT |
| | | FQITSVINSLASSTYSEYAEQANAEAASAATTAEKPATSVGTVVPTAATTPTESIDTWMP |
| | | NKHLQEAVLRELQALKLPDHQFKSVNDITKDDMQLLTQFYGENTYIDGHTPYSLEGLQYA |
| | | TNLKTIWLNNGLNALGGYYNGDVTDISPLAGLTKLTVLNIQHNRVSDLSPIAHLTNLQEL |
| | | DVAYNHIADLSVFKDLPNLKTTTYLGQTILEPLVYVDQDTTSATLKNRFYLPNGQQAVLK |
| | | SQAAILKPVQLTPNGQFYYRFYFNGAGKAVNGDLSNVVPDGQGGLTFNQLVPQIPGFTGD |
| | | ANGQFVTNGVSINVVPNDKNFYLVAQGSDGSSPVFHVFQPYVLAAKAAPVTIHHIDRNGA |
| | | ALRDSEELTGLVGEDYQSTPADITNYTHVETQGAPQGTFSAEPQAVTYVYDKTAGAPVTV |
| | | SYQDEQGKTLQPDTTCNGLAGDPYTTKPLEIAGYDLTKTPDNAAGTFTAEPQHVIYIYTK |
| | | QVPQPVTASYQDEDGKALQPDITHTGEIGAAYETKALEIPDYDLVKTIGNATGTFTKEPQ |
| | | QVTYIYTKQIPQPVTASYQDEDGKTLQPDITHTGEIGAAYETKALEIPGYQLIKTPTNAT |
| | | GSFTKEPQHVLYVYEKQAVLPVTVSYQDADGKPLHADIVLSGDFGQNYQTEQLSIPGYVF |
| | | NKVVGPTIGTFGTTAQHVVYTYTPEPSGPEQPTPGPEPEPVPEPQPTPAPQPEPTPQPSP |
| | | TPQPSPAPQPNPAPQPSPVPQPNPAPQPGSSLLAKAPVSQGTTTSQSSPTTSPQPTPIAP |
| | | VSALAQPGKQQAPATVATHNSGQLPQTSEQSEHGATLGGILAALFTGLGWLGLAAKFKKR |
| | | E |
| P12 | Lactobacillus | LACPL Cell surface protein, LPXTG-motif (SEQ ID NO: 67) cell wall |
| F9UTX0 | plantarum | anchor OS = Lactiplantibacillus plantarum (strain ATCC BAA-793) |
| (SEQ ID | WCFS1 | MYTENTGKHHRNGLPVWLLPLLVVISFWGVSQNIMVVDASSSVTVLPGNGGTLPLVNQLV |
| NO: 12) | | IKQNDTALQGITNNAGDRGSLTPKNGAQRVLIHKVKDSDTITSTYGTVGTFHGQEVTAKV |
| | | TISHIKVHDDSHKAPSGMKQTDGAFQIGPGFSSDTTMSNVAQFNVSYEFYYADTHAAVNI |
| | | QNAFITLSSLDGPVAGTSTGFEYTAYLGAGKIYTVENSIVKQIANPLGGGQLVMAGQTAR |
| | | DASWPYTSSTAATFGVSGTKLEFIYGTTRVNSGNSWLQPVYNVSTITLGTPAIATPTLSA |
| | | TQSATDKQNRTLTYDLQQKVNVLDQDLMTKYKDWSENITIPANAKYTKGEVVNDAGQALP |
| | | STAYQVSYDEKTHQVKWHLTDAGIKSLPFKGETYHFKAQVQFSDDVDDQTKVTATGQTAI |
| | | DKQTKTSNTVTNTIDNQATITVHHYMTDSTDKVAPDETVKVGYGKAYDVTKQVKTITGYK |
| | | RNATLDEHTRGTASKTTKEAVMYYDPLPYNIHVNYLLTDGQKLDELDVTGLYGDTYTTEA |
| | | TDFEDLYTVDTDRLPTNAQGTVTEKPTTVNYYYQPTTGQWVDVGNQSSVLVRQDTKHNVR |
| | | SVSQIYANDSGFTVKYNQDAAQVAIAASDTNGTQDNSLVFDYNSKYTFELSKNETVTFKV |
| | | DDQGQVTATRVLGAEQTVTTFDKSGQLKTVTTVTNANGTKSQQTNTVDGLKSMVTGEQYD |
| | | LGLLNGLKVTAQKEINPSQAATTESKTTTDTSQSGSNQSTSTTATDQTETNESTAGSSTN |
| | | ATNASSSVDASSANSQGDTEATSQSGTSASADSKTDSSVASSTSQTTDGKTDGETTNTGD |
| | | TTTGTTTDSGLGFKSPFTEDQNTSSALGSAQTSSSLNSDTSAAVQALIAEPNSTPVVLGE |
| | | DASFEEGVPVNDPVFSNDEGVSPNNNPSSAATPLAQATNTRARLTQNGKLLYEGTLKADQ |
| | | GEQNLYVSPDTTVEVDGGDDGDGFYLDTYDGDKGMAYTLGSGYAWAAENNDVTAAPASSA |
| | | TTSSESAASESNTNSSDSSRTASSAVDHSTSSASTSDASQSSHSTSSGESSHPESSSGSS |
| | | TTSDSADADKQAAARSSQTQSNSVNGSSQAVSSSTVTSQSSVPTKANTKQASSTPTTKAN |
| | | RATVAAATSSTAPRQSRATTASASVPSVTSASAVAASRDKQQSAFKKQHPILNQILPKTN |
| | | SAVATWLVWLGVGLLLLTVAITMVIKKRGRD |
| P13 | Lactobacillus | LACPL Mucus-binding protein, LPXTG-motif (SEQ ID NO: 67) cell wall |
| F9UP14 | plantarum | anchor OS = Lactiplantibacillus plantarum (strain ATCC BAA-793/ |
| (SEQ ID | WCFS1 | NCIMB 8826/WCFS1) |
| NO: 13) | | MRNRLNRLGLESKSHYKLYKSGRRWVAASITVFSVGIGLTFSQVEQVKAATGTGVDTADN |
| | | SASVSSDMAEPSNAVVLKSASTATATKTATQDAKAATDVTAATQDTKATTDSTGATSASS |
| | | NRQSTAATKPAAEVGTASSSADSSASISSTDGASASAPSVTSKFTNTEATSASATKTATT |
| | | SADTDVLNTETTSSSVANDLTDATTASQTRTETGKTASIPTAEAPTITTAVTSRALPLTG |
| | | ALASRSANTPVTKSAVQAVSAITSEAETKPTVSLVTTGTVSMDYGEASLADLESHISSPD |
| | | ETPANDVAYYIQDAAGNYLEDVNGNKVNLLYALFLDSADVNDYVDVVYTDEHGQVTKYSG |
| | | DTDFSTLDQIGSYSVTINAAGKAGMSRVMQDYNAYDTSTSDLDDFVPTFSTGASDYTFTI |
| | | NIVPVKITATTGKNGLIILRPSQLYTGSLTMLPVVTVKNATKQNILQISNGEIGDAKPGV |
| | | AGKVGQRVLTLADFTYTYQGTETNLTGADTGKYAITLNDAGRKAVQAALGSNYILDDAAV |
| | | FTTTGAVQAAGLGLTIANDTVTYNGKPQGTTVAITAGTAYDHFDFTTTTDTNVGTYDDLT |
| | | YALADPTQAAILAKNYTVTTTDGTLVITPADLTVTVKDDNPVYDGRAHGMTATVTSGTNY |
| | | DQLAFTAVAADGSGATTYTTVGTYAMTGTTAADTSNYKISYVNGTLTIDPAKATITIPNK |
| | | IYWSDGTQKNLAAVVTGTVNGETLKYRVTNGMSAVGTKTITATPDADDSVNKNYTISVIP |
| | | GTLTIGDIAVKYLYEHVDANGETQVDASETGTATHATDATATDYLTYTTAAKPKTGYVLA |
| | | PNTGLAYNGTLTDQGGTVTYRYLAKTETAIVTYFDQTDNKVIKTEPLQGAYGTTDAYRTA |
| | | DTIAAYENAGYDLVSDDYPTAGVVYDQDGSVQEYQVTLVHKFVTRTPDNPGTPGEPIDPD |
| | | NPNGPTYPVGTDFEDLTEQVSQTIQYLYKDGRTAKPNNVQAVNFGRNVTVDEVNGTVVYT |
| | | DWLTDDGAVTGRFEAVDSPLITGYTADPTSVAGNPGVVWQDDDTTIPVTYTVNTEYATVT |
| | | YFDQTDNKVIKTEPLQGAYGTTDAYRTADTIAAYENAGYQLYRDDYPTAGVIYDHDGSVQ |
| | | KYQVTLVHKFVTRTPDNPGTPGEPIDPDNPNGPTYPVGTDFEDLTEQVSQTIQYLYKDGR |
| | | TAKPNNVQAVNFSRNVTVDEVNGTVVYTDWLTDDGTMTGRFEAVDSPSITGYTADPTSVA |
| | | GRDTVSGTDLSPDVQVYYQANPEKATVTYEDTTTGAVLTTDPVTGDYQTVSNYRTADRIA |
| | | QYLNMGYELVSDDYPTSGAVFDKDGSTQAYTVKLQHKLLPLTPENPGTPGEPIDPDNPNG |
| | | PTYPAGTAVQDLIKQVGQTIHYQYQDKSTAADANTQTITFKRSVTVDEVNNKLTYTDWLT |
| | | GTATTGHYMPVDSPEIKGYVADSTRIAGNDEVRNADADTNIVVTYQAKPENATVTYVDVT |
| | | TGKTLAIKSLTGDYQTTSSYRTAETIASYVKNGYQLVRDNYPTSGAVFDVNNFAKTYTVT |
| | | LKHKLVTVTPENPGTPGQPIDPDNPDGPKYPVGTTAQDLTKQVSQTIKYRYQNGASAGTD |
| | | NVQLITENRDATIDEVEPTAVYTDWLNGTSATGRYTTVMSPVITGYTADKTQVAGRDSVA |
| | | NTDSDTQVVVTYAAKPEKATVTYVDVTTGKTLVTANLTGDYRTQSNYRTAETIAGYVKNG |
| | | YELVRDNYPVSGMLFDVDDFAKTYTVTLKHKLVTVTPGNPGTPGQPIDPDNPDGPKYPVG |
| | | TTAQDLTKQVSQTIKYRYQNGASAGTDSVQLITENRDATIDEVEPTVVYTDWLDGTSATG |
| | | RYTTVTSPVIIGYTADRARVTGNDAVTSAAQPTNIIVTYAINAEKATVTYVDVTTDKTLA |
| | | TVSLTGDYQTSSDYRTANTIADYSNQGYVLVRDSYPVSGAIFNDDGVVHSYLVQLAHVTT |
| | | ATTETKTITQTVHYQSTTGTQLHDDTVRAMTFTRTKRVDQVTGDVTYSNWSTNQADHTFE |
| | | RVAAFSIPGYHAVVTGTQAVMVTPASVDDVQTIRYVTDRLSTGETPKTPVKTVTVNKSDK |
| | | IKTTDTPDKVATVKTPDKAQTVATTTAKQASVKRSVDLKQAQAVEQPAQTRPANVKTVKL |
| | | AKTTKSVKPTAAHQSATHKQATLPQTNDDRQASVAAELLGLTAATLLVGVSAILKKRHN |
| P14 | Lactobacillus | Predicted outer membrane protein OS = Lacticaseibacillus paracasei |
| Q034X4 | casei | (strain ATCC 334/BCRC 17002/CCUG 31169/CIP 107868/KCTC 3260/ |
| (SEQ ID | ATCC334 | NRRL B-441) |
| NO: 14) | | MRELGVKKTGHFMLKVGIYLTVILGMIVQLISPALALAAENPTQAVTGTLTIKNQDEQGS |
| | | PLNGAKYEIQNESHQVVANSEISKDGQATVPNLPVGNYTVTEKQSVSGYTALEQTKNFSV |
| | | TASGNVTLLFKSRASATLDSGSSSSTAAKPAAAKTPEAEPSATPDAKADTELPNIFTKVA |
| | | LKDGNDQPLGTEVDQQSAVKMEMTFTLPATSTPFPAGASFTTTLPKDQIAFPESGGGNES |
| | | GDVASYYFDATTGQLTIKLLKATSNGSWLVHIAASFKALTANDSLNQTLVFHTKDQDTKF |
| | | PIMFRSNAKPVVVYAHTTTPQSLNPTGIAGTAKENLNGNETSKTDPTKWDSDPAKRSKNA |
| | | DMALTLTARGSGTDYLKSLTFSDSDLAKIKVSSAPVNILGGFSEELKPLVAGQDFHAVLS |
| | | DDKRTVKIYLTGGFKKTTGYQVDYTATIDRSLDDTGKVGSALVEGYRYLTGSQSSDGYDY |
| | | DSVTMRNSGVAITKSGDITNNFRALNWKINWNYSMDTMKAGATLTDRFGKQTSGKDEHDQ |
| | | PNIETDGNQTLDTKSLKVFQVTFDEWATPIVSKVDIAQYFKLTEKGDGEFTLTYLGGGDL |
| | | PENASFQIQYQTKLKNTPKNGDNLTNIVNDQKNHYDHATYPVRLPSGITKVGGKIDAYNG |
| | | QMTWRINANRVFRNMKNGKIFDLFPDGVDKLDNDPTADNINTISGENVSANVDDGANDGI |
| | | LVYAQNPDGARTLLKPGTDYDMSTQDADVQSAVKQYNDKDKTNPINANGQEKGIRGFVVT |
| | | LKGAYAETDSQIVIYTHTKLDMLKLGQVGHDPDALKKALNNRAFFFFDLPPGDDDVASGD |
| | | SSSTPTPEEGAFSGALKNSWSDAPDTQYWGVLVNQLGLPYGHMHLTDILPRFDGVNYELI |
| | | PDSIKFYEVTGPDGVDPSNTGDPASSNDVKEIKTSPYYGTGGWSSTALKAEDAAQQRLLP |
| | | TNTPNTWLKNNPNLAQQLDFDFPNIGTGRVWVVFKTMRANQWNYNDPNFANNATVTDTEP |
| | | TTAIPTFNPSASKSAQSYWTPISKTVSADTKLKNVLNWKVNLINIQDKYRPMVNPVIEDT |
| | | LDPRGTGAEINATSFVVTLKVGIADPDTLEEGKDYSLSLDGKKFTITFNRTFGNLVQTAN |
| | | SPLNNYEVSVAYSTSSKSSGWAYNSSSVEWDGSQTTQKPSDGVPPDARIANANGYLPYWG |
| | | SGISGETLTQLANLVVEKKDSVSGTPIPGVKFRLSDGTHTFEATTKLDSATNKALATFQG |
| | | LPIGIDYTLTELSTPAGYKPLAPQTIRLNATSDTGTAIQTEAVENEPYQITLSKYDNRAK |
| | | GQSETDNKHYLLPGATYDLVDTDTQKTLKSGMKTNADGKITIGTASSFSGQYAGDKFTPD |
| | | LKDGEYVLEDLKPGNYKLVETQAPDHYRGDAHDQATITSGPDKQVWEDSLKAGSVAAIIS |
| | | NKAPSATVTAYNQKKPGQLDIKKQAETITDDKFSDRQPMTGAEFKLYRYGDDGKVDQSKS |
| | | WDATIISQDGTFIFDSPDLYEGKYQLVETKAPEGYVIPDDLAKGVDVNITGDETLKLPTI |
| | | TEPVYRRALQVAKTDGNFGNPIAGITYALYQNDGTEIAKDLVTDENGQVNLPFNLPAGKY |
| | | YIQETKSLPPYRPNSDKHPFEVKQTDQTQTAGNLETENKEHPIKVNVTNYQAKTLNVKKV |
| | | DRTYATHVLPGAVFRLTNSAGYTRDVTTDENGIASFGDLLLGSYSLTEIKAPAGYRLDNT |
| | | VYPIALSSAETPTAITVNKEIADDPYQVNLTKYDNRVKKDDPASQKKYLLPNAVYKLVDV |
| | | AANKTLKADMKTNADGQLTFGAASSFDSPLKDGEYAIEGLKPDTSYRLVETEAPEHYEGD |
| | | AADQANATSGTQKQAWEDSLAAGSVDFNIKADQTQVKLTATNQKKPGQLDLKKQAETIKD |
| | | DHFPDRQPMTGAEFKLYRYDEAGKVDRSRSWDATITNNDGTVSFKDSDLYEGKYQLVETK |
| | | APDGYVIPDELAKGVDVDITGDQTLTLPTITEPVYRRTVSVAKTDGNFGNPIAGITYALY |
| | | REDGTELAKDLVTDKNGQVNVPFSLPVGHYYIQETKTLPPYRPNTDKHAFEVKQTDQTQT |
| | | ASSLATENKQQPIRVNVTNYQVKTLNVKKVDRTFAAHVLPGAVFRLTNSAGYSRDITTDE |
| | | NGLASFGDLLLGSYSLTEVRAPAGYRLDKTVHAITLSSAITPTPITIDK |
| P15 | Lactobacillus | KxYKxGKxW signal domain protein OS = Lactiplantibacillus plantarum |
| D7VA43 | plantarum | subsp. plantarum ATCC 14917 |
| (SEQ ID | ATCC14917 | MNRFITSKQHYKMYKKGRFWVFAGITVATFTLNPLISRADTETTTAATAATTTAGASSSS |
| NO: 15) | | NSQVLRTTTTSTTGATTQSSATAINAATTNTSAQKKQAVSGTTTDSKTEQPVTAVGENEN |
| | | ATSNLSTSDSASASSQAKTGSGDSLDQTSNSSVSVASSSQKVTTQNSDYQNDQGTGSESG |
| | | IQSNVTDTVVADESLQTNRSSVASPSTSTMASISDSDSKDSNETEKVVDSETSPIAVTAT |
| | | TNTITTTNDKVQLNRALLARAATPATVVSTGTLGTSAWQYTDDGVLTIHAGDWTGVGDVS |
| | | DVPGDFGSELTKVVIDGPINAGTDTSYMFRYNPNLASIDGLENLDTSKVTDFSMMEMGTK |
| | | IADFSGLAHWNVSSGTSFDSMFASDSRVQSYDLSQWQLNTVQPVSLKRMFSENTALISIV |
| | | LSTWNVRMVTDIDGLFNGDKSLTTADLHGWNLLNVTALSSMFLNDTNLTDLDITGWQTGS |
| | | TLTSTKFMFEGTPGLKAINIASLDMSNFAAVTEADMNKEPADHDMFLNQDSSGNPLPMNL |
| | | NALTVGSKTYLVGSSLPDIPTGTGYTGKWVNQADATQTYTSSELMALYNGVDNPADTITW |
| | | VWETSPSYADFTSKNVTGLIAGPKTTWRVADSVATLKDVNGTDIYATADTVVKVISVNGD |
| | | TAVTTVDTQTTGTYQVDLQYTDAYGKVWQQTSTVAVAVNQGKLVGKPLTIKMGAKPTYTI |
| | | NDLIDTDNSRNAAGDKLSADELATATVTGLDTSKAGTQTVTLAYTDDATGMVHTTTTTVT |
| | | MVATKADLTMRNSTIIKGPKNSSWDYRQYVTSVTDFDGNPVSLDGLNIVVDQQPDLTQIG |
| | | SQTVTLTYTDTLGNVISVPTQVTVVASRAQVTTKAPLTIWPSEVAQLKVADLVTITAANG |
| | | NPVDTSTDLTDVTMSSIDTSKGGAQTVTITYTDEAGNLVTAYAKVTVDQSDLKTKLTNPI |
| | | AGPKAKWDYLAGLEWVKDANGKLLDNLATADIKVVTEPDLSVAMVGHDQTVTLSYTDELG |
| | | KEHLVTAVVNTVASKAKITAVSDQIIIPDEAKKLTATDLVSELIDAAGNKATNFDDVTMS |
| | | GFDAKAIGPQTVTLMYTDAYGNQTTDSTTVTVDFATITGQATHPIAGPTATWDYRDSVTQ |
| | | VIDANGKIIDVGDADITAMTPDLTPAKVGKPQTVTLTYTDSLGKVHTTDVIVTTTLSEAK |
| | | ITAVADQIIIPDEAKKLTATDLVSELIDAAGNKITNFDGVTMSGFDAKAIGPQTVTLTYS |
| | | DAYGNQTTDSTTVTVDSATLTLQNHTQVAGPKATWNYADNIKAITDSKGQSLTLSDAKIT |
| | | VVQRPDLSVAGTYKIVLEYTDDLGQAHTETADVEVTASKAAITAVSKQVILAEKATMVTA |
| | | SSLVSTLYDADGVQIYNFDDVTMSGFNAKAIGPQTVTLAYTDAYGNQTTVSTTVTVDFAT |
| | | LTLQNHTQVAGSKATWNYADNIKAVTDSKGQSLTLSNAKITVVQHPDLSVAGTYPIVIEY |
| | | TDDLGQVHTKTANVEATASKASITAVSKQVILAENANMVTASSLVSALYDVDGFQIHNED |
| | | DVTMSGFDAQAIGPQTVTLTYTDAYGNQMTDSTTVIVDLATITGQATHPIAGPAATWDYR |
| | | DSVTQVIDANGKTIDVDTADITATTPNLTLAKAGKPQTVMLTYTDSLGKVHTTDVIVTTT |
| | | LSKAKITAVADQVIWPDQAKQLTATDLVDRLYDAEGHLITNYDNVEMSVLDSKLAGQQRL |
| | | TLTYTDVAGNQSVAYANVTVDQAKLVTKPSTVIAGPTATWSYEAGISQLTNAAGQLITVQ |
| | | PGTIKVLNRPDLNVDSVGQQQLITLIYTDELGKSQSVTAMVTAEASQATLTAKKAVILQP |
| | | DAAAKLTANDLVTSLTDASGQAVTDYQIVQMSKLDATRPGVQPVSLTYTDAAGNEVSTVV |
| | | KVTVDQAKMESQNRTQIWGPSMTWDYRQQLATVTDSQGHQLNPDQAKITVITGPQLTAKM |
| | | IDKPQTVTLMYTDDLQQTHTVSATLTLTASQAALVPRPAQIVWAKDAGQLTPANFLQTIT |
| | | GADGTQVSSLTNVKMSAVDASQPGAQTVTLTYTDDYGNEVTTTAQVTVDQAALTTQTARP |
| | | VAGPTAHWDYQTNFKTVTNAAGEVINVGDANLKVLTGPDLSTAMVGRPQVVTFSYTDELG |
| | | LTQTTTAEVTTVASRAHMTTSADQVIWPAVVGKLTVADLVTGLTDAWGQTSQNYQSVTMT |
| | | TINAQQAGKQQVTLTYTDEVGNVKTATTTVTVDQAALTTQPQTVIAGPTAKWDYHQGIGT |
| | | ITDGMGQPIAVNNAAITVVAMPDLTVAHIGQPQTVQLVYTDSLGQQQTALVQVTTVATQA |
| | | KISTRPVTVIAGPKTTWSLNDSVDWSTSLAADGTLLTAAQRQRVTVDGTLNLRRASNYPL |
| | | TLSYMDRAGNLITVTTSINVLASQAQLQVRDSQLTVGNAWTAQDNFERATDAQGQALTLA |
| | | DIAVDGTVNTQRAGQYTLTYHYTDVAGNQLTKTAVVTVVLPEDDHINTTDPDNNDHGETT |
| | | NPDNNDHAGIADPSETPKPSERPNDSDGHTVDWGVDDRITTKQQPAAATRAQTKVKTTAE |
| | | PALPANNEHTSAAKAAATPVTRVTDTTADTLPQTGERDRSAQQGAVVLGLTGLLGLMGLG |
| | | RRRHTHED |
| P16 | Lactobacillus | LPXTG-motif (SEQ ID NO: 67) cell wall anchor domain protein |
| D7VF49 | plantarum | OS = Lactiplantibacillus plantarum subsp. plantarum ATCC 14917 |
| (SEQ ID | ATCC14917 | MRYTRGKWRVTNPKVWLFSSVLILGWRIVPTVAQASEAETVTMSSHSVQLETDSQDQLTE |
| NO: 16) | | VARISKTAVTRDRHSVTAQSSKSADRTSSEQSATTGTVEAVSPTTSEAQQRSTQQDKTAV |
| | | DQQASDSTAASAGASTNQASAATSSDQAPAANSTGTHHAIDMASSASALGADSGAHSESL |
| | | SEAQHSGGQGKTIDSDLSGTVHSQSSVSTVTTATPVNSNSSRAVAATDQMSSRVEKRALN |
| | | KTNVTKSINIPVATKQPSKQRTVTASSFLTTAKNLADKNYLDQYAKQHGQAALIALIQDW |
| | | LSTYRIIALTGITIVNSSFDGSVATISGGLHVINTGATIRSGQDDEWETIINGGLSVTNN |
| | | TITFTTTNGLVDRPVANQDMDFTKPRPTGNGAIKGLPSVTVDSSLINAQEFSQAQINISD |
| | | FYDQLVTAGTILSATNGGTLSKMLIGESGTADLGSYQGHHYYAVNIDLNDWHSGIRTTGF |
| | | NNDDVVIYNVVTAAPALTIGGGFSSSTPNLVWNFNHAMRIQNTTMITGKIVAPHAVETTN |
| | | QNVDSAAVLQYGYGDVDSAIRETITSQNEHNYGFGQVVTDDPLDYLIAVIKSDGTSIDTL |
| | | AGFRHLLATGQLKITITDAAGTRLSGLNAVDTHIAGQHCYLITYQFGDQTATTWLNVQPS |
| | | HEPIIPISRIPEYSAITRTINYQDERTGAVLAGPVIQNVRVVRFAIFNAKTHELLGYDTN |
| | | GDGIVDTSDGTIAWLLVPPTDQDWVQVVSPDLSAQGYQAPDIPVVAGQTVIINGGDRTMN |
| | | TNVIVKYQQQTHIATTQRTVTRTINYIDGGTLQPIASLHAVVQTVKYQLLAVVAHDGTIL |
| | | GYDTNGDGQIETQLADEAWLIVGSGPWFGAVKSPDLSHEGYAAPDLKVVPEQMVAGVDDK |
| | | DVTINVYYRLATQAVTVYQNKRRVISYIDRQTHQSIATTVQQLVIYQRTAIIEKKTGKCL |
| | | GYDLNGDGLVDTSQADYAWILVGSGQFAAVTSPTLVVQGYTDPDIRTVAAQTVAITDPDL |
| | | MTTIVTYDHRIITVTPGNPARPGQPVDPDNPNILFPDEGGDTDLTHTVTRIIHYVYEDGT |
| | | TAAASVLQTVQFQRNAMIDLVTGEVTYQEWVPVSVTEMAGVISPIVAGATTTLTEVAAQQ |
| | | VSVTTADQVVVVTYKKSAIKPEEPGQPEQPSQPEEPGQPEQPSQPEEPGQPEQPSQPEEP |
| | | GQPEQPSQPEEPGHPEQPSQPEEPGQPEQPSQPEEPGQSEKPGELQKPSQPADSEQPDGL |
| | | SDQANLSRNQAEQSRTSQPSQAESDQSVVQTNQQKTAASVSGIGWVSAPAVSKRTTKHHR |
| | | MTTLPQTDEQNTQLSLLGMIGLALSSILGWLKIKSRD |
| P17 | Lactobacillus | Mucus-binding protein, LPXTG-motif (SEQ ID NO: 67) cell wall anchor |
| F9UME2 | plantarum | OS = Lactiplantibacillus plantarum (strain ATCC BAA-793/NCIMB 8826/ |
| (SEQ ID | WCFS1 | WCFS1) |
| NO: 17) | | MKPNNVNNQNKRHQSRWVITSATAMILTTLTIASQAAAADDTVTTTTNEPTNSQLNTNTQ |
| | | VNATQVNLKADTSTSVSTIKSDQSAVAATSPTTSTGSPSEHSSSVNTNPQQQSANPASQS |
| | | QATTTSESTPTTDIKHPTQTAPAQTTSASTTEPTTESNTESATDSQAKATTTDNQASKQP |
| | | SQQAVPASSNSTTTEVNTQSATSSASTDDKIVTNVNQEKLVLKTNQPVVRAISRTASENI |
| | | NDWMPNTLLQQEVLSQLRKQNPDRTWNSAADITKADMLLLTTYYGKDTYIDGKTSYSLEG |
| | | LQYATNLTTVWLNNNLNAPSGSYYSDVTDISPLANLQKLQVVNIQQNRITDISPLANLKN |
| | | LTEVDAAYNHISDFSPLKGFKNLKGTFSNQFITLPPAYISADNNIATLAIDCYLPDGSKV |
| | | QLKPNNGVGETVFYKNGQLYVRWYFNGAGGGNYDSNGHIYYTNMKPQQPGLTGPTENGTT |
| | | VIPMDDYYFMTAASDGNNFVVVRPYVLAATAAPITVKYVDALTGESLVTADLTLNGIVGQ |
| | | PYTTQRIDDELPNYDFTNIVGNASGVFTADAQTVTYYYTRKDAGDITIHMVDTNGNLVYE |
| | | PQILPGKHNLGNAYNLDAPTFDHFKLHQTIGNAAGVFTTDPQSITFVYVRLDAGNITVKY |
| | | QDKQGHQLKPDKTVSGSQSLGQTYTTEPLGIENYTLMTTPANATGTFTDQEQTVIYVYVR |
| | | RDAGQIVVKYQDSAGNPLAPDKLLDGKEQLGVAYQTAAISIPNFYLVATPANATGTESTD |
| | | TQTVIYQYARSNAGHITVKYQDANGTTLAPDDVLTGDGQLGRPYQTSAKTIENYRLIQTP |
| | | ANATGQFSDQAQTVIYVYTREDAGDITVQYLDENGQQLAADSVLSGQGQLGQPYETSPLN |
| | | INGYTVKSTQGNTTGTYTVQPQRVVYIYERTAGQPVTAKYQDQDGKSIHPDVVHSGYLGD |
| | | NYSTEQLVIDGYTFKAVQGDVSGTFGTSAKTVTYVYTESTPTIPDTQGTVTVHYVTKDGI |
| | | KLNEPTVLSGKTGTTYQTVPLTFTDHELVGQPENATGLFTADNVDVTYVYQATDTTGTDD |
| | | IIDPEEPEQPTKPIKPTTPETPNEPGTTVTQPDRIKPTQPAVAVKPAATVKPTLKPAAAQ |
| | | ASLVKTTSPVTEHSAQLPQTDEQTGKLAVILGLLLSVVTLGFYGKNRQS |
| P18 | Lactobacillus | Mucus-binding protein, LPXTG-motif (SEQ ID NO: 67) cell wall anchor |
| F9USM7 | plantarum | OS = Lactiplantibacillus plantarum (strain ATCC BAA-793/NCIMB 8826/ |
| (SEQ ID | WCFS1 | WCFS1) |
| NO: 18) | | MQRRRLQRAQLTEKRTYKMYKKGRLWLIAGLSTFTLGASLLPMTGRADTTSTPAEKQGTR |
| | | TETTGNQITLASKSVGSSSMANDGEEKTNNSQVETSSEASNVTASTEAKSTESTTQTVVD |
| | | STVTSTATETTRANGATNQTSKMSIVDTTSNNTEQNQAVGGTTDSTASTATIEDQAKAAN |
| | | RATTDGKINTATVATKTTTTASYATADISTINTIRSAQKLARATVATVATVNSATKTYDGK |
| | | IDTPNRYTITLTDGTKAPSDWAVTSTANVYTVTDLTDVDTSKFGSSVGTYTLALSTAGIT |
| | | KLAEANSSADITAANVVTGTLTIKQAPVPTAIITIGSASIDYGDAKPSTYTITVPSQYAV |
| | | PSTWTLASSATDGTTNTYMIASSSGDVIVPTATQSGTYQLVLSDQGLTALQQANPNAAIT |
| | | ADTIIAGSLVIAAHDIITMGATTIVVNKTTSTVPVTVNSRTIVVPTGWTIRYDDIQTDAI |
| | | VYDVPVSDTTYSEAVNTAVVDKYTITLTDDTIETLANLNSSTTFNSTTVGKGVVLVKASA |
| | | AVAISPANYGAQASAETPVTGLTISHARTKGIDLAYGQALYLILPLINMNPSGMTVANLT |
| | | DYVIIPSGFKVATNSEGAINIATDPSSVLTSAIEAMMTKNDVTYQGLKVTQLTDYRGRQT |
| | | FKIHFDKTTVYDGGAFATLKYALLPVIAVQNTGVTSGLIGNQVSSPDSAVVYVTDDSNEN |
| | | NGSYSLNLQNYTNIDSVADALGIADAVTIGSGFTSYLYHYTLSAKTITDTYSLVGNDGTS |
| | | LGEVTFTGDSGKTYVPMTKLPMTITQNGVTYYLNTSAVSLTQTYSGDSNSNYTVTYQRYV |
| | | TTTTDTAAKITIAPASKVYDNNATTDPSRYTVYLPTEYTAPSDWTADSAATAVDGTTAYQ |
| | | VSTDYLNTTAIDQNVGTYAVTLNSAGMAALSAANPDFLIAGDVNVGGTLTITQRPVTITL |
| | | PDTILWANGQEQNITPVITGVVAVQSLDYTLTSGLTDPDTTTITATLTNAAANSNYKLTN |
| | | SPSGQLTVGAVTVVYQYGYRDKAGTLHVVTTANGTATHGTDVTAKDYLSYTTSDTTATHA |
| | | KTGYTLQPESTGYQADGTLADVGGQVVYTYLANTEKIAVVYVDQDKNNVILKQIPLSGSF |
| | | GTPTNYTTAQDIAAYEKLGYVLASDKVPAPLEFDQDTEQTYYVYLKHGTITATVDQPGNV |
| | | AVSDLMKTSQRTIHYVYADNTPTDLADVLQTVTYTRTATGDAVDRTVLSYGNWTTNVNSY |
| | | PAIESPTITGYTADQTTIAAAVPASMGETTETTVRYSVNSETIRVQFVDGTTDNQVLSYI |
| | | DLNGKYGDAADYTVTADIAKYAKLGYEPVNSDLPDQLIYKQNTQVYTVTLAHRHVTVSVD |
| | | HPGQPGQAIDADYPAGPKYPAGTGRDSLEQTVTRTITYQYASGESAAETVNQSVTFNRTA |
| | | TFDMATGKQLTYGDWTVAPGQSALLAAVTSPTITGYQASVTEVEAASVTSHDKPHLIAIT |
| | | YTAKSQTATVAFVDVTSGKTLPTTVVTGAYGTTNSYSPVSQIAAYEKLGYRLVSNNVPTT |
| | | GITFDQNDVIKSYTVKLAHQMTTVTPTKPGQPGQPVDPAHPEGPKYPAGTGLKDLTTSVQ |
| | | RVITYVYNDGQTAAPTVTQTVSFERKATFDQVTKVVTYTDWRTPESALTGAYAVVESPII |
| | | AGYTPNATRVASVTVSAKDTESRQTVTYQANLETATVTYVDATTGHRLGTSVTLTGRFGT |
| | | QADYQPTTMIAQYTQAGYVLMGSDYPATGVTFNQAGVVQKYTVYLAHNKIVITAPDQLTK |
| | | TITQTVHYQDQAGHTLQADTIRALTFTRSGMKDAVTGVATYRDWAPTGLNFTAVSAPTIA |
| | | KYHALTATTQAVAITAASADDVQTLTYALDVPTPTKPVKLTKPAKPTKPTTSDDLIKPTT |
| | | KPITAAKPTQLTKPATVVKDFQATTGNQTPAKSTRTLVSSRIKAVKTAPASAIIKPGSKV |
| | | TEPAHKAQADTTSRLPQTGETRWSEMAAETLGLTLATLLLGFGGLKRKRHEK |
| P19 | Lactobacillus | Adhesion Exoprotein Lactobacillus gasseri (strain ATCC 33323/DSM |
| Q045Q7 | gasseri | 20243/JCM 1131/ |
| (SEQ ID | ATCC33323 | MVPQFTWGGV NAQAVRADSV NEDATEQVEK KDEANVKAAE VKTTEQKQEN NKTAVSATNE |
| NO: 19) | | NAKQNVAENT SDSKKVASNR DVNVIKNDVT TDEKAAAKSS VQTDKDVNAN KLNTNTVSVN |
| | | KLQRNVNVAG LAESKATSEI NSTLSVRESM QQKAVSLKAN EIARTVIMNK PAGPDQITQS |
| | | VKLGTMLGSS NGQIIDGKTT KIYTATVIAV GSSTDMKKYR VTVDSDTGEI LAGQDLYDTF |
| | | MNLQPSDFKV NLDAIDQSQI DVPGYTWKIT SATPAGANIG KEDYTFGNPQ TITIDYTRDV |
| | | EGNIKKKVTE ITDKLVNNQM TTEPARTVIL KKTTTGAAND ETIVQKADIR GLARTSSKTV |
| | | AGITEKKIEV AIAPYVEPDK PSSQYYKQYT ITFNPDTGQI ISGQNDYDQL MALKRSDFKA |
| | | DLPAIEDSQI DVPGYTAIIT SATPAGAGLE AETYTFGHPQ TITIDYTKVK HTVTYQFKDP |
| | | FGNQVGTSVP VTGAVGSNQS VNLTLPDGYQ LASGSLPTSV TIPESDKIIP IPVKHQLTIT |
| | | LSGESVFNYA DDNWQNLVET NELPASGYYV EFNDANARVQ LNDGDVTYNE NRNAGTYTVS |
| | | LTEKGLNDIK DQSHDNFIYP DLKDVKSEAK FIINKGNKTI SLMGGDTKVF DNTSTLPDQG |
| | | TFYSGLGLAD NDQGRISVYN SDGNPRTIQL TPADVEFWEN GHKIAKDQAK NVGNYNLRLT |
| | | DDFINKVKAA DGNNGNNYEW AYGTNTPTGS DTYTADYVIY QATGKAKLSG NNSKLYDGNA |
| | | VTTDDVNKGR KITIDLTLPV YKQADEPGDE PQLLGTVDLG KYTLQDGDYT WANGTAPTKG |
| | | GSYTINLNKD KILAHLQDRL VALAGKGTDP DDSTKSLSNV TISADDMAGQ ATFAIETTTT |
| | | YQFVDDDDNG SKVGTPVSKT GLKGESSNIS LTVPTNYVLA AGQTLPTSVT FGDTNTTVDI |
| | | HLKHATKTVD KNNVPDGYTK DDFAETINRT ITAKEPTGDV DLSQTTELTR TGTYDEVTKK |
| | | VISYGNWTTG NFDEVTAPEV AGYTPSQANV AAVTGVTPDY VDPKVVITYA PNDQTGKISY |
| | | VDVNTGTEVG NTPLTGKTDE EVTINPVAPT GWKIVDGQSI PRTEKATPTG IPPVTVKVEH |
| | | KTTVVPPTDP KTPKDKLPDN PDKHYPDGVG EKDLNKIIVR QITVVKPDGT REKHDQSVKL |
| | | TRNATVDEVT GEVIKYGDWT TSNFGEYDAP TVPGYTPSQA KVEGVKVTAD SDFAPVTITY |
| | | TANPHTLNIN YVDKDGNKIG NSYQVPGRTD ETVAVDVPGH VPANWELVPK QKYTTSITFG |
| | | SDDPQDQNYV IQHKTTTTDG RDHKDNQDLY REVTRTILMK VPNATSQGRE TETLSFYRIK |
| | | THDEVTGKDT YSDWASNVTG DKIAFDEFDV SKTNDGKEIA AGYTPTSNDV VLEDKNGDKF |
| | | VPSQSALKNG VPADSFTVEV AYTPNAQRTT VTYVDENGKE ITNPDGSVIP GSHYDLTGVT |
| | | DQSNVPTNIQ NNVPTNWHIT DPEVPATITF GADGHTPIKV HVAHNTKPVD KNDVPDGYKE |
| | | SDFSKTINRT ITANEPSKSV DLSQKTELTR TGTYDVVTKK VISYGNWTTG KFDEVKAPEV |
| | | AGYTPNPASV NAESVTADYV DPKLVINYTP NDQTGKISYV DVNTGTEVGI TPLTGKTDSD |
| | | VTITPSAPAG WKIVDGQNIP TTEKATPTGI ATVTVKVEHK TTTVPPTDPK TPKDKLPDNP |
| | | DKHYPDGVSE KDLNKTVVRQ ITVVKPDGTK ESHDQSIKLT RTATVDEVTG EVTKYSDWTT |
| | | GNFGEYDAPV IPGYTPSQAK VEGVKVTADS DFTPVEITYT PNAQKTTVTY VDENDKEITN |
| | | PDGSVIPGSH YDVTGVTNKK VDTNIQKNVP TNWHITDPEV PATITFGADG HTPITVHVAH |
| | | NTKPVDKNDL PDNYKESDFS KTINRTITAK EPNKDVDLSQ EIELTRTGTY DEVTKKVISY |
| | | SDWTTGKFDE VKAPEVAGYT PSQAKVDGVD KVTVDYVDPN VVITYIEDPV GQDITVKKGD |
| | | TPDPEDGVKN HGDLDKITDP KHPGTKTTYT WKKTPDTSVA GDVPATVVVH YPDGSDKPVD |
| | | ITVHVVDDTP VVPTKNPDPV GQDITVKKGD TPDPEDGVKN HGDLDKITDP KHPGTKTTYT |
| | | WKKTPDTSVA GDVPATVVVH YPDGSDKSVD ITVHVVDDTP VVPTKNPDPV GQDITVKKGD |
| | | TPDPEDGVNN HGDLDKITDP KHPGTKTTYT WKKTPDTSVA GDVPATVVVH YPDGSDKSVD |
| | | ITVHVVDDTP VVPTKNPDPT GQDIHTPQGK VPTPESAITN KDKMPDGTKY TWKEIPDVNT |
| | | LGKHPNVVVV TYPDGTAVEV KVNVFVDGTP EVKKETKAPV VKKQVVEPTK VETRQKLVNN |
| | | YVAPRAVEVQ RAQAKGKRQL PQTGAKENIA SEVLGMLSVG LGALTAGFAS KRRKKNR |
| P20 | Lactobacillus | KxYKxGKxW signal domain protein OS = Ligilactobacillus salivarius |
| C2EIY2 | salivarius | DSM 20555 = ATCC 11741 |
| (SEQ ID | ATCC11741 | MEKLLGEKRRYKLYKAKSKWVVSAIITISGVTFLVTSPVSNAQADTVTGSESVKTEATQA |
| NO: 20) | | SSSSVQNNTTAQTTVTTNSNSSNNVSNVQTDTVKEAATSNVDSVASQNQATTAQQAKTTA |
| | | DTADQTVPPTTYKDHVKGNVQTAWDNGYKGQGMVVAVIDSGADTNHKDFSKAPESPAISK |
| | | EDADKKISELGYGKYTSEKFPFVYNYASRDNNWVKDDGPDASEHGQHVAGIIGADGQPNG |
| | | NERYAVGVAPETQLMMMRVENDQFADENTDDIAQAIYDAVKLGANVIQMSLGQGVAAANL |
| | | NDVEQKAVEYATQHGVFVSISASNNGNSASVTGEEVPYEPGGADGNFEPFSSSTVANPGA |
| | | SRNAMTVAAENSVVGAGDDMADFSSWGPLQDFTLKPDVSAPGVSVTSTGNDNRYNTMSGT |
| | | SMAGPFNAGVAALVMQRLKSTTNLSGADLVQATKALIMNTAKPMTQQGYDTPVSPRRQGA |
| | | GEIDAGAATESPVYVVAADGTSSVSLRKVGDSTQFALTFKNLSDKDQTYTFDDFGGGLTE |
| | | VRDADTGTFHDVYLAGAHVYGNKTVTVKAGQSATYNFTLSLTGLKENQLVEGWLRFVGND |
| | | GQNQLVVPYLAYYGDMTSEDVFDKAANQEGTVYGGNYFVNEDNYPRGIADENSLKALVNL |
| | | EGNYNWQQVAKLYQDGKVAFSPNADGKSDLLKPYAFVKQNLKDLKVEVLDKNGKVVRVVA |
| | | DEQGLDKSYYESGVNKDVTLSVSMRNNPNTLAWDGKVYDDKTGEMVNAADGEYTYRYVAT |
| | | LYNDGANRVQTADYPVVIDTTAPVLSNVKYDATTHTLSFDYKDTGSGFTDYSYAVVKVND |
| | | KTFGYKLNDGKNSKFLNAAKTSGTFKAVLDSDTLAALTAAKNALSVAVSDVADNTSTVTL |
| | | LVNGNNDATTKVSVWNATNGLELDQSSPDYQAATSTYNLRGNATSDFYYNGALVQGDNSG |
| | | NFVVPVSTSDTAVVFTSDAAGKNVVYKLNTATPKAVFAWQVNNTVKENFGIVLDTVVSNN |
| | | KDDVVVQAAVTKGDNVEAYARDYFTGAVYKADVKDGLATFHVKVTNNSGRTVLLGWTEVV |
| | | GPTFNDVQRTSANGVYLGVDTDTENPTPAPAFTSADQLGTNVVQEKADSATIGNPGDLPG |
| | | HSLKDLTTRADANPDIHFDYLKDNDYNWVGAQAVKDGVYNPSTQVFTLTGKVDPNVKSLV |
| | | VLGDSYNEDDPVNKVNLNSDGTFSFQFHTAPTSQRPVAYIYTKDDGSTTRGTMELILDTV |
| | | LPTLSLNNVANLQLDSNGDYQVYTNNKDFSVSGEATDNLDGYRFFENGDNDYREFHNSGV |
| | | NFVTEAHQDGSTVTNPYPAYKFSKTFNLADATGETTHVYTLSVVDLTGNTVTRKFYVHYQ |
| | | PASDTVKTVTTDKDGVTKVLVDYNNNTLQVKDSTGNWVNATGVEAAKDYRVVNEYGNVVL |
| | | LLNVLADKEQDNNKVQVNEVTNNKVEQTVVTKTVSNKSVAKVGKKAAEPVKVLPQTGENN |
| | | SKSTSVLGAVLASIAGFLGALGLRRVKKD |
| P21 | Lactobacillus | Cell surface protein, CscB family OS = Lactiplantibacillus plantarum |
| F9UU91 | plantarum | (strain ATCC BAA-793/NCIMB 8826/WCFS1) |
| (SEQ ID | WCFS1 | MMVLLQVIAAGATVSLGADMTAQAATLPQLTFAKSTASDNILTNQHFDVELQVGDTASKI |
| NO: 21) | | NTIDLPNEVNLDGPEEFKQIKRVFDDSQYTTGDNGAFTITAKHLTVAYNPDKRRITVQWS |
| | | DEYPQTKVPIRLTAVKAEKLALVAVADDQKGPALNVEIKQPQTQADQASTSSASSSAATD |
| | | TNSSTASSSRQATSSAASLDSSRSAATTLSSQAVNQTSASSSEPSQETAANQSSAVTESA |
| | | GETTDSSASISSSSTASQVFSSAPTKQATASAKSSPLIPVTRLAQLSSNVVDVSQWSQLV |
| | | DAWKDASVDEINITADISNPTAASGALDSRLSGNIIVNGNGHSVNIGRAGFHTRNNTATS |
| | | GTMYTATFMNFASLIGSFGNDAGLIGSSTGGDGAGGALNWTFNVSNITVPSGTSYTNTSR |
| | | RFVSAQGNQVNITGNCRVTTVRENILCGGLDVAAGQTFTGSKIANGDDNSFIWFVYDYQG |
| | | TGNRQVNVEEGATLNCIRRPASSTSTAYTTYPVIFDAYESINVGKNATFNASVPGNAYSN |
| | | KYFYGSQYHRNFYADTGSTVNLTSLARSQSPISFSDNATSTIQSSSGANIYVIAATGAPL |
| | | ISGNYARLATVRFINPNNLDLRNSSTGTTAAASSINQDNVGTFEIQDSNISLWKLASSVT |
| | | GGADYSYSNVSQLLQQGSAVTATDSNLQSNYLSSKMRRISATNQKPQLAFNNPYDGTTKL |
| | | TDADQKLRTRVIVAMVPDTNGVQDDGTVNYIPQYASAGQLTVSYSVNGKTITAQTDSNGY |
| | | ATANVGTFLKAGTTVTASTSNTSGTTVTATGTVVDVTPPNPATMVSPDPIRVSTGTVSGQ |
| | | NGEPGAQVTLALNGQIQTNVKTVVNANGTWSLNLTGLSLKIGDKIIIYMADSLGNRNPDP |
| | | NSYPNGQQYHDATFQPAPIFTVAKDLIVNPIDPDDPSKPGTGGTNNLGPLSLDAVPTHLN |
| | | FGQHSIPTMDTAYPLLSPSAAEDQLATATDGQKYATVGGQKNGQDSVYTQVTDTRDTPSG |
| | | WQLTAQLSALTATDGTTMTGSYVTLTSGTAQYLNASTSKWVTATDQNQATLPAVIKLTPG |
| | | ATQQTLIAGTTSQQGVGTNQQIWNVNNVALHVKGGRVMAKNYSGTITWQLNSLPSQ |
| P22 | Lactobacillus | LPXTG-motif (SEQ ID NO: 67) cell wall anchor domain protein |
| C2EIP8 | salivarius | OS = Ligilactobacillus salivarius DSM 20555 = ATCC 11741 |
| (SEQ ID | ATCC11741 | MEKPTPIDVTYHYDRMNPASIEDRTDISYHYNKISVPIPNPTKKADKEGKTLIAGDESTQ |
| NO: 22) | | HISQYTGVNQKLDKFAVGDAIQYTNDGRLPVSFDLSKWTVTTSNGTNVTAQGKFTQYDKT |
| | | FEGKKYHVVSWSPTNVSSLKDNETYTLNTILKTLNDGITDGEIDRAVGGGDGVTFGEAHG |
| | | YDEFNPTTDKAWKEGSQTVNGKIEINEDIAHAKVTMTMPDPAKLANKLSNVAITDNYSKF |
| | | ANLVTVTGANVYENERNATSDYTIVNNNKVVTATRKNPATANGGTVSLVVDFKVNPDVPS |
| | | GTKLVNSGSGTINTQTVPTPDAQIVTFTQTPTKHWVEGSQVVDGKTYINDDIVTTQVDMN |
| | | LPDPKALAKTLSYVSVGDNYRDFADKTVLQSYKVLENGTDVTSQYTITNQGGILQAVRKN |
| | | AATAPGGKVSLIATFAINHDVKSGTKLTNRGFGRINNHTVDTNTPQIVTFKQDTSKHWVE |
| | | GSQVVDDKTYINEDMVHGQVTMTLPNKDSLAKSLTDVALVDDYSDYANKVSYVNAQVFEN |
| | | NTDVTSQYNITNAGNKITATRKNPGATPSGSVRLVANFKLNSNLPSGTKLINRGSGRINN |
| | | NTVNTNEAKILTYVQSTDKHWVEGSQKVDGKTYIDGDTIHGQVTMTLPDKNTLAKALSTV |
| | | QVIDDYSKFAKMVDYKSAQVLENGKDVTSEYNISNVYGQVVATRKNATATPSGNVTLNVT |
| | | WTIHKDVPSGTQLVNSGSGRINSHTVPTPDRNIVTYKQDGLKDWINAQGQIVNGKTVIDN |
| | | DTVHAKLVMTLPDPKTLATPLTKVQLDDNYSKFAGLVDYVSSQVLENGTDVTSQYNITNA |
| | | NDHVIATRKDASKTPGGKVEFRVNFKIHTDVPSGTTLMNSGEVTLNSETVPTPTPNIVTY |
| | | KPDTDKHWVLDNNVTDNKIYFSGDKAVAQVSVDLPDASKLATPLSKLVLVDNYSDFADKV |
| | | KLDSAKVLENGKDVTSEYDLTNKDGKVFATRKDAAKTPSGKAVLVTTFTINNGIENATAL |
| | | HNKGSVTVDSITDEVPDTPIVVFTPKAHKDVELGGDVKGDTENSVDGSLILNGSVVTYPI |
| | | TTSDLPAERAEDITKRVVKDTLDKNAEFVGFKAWIENDKGELEDVTSHYKLDKNGQDLTF |
| | | TEDSYLLGLYNKDKSKQTHTPIIDLVVKVKGDAQKINNKATVLTNDNVTETNEVSVDTPA |
| | | KPTPTKVDKNEKGVNIDGKNVLPGSVNNYELTMDLAKFKGIKVTDQDLAKGFYFVDDYPE |
| | | EALDVDPQTFTYKTVDGKTVKGLSAKVYQSLSEVSENVATALKANGITPNGAFVLISADD |
| | | PAQFFKDYVETGTNIVVNAPMKVKEGFAGKYQNKAWQLTFGQGEATDIVSNNVPKIDPKK |
| | | DIVISADNRTSLNNHTIELGQNFDYLLKGGILDKDQGHDIYEYKWVDDYDENHDQYNGQF |
| | | IAPLTVDVTLKDGTVLKAGTDISNHVSQNIDTKTGSVEFSVDKDFLDKVDFDKSGFAADI |
| | | LMSVKRIKAGEVDNTYTNIINGQKFGSNTVHSTTPEPKEPETPATPKTHETPSVPVAQTQ |
| | | TPATPQPVKMVTSTPAPKAPESPALPQTGEANDTLAEEVVGFAAIVAALGMAGTSLKKRE |
| | | D |
| P23 | Lactobacillus | KxYKxGKxW signal domain protein OS = Lactiplantibacillus plantarum |
| D7V951 | plantarum | subsp. plantarum ATCC 14917 |
| (SEQ ID | ATCC14917 | MRNRLNRLGLESKSHYKLYKSGRRWVAASITVFSVGIGLTFSQVEQVKAATGTGVDTADN |
| NO: 23) | | SASVSSDMAEPSNAVVLKSASTATATKTATQDAKAATDVTAATQDTKATTDSTGATSASS |
| | | NRQSTAATKPAAEVGTASSSADSSASISSTDGASASAPSVTSKSTNTEATSASATKTATT |
| | | SADTDVLNTETTSSSVANDLTDATTASQTRTETGKTASIPTAEAPTITTAVTSRALPLTG |
| | | ALASRSANTPVTKSAVQAVSAITSEAETKPTVSLVTTGTVSMDYGEASLADLESHISSPD |
| | | ETPANDVAYYIQDAAGNYLEDVNGNKVNLLYALFLDSADVNDYVDVVYTDEHGQVTKYSG |
| | | DTDFSTLDQIGSYSVTINAAGKAGMSRVMQDYNAYDTSTSDLDDFVPTFSTGASDYTFTI |
| | | NIVPVKITATTGKNGLIILRPSQLYTGSLTMLPVVTVKNATKQNILQISNGEIGDAKPGV |
| | | AGKVGQRVLTLADFTYTYQGTETNLTGADTGKYAITLNDAGRKAVQAALGSNYILDDAAV |
| | | FTTTGAVQAAGLELKIASGTVTYNGKPQGTSVTTGTVYDHFDFTTTTDTNVGTYDDLTYA |
| | | LADPTQAAILAKNYTVTTTDGTLVITPADLTVTVKDDNAVYDGRSHGTTATVTSGTNYDQ |
| | | LVFTAVAADGSGATTYTTVGTYAMTGTTAADTSNYKISYVNGTLTIDPAKATITIPNKIY |
| | | WSDGTQKNLAAVVTGTVNGETLKYRVTNGMSAVGTKTITATPDADDSVNKNYTISVIPGT |
| | | LTIGDIAVKYLYEHVDANGETQVDASETGTATHATDATATDYLTYTTAAKPKTGYVLAPN |
| | | TGLAYNGTLTDQGGTVTYRYLAKTETAIVTYFDQTDNKVIKTEPLQGAYGTTDAYRTADT |
| | | IAAYENAGYDLVDDDYPTAGGVYDQDGIVQKYQVTLVHKFVTRTPDNPGTPGEPIDPDNP |
| | | NGPTYPVGTDFEDLTEQVSRTIQYLYKDGRTAKPDNVQAVNFGRNVTVDEVNGTVVYTDW |
| | | LTDDGAVTGRFEAVDSPLITGYTADSTSIAGNPAVVWQDDDTTIPVTYTVNKEYATVTYF |
| | | DQTDNKVIKTEPLQGAYGTTDAYRTADTIAAYENAGYQLYRDDYPTAGVVYDQDGSVQKY |
| | | QVTLVHKFVTRTPDNPGTPGEPIDPDNPNGPTYPVGTDFEDLTEQVSQTIQYLYKDGRTA |
| | | KPNNVQAVNFSRNVTVDEVNGTVVYTDWLTDDGTMTGRFEAVDSPSITGYTADPTSVAGR |
| | | DTVSGTDLSPDVQVYYQANPEKATVTYEDMTTGAVLTTDPITGDYQTVSNYRTADRIAQY |
| | | LNMGYELVSDDYPTSGAVFDKDGSTQAYTVKLQHKLLPLTPENPGTPGEPIDPDNPNGPT |
| | | YPAGTAVQDLIKQVDQTIHYQYQDKSTAADANTQTITFKRSVTVDEVNNKLTYTDWLTGT |
| | | ATTGRYMPVDSPEIKGYVADSTRIAGNDEVHNADADTNIVVTYQAKPENATVTYVDVTTG |
| | | KTLAIKSLTGDYQTTSSYRTAETIASYVKNGYQLVRDNYPTSGAVFDVDNFAKTYTVTLK |
| | | HKLATVTPENPGTPGQPIDPDNPDGPKYPVGTTAQDLTKQVSQTIKYRYQNGASAGTDNV |
| | | QLITFNRDATIDEVDPTAVYTDWINGTSASGRYTTVMSPVITGYTADKTQVAGRDSVANT |
| | | DSDTQVVVTYAAKPEKATVTYVDVTAGKTLATANLTGDYRTQSNYRTAETIAGYVKNGYE |
| | | LVRDNYPVSGMLFDVDDFAKTYTVTLKHKLVTVTPGNPGTPGQPIDPDNPDGPKYPVGTT |
| | | AQDLTKQVSQTIKYRYQNGASAGTDSVQLITENRDATIDEVEPTVVYTDWLDGTSATGRY |
| | | TTVTSPVIIGYTADRARVTGNDAVTSAAQPTNIIVTYALNAEKATVTYVDVTTDKTLATV |
| | | SLTGDYQTSSDYRTANTIADYSNQGYVLVRDSYPVSGAIFNDDGVVHSYLVQLAHVTTAT |
| | | TETKTITQTVHYQSTTGTQLHDDTVRAMTFTRTKRVDQVTGDVTYSNWSTNQADHTFERV |
| | | AAFSIPGYHAVVTGTQAVMVTPASVDDVQTIRYVTDRLSTGETPKTPVKTVTVNKSDKIK |
| | | TTDTPDKVATVKTPDKAQTVATTTAKQASVKRSVDLKQAQAVEQPAQTRPANVKTVKLAK |
| | | TTKSVKPTAAHQSATHKQATLPQTNDDRQASVAAELLGLTAATLLVGVSAILKKRHN |
| P24 | Lactobacillus | Cell surface protein OS = Lactiplantibacillus plantarum subsp. |
| D7VF97 | plantarum | plantarum ATCC 14917 |
| (SEQ ID | ATCC14917 | MQRRRLQRAQLTEKRTYKMYKKGRLWLIAGLSTFTLGASLLPMTGRADTTSTPAEKQGTR |
| NO: 24) | | TETTGNQITLASKSVGSSSMANDGEEKTNNSQVETSSEASNVTASTEAKSTESTTQTVVD |
| | | STVTSTATETTRANGATNQTSKMSIVDTTSNNTEQNQAVGGTTDSTASTATIEDQAKAAN |
| | | RATTDGKINTATVATKTTTTASYATADISTNTIRSAQKLARATVATVATVNSATKTYDGK |
| | | IDTPNRYTITLTDGTKAPSDWAVTSTANVYTVTDLTDVDTSKFGSSVGTYTLALSTAGIT |
| | | KLAEANSSADITAANVVTGTLTIKQAPVPTAIITIGSASIDYGDAKPSTYTITVPSQYAV |
| | | PSTWTLASSATDGTTNTYMIASSSGDVIVPTATQSGTYQLVLSDQGLTALQQANPNAAIT |
| | | ADTIIAGSLVIAAHDIITMGATTIVVNKTTSTVPVTVNSRTIVVPTGWTIRYDDIQTDAI |
| | | VYDVPVSDTTYSEAVNTAVVDKYTITLTDDTIETLANLNSSTTFNSTTVGKGVVLVKASA |
| | | AVAISPANYGAQASAETPVTGLTISHARTKGIDLAYGQALYLILPLINMNPSGMTVANLT |
| | | DYVIIPSGFKVATNSEGAINIATDPSSVLTSAIEAMMTKNDVTYQGLKVTQLTDYRGRQT |
| | | FKIHFDKTTVYDGGAFATLKYALLPVIAVQNTGVTSGLIGNQVSSPDSAVVYVTDDSNEN |
| | | NGSYSLNLQNYTNIDSVADALGIADAVTIGSGFTSYLYHYTLSAKTITDTYSLVGNDGTS |
| | | LGEVTFTGDSGKTYVPMTKLPMTITQNGVTYYLNTSAVSLTQTYSGDSNSNYTVTYQRYV |
| | | TTTTDTAAKITIAPASKVYDNNATTDPSRYTVYLPTEYTAPSDWTADSAATAVDGTTAYQ |
| | | VSTDYLNTTAIDQNVGTYAVTLNSAGMAALSAANPDFLIAGDVNVGGTLTITQRPVTITL |
| | | PDTILWANGQEQNITPVITGVVAVQSLDYTLTSGLTDPDTTTITATLTNAAANSNYKLTN |
| | | SPSGQLTVGAVTVVYQYGYRDKAGTLHVVTTANGTATHGTDVTAKDYLSYTTSDTTATHA |
| | | KTGYTLQPESTGYQADGTLADVGGQVVYTYLANTEKIAVVYVDQDKNNVILKQIPLSGSF |
| | | GTPTNYTTAQDIAAYEKLGYVLASDKVPAPLEFDQDTEQTYYVYLKHGTITATVDQPGNV |
| | | AVSDLMKTSQRTIHYVYADNTPTDLADVLQTVTYTRTATVDAVDRTVLSYGNWTTNVNSY |
| | | PAIESPTITGYTADQTTIAAAVPASMGETTETTVRYSVNSETIRVQFVDGTTDNQVLSYI |
| | | DLNGKYGDAADYTVTADIAKYAKLGYEPVNSDLPDQLIYKQNTQVYTVTLAHRHVTVSVD |
| | | HPGQPGQAIDADYPAGPKYPAGTGRDSLEQTVTRTITYQYASGESAAETVNQSVTFNRTA |
| | | TFDMATGKQLTYGDWTVAPGQSALLAAVTSPTITGYQASVTEVEAASVTSHDKPHLIAIT |
| | | YTAKSQTATVAFVDVTSGKTLPTMVVTGAYGTTNSYSPVSQIAAYEQLGYRLVSNNVPTT |
| | | GITFDQNDVIKSYTVKLAHQMTTVTPTKPGQPGQPVDSAHPEGPKYPAGTGLKDLTTSVQ |
| | | RVITYVYNDGQTAAPTVTQTVSFERKATFDQVTKVVTYMDWRTPESALTGAYAVVESPII |
| | | AGYTPNATRVASVTVSAKDTESRQTVTYQANLETAMVTYVDATTGHRLGTSVTLTGRFGT |
| | | QADYQPTTMIAQYTQAGYVLMGSDYPATGVTFNQAGVVQKYTVYLAHNKIVITAPDQLTK |
| | | TITQTVHYQDQARHTLQADTIRTLTFTRSGIEDAVTGVATYRDWAPTGLNFTAISAPTIA |
| | | KYHALTATTQAVAITAASADDVQTLTYALDVPTSIKPGKPTTSDDLIKPTTKPITAAKPT |
| | | QLTKPAMVVKAVQATTGNQTPAKSTRTLVSSRIKAVKTAPVSAVIKPGSKVTEPAHKAQA |
| | | DTTSRLPQTGETRWSEMAAETLGLTLATLLLGFGGLKRKRHEK |
| P25 | Lactobacillus | Cell surface protein OS = Levilactobacillus brevis (strain ATCC 367/ |
| Q03T21 | brevis | BCRC 12310/CIP 105137/JCM 1170/LMG 11437/NCIMB 947/NCTC 947) |
| (SEQ ID | ATCC367 | MRNRLNKMEPEGKTHYKLYKSGRRWVTAGITVFSVGIGLTLSQVGQAKAATNSDTDETEN |
| NO: 25) | | SATVSSSSPTETKNAVVLKSSSAAATSTAAAAVSASTASDSQSTATPAASTSRAVSGAAT |
| | | GAAASDSAATQPTVSSADSQSTENTRWSAASDTTSNAASDQESQQAAGTTDNANSDAASS |
| | | ATTATNTNAMPMTNRITSRAMNVTAAVSEAEAQPTVSLVTTGTVAMSYGDASLADIGLHI |
| | | SSPDETPANNVAYYIQDAAGNYLEDVNGNKVNLLYAFFLDSVDVNGYFDVMYTDVHGHVT |
| | | KYSEDTDLSTLNQIGSYAVTINAAGKAAMSQVMQRYNAYDTTTNVFVDFVPTFSTGTSDY |
| | | TFTINIVPAKITATTGVNGLTMLRPSQAYVGSLTMIPLVTVKDSEKKNVLQISNGEIDYA |
| | | AEDVVGKAGQSILTPADFTYTYQGTETNLTGADTGKYTITLNNAGRAAVQAALGPNYILD |
| | | DTAIFTTTGAVKAADLGLTIASDTVTYNGQAQGTSVAVTNGTAYDHLDFTTTTGKDVGTY |
| | | DDLTYALADPTQAAILAKNYNVTTTDGTLVITPADLTVTVKDDHAVYDGRAHGATATVTS |
| | | GTNYDQLAFTTVAADGSGATAYTKVGTYAMTGTTVADTSNYQISYVNGTLTIDPAKATIT |
| | | IPSQVYWADGTQKNLTAVVTGTVDGETLKYRVTDGMSAVGTKTITATPDADDLVNKNYTI |
| | | SVIPGTLTIGDIAVKYLYEHVDANGETQVDATETGTATHATDATAADYLTYTTVDKPKTG |
| | | YALAPNTGLAYNGTLTDQGGTVTYLYLAKTETAIVTYFDQTDNKVIKTETLQGAYGTTDA |
| | | YRTADTIAAYENAGYDLVIDDYPTAGVVYDQDGSIQKYQVTLDHKFVTRTPDNPGTPGEP |
| | | IDPDNPNGPTYPVGTDFEDLTEQVSRTIQYLYKDGRTAKPDNVQAVNFSRNVTVDEVNGA |
| | | VVYTDWLTDDGAVTGCFEAVDSPVITGYTADSTSVAGRDTVSGTDLSPDVQVYYQANPEK |
| | | ATVTYEDTTTGVVLTTDWLTGDYQTVSNYRTAERIAQYIKAGYELDVDGYPAAGVVYDQD |
| | | GIVQAYTVTLKHKFITVTPDNPGVAGDPINPDNPDGPKYPNGTAAKDLSKKVSRTIRYQF |
| | | ENGELAGMDNVQTISFSRNVTIDVVAGTKVYTDWLNDSSLTGSYKAVDSPMIAGYTADIL |
| | | RVAGNTSVLGTDQDNDIVVTYTASSKEATVTYVDTTTGAVLATVSLSGTPDTPSDYRTAT |
| | | TIAAYVKQGYELVSDDYPTSGAPFSEGGVNYTVRLAHATDTTPETKTITQTVHYQASNGT |
| | | PLHTDTISTITFTRTKVVDHVTGTVVYSGWVTSKDDNTFVSVPAIAISGYHPSVTGTQAV |
| | | TVTPDSADDVQTIDYVADTVTIKTPDQPLKVKKSQKKQKKVVQVKQLKKIKQPVQMAGAT |
| | | AAALELGKTIRPIKQAAKNKQAVENKQVTTREQATTQKRATLPQTNDNRQASVTAEILGL |
| | | IVAALLAGLSAMLKRRHEG |
| P26 | Lactobacillus | Cell surface protein OS = Levilactobacillus brevis (strain ATCC 367/ |
| Q03P66 | brevis | BCRC 12310/CIP 105137/JCM 1170/LMG 11437/NCIMB 947/NCTC 947) |
| (SEQ ID | ATCC367 | MRNRLNKMGLEGKTHYKLYKSGRNWIAAGITVFSVGMGLAFSQTDQVQAATNTSADGVEN |
| NO: 26) | | SATVSSSSPTETKNTVVLNASSAAATSTAASKDDAAAATSVATAGDSQSTVTSAASASRA |
| | | VSGAAMEATASDSAATQPTASSADSQSAQSVYESAASGTTSQTAASQESQQVADNAASDA |
| | | ASSATTATNTSPLPKIKMSRAMNATALASEAEAKPTVSLVTTGTVSMNYGDASLADLESY |
| | | ISSPDETPVNDIAYYIQDAAGNYLEDVNGNKVNLLYALFLDSTEVNDYVDIVYTDEHGQV |
| | | TKYSGDVDLSTLTQIGSYTVTINDAGKAAMNRVMQDYNAYDTLTSDLNGFIPTESTGAAD |
| | | YSFTVNIVPIKITATTGMNGLNMLRLSQSYTGSLTMLPVVTIKNSQKRNILQINNGEISD |
| | | AQLGVAGKVGQRILTLADFTYTYQGTETNFTGADAGQYTITLNDAGRKAVQAALGSNYIL |
| | | DDAATFTTTGTVKAADLGLTVASDTVTYNGQAQGTSVAVTSGTAYDHFDFTTTTGKNVGT |
| | | YNDLTYALTDSTQAAILAKNYNVTTTDGTLVITPAELTVTVNDDHVVYNGQAQKTTATVT |
| | | SGTNYDDLAFTAVAADGSGASAYTKVGTYAMTGTTAADTSNYKVSYVNGTLTIDPAKATI |
| | | TIPNQVYWADGTQKSLSAVVTGTVNGETLKYRVTDGMSAVGTKTITATPDANDSVNKNYT |
| | | ISVVPGTLTIGDITVKYLYEHVDADGQTQIDATEIGTAAHATDATATDYLTYTTAAKPKT |
| | | GYALAPNTGLAYNGTLTDQGGTVTYLYLAKNATATVTYIDTTTGSVLHTKNLTGMLDTQS |
| | | SYQTADTIANYVKKGYVLVSDDYPTSGAIFSEDSANYTVRLAHATDVTAETKTVTQTVHY |
| | | QDSTGKPLHADTVNTITFTRTKVADQVTGEVTYSDWSSSKGGNTFDVVSVPNVSGYRPDT |
| | | TKIQAVMVTPASADDVQTVTYSVAESGTGYDVVNPKVPGDPIAEPEPYVPFAGTKKVKAG |
| | | DTGKLVNKQKVVKAGAAVQTAGKQTVKLSATKSVKPVKTQVDANRVNLTETKRLPQTGEA |
| | | QSHTETAGLIGLGLATLLAGLGLGCNRRKED |
| P27 | Lactobacillus | Cell surface protein, CscC family OS = Lactiplantibacillus plantarum |
| F9USJ2 | plantarum | (strain ATCC BAA-793/NCIMB 8826/WCFS1) |
| (SEQ ID | WCFS1 | MQHRQLWYRGGLGLALALVVVGYRGSRTVIRAVPRAQLSVDQKMPSTSSVFSASKLTLQD |
| NO: 27) | | EANNSAPQVSPEAQESSGPDKQSDLTSGSSTSSSGISSGNSSGSTILENAKNNQTSETAT |
| | | TKAAEMVNGTVKMTLDTNGTLHLSGGSFGASLGSATGSWIVKTLTANGYQPTQVSKIVID |
| | | GKITATTMTNYSYLFANLPNVTAIDGLANLNLTGVTDISWLFLNCSQLGALDLNSWDVSS |
| | | VIRMEGTFQNCAKLVTLNVANWNTDSLQYLIDTFNGDSSLTSLPVGKWNTSKVATMMRTF |
| | | TDCSSLTSLDIANWDTRVVTNMSAIFRGMSKVKSLPIDKWQTGRVVNMQLVFSGDTSLES |
| | | INVANWDTSRATALDGTFAKLPNIKSLPLDNWNTSNVQTIRSTFYGDTNLTQLPIDNWNV |
| | | GKVFDFNSTFSGCASLTTAPVANWNTQSATNLGYTFEGMTSLTSLPVDNWQTGTVTNMAG |
| | | TFSGVSQLKSLPISKWNTKNVQNMAGTFSKMSSVTALPVDNWQTGNVTTMRGIFTKVSQV |
| | | KNLPVGKWNTAKVVDMGQVFYGNPQLTSLPIENWNTSSATDFSQLFAEDSGLQTLSLGAW |
| | | NTTKVTNFESVFQNTSLDKLDLTGWNTNSAQTYTNAFSSKLPPKRLLLGPSFNFFKSESW |
| | | HLPNPSSEAPYIGKWRSLNNKKVYTSADLMTKYDGKTIVGEFEWATGNTITVKYVDAAGK |
| | | YLAPDTKISGATGDAYHIKPIEIQGYVPDQPDGVQGNFTDKDETITLMYNPGGLMFVSAP |
| | | QTINFGQNPITGKSENYGASYDTGLVIQDGRSIGSTWSLNATLSASGFTSKQSARPLAAV |
| | | LSYKDQQTGGGSILTPGVARLIVNNHQTVSNQGVNILGQKTALGALSLQVPTDRALTDTY |
| | | QATVTWTLNQGVPNR |
| P28 | Lactobacillus | Uncharacterized protein OS = Lactiplantibacillus plantarum (strain |
| F9UN47 | plantarum | ATCC BAA-793/NCIMB 8826/WCFS1) |
| (SEQ ID | WCFS1 | MSFLDRLKGMLQALNSTEAATSATEAPRSIAAQTAAAPTVNQTEALVLVHHLDQDGNELQ |
| NO: 28) | | AADMIAGTIGEEIHLPAVSITGYHLVHIEGLTRWFTTPQASITLTYERQAGQPVWMYAYD |
| | | IDRRELIGRPTMYRGKLGTPYEVSAPTVAGFKLLRSVGDVTGEYTTTSKTVLFFYRNQNW |
| | | QQTDLSTGFVQVNKLTAVYPYPGATTTNYLTKLQPGSTYKTYMRVRLVTHETWYAIGDDQ |
| | | WIPETHLQLTTGDTLLLKLPAGYRVQNKRPVRQTGVVSFVPGKQVHTYIEPYGRYLTTVT |
| | | HGDTVNLIERMADDNGVVWYRLQDQGYLPGRYLTKLDPPFA |
| P29 | Lactobacillus | Cell surface protein, LPXTG-motif (SEQ ID NO: 67) cell wall anchor |
| F9UT05 | plantarum | OS = Lactiplantibacillus plantarum (strain ATCC BAA-793/NCIMB 8826/ |
| (SEQ ID | WCFS1 | WCFS1) |
| NO: 29) | | MRLIDFKTWIMGTAAMLTLIVTNQTVSAADTATTATETTQTSGSSTLANQVVLRQTTSSS |
| | | SSSSSSSSSSSSSSSSSSSSSSTKASATGAATETATSKAVTTSESSTQSSSTTATSQTTS |
| | | GVTAAQATTDSTDTTATSRATANAKADQRAASAKANNEQATTQNQQQTTNMYSGVVTSQK |
| | | DSARTATTTDQATASVATLSRMSRASLRSLAQRATVAVQGLDATDATVTDDDGVTYSATD |
| | | VLSLYANYIAKYHWSIADDVSVTAGSTATVTLPENVVFTNGTQHIDVQKSDGTVVGTFTA |
| | | ETGSQTGTLTENDYYATSDRYNRQGDLTFYVTGTSATTGSSTTGINKVGWADSNSLDADG |
| | | NPTKMIWQVVANINSEKWQQVAIVDQLGLYQTHEGTMTLETGHYTDGAFVKDAALGTYGF |
| | | ATQQFTYADGVSTPQVTVTVVGQQMTINIDQLDVAVNIFYEVGLTVGHTYTNNAGVTYAP |
| | | VIGDATDPNEGSSTGEPKSEQSNVAVRFGGSGTASDDIQSYSLVINKTDGDGQSVAGATY |
| | | QLEDSTGTVLRTDLVTDSVGQLRIGNLSAGTYMLVETAAPSGYQIDTAKHVFTVSAAQAT |
| | | ANVVTGSVVDKRIAKTALTVNKVWADVPAGVQTPTVEVTLQRNGQAYQTLQLTSANGYTG |
| | | TFSDLDVTDVYGNAYTYTVIETAIAGYISSQTTSGETVTLTNTYQTGKLTVIKTDSSGAN |
| | | RLAGAVFAVKNAAGTLVAQLTTDATGQAQLTGLTQGAYTVSEIQAPDGYLINTQAQVLVL |
| | | NEQSAYQGQLVFADEVEPSEPSEPSEPSEPSEPSEPSEPSEPSEPSEPSEPSEPSEPSEP |
| | | SEPSEPSEPSEPSEPSEPSEPSEPSEPSEPSEPSEPSEPSEPSEPSEPSEPSEPSEPSEP |
| | | SEPSEPVLPGHADEDSDSDQVVTTKTETAKLVKQTNLVTTTRPTKLLGQPIKLVATSKPV |
| | | VKVTKATNRKSAQQLPQTSEQSMDWLMILGWFLLGLTVVSRQRREN |
| P30 | Lactobacillus | Cell surface adherence protein, collagen-binding domain, LPXTG-motif |
| F9UR90 | plantarum | (SEQ ID NO: 67) cell wall anchor OS = Lactiplantibacillus plantarum |
| (SEQ ID | WCFS1 | (strain ATCC BAA-793/NCIMB 8826/WCFS1) |
| NO: 30) | | MRRKLVGYMLSMLTVILALFMLGSTAHAKEISVTGLTAGNAIVLDANGKPVTDTSTLNDK |
| | | AGYQLTYHWSIPDSEVIKAGDTATVEIPTYVSIDHDVVMPLTDSAGQTLGTFTYTKGAST |
| | | GTITFTDALGTLNSRAGTLSMNAKGNATATEGSAEIAKSGLVVSSESDGAPTVLGWHITV |
| | | TPGNNSTVVVTDTLGPNQTFIPDSVAAQAVQIINGIQVPQQPLTPTVATNGNVITETENN |
| | | IHSPFVITYNTKVENFNPADTAKWHNTAALDGLGVDATADITYGGNGTAGMTYTIELTKH |
| | | DAATKAVLAGAVYELQDSTGKVIQTGLTTDSQGQLIVKNLRAGDYQFVETKAPLGYELNT |
| | | TPVKFTLGGIKPEVAFQVSQDDVKQPVVPTTGDVTLTKTDATTKAALAGAVYELQDATGK |
| | | VLKMGLTTDTTGQLTVSGLTAGNYQFVETKAPSGYQLNAAPLSFTIKPNQTAVVTVAATD |
| | | EPVTEPGTTEPSKPGEPGTTEPSKPGEPGTTEPSKPGEPGTTEPSKPGEPGTTEPSKPGE |
| | | PGTTEPSQPGEPGTTEPSKPDEPGTTEPSQPGKPGKPGEPGTTEPGNPGTTGPTAPQPER |
| | | PAVPGPSQPAAPKPGQSGLGQPALPGLIKQPSTGVNGAGGTVGNGVTTGMNGFGTPTGSD |
| | | QSTSAGYNHGTLPQTSEKQSPIWVIFAGLIGLLIAAVGIGYRRRA |
| P31 | Lactobacillus | Cell surface protein, LPXTG-motif (SEQ ID NO: 67) cell wall anchor |
| F9UNI8 | plantarum | OS = Lactiplantibacillus plantarum |
| (SEQ ID | WCFS1 | (strain ATCC BAA-793/NCIMB 8826/WCFS1) |
| NO: 31) | | MIKPRVLTTLLVCSAILTTTVTPAVAAVtPMATPSEQVAEPVASPAVPTAILSLAIQNQQ |
| | | LVDLIGQTQWQTYGQPAVTKDPEFNDQVLNLDGKSAFYTTFTDQQFAKLQNGMAIEAYFK |
| | | YDPAADANGEHEIFSSQQGGGLGLGVQNNQVVFFAHDGSGYKTPKGTLHKGQWVHAVGVI |
| | | DKNKTASLYLDGQLVQQVAMPGDLKLAQGTKDFVLGGDAVPGSHVQSMMTGQIRQARLYD |
| | | QTLTSQQVSQLNVEAQVGKQPVAPVPVDQTIATKLVGPKRIASGHTYGLNVHARQIKATG |
| | | AAPITMDVVYDAAKFDYVGAERLLQGGKTQIQLIAPGRIRLTTTANLSKAEFKMYAQTRL |
| | | AHLNLKAKAAGETQIKFEQLTKDTTIELGPAQTVEIQGKYALDYNGDGIIGVGDVALANA |
| | | ADKVAAAKAAEIKPYKHVVVLTTDGGGNPWDPKGMYYAQGAEQGTKTPVWTTNPEIMKKR |
| | | RNTYTMDLFNKQFAMSTSARAVSPAISAQNYISMLHGRPWDTLPKEYQGTNATMGQEYFA |
| | | DFNKPQAMFPSVFKMLQADNPTRGAAAFSEWGPIVNSIIEPDAAVTTKQSASLKSFDDVA |
| | | NYIGTPEFQSTGLVYMQSDYMDGQGHGHGWYNDNYWDKYAQYDALFKRVMDKLEATGHIH |
| | | DTLVIANADHGGSGKNHGGWDEYNRSIFMALGGETVDNGRRLHGGSNADISALILNALQV |
| | | PQTPQMFDSQVFDSLAFLKQTDLSKKKRSVETLKLSRNDQEAKVQLTHNQNRQLTAFDLQ |
| | | LDLAGREVADVKVPTGVQILRQTVANGQLRLTVSASQPVTDLVTIELVPSKTRAAKTIML |
| | | SQAMAATADGTEVLVDLDNDNPLTSTAKPDENGSTTTKPDGNGTAVKPDENGSTTTKPDG |
| | | NGTAVKPDENGSTTTKPDGNGTAVKPDENGSNTTKPGGNGTTVKPDKNGSSTTKPNGNGT |
| | | AVKPDKHETSTTGSGTVNTSGADKTSTNDNGTSMTAGTASSHASTVTDRVTSGTVLPETS |
| | | SSAATNHGSHSTGHHGSGWLPQTGEAVQRWLAVAGGVFLMLTGAIAVWWRKRRA |
| P32 | Lactobacillus | Cell surface protein, LPXTG-motif (SEQ ID NO: 67) cell wall anchor |
| F9USD0 | plantarum | OS = Lactiplantibacillus plantarum (strain ATCC BAA-793/NCIMB 8826/ |
| (SEQ ID | WCFS1 | WCFS1) |
| NO: 32) | | MIKPRVLTTLLVCSAILTTTVTPAVAAVTPMATPSEQVAEPVASPAVPTAILSLAIQNQQ |
| | | LVDLIGQTQWQTYGQPAVTKDPEFNDQVLNLDGKSAFYTTFTDQQFAKLQNGMAIEAYFK |
| | | YDPAADANGEHEIFSSQQGGGLGLGVQNNQVVFFAHDGSGYKTPKGTLHKGQWVHAVGVI |
| | | DKNKTASLYLDGQLVQQVAMPGDLKLAQGTKDFVLGGDAVPGSHVQSMMTGQIRQARLYD |
| | | QTLTSQQVSQLNVEAQVGKQPVAPVPVDQTIATKLVGPKRIASGHTYGLNVHARQIKATG |
| | | AAPITMDVVYDAAKFDYVGAERLLQGGKTQIQLIAPGRIRLTTTANLSKAEFKMYAQTRL |
| | | AHLNLKAKAAGETQIKFEQLTKDTTIELGPAQTVEIQGKYALDYNGDGIIGVGDVALANA |
| | | ADKVAAAKAAEIKPYKHVVVLTTDGGGNPWDPKGMYYAQGAEQGTKTPVWTTNPEIMKKR |
| | | RNTYTMDLFNKQFAMSTSARAVSPAISAQNYISMLHGRPWDTLPKEYQGTNATMGQEYFA |
| | | DFNKPQAMFPSVFKMLQADNPTRGAAAFSEWGPIVNSIIEPDAAVTTKQSASLKSFDDVA |
| | | NYIGTPEFQSTGLVYMQSDYMDGQGHGHGWYNDNYWDKYAQYDALFKRVMDKLEATGHIH |
| | | DTLVIANADHGGSGKNHGGWDEYNRSIFMALGGETVDNGRRLHGGSNADISALILNALQV |
| | | PQTPQMFDSQVFDSLAFLKQTDLSKKKRSVETLKLSRNDQEAKVQLTHNQNRQLTAFDLQ |
| | | LDLAGREVADVKVPTGVQILRQTVANGQLRLTVSASQPVTDLVTIELVPSKTRAAKTIML |
| | | SQAMAATADGTEVLVDLDNDNPLTSTAKPDENGSTTTKPDGNGTAVKPDENGSTTTKPDG |
| | | NGTAVKPDENGSTTTKPDGNGTAVKPDENGSNTTKPGGNGTTVKPDKNGSSTTKPNGNGT |
| | | AVKPDKHETSTTGSGTVNTSGADKTSTNDNGTSMTAGTASSHASTVTDRVTSGTVLPETS |
| | | SSAATNHGSHSTGHHGSGWLPQTGEAVQRWLAVAGGVFLMLTGAIAVWWRKRRA |
| P33 | Lactobacillus | Cell surface protein, LPXTG-motif (SEQ ID NO: 67) cell wall anchor |
| F9URR1 | plantarum | OS = Lactiplantibacillus plantarum (strain ATCC BAA-793/NCIMB 8826/ |
| (SEQ ID | WCFS1 | WCFS1) |
| NO:33) | | MEQVKKRYKMYKSGKMWLFAGITLVTLNMNVVTGRADESTHVEALTEPAVATLSEGNAEQ |
| | | QSPVTDAMDESAMSELVTEAQPIKVQAAEEQYTDEIVNQSDDEHANSDQVSVPVTDQVDS |
| | | ETPVPSDEHTATLDTHPNQSTTDDSEQPVSADEQSQDIDTDSTAKVLSSQHKTETINERG |
| | | SGDLAGVIRNPERPHLTDGYRNDDMEDDDSMAGIWGAGYNADGIKWHFDADSGVLVLDGG |
| | | DIYDCYGDSPWQSKSWVLQIVKVVISKPIRIIGDSGGFFENLTNVEHYEGLEKIDVSSAT |
| | | DLRYFFSENTHVKELDLSSWQVGNVTDMSYLFFNSPGTSQLTTINISGWDTRRVSEADYM |
| | | FGPNEKLTRIIGIENLNFESLKEAGGLFIKTGLSELDLSKWKTDSLDNMAAWFMDMHNLT |
| | | SVKFGSQFKTDQVTWIHLLFSGCSNLTEVDLSGENLHRVEQNLDMFAGCERLQKITLGPD |
| | | TDLTPAKIESVGLMDIEANDQYTGYWINVANPQQRLTSAELMNLYSEKNTPIGTYIWEAN |
| | | QAVIDANDITLEVGDDWNWTDSIESLTDQFGQKVDVQALYVANPQAVKLSGDRVNTSQPG |
| | | TYQVTFKYAGKTVTALVIVKADQTSLTVHDTELHAGGTWHAQDGFDGATDKDGHAIDEND |
| | | VTITGEVNTMVPGDYQITYTYGSQTQTITVTVKENQASLNLYQNHATVHTDGQGTSTWQP |
| | | QSNFQNATDSDGQTLDWSAIEVVGTPDWTTAGDYRLTYQFTDKTGQLVTATMTVTVVIEE |
| | | ADEQAESQSDLQIHDSTITVGESWQPSDNLVLATDVNGGELSLADLVVTGTVDTNQAGVY |
| | | QVTYQYTDASGQVFTRVATVTVVAASDGDTNTEQPGATNTNDDVNGGSTGSIDGDDQAEI |
| | | PTDDADQMEGDAADVDANAVIDDATPAVGTNHGKGADRNSGMQTTANGAKSVVTSWTHRS |
| | | QMTNTASLQHAQTIVGGHHQESRPTESASVAVQPVTAKLGTSALPQTGEAPSRANVMGTV |
| | | LLGLTMFGSWLGFRRVKRH |
| P34 | Lactobacillus | Cell surface protein, LPXTG-motif (SEQ ID NO: 67) cell wall anchor |
| F9URR2 | plantarum | OS = Lactiplantibacillus plantarum (strain ATCC BAA-793/NCIMB 8826/ |
| (SEQ ID | WCFS1 | WCFS1) |
| NO: 34) | | MRLIVRSVRLFLKKWGITINYRESEVKCYKMYKSGKMWLLASASLLLLNTQLLTAHADEP |
| | | TSASTSETSVVATNGVSIQNQGSSNQTLASSVSKTDNVVVANDENASITNQTVIDAQPAT |
| | | NDEPQSAASTAALNGTSGAPNSEVAADSMAAVNGLNTVAPATNSYEASRTDDLESNAAES |
| | | TVSEQQPEASEQLLLDTADASERKPAADLQHVEQHQLVDDLKVESQHVDTRAVTRADEDE |
| | | MSGNFGVDWHFDASTGTLTLNGGTLNNSYGDNPWRRKSWAPMIKCIVIADKIVAGTNMNS |
| | | LFANLDSVTRYEGLEKIDTSAVTNMQSLFKENTSLERLDLSAWQVGNVTTMVNMFMGNFM |
| | | GTELKYLNLSGWDTHNVANMQNMFQFNGQLRTIDGLTDWDTRSVTTMANMFARTGVRHLN |
| | | LTSFDSASLVEIDGAFAQMSDLERIEFGTQFTVAKVTQINSLFNDDAKLKVLDLSHENMQ |
| | | NIEQNWQMLAGLTSLQTLTLGPGLDFSQHGTQPLVDLPEVPKNSKYTGKWVNVADSSQTF |
| | | TSAELLAQYSGNHANTATFVWETVSAAVITGKDSTLFLNQKWDWTQNIAQLVDQNGQLVD |
| | | PGVLFNTDPQAVTVSGEPVDTSQPGSYHVILTYAGRQTTVVVTVVANQSQLNLHAQEVAV |
| | | EIDLATGSAVWRPRDNFASATDADGRSVEWQNVTVLGEPDLTRPGTYEVVYQFTDLTGQL |
| | | VTATTTVTVTEQEADVEDLTELVVQDTTVTVGDHWQAADNFVSASDATGRLLTLADLVVI |
| | | GDVDTTQPGTYEITYQYTNANGLQWTQTATITVVEGAGNGETPLPGEPAEPELPEEPGTP |
| | | EQPETPETPETPETPETPETPETPETPETPETPETPETPETPETPGEPSAPGTPDQPELP |
| | | EVPEQSEQPGTTEHPDTSDPNSGLTGANAGSSSQREQADTIVRPEFNGGLEKQVTTVERD |
| | | NLKLNTAERNEDGIDAKRYAKADTAKPEVTMAPVSHPASVAGELPQTSEQVNRFGLLGLM |
| | | MLMVTGLASIVGIKRRQG |
| P35 | Lactobacillus | Cell surface hydrolase, LPXTG-motif (SEQ ID NO: 67) cell wall anchor |
| F9UMT1 | plantarum | OS = Lactiplantibacillus plantarum (strain ATCC BAA-793/NCIMB 8826/ |
| (SEQ ID NO: | WCFS1 | WCFS1) |
| 35±) | | MKRNSQQSTTVDHYKMFKDGKHWVYAGITIAGLGSTLMLTTNALAATATPVSATTTSAAN |
| | | APASVASQLSQAAGATATESTTTSSMTTGEDSNTTSNTDSSATTDTNQITTSTNATETSA |
| | | TEQATSAASATDQASEVANSASGTVTSQTTSATNSTAANTISGNEQAASSATSDATQVTD |
| | | MVTATTKSTTDSAIDSTDDTSTNTNSTAAATPTSVATTSAASAATSDSGHGLIYETNDTT |
| | | GNQKSTVTITQSGPYSVTWKKVTTSDKTDTTTVTLDASDIVAVVNTIKDLANQAATPSGK |
| | | EQLAAAKAKLTTILDELKELPTDIASTIVGNVLYPIVFTGTGSEALSNLRTEMNQHRYDI |
| | | SNTWTGLDPVAYAADRAAAEEYYPTTVTWWDNVTKETWTLPEYNDPTQSVRAYYIQNGDS |
| | | TKTVIIGQGWTEHVDWIGYVSKIWYDMGYNVLMPSQRGQFLSDGDNLTFGYQDKYDWLNW |
| | | VKMVDERNGADSQVVFYGQSLGADTVLEAASVPGLSKSVKAVVSDAGYATLPELGSSLYN |
| | | KAITAVSNALQSIGLPAITSLPFLSYDKIVAAMNARLIKEQGFSVDDLSATDAASKITIP |
| | | LLLIHTQDDAFIPYTQSLELAAANHSANQEVWILPGTVGGHAAANNAILQYRQHLLAFLT |
| | | PLLSVADAEDEAVDVDQVTDNRNQGAADNGTTTDSTAQDNVTDETTADEAISDHQTIVDN |
| | | TTTDTTNITSDTTPDTTNHAKPNDDSTTSYVDLNDTDNAVDNDSDTAVDATRATTTVNQT |
| | | STIDQSSVIKGQVSDSIMVSSNATTNTDWLVNHDDSGSAVTASLLQDYSDQEASVTTPAT |
| | | VSATTTNTDSADLVAVSSPASKATTELPQTDETTQSWLATLGTSLLALATGIWAQVRRRF |
| | | N |
| P36 | Lactobacillus | Cell surface protein, LPXTG-motif (SEQ ID NO: 67) cell wall anchor |
| F9US12 | plantarum | OS = Lactiplantibacillus plantarum (strain ATCC BAA-793/NCIMB 8826/ |
| (SEQ ID | WCFS1 | WCFS1) |
| NO: 36) | | MERKRTNFKMYKIGRRWAFACAVILTMGTTTLVARADDGTTATGTDTASTSSSTTKSVTA |
| | | KTQTLKTAATTEADVTNQNQPVLDTDGSNSKTAAGTVAGTKAATDTDTNATTNLDETTSA |
| | | NTETGSDTTAGSKTAKETNATTGSESTKETSTITDSATATAARTTTSSNKGATTDSTTSH |
| | | DTAATATKTTDASSKIAGTTTSDSVAQQTTTTKDQSTTTATPQTAAVALSQAVTHANDAV |
| | | ADGGNVTDDYPDLHNMLRVSSQFHIFAREAELHAHTNGNVAVQNLVGNVNFGTNIIEELL |
| | | DKDISYIQNISNIAGSSFVSAGETRSNKVIFGENIEIDISNPNRPMVNGVYIDHLLASEV |
| | | YQDKDGNVYIDFDKEFAKLEQLSASLSEASANVTYTSDSFEDMNQRVIDVTDMQPDADGH |
| | | IVINLSADVLNTSTPLTIKGLSADADGNTVIINVDTAGATNYQVNSQIKIIYDDGTERNN |
| | | KETEDFGDNHLLWNFYDSTASDKLATGVINVDRPFQGSILAPAAEIDANQNIDGNIIANK |
| | | VNVKAETHRWDLQDNVDNENDPEPVPDYEKPVHPSIDAELPDGGEGEEPEYDKPVHPSID |
| | | IEMPDDGEGEEPEYDKPVHPSIDIEMPDDGEEEEPEYDKPVHPSIDIEMPDNGEEEEEYD |
| | | KPVHPSIDVEMPDFDEIEDEEEAEDAEEEFEDDIEDEIEAGVTPDEVVDQIEEEVDNEIT |
| | | ADWVTDETATELETAFEEVQKEAVVGDQIKDEETLINLIDRAIAQAKAHHNTALVAQLQA |
| | | LRTKVASALAVAKGQALPQTDEAPSQMISLAGIALASTLVLGAAAVSRRKRQY |
| P37 | Lactobacillus | Cell surface protein, LPXTG-motif (SEQ ID NO: 67) cell wall anchor |
| F9UMC2 | plantarum | OS = Lactiplantibacillus plantarum (strain ATCC BAA-793/NCIMB 8826/ |
| (SEQ ID | WCFS1 | WCFS1) |
| NO: 37) | | MNKKLLYTSITTAALFVGTQLGVNNAQADTATDNSDTTNQTSATQGSAQTATNEKLATVK |
| | | PTSQQQYQANVQTAKGNVATAQNQVNTTQTKVATAQGQVTNQSQLVAIGQSQYDAGKAQV |
| | | DRAQQTLDANNQVLAEAENKVDAAKSQTAAAETQIPADQQQIAANKVAIANQPATEKKAQ |
| | | TAKDAAVTALTQAKTEQATAQSDADAASAVTAAKQATVDQASAAQQKAATQANQAKVAVA |
| | | SAQDAVNKNTQAINSAKTAIQNTTSQINANNQAVSTAQAKVTAAQAALAAAERPTTTTES |
| | | QNKYDAAEFPQSQLTGAETVSVAYPSNGKYVPNADKINQYMFEYINQLRALNGQPALKQT |
| | | STLQNNAIARAAAQVDGGLDHTGSSYAENLTQVYPQWFMSDQETAYNAVMGWYDESNNVE |
| | | SGSFGHRVNLIYSTGDAGVAINLAKHVAAFEVDNAGMTEAQQDKYVDLEDNAHTNAATGT |
| | | KALPAVTFNYVQTTPADPKKIAAANATLIAATASLNGLQNTGKTLATTLANQNASLQALQ |
| | | NQTSGLQATVTTKQAQVQVAATSLKAANVALTQAQGQLATAQQQQLSPVRNLKTSIAKTA |
| | | AAQVTATQAAKNLASTKTLIADLTAENARLAAVLAQGQAQVDTANEQLAAGKAQLDRKKT |
| | | DLAQFKQVLGAARVDLAVAQGDLTATKAFLARVEANKFTTTTAAAADGIAETTNVDQSTG |
| | | VTAPHATATKTVANSNGTINATSTSVDVSDGDVTTKLVAGAKQQPVAAQATALPQTDEKQ |
| | | SASLTVVGLLAAGFSLLGLTKLRKRA |
| P38 | Lactobacillus | Cell surface protein, LPXTG-motif (SEQ ID NO: 67) cell wall anchor |
| F9US93 | plantarum | OS = Lactiplantibacillus plantarum (strain ATCC BAA-793/NCIMB 8826/ |
| (SEQ ID | WCFS1 | WCFS1) |
| NO: 38) | | MKLSKRGLFWLLGLVSFAILLLFSQPLGAQAATNYHAKDYTTAASVINGPDFKHADTIQI |
| | | QYQMSFGDTTFKAGDTVTIDMPANLEPRTVGATFDVTDAETGTVIGTGVVGGDGQVVLTM |
| | | NSAIEGKTNVKIDVNLGMKYRYDDLGEQDVVEDTQDGQDTSVINMVANEANMSKKGTIDK |
| | | ENGTIKWTLLVDRREITMKNLSIADTIGDHQQMIKGIEVYNGEWSSANTYKRRDKLSDDA |
| | | YQVNYSDNGFDLKENDTVSNLVVIDYYTKITDTELIDQNYHFKNKAVMEWGGGTSGGKNS |
| | | EEANGKVYEKVVNGGSGTGDLSSSSSSNSSSSNNSSDVDSSSDDSNSESSSAVDSSSDDS |
| | | SSESSSAVDSSSDHSSSESSSAVDSSSDDSSSESSSAVDSSSDHSSSESSSAVDSSSDHS |
| | | SSESSSVVDSSSDHSSSESSSAVDSSSDHSGSESSSDVNTSSESSDNTTTEPDNGHQTGD |
| | | IEDPEDNTAVYPDIDEDTGTIDVDGGFDSNYDGSTTSNSTNSSKPLKDSTSSVFTSTPAN |
| | | TTTGQDGVDQTPAADTKKSSAKTTVSESDALTPSTPNQVAKLPQTNEAKMDSQALRSVGI |
| | | LLGVLTLGGGALIRHWF |
| P39 | Lactobacillus | Cell surface adherence protein, collagen-binding domain, |
| F9UR97 | plantarum | LPXTG-motif (SEQ ID NO: 67) cell wall anchor OS = |
| (SEQ ID | WCFS1 | Lactiplantibacillus plantarum (strain ATCC BAA-793/ |
| NO: 39) | | NCIMB 8826/WCFS1) |
| | | MRKKWRWLLLALTGIFFLMFGPPLVSQARNVIEATGNDVNSAVIKDSKGKIMAHDAQLPE |
| | | DQEYTVNYNWRIPDNLKIKAGDTMAFQVPENVRIPHDEAFPMKGTTAGTIGTFFIAAGAH |
| | | TGLVTFNQAYQTRPRNRKGFVQLDAFGTVPSHPGNLAPILLEKSAEWADEANPRRINWTI |
| | | RVLPNNNQLVDPTFVDTLSPNQTYVNGSAVLRDETGNIIPVNTSVNGNQLTFNATGSFTS |
| | | ELALTYQTKTNEPTGDATFENNVTYTDKNGNKGSATATISRPVTEPDVPENPGISEPTDP |
| | | DEDEEPGVTEPEKPGTTEPEKPGVTEPEKPGTTEPEKPGVTEPEKPGTTEPEKPGVTEPE |
| | | KPGTTEPEKPGVTEPEKPGTTEPEKPGVTEPEKPGTTEPEKPGVTEPEKPGTTEPEKPGV |
| | | TEPEKPGTTEPEKPGITEPEKPGTVSPEQPSGPKPTNPGTVTPEKPTAVTPAVPNESSPS |
| | | TPEPSVSGNLSAPANPATNSTNTTATTVPATNPLPASAATAFAGSAPMNKSLPQTNEHSA |
| | | SWSVAIGLALLIGLLGSAFVLTRRTKHRHS |
| P40 | Lactobacillus | Mannose-specific adhesin, LPXTG-motif (SEQ ID NO: 67) cell wall |
| F9UN23 | plantarum | anchor OS = Lactiplantibacillus plantarum (strain ATCC BAA-793/ |
| (SEQ ID | WCFS1 | NCIMB 8826/WCFS1) |
| NO:40) | | MLKKDNFGEHKTHYKLYKCGKNWAIMGITLVSLGVGTVTMTRAAAADSEVINDSASQHVT |
| | | SISTDASKNQHTSSNVILTNDDKSVSASINQDASASVVNKAVSATSQENSSVQNTSQATS |
| | | TSKQESSSTKNTSQTTSTSNQEANSAKSINQTTRTSKQESSSTKNTSQTTSTSNQEANSA |
| | | KSINQTTRTSNQESSSAKNTSQTTSTSSRKINSTKSQAQSLTITTTGKAVRATSTSVKKY |
| | | STKTKVSYSTLLQQLRTSKALISDEAALTHVDKDNFLKYFSLNGSATYDAKTGIVTITPN |
| | | QNNQVGNFSLTSKIDMNKSFTLTGQVNLGSNPNGADGIGFAFHSGNTTDVGNAGGNLGIG |
| | | GLQDAIGFKLDTWFNSYQAPSSDKNGSEISSTNSNGFGWNGDSANAPYGTFVKTSNQEIS |
| | | TANGSKVQRWWAQDTGESQALSKADIDGNFHDFVVNYDGATRTLTVSYTQASGKVLTWKT |
| | | TVDSSYQAMAMVVSASTGAAKNLQQFKLTSFDFQEAATVNVKYVDTTGHQLAQGTANYPD |
| | | GAYVNGRYTTKQLIIPNYRFIKMDDGSVTGTKSLDANGTLIQSGDNGTVIYVYVPEYMAI |
| | | VKTVNETINYVDENGHALTTSYTANPIHILTVTNPVDGTTTTYYSTITTSIELDATTGRP |
| | | VDSGWVLGNSQDFDAVTNPQIKGYTVTSTDAPNSDLQHVSAQTVTGDSGDLEFTVVYTKN |
| | | APIVTTESKTVNETIHYVYTDGTTAHDDYVAQPITFTRTVFTDAVTGEKTYGGWSAAQQF |
| | | AAVDSPAIKGYTPDQSKISTQTVTGDSSDLEFTIVYTKNAPTVTTESKTVNETIHYVYTD |
| | | GTIAHDDYVAQPITFTRTVSTDAVTGEKTYGGWSAAQQFAAVDSPAIKGYTPDQSKISTQ |
| | | TVTGDSSDLEFTVVYKADSTSTKPVKPEQPTIPTTPTEPVKPGQLTTPAKPDQPMTSDKS |
| | | VQTITIKFVGQRLPQTNETDQQHMTLSGLLLLAMSGLLGLLGMAKRQHKE |
| P41 | Lactobacillus | Cell surface protein, LPXTG-motif (SEQ ID NO: 67) cell wall |
| F9US24 | plantarum | anchor OS = Lactiplantibacillus plantarum (strain ATCC BAA-793/ |
| (SEQ ID | WCFS1 | NCIMB 8826/WCFS1) |
| NO:41) | | MSKALKIVMGITMLTGGIMAQKMTVHAAESNTRTGQAVRMNGTVSLASQVENNPAVKAAH |
| | | YQVTQAVQALTMATTAVKTAMSDLQAAQTTLDAANKTLAKNQKIQTHMGVLKQAATDRHV |
| | | KATKALDEQLATKKTSQTAVTTAQAAVTKSQAAVQVAQSNFDKDNSAANKVTLQTTQAKL |
| | | KTVQETLTAAQANLDKTNEHVMMAEEELANAKIEVSGTSRDFQMAQRDYDIVQPQAAVNQ |
| | | AKAAVTAKLQRVAGTQDQVVTAQRELSQAQAGLTTVRARTLATLTAAAEKPMTEKPVGER |
| | | PVVSHSTGTSTSTNQSAAPQATPAKPTLNQSSSASVPTAQRVVTTQPRQATTVLRTTTSP |
| | | AMAKPVTQQTVPTTATKTATLPQTGEQTNRVLTVLGFVLLAATSLEGESKQQRRHKTTD |
| P42 | Lactobacillus | Cell surface protein, LPXTG-motif (SEQ ID NO: 67) cell wall anchor |
| F9UM21 | plantarum | OS = Lactiplantibacillus plantarum (strain ATCC BAA-793/NCIMB 8826/ |
| (SEQ ID | WCFS1 | WCFS1) |
| NO:42) | | MNRFITSKQHYKMYKKGRFWVFAGITVATFTLNPLISRADTETTTAATAATTTAGASSSS |
| | | NSQVLRTTTTSTTGATTQSSATAINAATTNTSAQKKQAVSGTTTDSKAEQPVTAVGENEN |
| | | ATSNLSTSDSASASSQAKTGSGNSLDQTSNSSVSVASSSQKVTTQNSDYQNDQGTGSESG |
| | | IQSNVTDTVVADESLQTNRSSVASPSTSTMASIGDSDSKDSNETEKVVDSETSPIVVTAT |
| | | TNTITTTNDKVQLNRALLARAAIPAIVQSGTLGTSQWTMNSDGVVTIGAGDWSNVDDVSA |
| | | LFYTLGSTVTGVVIDGKVNAGEDLSYLFFKSPNLATITGFQNIDTSKVTDFSYMFCGTSV |
| | | ADFSSISHWDVSDSENFDSMFTSNSKVQSIDLSHWELSQAQSIKMRRMFAADTALISMDL |
| | | SAWNMSMVTNINGMFAGNDLNTMALKSVDLHGWNLKNVTDMGTMFNFDNSLTSVNMSGWQ |
| | | TSSNLSSVDSMFRGTSSLASLDLSSIDLQGVTRKYMLLSQNKLYDPISSSLSTLTLGTMS |
| | | VLTDTGLPDIPTGTGYTGKWVNQADATQTYTSSELMALYNGVDSPADTITWVWETSPSYA |
| | | DFTSKNVTGLIAGPKTTWRVADSVATLKDVNGTDIYATADTVVKVISVNGDTAVTTVDTQ |
| | | TAGTYQVDLQYTDAYGKVWQQTSTVAVAVNQGKLVGKPLTIKMGAKPTYTINDLIDTDNS |
| | | RNAAGDKLSADELATATVTGLDTSKAGAQTVTLAYTDDATGMVHTTTTTVTMVATKADLT |
| | | MRNSTIIKGPKNSSWDYRQYVTSVTDFDGNPVSLDGLNIVVDQQPDLTQIGSQTVTLTYT |
| | | DALGNVISVPTQVTVVASRAQVTTKAPLTIWPSEVAQLKVADLVTITAANGNPVDTSTDL |
| | | TDVTMSSIDTSKGGAQTVTITYTDEAGNLVTAYAKVTVDQSDLKTKLTNPIAGPKAKWDY |
| | | LAGLEWVKDANGKLLDNLATADIKVVTEPDLSVAMVGHDQTVTLSYMDELGKEHLVTAVV |
| | | NTVASKAKITAVSDQIIIPDEAKKLTATDLVSELIDAAGNKATNFDDVTMSGFDAKAIGP |
| | | QTVTLTYSDAYGNQTTDSTTVTVDFATITGQATHPIAGPTATWDYRDSVTQVIDANGKII |
| | | DVGDADITATTPDLTPAKVGKPQTVTLTYTDSLGKVHTTDVIVTTTLSKAKITAVADQII |
| | | WPDQAKQLTATDLVDRLYDAEGHLITNHDNVKMSVLDSKLAGQQRLTLTYTDVAGNQSVA |
| | | YANVTVDQAKLVTKPSTVIAGPTATWSYEAGISQLTNAAGQLITVQPGTIKVLNRPDLNV |
| | | DSVGQQQLITLIYTDELGKSQSVTAMVTAEASQAMLTAKAAVIVQPDASAKLTANDLVTS |
| | | LTDASGQQVTDYQIVRMSKLDATWPGVQPVSLTYTDAAGNEVSTVVKVTVDQAKIDSQNR |
| | | TQIWGPSMTWDYRQQLATVTDSQGHQFNPDQAKITVITGPQLTAKMIDKPQTVTLMYTDD |
| | | LQQTHTVSATLTLTASQAALVPRPAQIVWAKDAGLLTPANFSQTITGADGTQVSSLTNVK |
| | | MSAVDASQPGAQTVTLTYIDDYGNEVTTTAQVTVDQAALTTQTARPVAGPTAKWDYQTNF |
| | | KTVTNAAGEVINVGDANIKVLTGPDLSTAMVGRPQVVTFSYTDELGLTQTATAKVTTVAS |
| | | RAHMTTSADQVTWPATVGKLTVADLVTGLTDAWGQTSQNYQNVTMTTINAQQAGKQQVTL |
| | | TYTDEVGNVKTATTTVTVDQAALTTQPQTVIAGPTAKWDYHQGIGTITDGMGQPIAVNNA |
| | | AITVVAMPDLTVAHIGQPQTVQLVYTDSLGQQQTALVQVTTVATQAKISTRPVTVIAGPK |
| | | TTWSLNDSVDWSTSLAADGTLLTAAQRQRVTVDGTLNLRRAGNYPLTLSYMDRAGNLITV |
| | | TTSIDVLASQAQLQVRDSQLTVGNTWAAQDNFERATDAQGQALTLADIAVDGTVNTQHAG |
| | | RYTLTYHYTDVAGNQLTKTAVVTVVLPEDDHINTADPDNNDHAGITNPSETPKPSEQPND |
| | | SDGHTVDWGVDDRITTKQQPAAATRAQTKVKMTAEPALPANNERTSATKAVTRVTDTTAD |
| | | TLPQTGERDRSAQQGAVVLGLTGLLGLMGLGRRRHTHED |
| P43 | Lactobacillus | Mucus binding protein Mub OS = Lactobacillus acidophilus (strain |
| Q5FJA7 | acidophilus | ATCC 700396/NCK56/N2/NCFM) |
| (SEQ ID | NCFM | MVSKNNRAKQMENVAERQPHFSIRKLTIGAASVLLSTTLWMSVNTSSVHAENIDNSDNDA |
| NO: 43) | | HEATESNTETPSINDDTKVVVESNSNITSSNDVNAGNNGAETNDTNNEVTASEDTSKGLT |
| | | VDNKDASVQSTVKSSDEVKKSESTEQKSAKTAQNSTLNNNTVNTEKAESNVAAKSNADTA |
| | | KSTQQSSAASSANQVSSNADLTQNQAINSTTQVEANNSTNDKKANNDTADLSNIGLKGIE |
| | | TNKIPETTDLPVSELIKSYNNNSNSNEVNVNQVSGLRAAQLFAASFIATQNTGTGNNGAV |
| | | NIDTYKPDFNLTENPAYQQYFAAIPADQYAFQSYEVVSTGQKIVVTTDRNNIGNNIRFYN |
| | | VRNGSAQLVYQMTRDTQTNASGSVVKNRPSLQGTFTTAGVASNSTYKGGTYNWSLNQTDT |
| | | VNFPGIGNLKIGRIDITAGSSNSPVDNGTGAFVTDNSHRITPTWDQGLPIEGIVSGKTWN |
| | | SAGSNIPDKVTQNIWYVDAETGKVLSHKTSDEAFNGSSYDSTDNGVKTISKDGKAYQLID |
| | | RGSDGLYDPSDFSDILNKQLATNNGLPITIGDVLSTPLKGTLRDGRIGNIKGSITNFQGT |
| | | RAYMRLQTKTDGTIDLNTYTFDPGSTRGNLNTGLSQADVAPGQTVMGAGDTSGSGAFYNG |
| | | TRPGNRDIIFLYNAEANKQNANITFVNDDTGASLSPQQNSSGDAGSQITFDNAGTTVTNL |
| | | ISQGYVYNGTTGNGVTNGSAGGSFTSVGFPAYDNDDNTNQAFVVHFKNPVQTTTYRQGTE |
| | | ESKTINRTINYYDKVTGEKIPSNLISQNPVTDSVTFTRTQVLDQDGKVVGYGTISTDGKS |
| | | FRNQDWHTAAGESSTQFDAKRSSDLSAYNYTAPEFQDGTNASIVAAHEVTPTTQDLVYNV |
| | | YYGHQTQQVTTNEDVTRRFHYIFTDGTTPESHLTPQADQKVTFTGTATKDLVTGKTGDTV |
| | | WTPSTGTLAQVAGQTVAGYHITGNVNANADGSANAVTVNPDSGDIDVTVVYTPDAKTPDT |
| | | PQKAKVTIYDKTENNKQLSNFENNNGTKGSAISFDGEPQTLQAYLNSGYVFDSATDANGN |
| | | SIGTASNITFGNFDSVDGNVQSFNIYLVHGTDTKTEKATTNAHVHYVVAGNEANKPAAPA |
| | | DSPTQTINWTRTNTTDKVTGATTEGTWTPDKNGFTSVTSPDLTNYTPDQAVANFTTPQPN |
| | | RDQVVTVVYNPNPEVAQKADLVVYDKTDNNKELNNFDNSGKTGTQISFSGSANYVADLIA |
| | | KGYKIDSFVNDQNQTSNPTSYDQISFSNFDNNSASDQHFKLYLVHDTENVTDKKTTTSTV |
| | | HYVVSDGKTNPPSDNTQTITWTRPGTKDKVTGVTTPTGNWTTPDNYTDVPTPNLDGYTPD |
| | | KTNVPAPTPDPNQNPTTVVTYNPKTPEAPTYTGTTENKTVTRTINYYDKVTGEKIPANLI |
| | | SDNPTTQNVTLSRTHVVSSTGQDMGYGTVSADGKTFTKATTVDGWNTGDWAQVTSPDLSN |
| | | AGYTAPDLAQADQVTVDANTKDAVVNVYYGHQTEVITPKTPHNPGGSINPNDPRNKPSVY |
| | | PDGLTKEALTTEVTRHINYVGVNEDGTTTPVNGSPDGKNTYTQTVSFERNAVIDKVTG?I |
| | | LGYSTDGTTNVTITDKDRAWTPTTQNMDSVASKTPSEVGYDKVDISTVGGVTVYPGQKVN |
| | | DVTVTYTKNKSPEVTQKATLEIIDNNDTNAPKQLASFSNEGKSEDQINFANSNEILQSYL |
| | | SQGYKVQKTAGNLSGDAQSGYTYPTYGNTTQDFKIYLIHDIADKTETATATAQVHYVVAD |
| | | NGVQAPADSDLQTITYTRTNRVDKVTGATVNEGTWQADKSVFTDVKSPDLSKDGYTPSLE |
| | | NVQFNAPERNVNQRVTVVYNRSAQAADLQIIDDNDPQNQRVLATYSAGGESGKQISEDGS |
| | | NTQLQTYLNNGYTFEKYEGQGMSGDAQNGFTYPSFDNDSQSNQSFKIYLKHATANKTATA |
| | | TTTAHVHYIMADGTKAPDDSAIQTINWTQTNTVDRVTGATINEGTWSSDKNAFTDVDSPT |
| | | VTGYTPGTKTVKFATPERGVNQVVNVVYTKDAPTPDRQNALVVYQDVNDPAHPVDLGQSD |
| | | QLTGQAGYSINYSTANKIDEYEKQGYVLVSNGFDANGTKPSFDNVNGNTQTFYVTFKHGI |
| | | QPVTPTTPGTPDQPINPDNPDGPKYPSGTDQTSLTKDVTRTVTYEGAGNQTPSPVTDTLH |
| | | FQGTGYLDKVTGKWTDANGKKLSDQTKGITWTITDGTKDEGSFNLVPTKHIDGYTSKVVT |
| | | NGADDGNGNVKSYTGITHTSDNINVVVQYNPIVAEQGNLIVKFHDDTDNKDLTGVGTDTG |
| | | TQDVGTQVTYNPSTDLTNLENKGYVYVSTDGNIPSSIVKGTTTVTIHVKHGTVPVTPDNP |
| | | GTPDQPINPNDPDPNGPKYPTGTDKASIDKTITRIVHYEGADQYTPNDVKQPVHFTAKGV |
| | | LDKVTGEWITPLAWSEDQTFNGVNSPKIPGYHVESVDKDTTDNQNVDSAKISHTGADYTV |
| | | TVKYAKDAAPTPDATTGKVAYIDDTTKNTLRTDSLSGNVDANIDYTTQDKISNYINMGYK |
| | | LVSNNFTDGKEIFNKDASKNSFEVHLVHDTVPVTPDNPGTPDKPINPNDPRPRSEQPKYP |
| | | TGTSETDLTKDITRTVHYSGADEYTPNDVKQPVHFTAKGVLDKVTGEWITPLTWSEDQTF |
| | | NGVNSPKIPGYHVVSVDKDADGTNVASSNVSHTGSDYTVNVVYAKDAVKQAENANLHIID |
| | | LSDNNKEIANFNDSGDDNAAINFNGAQTTVDALIKGGYKVNSIVQATSDPNNPTKYGTEY |
| | | SSAASQWMFDDKPGVDQSFYVYVEHDYAPINPENAYGRTDLTQTVTETVHYIDEATNKPV |
| | | ATDYTNTLTFKGQGRVDKVTGKMLKIKSIENGQITYDYNVANEIDISSAKLSDFAWSTPT |
| | | TLQKVTSPTIAGYTIDAAKTTPSELADGNDIKEIQNVAYDHGNVEATVYYKANPVETHKA |
| | | GLTIYANGNQVGTASVTGAKDTAINESSASDIVAAYISNGYKFDHAQDVTNNKEMTGKSY |
| | | NELNFGNFATTNNSDQQFAIYLTKDETPAKTQQNAQLTVRDVTPGQEMDLGNYTQPGLEG |
| | | DTISFSSAQEFVQNLLNKGYVWDGASYNGTNLEATNYAGINFGNYDNTDDKNGISQKWVI |
| | | NLVHGVTPVNPDHPDDKDGFTKDYLDRTITRDVTYVYEDGSQAAAPVHQEAHYQGSGYLD |
| | | NVTGKWVTVENGKITGLAQGLTWTPDQDSTFDQIGAKNIEGYHVSSVSGNGISGFTVGQD |
| | | GTVGQQTVTKDTPSSTIRVVYVKTPVTPVPANGSIVYIDDTTGNNLENATFGGTVGAKID |
| | | YTTADRISYYQGKGYKLVSNNFTDGSQTFKQGENKFEVHLTHVTETKDATKTITRDVTYV |
| | | YEDGSQADTPVQQTITFTGKTTSDKVTGSEKTTWNNESQTFGATKAIDTTKYQIVGINER |
| | | NTTANVDRDTGVVASETITPNSQNSAVVITLANKPETPIPANGSITYYDDTTGTTLESAG |
| | | FSGSVGQKINYTTADRIINYVNKGYDVVSNNFTDGNETFKQGDNKFEVHLVHATTPITPE |
| | | NPGKPGQEVPNPNDPEHPHTIPANFVPQTLTHTVTRDVTYVYADGSQASAPVHQTFTENG |
| | | NGVIDLVTGQLVTVENGKITGAGKITWNADSHNFDAIDAIDHDGYYISNVSENNTTANVD |
| | | TNTGAVAGETITPNSQNSTIIITLTKKPDVPTPVPEQGSIKVTVHDVKTNQDVPGYDKDS |
| | | GKQNTGTSFTYDKTTTITDLENKGYKVINPNVDIPTKVSNIDQHIVIYVDHNVIPVTPDK |
| | | PGNGLSENDLNKTVTETVHYVVNGGATEAPADKTTSLKFTGTAYYDSVTKKWTDANGNEL |
| | | SDQSKNVTWTAENGNKFAVVVTPTLEGYTPSVQSGYDDGNKNVKEINNITPDSGNVEVTV |
| | | TYNKNNVPTPVKQGTIEIIYHDTTDNVDIPGYGQSRIKEDEGTSFSYNPNAKDLPALESK |
| | | GYVLDGELPTIPTKFTDGDQRVVINVKHGTTTVTPDKPGKPGDPIDPNNPDGPKYPEGTG |
| | | ENNLKVTGTQTIHYIGAGDKTPKDNTQSFEFTKQITFDNVTGKIINDSGWNVTSHTFGSE |
| | | ATPVIDGYHADKTTAGGTTVTPNDLHKTVTVTYTPNVPAVPTPTPTPSPEPKPENTPVEP |
| | | NTPTPTPDIPDNVTPTPEPENNNVKPHGESIVQKNNDNPKVVSHGQSGNNWTAPHGQHVD |
| | | QRGNIVTSDNRVVGYVDQNGKAHYTKLPQTGDDQTNDVAAALLGGAAVSLGLIGLAGVKK |
| | | RRKEDK |
| P44 | Lactobacillus | Mucus binding protein OS = Lactobacillus acidophilus (strain ATCC |
| Q5FKA6 | acidophilus | 700396/NCK56/N2/NCFM) |
| (SEQ ID | NCFM | MISKNNRIKRMEATSERKQHHGIRTLSVGAVSVLLGTTLWISIPTSTVHADEINIDDNQP |
| NO:44) | | KTNLESNESASTDHVEKVIVEQNQSSSEGAQQDINAANDVSAQNDQKSVNKINDEIIKNE |
| | | NVDADIKTNTDNSHAETSYGQTESQEIIENKQKTDVEKNKTQTTDNITPVEQTGNSSENT |
| | | STNVTTQSPVDNSTNNDVNVNNSNLADTQAELIDSNTQFYESSPLIDQIGQQGKTTVNSS |
| | | NNTSSKLNIDDLSPDLSDEVLKANLTQGNQILLNQSNSSDTMAGKNADPTKQLEAMARTA |
| | | TLVAASPNADNYTTVNNYNDLQRAVSNYSVSGVNIDGDIYVFGNLTINRAFTIKGTNNAK |
| | | LNLNQNAIINNSTLTLEDITVNGSIMGNGTVNIKGDVISNVNESNGYTLTNSEKATPGVK |
| | | VNWTQTKGYNIQSSTVNVDDNASLTINRSSVGDGIHLLSNGIVNVGNYSQLTINMNTNNE |
| | | LGTGATARYHDAGIFAESNGSFTTGYKSVVTLNTSIGQGIAMTGLRPNVTDNDRFGGYTR |
| | | DRANGAGQINLGQYSTLNFTGRDGVILGNNSNFNVGEYANVHFENKGRGVALDLANNSNI |
| | | NIADHAVTYFHSVGKNTTNAIGVVVGPSGSYEGYNYIGVNEAGNITIGEDATFRVIMENR |
| | | GDNAWDDVISLDSQLATTNAAFTSKKGAIIDIRDDNTNFYAELISFPLGAANSRIDIQDP |
| | | LLLNLQRYSAGGETTGWMAGVGGVAINSTSEKYTANLIYMGGTKGVLSIGGTNYVVYQQI |
| | | KSDGAQQIWTDVDSVEFHKNGFASQDIFNNGANSDVSISGNGFTSGIRANQIRDNQTDPT |
| | | LVNLQNSPAYGISTMRASHQIWIPHETSTQIKGTHTNTISYVYEDGTPVMGADSQPLVVT |
| | | QNLNLARDLTLDLTSEQIKTIQDYALGHTADETLNYIRSGYSVTQDSGWTYTNDQGQKVT |
| | | DPYASVTSPVKEGYIITIQSTNAPGVTLGADGQTVKANFVFDAANDVVQNGQLSAGYRNQ |
| | | GITGIPDNYQTIVVYKKAEKGSVQVIFYDDTTNDAIPSVGENSGTEEAGTPVTYTTAQNI |
| | | SDLEKQGYVYVSTDGVIPTTIPNNATLITVHMKHGTNPVNPDQPTDKYTKEDLQKTVTRT |
| | | INYIDTAGNIIADSVTSTVVFTGSGTIDTVTGNLVTVDASGNIVDQNGQLTWTYSVDGDS |
| | | AQSGNSYTFAETAAKPSIDYNGSTYNFVSVTPGNYSAGNGSVTSYEVNTNNSHDLTVDVI |
| | | YNEGATYHTGKTDTKNVTRIINYLDGKTDEKIPINLILANPVEQTVSMYRTEILDSTGKV |
| | | IGYGTVSQDGKMYTLNNNWIIDGIWESVNSPDLTTNGYKAPRFEDSSLAAIVAEYIVNAD |
| | | TKNATVNVYYDHQVIPIGPDTPDKHGVDINQVEKVVKETVHYVGAGDKTPADQVQTSKWI |
| | | RTVTVDVVTNEVVPDGEFTTDWTIPSDEKSTYDQVDTPVVNGYYADQANVPATAVTQNDI |
| | | EKTITYKQIGKVIPVDPSGNQIPGIDTPHFPNDPNDPTKVIPGEKPYVPGYHPETGKPGD |
| | | AVDPAPGDPSKDVEVPYTPETPIVDQKAVVNYIDSDEENKVITSSGDLIGKPGEQIDYTT |
| | | IPTITDLTNKGYVLIYDGFPTRVTFDDDDGITQIFTVVLKHGTQTVTPEKPGIPGDPINP |
| | | NDPDGPKWSDETGKDSLIKTGTQTIHYEGAGSKTPTDNVQNFEFTRTAVIDKVTGEVIST |
| | | SGWNVTSYTFGNVDTPIVEGYHADKRNAGGTTITPDDLNKMLVVRYTPNGKIIPVDPAGN |
| | | PIPNVPTPQYPTDPTDPTKVVPDEPVPAIPGYRPSTPIVTPTDPDKDTPVPYAPIQGSIQ |
| | | VIFHDDTSNQTIPDVGYNSGVQDEGTRIDYTTNKNITDLINKGYVYVGTDGNVPAEIVAD |
| | | QNITITVHMKHGTTTITPDQPGKPGEPINPNDPNGPKWPSDTDTKGLTKQGNQTIHYVYV |
| | | DGNKAADDNVQNVTFVHTLVFDNVTGQVIDDRGWTPESHKENNVFSPTIDGHHADKIVVD |
| | | GVTVTVDNPTSETTVVYAKNGQVIREQQEVKASQIVKYVDDEGNELHKSELQEFTFTYTG |
| | | DAYDEVTGAKVQTGTWNAISTDFPVVDVPVITGYVAVSGYTNNNGKYMAGGFTTTRESSE |
| | | DQRNRVFTVLYKKVGNIVPVGPDGTTPIPDAPTPSYKNDPTNPTKVIPDEPVPKVPGYTP |
| | | NTPTVTPGDPTTDTLVPYTPGNPITDQKAVVNYIDADEGNKVIISSGNLIGKAGDKVDYN |
| | | TSDTIKNLENKGYVLVHNGFPDGVTFDNDDSTIQTYTVILKHGTTTVIPDKPGKPGEPIN |
| | | PNDPDGPKWPDTTGKDNLSKTGTQTIHYTGAGNNTPKDNVQSFTFTRTAVVDNVTGKVIS |
| | | TGAWNVTSHTFGNVDTPVVEGYHADKRTAGNTTITPEDLNKIVTVNYTANGKIIPVDPNG |
| | | KPIPNVPTPTYPTDPNDPTKVVPNEPVPTIPGYKPSVPTVTPSDPGKDTPVPYAPQTTPV |
| | | TPNIPVTPNEPSTPTTPDTSAPTPHGEDVPVTPNEPDTPAPAPHGEKPEEPDRPAPAPHA |
| | | PKAPTAKGNNTPEKEDKTVPTAAAVVKNEQTPEAELPQTGEKNDSAAAILGATAGMIGLI |
| | | GLSGVKKKKS |
| P45 | Lactobacillus | Mucus binding protein Mub OS = Lactobacillus acidophilus |
| Q5FIF3 | acidophilus | (strain ATCC 700396/NCK56/N2/NCFM) |
| (SEQ ID | NCFM | MDKKEVKNRFSFRKLSTGLATVELGSIFFWTNGQTVQADSVEPASEQAVQNVDSQVQADN |
| NO: 45) | | TVSENTVNEENGSTSETTTEVKTEMPSVDTTSQAKDAVETSDNKKVELPQGEADKQVPQK |
| | | LEVNKSNQAAETTDKDTKQNATSATPAQLNENTAPVVVKAKSEGKEVVKATDPTDYPTEV |
| | | GQIIDQDKYIYQILSLNDRSGRPSDSKLVLTTNRNDHNDKNIYAYVVDRNNRRVSQSVTV |
| | | GVDQHTIISVNGRGYQISNTGGSNVIVDGKEVPTQNTSTVTSGNGTTSPIYGLGNTTRGD |
| | | YSAIGEIPPVYTENSVIKYYYRDENGNLKEAESSDQYPNVNVSGLTGQEFVIPNVDQYKR |
| | | VIKGRYLNSDNLPTGDFTGTISQFGEGKYYKKVYYDYGTDDVDYYVVYNQVSPDGTMDVS |
| | | LFRGDNNTPIESRRVGPGRSIRFTSRNYTARNPYVTETPHEVQFIYDKLGSIVPVDEDGN |
| | | VIGDLVQFNNSTDPTKAAVTDSPVIAGYTIKDPTQREITPHDPGKNIKVVYVRNHVTAAI |
| | | KYIDDTAGDDLSAYNKSITAKPGEALNYTTKDSITELQNKGYVLVSDNFNVTTMPENGGN |
| | | YEVHVKHGTKTIDPDNPTDKYTKKDLQKTATRTINYVDDQGNKIAESVTSTVVFTGTGTV |
| | | DAVTGNLVNLHPDGSIKDQNGKLTWTYSVDGGVVQKSDTYTFSATTARPTIDHNNSTYNF |
| | | TSTTPADYNAGNGAVSSYRVNSTDPQNLIVNVVYTKQAIYHAGKTETKSVTRTINYLDGK |
| | | TGEKIPTDLIATNPVAQTVNLHRTEIIDDNGKVIGYGTISKDGKSYTINNDWVVDGKWAS |
| | | VTSPDLSAKGYKAPRFENGTSAARVDEVIVGSGTKDATVNVYYDHNLIPIGPDNFDKHGV |
| | | DRSQIEKQVKETVHYVGAGDKTPADHVQTSKWTRTITIDAVTKEVVPNGQYTTDWTIPKG |
| | | EKTEYAQVNTPVVNGYYADQANVPATTVTQNDIEKTVTYKQIGRIVPVDPNGKPIPDAPT |
| | | PQYPNDPTDPTKVLPNVPVPNIPGYKPSVPTVTPTDPGKDTQVPYTPVTPTNPDNPVIPT |
| | | PQPEPNPDNGKDKPVDPSKPSDDPVHPEYPGIKRGQDKPDKEKTDKKRNGKTKGKENTPT |
| | | GRDAVKRAGRSDDALKLASEAKNRRMTIQGKNEELPQAGEDHNAMALIGLAFATLAGSVV |
| | | FATDRKRR |

P_{nisA}/nisK/nisR Systems

An expression cassette can comprise a P_{nisA}/P_{nisA}/nisK/nisR system. Biosynthesis of nisin is encoded by a cluster of 11 genes, of which the first gene, nisA, encodes the precursor of nisin. Other genes include genes involved in the regulation of the expression of nisin genes (nisR and nisK). NisR and NisK belong to the family of bacterial two-component signal transduction systems. NisK is a histidine-protein kinase that acts as a receptor for the mature nisin molecule. Upon binding of nisin to NisK, it autophosphorylates and transfers the phosphate group to NisR, which is a response regulator that becomes activated upon phosphorylation by NisK. Activated NisR induces transcription of two out of three promoters in the nisin gene cluster: P_{nisA} and P_{nisF}. The promoter driving the expression of nisR and nisK is not affected. Since nisin induces its own expression the accumulation of small amounts of nisin in a growing culture leads to an auto-induction process.

The genes for the signal transduction system nisK and nisR can be used in an expression cassette. When a gene of interest, e.g., a biofilm assembly gene or a functional gene or a marker gene is placed downstream of the inducible promoter P_{nisA} or P_{nisF} in a vector or on the chromosome of a host cell, expression of that gene can be induced by the addition of sub-inhibitory amounts of nisin (e.g., about 0.1-10 ng/ml) to the culture medium. Depending on the presence or absence of targeting signals, protein can be expressed into the cytoplasm, into the membrane, or secreted into the medium.

A marker gene encodes a marker protein such as a fluorescent protein or an antibiotic resistance protein. A functional gene or recombinant gene is not limited in any way and encodes any protein or polypeptide that is desired to be expressed by a population of host cells.

In one embodiment, one expression cassette or vector carries both the nisR and nisK genes and a second expression cassette or vector carries the nisA promoter and the biofilm assembly gene or the functional gene. Alternatively, one expression cassette or vector carries the nisR and nisK genes, the nisA promoter, and the biofilm assembly gene or the functional gene.

In an aspect, the nisK and nisR genes are from L. lactis and are shown in GenBank: Z22813.1. In an aspect nisR is shown in UniProt Q07597. In an aspect, nisK is shown in UniProt Q48675. In an aspect P_{nisA} and P_{nisF} is shown in DeRuyter et al., J. Bact. 178:3434 (1996) or Eichenbaum et al., Appl. Environ. Microbiol. 64:2763 (1998) (all incorporated by reference herein).

P_{sczD}/sczA/P_{sczA} Promoter Systems

An expression cassette can comprise a P_{sczD}/sczA/P_{sczA} system. Pneumococcal repressor SczA and P_{sczD} (also called P_{czcD}) and P_{sczA} (also called P_{czcA}) tightly regulates the expression of genes under their control.

In an aspect a SczA gene is shown in SEQ ID NO:47 NCBI Reference Sequence: WP_238893273.1 and is described in Kloosterman et al., Mol. Microbiol., 65:1365 (2007) and Mu et al., Appl Environ Microbiol. (2013) July; 79: 4503-4508. A P_{sczA} promoter is also shown in SEQ ID NO:47.

P_{zitR} zitR Systems

A PzitR/zitR expression uses a P_{zitR} promoter (also called P_{zn} promoter) and a zitR regulator gene from, for example the L. lactis MG1363 zit (zitRSQP) operon. A P_{zitR} promoter and a zitR regulator gene are show in SEQ ID NO:46. Expression of genes under P_{zitR} and zitR control are regulated by metallic cations, particularly Zn²⁺. Divalent cation starvation (Zn²⁺ concentration of <10 nM) leads to upregulation, whereas concentrated Zn²⁺ (Zn²⁺ concentration of >10 nM) maintains repression. See, e.g., Llull et al., Appl. Environ. Microbiol. 70:5398 (2004)(incorporated herein by reference).

dCas/gRNA Systems

Cas, such as Cas9, can be modified to render both catalytic domains (RuVC and HNH) of the protein inactive, resulting in a catalytically-dead Cas (dCas). The dCas is unable to cleave DNA, but maintains its ability to specifically bind to DNA when guided by a guide RNA (gRNA). This allows the CRISPR/dCas system to be used as a sequence-specific, non-mutagenic gene regulation tool. In this case gRNA can be targeted to a promoter, e.g., a constitutive promoter, to block the promoter such that transcription of any genes operably linked to the promoter does not occur.

Therefore, the CRISPR/dCas system is effective to modulate gene expression and includes a dCas protein and at least one guide RNA (gRNA) molecule. In some embodiments, the one or more gRNA molecules includes a CRISPR-associated (Cas) protein binding site and a targeting RNA sequence. In some embodiments, the one or more gRNA molecules specifically targets a promoter. This is possible by designing a gRNA to include a targeting nucleic acid sequence that is complementary to a target promoter. Given the promoter sequence a gRNA can be designed and generated. An example of a gRNA targeting a promoter is shown in SEQ ID NO:48.

In some embodiments, the one or more gRNA molecules specifically bind to the target sequence (e.g., a promoter sequence), which then guide the dCas to the target sequence, where it can interfere with transcription elongation by blocking RNA polymerase or transcription initiation by blocking RNA polymerase binding and/or transcriptions factor binding. This CRISPR/dCas system is highly efficient in suppressing genes, as it is specific, with minimal off-target effects, and is multiplexable, thus allowing for the interference with multiple promoters if desired.

In some embodiments, the dCas9 endonuclease is a Streptococcus pyogenes dCas9, a Streptococcus thermophilus dCas9, a Staphylococcus aureus dCas9, a Brackiella oedipodis dCas9, a Neisseria meningitidis dCas9, a Haemophilus influenzae dCas9, a Simonsiella muelleri dCas9, a Ralstonia solanacearum dCas9, a Francisella novicida dCas9, or a Listeria monocytogenes dCas9, or a derivative of any thereof.

As used herein, “single guide RNA,” “guide RNA (gRNA),” “guide sequence” and “sgRNA” can be used interchangeably herein and refer to a single RNA species capable of directing RNA-guided endonuclease mediated cleavage of target nucleic acid molecule (e.g. a promoter).

A gRNA can comprise any single stranded polynucleotide sequence of about 20 to 300 nucleotides having sufficient complementarity with a target sequence (e.g., a promoter sequence) to hybridize with the target sequence and to direct sequence-specific binding of an RNP complex comprising the gRNA and a CRISPR effector protein, such as dCas9, to the target sequence. A gRNA contains a spacer. The spacer can comprise a plurality of bases that are complementary to the target sequence (such as target 1 or target 2). For example, a spacer can contain about 2, 3, 4, 5, 6, 7, 8, 9, 10, 11, 12, 13, 14, 15, 20, 25, 30, 35, 40, 45, 50, 55, 60, or more bases. The portion of the target sequence that is complementary to the guide sequence is known as the protospacer. When a gRNA molecule is specific for a target sequence (e.g., a promoter), the gRNA spacer pairs with a portion of the target sequence called the protospacer. The protospacer is the section of the target sequence that will be cut. The protospacer located next to a PAM sequence.

In some embodiments, the degree of complementarity between a guide sequence and its corresponding target sequence (e.g., a promoter), when optimally aligned using a suitable alignment algorithm, is about or more than about 50%, 60%, 75%, 80%, 85%, 90%, 95%, 97.5%, 99%, or more. Optimal alignment can be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at novocraft.com), ELAND (Illumina, San Diego, Calif.), SOAP (available at soap.genomics.org.cn), and Maq (available at maq.sourceforge.net).

In some embodiments, a gRNAs can be synthetically generated or by making the sgRNA in vivo or in vitro, starting from a DNA template.

In some embodiments, a gRNA that is capable of binding a target sequence (e.g., a promoter) and binding an RNA-guided DNA endonuclease protein can be expressed from a vector comprising a type II promoter or a type III promoter.

Protease Genes

A protease gene can be used in the disclosed systems to breakdown a biofilm. Suitable protease genes include, for example, Protease A (neutral protease B), B (bacillolysin) and C (subtilisin E) (Table 2), however, any suitable protease can be used. Numerous organisms produce proteases and can be used as sources of protases. For example, Bacillus subtilis 168 produces many proteases. Based on the mechanism of catalysis, proteases are classified into six distinct classes, aspartic (e.g., pepsins, cathepsins, and renins), glutamic (e.g., scytalidoglutamic peptidase), and metalloproteases (e.g., mammalian sterol-regulatory element binding protein (SREBP) site 2 protease and Escherichia coli protease EcfE, stage IV sporulation protein FB), cysteine (e.g., papain, caspase-1), serine (e.g., subtilisin, Lon-A peptidase, Cp protease), and threonine proteases (e.g., omithine acetyltransferase). Any suitable protease can be used in the compositions and methods described herein.

In an aspect an insertion sequence comprising one or more target cleavage sites for one or more proteases can be added to a biofilm assembly gene sequence. An insertion sequence can comprise 2, 3, 4, 5, or more target cleavage sites for two or more (2, 3, 4, 5, or more) different proteases. An insertion sequence can be added to the biofilm assembly gene sequence such that the expressed biofilm assembly protein can be cleaved in the presence of a protease. This can inactivate the biofilm assembly protein such that a biofilm is not produced or a biofilm is broken down. An insertion sequence can be present in the biofilm assembly gene at any position such that when the biofilm assembly protein is expressed, the insertion sequence is available to the protease and such that the insertion sequence does not interfere with the biological function of the biofilm assembly protein. For example, the insertion sequence shown in SEQ ID NO:49 and 50 was added into the linker regions of P45.

Methods

Provided herein are methods of controlling transition between planktonic growth phase and biofilm growth phase in a host cell, such as a bacterial host cell. A host cell can be transitioned to planktonic growth, then to biofilm growth, and back to planktonic growth if desired. A host cell can be transitioned to biofilm growth, then to planktonic growth, and back to biofilm growth if desired. The methods comprise growing a bacterial host cell in a medium, wherein the bacterial host cell comprises:
(i) a recombinant polynucleotide encoding one or more biofilm assembly proteins operably linked to a first repressible promoter; and
(ii) a recombinant polynucleotide encoding a protease capable of breaking down the one or more biofilm assembly proteins operably linked to a second repressible promoter.

The addition of a repressor for the first repressible promoter to the medium results in suppression of the expression of the recombinant polynucleotide encoding one or more biofilm assembly proteins and expression of the recombinant polynucleotide encoding a protease such that the bacterial host cell exhibits planktonic growth phase. In the absence of the repressor for the first repressible promoter and the presence of the repressor for the second repressible promoter in the medium results in expression of the recombinant polynucleotide encoding one or more biofilm assembly proteins and suppression of the expression of the recombinant polynucleotide encoding a protease such that the bacterial host cell exhibits biofilm growth phase.

In an aspect, The addition of a repressor for the first repressible promoter and a repressor for the second repressible promoter to the medium results in suppression of the expression of the recombinant polynucleotide encoding one or more biofilm assembly proteins and expression of the recombinant polynucleotide encoding a protease such that the bacterial host cell exhibits planktonic growth phase. In the absence of the repressor for the first repressible promoter and the repressor for the second repressible promoter in the medium results in expression of the recombinant polynucleotide encoding one or more biofilm assembly proteins and suppression of the expression of the recombinant polynucleotide encoding a protease such that the bacterial host cell exhibits biofilm growth phase.

In some aspects the bacterial host cell additionally comprises a recombinant polynucleotide encoding a protein operably linked to an inducible promoter for orthogonal expression in both biofilm growth phase and planktonic growth phase, wherein when an inducer is added to the medium, the bacterial host cell expresses the protein in both biofilm growth phase and planktonic growth phase. The bacterial host can cell additionally comprise a recombinant polynucleotide encoding a protein operably linked to the second repressible promoter for protein expression in planktonic growth phase. A second repressible promoter can be P_{sczD}, wherein the host cell additionally comprises a polynucleotide encoding a sczA operably linked to a P_{sczA} promoter. The first repressible promoter can be P_{zitR}, wherein the bacterial host cell additionally comprises a polynucleotide encoding zitR operably linked to the P_{zitR} promoter. The repressor can be zinc. The one or more biofilm assembly genes can encode P1, P2, P3, P4, P5, P6, P7, P8, P9, P10, P11, P12, P13, P14, P15, P16, P17, P18, P19, P20, P21, P22, P23, P24, P25, P26, P27, P28, P29, P30, P31, P32, P33, P34, P35, P36, P37, P38, P39, P40, P41, P42, P43, P44, P45, P45IS1, P45IS2, P45IS3, P45IS4, or P45IS5. The protease can be Neutral protease B, Bacillolysin, or Subtilisin E. The inducible promoter can be P_{nisA}. The inducer can be nisin.

An aspect provides expression cassettes, vectors, and recombinant bacterial host cells comprising a recombinant polynucleotide encoding one or more biofilm assembly proteins operably linked to a first repressible promoter; and a recombinant polynucleotide encoding a protease capable of breaking down the one or more biofilm assembly proteins operably linked to a second repressible promoter. The expression cassettes, vectors, and recombinant bacterial host cells can further comprise a recombinant polynucleotide encoding a protein operably linked to an inducible promoter. The expression cassettes, vectors, and recombinant bacterial host cells can additionally comprise a recombinant polynucleotide encoding a protein operably linked to the second repressible promoter. The expression cassettes, vectors, and recombinant bacterial host cells can further comprise a recombinant polynucleotide encoding a protein operably linked to an inducible promoter and a recombinant polynucleotide encoding a protein operably linked to the second repressible promoter.

Also provided herein are expression cassettes comprising a polynucleotide encoding a biofilm assembly gene (e.g., P1-P45, P45 with one or more insertion sequences (e.g., P45IS1, P45IS2, P45IS3, P45IS4, P45IS5)) operably linked to an inducible or repressible promoter. An inducible promoter can be P_{nisA} and the expression cassette can further comprises a polynucleotide encoding nisK/nisR operably linked to a constitutive promoter.

A population of host cells can comprise a vector encompassing an expression cassette comprising a polynucleotide encoding a biofilm assembly gene (e.g., P1-P45), optionally, with one or more insertion sequences (e.g., P45IS1, P45IS2, P45IS3, P45IS4, P45IS5) operably linked to an inducible promoter. An inducible promoter can be P_{nisA} and the expression cassette can further comprise a polynucleotide encoding nisK/nisR operably linked to a constitutive promoter. This population of cells can be used to express a biofilm assembly gene such that the population host cells form a biofilm. The population of host cells can be grown in culture and nisin can be added to the culture such that the population of host cells expresses the biofilm assembly gene and forms a biofilm.

In some aspects a biofilm assembly gene (e.g., P1-P45), optionally, with one or more insertion sequences (e.g., P45IS1, P45IS2, P45IS3, P45IS4, P45IS5) is operably linked to a repressible promoter, e.g., P_{sczD}, and the expression cassette further comprises a polynucleotide encoding sczA operably linked to a P_{sczA} promoter. A population of host cells can comprise vectors comprising this expression cassette. Biofilm assembly genes can be expressed in this population of host cells such that the host cells form a biofilm. The population of host cells can be grown in culture. Zinc can be added to the population of host cells in culture such that the population of host cells expresses the biofilm assembly gene and forms a biofilm.

In some aspects a biofilm assembly gene (e.g., P1-P45), optionally, with one or more insertion sequences (e.g., P45IS1, P45IS2, P45IS3, P45IS4, P45IS5) is operably linked a repressible promoter, e.g., P_{zitR}. An expression cassette can further comprise a polynucleotide encoding zitR that is also operably linked to the repressible promoter P_{zitR}. A population of host cells can comprise a vector comprising this expression cassette. In some aspects expression of the biofilm assembly gene can be controlled in a population of host cells. The population of host cells can be grown in culture. Zinc can be added to the population of host cells in culture such that the population of host cells does not express the biofilm assembly gene. Zinc can optionally be removed such that the population of host cells expresses the biofilm assembly gene and forms a biofilm. A zitR transcriptional repressor protein can be a Lactococcus transcriptional repression protein.

In an aspect, an expression cassette comprises a biofilm assembly gene (e.g., P1-P45), optionally, with one or more insertion sequences (e.g., P45IS1, P45IS2, P45IS3, P45IS4, P45IS5)) operably linked to a constitutive promoter, a gRNA having specificity for the constitutive promoter, and a polynucleotide encoding a dCas, wherein the gRNA having specificity for the constitutive promoter and the polynucleotide encoding dCas are both operably linked to an inducible promoter. In an aspect an inducible promoter is P_{nisA} and the expression cassette further comprises a polynucleotide encoding nisK/nisR operably linked to a constitutive promoter. A population of host cells comprising a vector having such an expression cassette can be generated. The population of host cells can be used in a method of controlling expression a biofilm assembly gene by growing the population of host cells in culture, and adding nisin to the population of host cells in culture such that the population of host cells express the gRNA having specificity for the constitutive promoter and the dCas such that expression of the biofilm assembly gene is prevented; and, optionally, removing nisin such that the population of host cells expresses the biofilm assembly gene and forms a biofilm. Alternatively, the population of host cells can be cultured in the absence of nisin such that a biofilm is generated. Nisin can then be added to the culture of host cells so that they shift from biofilm growth to planktonic growth. Growth can then be shifted back to biofilm growth if desired by removing or stopping the addition of nisin to the cell culture.

In an aspect an expression cassette comprises a polynucleotide encoding a protease operably linked to repressible promoter P_{sczD}, a polynucleotide encoding sczA operably linked to a P_{sczA} promoter, and a polynucleotide encoding a biofilm assembly gene (e.g., P1-P45 optionally, with one or more insertion sequences (e.g., P45IS1, P45IS2, P45IS3, P45IS4, P45IS5)) and zitR operably linked to repressible promoter P_{zitR}. The polynucleotide encoding a protease operably linked to repressible promoter P_{sczD}, can further comprise one or more functional genes or marker genes also operably linked to the repressible promoter P_{sczD}. The expression cassette can further comprise a polynucleotide encoding one or more functional genes or marker genes operably linked to a P_{nisA} promoter. A protease can be, for example, Neutral protease B, Bacillolysin, or Subtilisin E.

In an aspect a population of host cells can comprise a vector comprising an expression cassette having a polynucleotide encoding a protease operably linked to repressible promoter P_{sczD}, a polynucleotide encoding sczA operably linked to a P_{sczA} promoter, a polynucleotide encoding a biofilm assembly gene (e.g., P1-P45), optionally, with one or more insertion sequences (e.g., P45IS1, P45IS2, P45IS3, P45IS4, P45IS5)) and zitR operably linked repressible promoter P_{zitR}. The polynucleotide encoding a protease operably linked to repressible promoter P_{sczD}, can further comprise one or more functional genes or marker genes also operably linked to the repressible promoter P_{sczD}. The expression cassette can further comprise a polynucleotide encoding one or more functional genes or marker genes operably linked to a P_{nisA} promoter. This population of host cells can be used in a method of controlling expression a biofilm assembly gene in a population of host cells. The population of host cells can form a biofilm when the cells are cultured in the absence of zinc. Zinc can be added to the population of host cells such that the population of host cells switches to planktonic growth. Alternatively, the population of host cells can grow in planktonic form when the cells are cultured with zinc. The zinc can then be removed or no more addition of zinc can used to move the cells to biofilm growth. Furthermore, nisin can be added to the culture to activate a P_{nisA} promoter to transcribe a polynucleotide encoding one or more functional genes or marker genes to which it is operably linked such that the polynucleotide encoding one or more functional genes or marker genes is expressed.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

Example 1. Mining matrix building blocks for orthogonal biofilm assembly. Biofilm formation is a foundational prerequisite for bacteria to alternate lifestyles; we thus started by searching for scaffold molecules that constitute biofilm extracellular matrix. We targeted those orthogonal to native counterparts because they promote the predictability of desired behaviors and flexibility of functionality programming by minimizing the crosstalk with endogenous circuitry. We also specifically chose protein as our potential building block over other extracellular polymeric substances such as polysaccharides, DNA and lipids due to its relative ease for production and modification.

Utilizing the UniProt protein database⁴⁴, we explored surface-related proteins of lactobacillus species, from which 45 candidates were identified (Table 1).

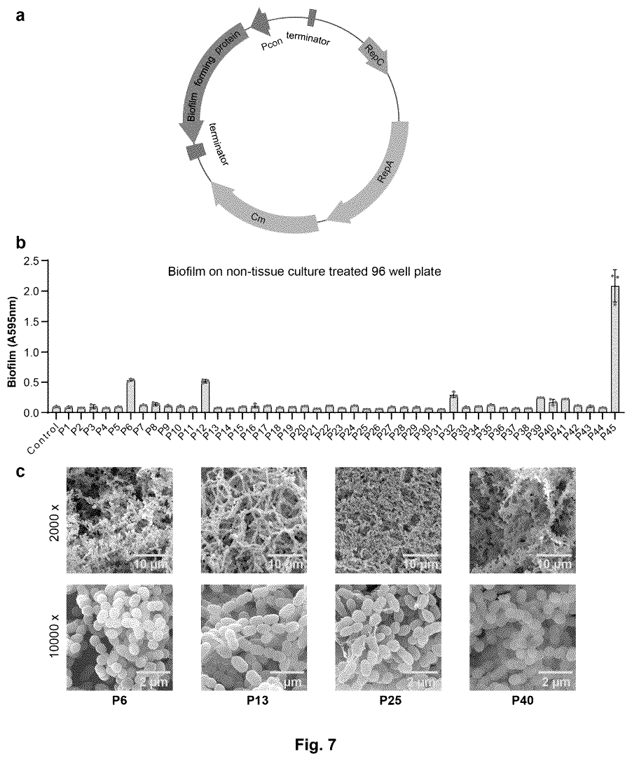

We cloned the candidate genes into the constitutive expression vector, pleiss-pcon-gfp (FIG. 7a), andtransformedthe resulting plasmids into L. lactis NZ9000, a cellular chassis deficient in biofilm formation (Table 2).

TABLE 2
Strains and plasmids used in this study.

| Strains | Features | Reference |
|---|---|---|
| Lactococcus lactis | Host for biofilm formation and nisin induction system; | (1) |
| NZ9000 | nisRK integrated into the chromosome | |
| Listeria monocytogenes | Foodborne pathogen and sensitive strain for Pediocin | (2) |
| 10403S | | |
| Plasmids | | |
| pleiss-Pcon-gfp | Plasmid for constitutive expression of gfp in Lactic | (3) |
| | acid bacteria; Used for constitutive expression of | |
| | biofilm forming proteins in this study; Cm resistance | |
| pleiss:nuc | Nisin induced expression of Nuc; PnisA promoter and | (4) |
| | Usp45 signal peptide; Cm resistance | |
| pZitR-P45 | Zinc limitation induced expression of P45 | This study |
| pZnin-P45 | Zinc induced expression of P45 | This study |
| pNis-P45 | Nisin induced expression of P45 | This study |
| pCon-P45-PnisA-gRNA- | Nisin repressed expression of P45 | This study |
| PnisF-dcas9 | | |
| pNis-protease a | Nisin induced expression of Neutral protease B | This study |
| | from Bacillus subtilis 168 | |
| pNis-protease b | Nisin induced expression of Bacillolysin cloned | This study |
| | from Bacillus subtilis 168 | |
| pNis-protease c | Nisin induced expression of Subtilisin E from | This study |
| | Bacillus subtilis 168 | |
| P45-Zn-gfp | Zinc induced expression of gfp; Zinc limitation | This study |
| | induced expression P45 | |
| IS5-Zn-gfp-prob | Zinc induced expression of gfp and protease b; | This study |
| | Zinc limitation induced expression P45IS5 | |
| IS5-Zn-gfp-proc | Zinc induced expression of gfp and protease c; | This study |
| | Zinc limitation induced expression P45IS5 | |
| P45-Zn-amylase | Zinc induced expression of amylase; Zinc limitation | This study |
| | induced expression P45 | |
| IS5-Zn-amylase-prob | Zinc induced expression of amylase and protease b; | This study |
| | Zinc limitation induced expression P45IS5 | |
| P45-Zn-mHO-1 | Zinc induced expression of mHO-1; Zinc limitation | This study |
| | induced expression P45 | |
| IS5-Zn-mHO-1-prob | Zinc induced expression of mHO-1 and protease b; | This study |
| | Zinc limitation induced expression P45IS5 | |
| P45-Zn-gusA | Zinc induced expression of gusA; Zinc limitation | This study |
| | induced expression P45 | |
| IS5-Zn-gusA-prob | Zinc induced expression of gusA and protease b; | This study |
| | Zinc limitation induced expression P45IS5 | |
| P45-lon-Zn-gusA-tag | Zinc induced expression of gusA with degradation | This study |
| | tag; Zinc limitation induced expression P45 and | |
| | mf-lon protease | |
| IS5-Zn-gusA-tag-prob- | Zinc induced expression of gusA with degradation | This study |
| Pcst-lon | tag and protease b; Zinc limitation induced | |
| | expression P45IS5 and mf-lon protease | |
| IS5-Zn-Prob-Pnis-bga | Zinc induced expression of protease b and zinc | This study |
| | limitation induced expression of P45IS5; Nisin | |
| | induced expression of bga | |
| IS5-Zn-Prob-Pnis-ped | Zinc induced expression of protease b and zinc | This study |
| | limitation induced expression of P45IS5; Nisin | |
| | induced expression of ped | |
| IS5-orf29-P7-Erm-Zn- | Zinc induced expression of gfp and protease b | This study |
| gfp-Prob | and zinc limitation induced expression of P45IS5 | |
| | and orf29; orf29 activated expression of P7-driven | |
| | erythromycin resistance protein | |

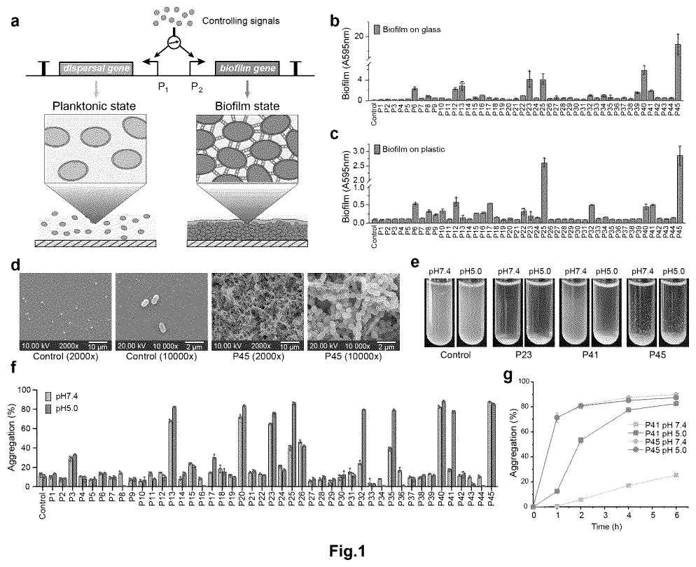

To characterize these proteins, we cultured the strains for 24 hours with GM17 medium in 12-well plates that contain 18 mm glass cover slips on wells' bottoms. Using crystal violet staining⁴⁵, we found that, compared to GFP encoded by the control strain, a large portion of the expressed proteins promoted biofilm formation on glass among which P6, P12, P13, P23, P25, P40 and P45 yielded densest biofilms (FIG. 1b). We also tested whether biofilms form on plastic surfaces by inoculating the strains into cell culture treated 96-well plates. The results showed that 14 out of the 45 proteins conferred clear biofilm formation (FIG. 1c). On non-treated plastic surfaces, biofilms were also observed and those of P6, P12, P32, P39, P40, P41 and P45 were among the thickest (FIG. 7b). Scanning electron microscope (SEM) images provided direct visual confirmation of strong biofilm assembly by these proteins (FIG. 1d and FIG. 7c).

Auto-aggregation enables planktonic cells to attach to each other and is often considered as another common trait of biofilms besides surface attachment⁴⁶. We thus cultured the 45 strains in test tubes and quantified their auto-aggregation. We found that auto-aggregation (FIG. 1e,f) is not always positively correlated with biofilm formation (FIG. 1b,c). For example, P6 enabled biofilm formation on glass and plastic surfaces but not cellular self-aggregation whereas P20, incapable of directing biofilm assembly, was effective for aggregation. In addition, these proteins exhibited varied pH dependence for aggregation. Notably, P41 allowed rapid aggregation at pH 7.4 but not at pH 5.0 while P45 conferred effective aggregation at both conditions (FIG. 1g). Collectively, these assays suggested P6, P25, P40 and P45 as the best scaffold candidates for building synthetic biofilms.

### Example 2. Controllable Biofilm Formation by External Signals

Controllability is a key trait for engineered organisms to realize desired behaviors. To regulate bacterial life cycle, we proceeded to construct gene circuits that direct the organization of planktonic cells into biofilms.

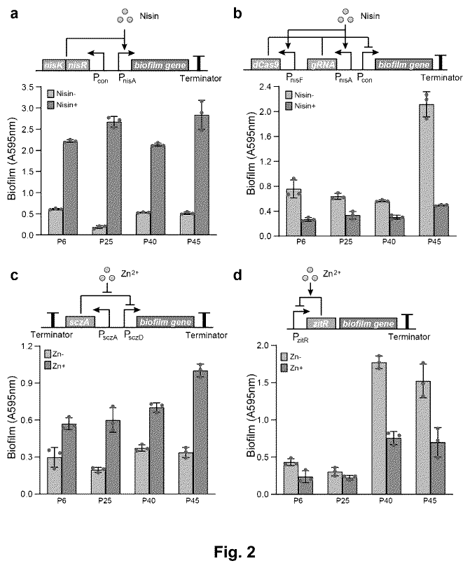

We set out to exploit the NICE system, an externally inducible module for L. lactis⁴⁷, by leveraging the integrated nisR/K cassette in the NZ9000 chromosome and using the nisin inducible promoter, P_{nisA}, to drive the scaffold protein genes (FIG. 2a, top). Our results showed that in all cases nisin induction resulted in successful development of synthetic biofilms (FIG. 2a, bottom). To examine if the regulation can be inverted, we also introduced a dcas9-gRNA module⁴⁸ into the nisin-inducible circuit (FIG. 2b, top). In this design, upon nisin induction the gRNA anneals to the promoter P_{con}, which is followed by the binding of dcas9 to gRNA-promoter complex to block transcription. Our subsequent experiment confirmed that biofilm formation can be suppressed in the presence of nisin with the design (FIG. 2b, bottom).

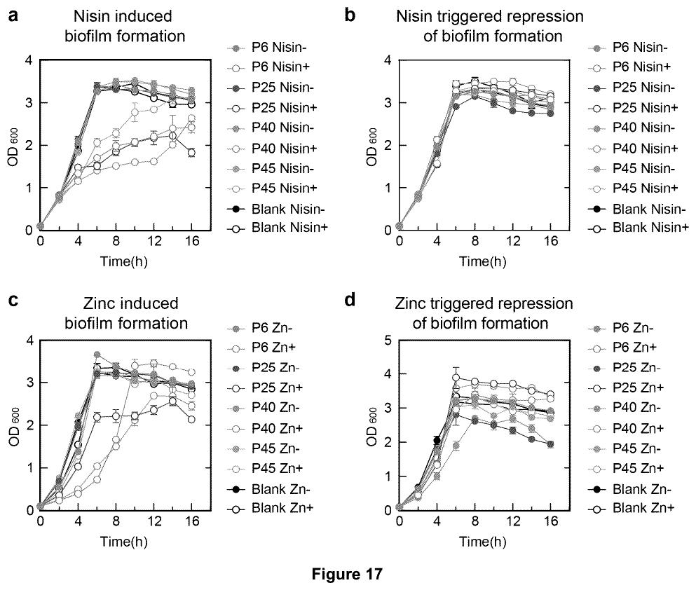

Additionally, we assessed whether synthetic biofilm assembly can be regulated by physiologically relevant variables akin to the formation of native biofilms triggered by nutrient limitation and stress. Adopting zinc as a responsive cue, we built a gene circuit involving the constitutively expressed transcriptional factor gene sczA⁴⁹ and its cognate promoter P_{sczD} driving the scaffold genes (FIG. 2c, top). Confirmed by our experiment (FIG. 2c, bottom), here zinc binds to SczA to release its suppression on P_{sczD} to activate scaffold protein synthesis. In a similar way, a zinc-repressive module was created by pairing the transcriptional repressor gene, zitR⁵⁰, with its cognate promoter P_{zitR} to form a negative auto-regulatory circuit (FIG. 2d, top). With this circuit, biofilms formed only in the absence of zinc (FIG. 2d, bottom). As heterologous protein production causes a metabolic burden, we further measured the growth of the strains harboring the circuits. The results (FIG. 17) revealed that encoding the scaffolds led to a growth reduction with the degree depending on the scaffold molecules and the induction systems, which suggested that the induction of scaffold synthesis overrode the growth disadvantage to generate efficient biofilm development as shown in FIG. 2. Together, we established four controllable modules for directing biofilm assembly.

### Example 3. Engineered Biofilm Decomposition Via Protein Design

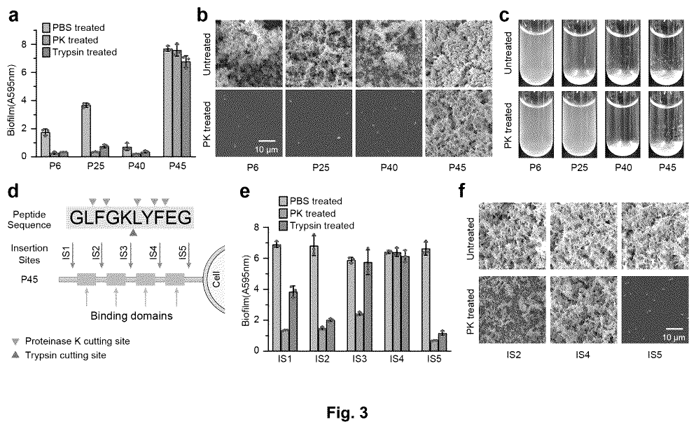

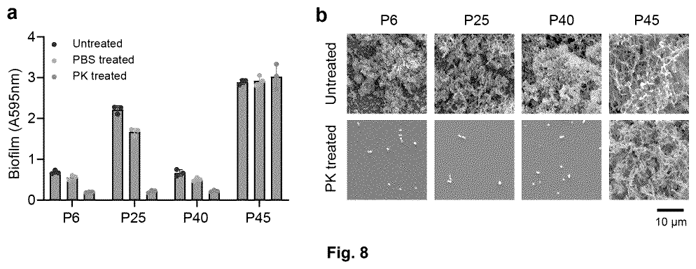

Opposite to biofilm assembly is its deconstruction, another key step of bacterial life cycle during which aggregated cells disperse from biofilms into single cells. Although engineering biofilm dispersal has been a long-standing challenge for researchers, microbes in nature have found remarkable strategies to break down matrix and release cells. For instance, they secrete enzymes to degrade polysaccharides and eDNA, common components of matrix, to achieve biofilm degradation. In our design, proteins are the building blocks of synthetic biofilms, so we were inspired to investigate protease for programmable biofilm destruction. Using Proteinase K and trypsin, we found that on both glass cover slips (FIG. 3a) and plastic surfaces (FIG. 8a) the biofilms assembled via P6, P25 and P40 were effectively broken down but that of P45 remained largely intact. These results were validated by corresponding SEM images (FIG. 3b and FIG. 8b). In addition, by comparing the bacterial cultures in the absence and presence of Proteinase K at pH 7.4, we found that P6 did not aggregate even without Proteinase K but the other three showed differential characteristics. Namely, upon the protease treatment, P25 lost aggregation, P40 remained aggregated but lost the attachment ability while P45 remained both aggregated and attached (FIG. 3c). Collectively, we concluded that protease supplementation is an effective strategy to eradicate P6, P25 and P40 biofilms. Meanwhile, consistent with our findings in FIG. 1, the results also indicated that cell aggregation and biofilm development are not always directly correlated.

One limitation of the trio, however, is that they are much weaker than P45 toward biofilm formation (FIG. 3a-c), which could hinder future use. We developed a controllable degradation of P45 while retaining its assembly performance. P45 is attached to cell wall through its C-terminal LPTG (SEQ ID NO: 68) sortase cleavage site⁴⁴ and has four tandem binding domains that are potentially involved in surface attachment and biofilm formation (FIG. 3d). We thereby designed a peptide sequence containing multiple protease recognition sites (FIG. 3d, green and blue triangles). By introducing the sequence into one of the linker regions that separate the binding domains, we obtained five P45 variants, named IS1 to IS5, corresponding to their insertion sites.

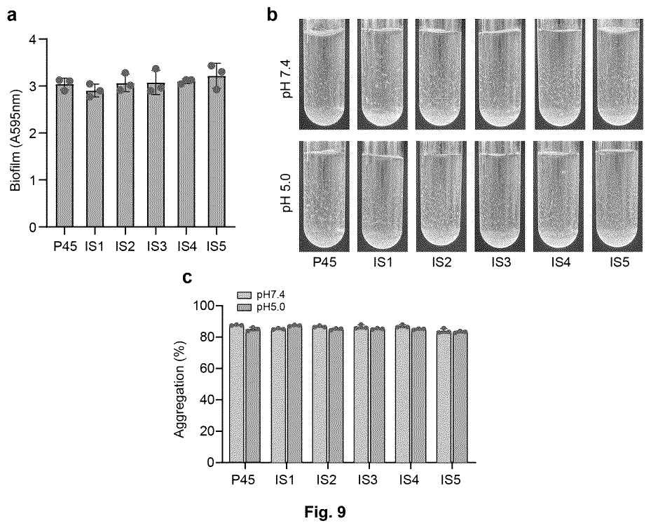

Subsequently, we measured the biofilm formation ability and sensitivity to protease treatment for the strains expressing the variants. Compared to the original P45 (FIG. 3a), all variants possessed a comparable biofilm forming ability on glass cover slips (FIG. 3e, yellow bars) and plastic surfaces (FIG. 9a) and a comparable aggregation ability (FIG. 9b,c), demonstrating that the insertions did not impair biofilm formation. Meanwhile, their protease sensitivity varied significantly. The variants IS1, IS3 and IS4 were partially or fully resistant to Proteinase K and trypsin treatments whereas IS2 and IS5 were sensitive to both treatments. We speculated the reason was that, in the folded structures of the variants, the IS1, IS3 and IS4 sites are partially or fully hided and the proteinases do not have the access to these sites. Supporting the finding, SEM images showed that, upon Proteinase K treatment, the IS4 biofilm remained intact, the IS2 biofilm was partially dispersed whereas the IS5 biofilm was completely decomposed (FIG. 3f). IS5 thus serves as the optimal scaffold building block. Pairing its gene expression with inducible circuits and protease supplementation, we achieved externally tunable cellular phase transition with effective biofilm formation and decomposition.

### Example 4. Autonomous Lifestyle Transition Between the Planktonic and Biofilm Modes

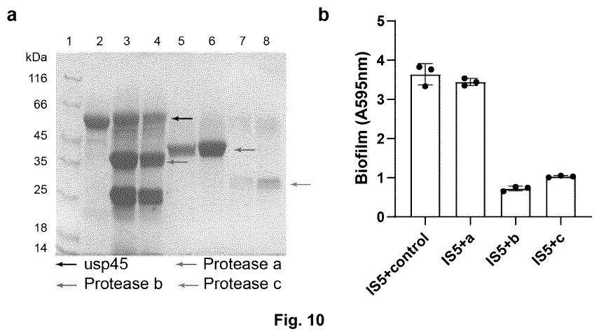

In nature, microbes dynamically and autonomously alternate their lifestyles in response to environmental cues, which allows them to match different physiological needs and harness the benefits of both phases. To empower synthetic bacteria with such a trait, we tested the feasibility of in vivo protease expression and secretion. Three protease genes from Bacillus subtilis 168, Protease A (neutral protease B), B (bacillolysin) and C (subtilisin E)⁵¹ (Table 2), were cloned along with their native signal peptides and placed under the nisin inducible promoter (P_{nisA}). Our SDS-PAGE results showed that all three proteases were secreted and cleaved correctly (FIG. 10a), among which Proteases B and C exhibited a degradation effect against IS5 (FIG. 10).

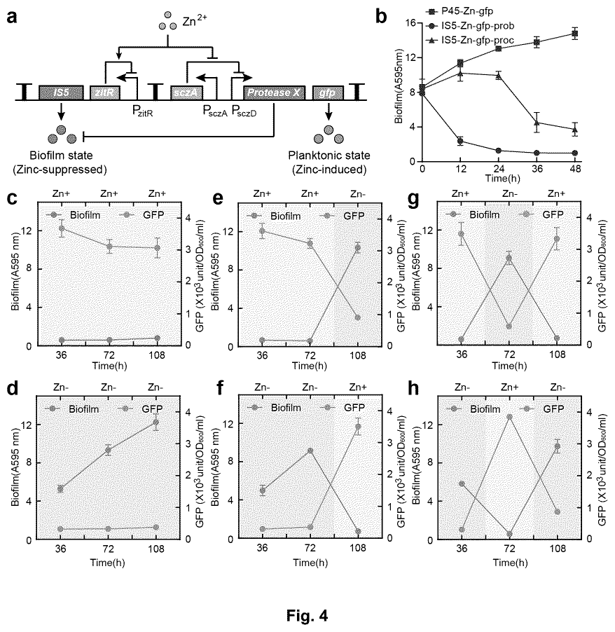

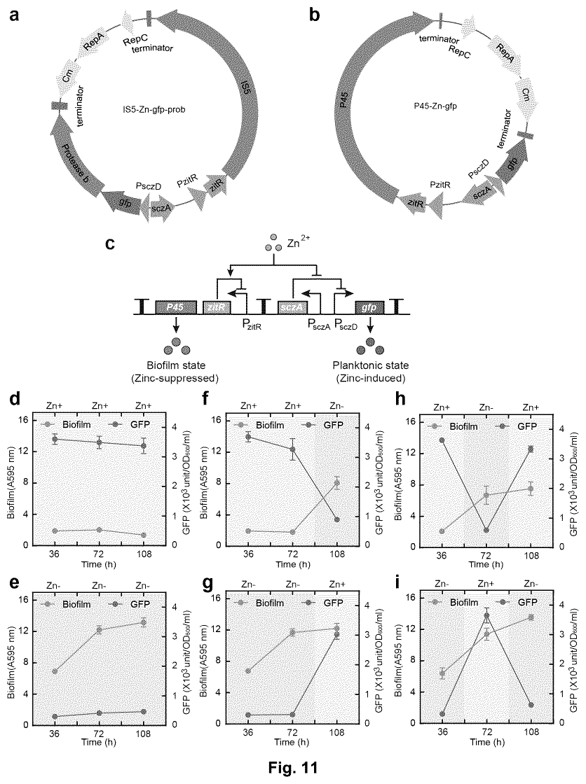

We then proposed an integrated gene circuit for environment-responsive autonomous planktonic-biofilm transition, which comprises the scaffold gene IS5, a zinc-repressed control module, a zinc-inducible control module, the protease gene X and the reporter gene gfp (FIG. 4a, FIG. 11a). In the absence of zinc, the scaffold protein IS5 in this design is produced but the protease expression is inhibited, leading to microbial assembly into biofilms. By contrast, in the presence of zinc, IS5 synthesis is halted but the protease is actively produced to digest IS5 in the matrix, which drives the cells to the planktonic form. To test the design, we built two versions which contain Protease B (IS5-Zn-gfp-prob) and Protease C (IS5-Zn-gfp-proc) respectively. The former was shown to outperform the latter in terms of biofilm dispersal although they both were effective (FIG. 4b).

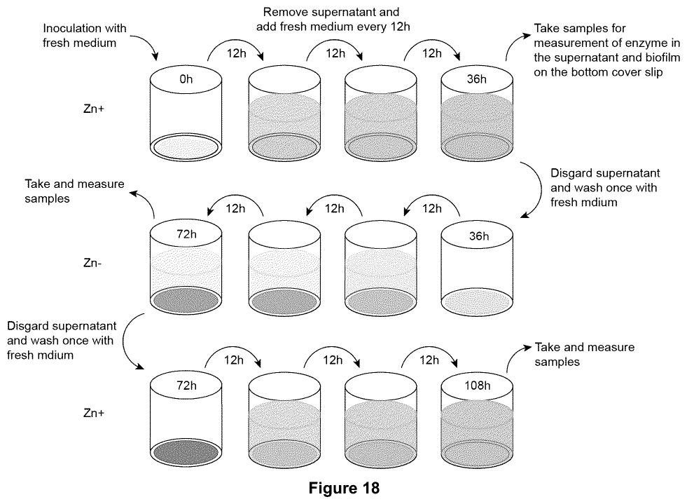

Next, we evaluated the autonomy of the circuit (IS5-Zn-gfp-prob)-loaded cells under different zinc-varying settings (FIG. 18). Our study showed that the cells remained planktonic and produced a high level of GFP when zinc was available (FIG. 4c); however, when zinc became deficient, the cells self-organized into biofilms without detectable GFP expression (FIG. 4d). Thus, the cells were locked in a single state, planktonic or biofilm, when the zinc concentration was static. In changing environments, the cells underwent zinc-responsive, anti-correlated alterations of biofilm thickness and GFP expression (FIG. 4e-h). For instance, the thickness of the biofilm shifted from low to high and back to low while the GFP level changed from high to low and to high as the zinc availability altered from abundance to deficiency and back to abundance (FIG. 4g). These results demonstrated that the cells harboring the circuit dynamically adjusted their lifestyles between the planktonic and biofilm states with regards to the zinc level. For comparison, we assembled a control circuit, P45-Zn-gfp, which encodes P45 as the scaffold and lacks protease synthesis (FIG. 11b,c). The cells carrying the control circuit formed biofilms but failed to dissociate from the biofilms (FIG. 11d-i), suggesting the need of the full circuit (IS5-Zn-gfp-prob, FIG. 4a) for bidirectional phase transition.

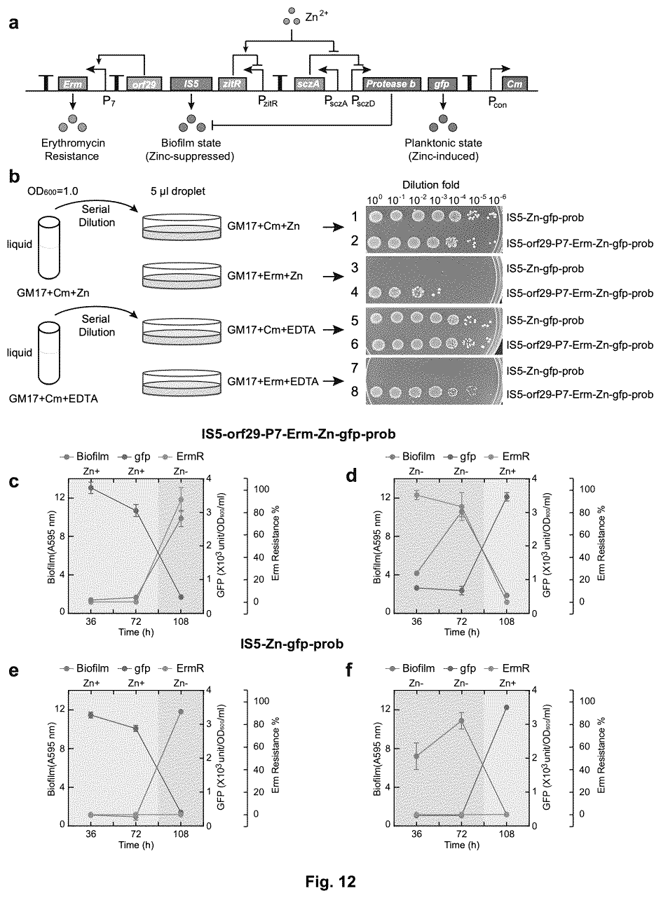

In nature, biofilm formation is often associated with the alteration of cellular functions through accompanied genetic, metabolic or signaling cascades. To demonstrate the potential of the lifestyle program for driving cellular functional phenotypes, we constructed a new circuit (IS5-orf29-P7-Erm-Zn-gfp-prob) that couples biofilm formation with erythromycin resistance, a model phenotype (FIG. 12a). With this design, Protease B shall be produced but IS5 would not when zinc is present, driving the cells to the planktonic state. However, in the absence of zinc, IS5 would be encoded but Protease B would not, which would induce biofilm formation; meanwhile, the transcriptional activator Orf29⁵² will be co-expressed to activate the promoter P7, which subsequently drives the expression of the erythromycin resistance gene and hence induces the antibiotic resistance. Our experiments showed that the erythromycin resistance of the strain containing IS5-orf29-P7-Erm-Zn-gfp-prob was 100 times higher in the biofilm state (colony row 8) than in the planktonic state (colony row line 4) (FIG. 12b). Additionally, the erythromycin resistance was tightly coupled to the biofilm state of the strain undergoing dynamic phase transition (FIG. 12c,d); by contrast, the control strain which carries the circuit IS5-Zn-gfp-prob did not yield any erythromycin resistance (FIG. 12e,f).

### Example 5. Platform Applications for Phase-Specific Function Execution

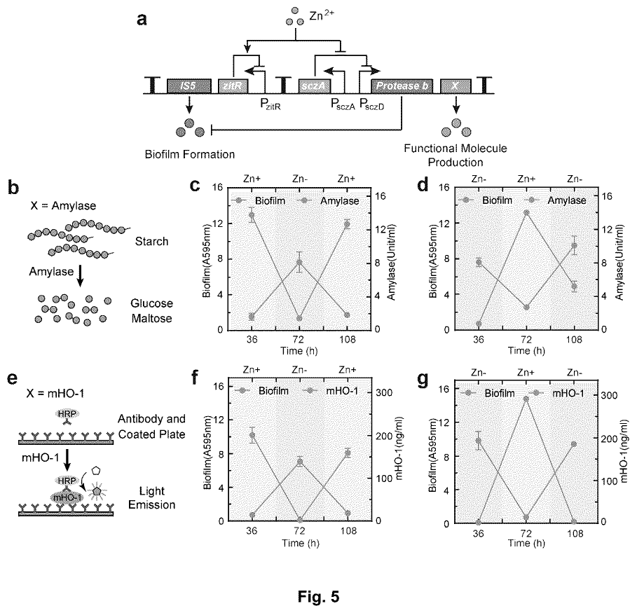

To illustrate the utility of this synthetic lifestyle program, we asked whether it can be utilized for phase-specific heterologous biosynthesis that aligns with the alteration of physiological homeostasis in changing environments. Explicitly, we targeted protein synthesis in the planktonic phase, as single cells have a better access than their biofilm counterparts to nutrient needed for biomolecule overproduction. Toward this goal, we created a modular design involving a generic functional cassette X that is substitutable for encoding different substances (FIG. 5a).

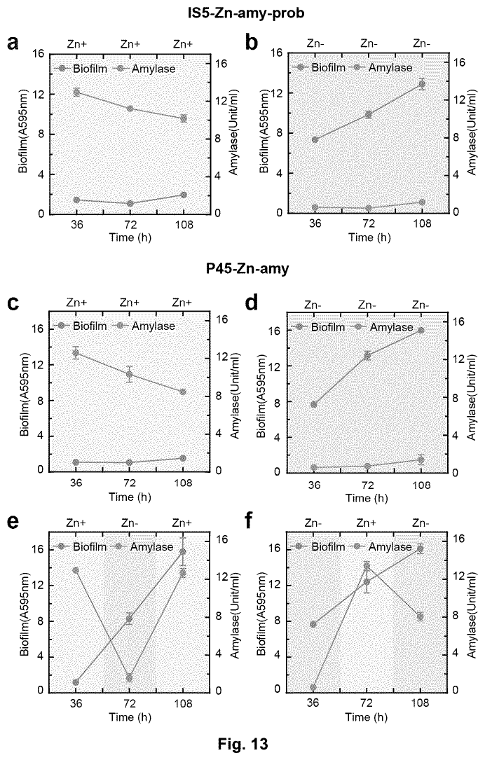

We specified X in the design with the amylase gene amyE⁵³, which produces a hydrolase secreted to convert polymeric starch into simple sugars (FIG. 5b). Our results showed that the cells stayed planktonic and simultaneously secreted amylase when and only when zinc was present (FIG. 13a,b). In addition, the level of secreted amylase varied in company with the shift of cellular phase in response to the change of environmental zinc availability (FIG. 5c,d). However, when P45 was used as the scaffold but Protease B was absent from the system, the program failed to drive the biofilm cells to the planktonic state even though their amylase synthesis remained active (FIG. 13c-f).

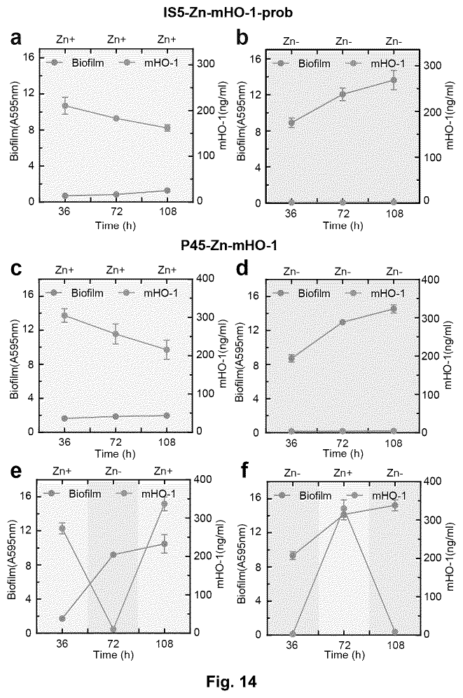

We continued the test by synthesizing and secreting the model therapeutic substance, mouse heme oxygenase 1 (mHO-1), which reduces superoxide and other reactive oxygen species and hence promotes the prevention of inflammation⁵⁴ (FIG. 5e). Similar to the above case, our experiment showed that the engineered cells were able to alternate between the biofilm and planktonic states depending upon the zinc level and produced functionally active mHO-1 only when the cells were planktonic (FIG. 14a,b and FIG. 5f,g). Again, IS5 and Protease B were indispensable for coordinated phase transition and phase-specific bioproduction (FIG. 14c-f).

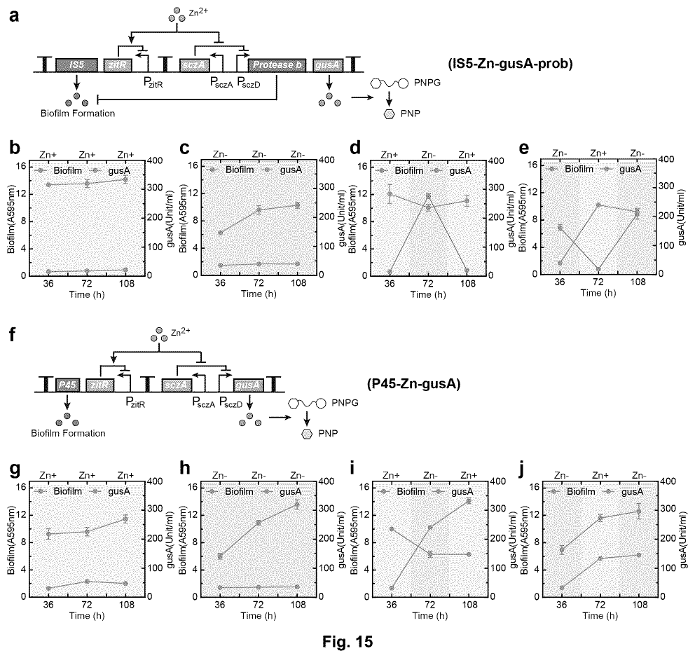

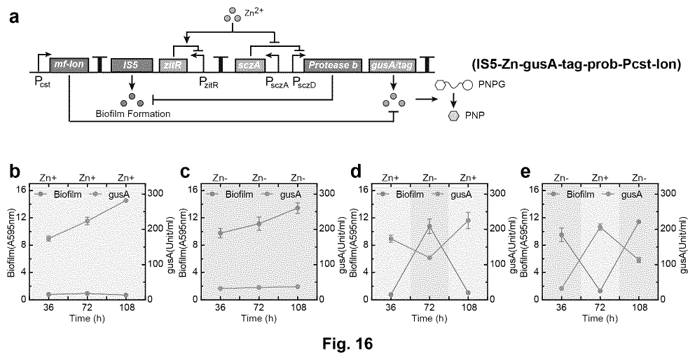

To explore if our synthetic program also confers dynamic, phase-specific modulation of intracellular, un-secreted molecules, we further adapted the circuit to encode GusA which catalyzes the hydrolysis of 3-D-glucuronic acid residues⁵⁵ (FIG. 15a). Different from amylase, PslG and mHO-1 that were secreted and washed out over time, GusA maintained at a high level inside the cell even 36 hours later after the removal of zinc, likely due to its high stability (FIG. 15b-e). Its lack of dynamic response was further exaggerated when P45 was adopted but Protease B was absent (FIG. 15f-j). To install fast response, we introduced a protein turnover circuitry by expressing the tag-specific protease gene mf-lon⁵⁶ and inserting a degradation tag pdt3 to gusA (FIG. 16a). Remarkably, the active degradation module indeed augmented the dynamic tunability of intracellular GusA abundance during cellular phase transition (FIG. 16b-e).

### Example 6. Independent Control Over Lifestyle Alteration and Function Delivery

To further showcase the platform, we sought to explore orthogonal control over cellular lifestyle and function realization. In theory, such a management fashion allows engineered strains to sense multiple environmental stimuli, yield adjustable responses and behave beyond the imitation of native organisms, thereby expanding the programmability of cellular functionality.

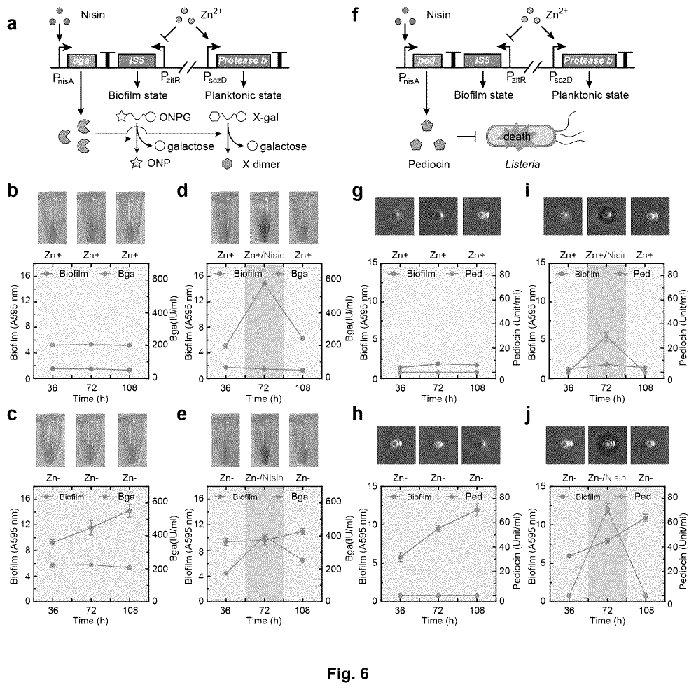

To that end, we devised a pair of regulatory modules, including one zinc-responsive and the other nisin-inducible, which independently drive lifestyle transition and the expression of functional genes (e.g., bga) respectively (FIG. 6a). This design allows functional substance synthesis with tunable production rate and time regardless of cellular phase, which is particularly important when the substances are expensive or toxic to synthesize and secrete.

Our first demonstration of the design involved the gene bga, which encodes a secreted beta-galactosidase that hydrolases lactose to glucose and galactose and helps to treat lactose intolerance⁵⁷. We quantified the Bga level and biofilm thickness of the cells under varied zinc and nisin conditions. Despite cellular phase variations, we found the Bga level remained low as long as nisin was absent (FIG. 6b,c) but rose rapidly when and only when nisin was present (FIG. 6d,e). Conversely, the cells formed biofilms upon zinc deprivation irrespective of the Bga level (FIG. 6c,e). These experiments confirmed uncoupled regulation of Bga synthesis and phase alternation. Additionally, because of its high molecular weight, Bga was not detected when the gene was driven by the zinc-inducible promoter due to its relative weak strength (data not shown); by contrast, the nisin-based induction yielded a high level of Bga synthesis (FIG. 6d,e), underscoring the additional benefit, expression level modulation, conferred by orthogonal control.

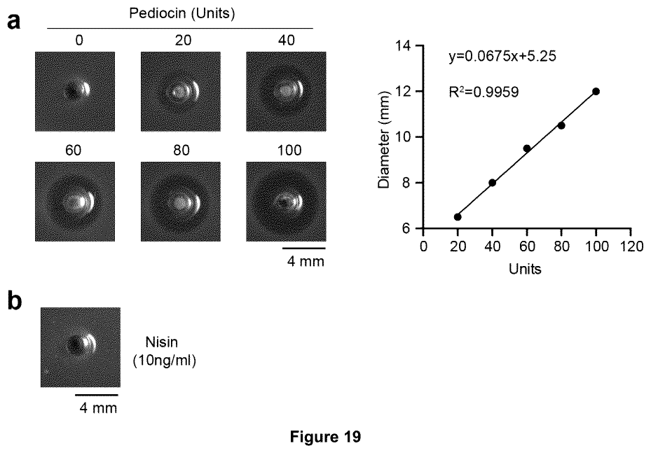

Our second demonstration included the synthesis of the pediocin PA-1 (FIG. 6f), a food preservative that inhibits the pathogen Listeria monocytogenes⁵⁸. Here, independent control of pediocin was achieved by placing the gene ped downstream of the nisin-inducible promoter P_{nisA}. We performed multiple zinc and nisin modulations and measured the corresponding biofilm thickness and pediocin concentration, whereby an agar diffusion assay was adopted (FIG. 19a). The results showed that pediocin production remained minimal without nisin induction regardless of cellular phase (FIG. 6g,h) and was turned on only when nisin was added to the culture (FIG. 6i,j). Notably, although nisin is an antimicrobial, its low dose used for induction did not suppress L. monocytogenes in the diffusion assay (FIG. 19b). Collectively, our examples (FIGS. 5 and 6) demonstrated that the orthogonal phase transition platform is independent of native regulation and versatile to deliver various functions through the plug and play of circuit modules and that both phase-specific and phase-independent gene expression can be programmed on top of the lifecycle to fulfill complex tasks.

### Example 7. Discussion

We established here a synthetic genetic program for bacterial lifestyle control that is orthogonal, tunable and programmable. The program utilizes an orthogonal mechanism centering around engineered surface proteins for matrix assembly. It is also highly controllable for biofilm formation and decomposition and accessible for responsive autonomous planktonic-biofilm transitions. The platform is further programmable for advanced function realization such as phase-coordinated and phase-independent biomolecule production.

Rapid advances in synthetic biology have brought the engineering of living organisms from concept demonstration to the exciting stage for applications. Our synthetic system provides a promising platform for engineering microbes that are adaptive to changing habitats and capable of fulfilling tasks across physiologically distinct regimes. One potential application lies in industrial practices relating to biomanufacturing, biocatalysis and food production, by creating a genetic program that drives cells to switch between active product synthesis and sessile biofilm development in response to external signals for long-term, multi-round fermentations. Additionally, the system can be utilized to enhance and prolong the therapeutic effects of probiotics for chronic inflammation and infection by establishing a genetic system that enables custom-tailored strains to colonize in the gastrointestinal tract and secret therapeutic agents as needed. Meanwhile, to fully unlock biofilms for future use, our platform can be further augmented by introducing self-recognition circuits to facilitate rapid autonomous lifecycle transition and by extending the biofilm engineering of mono-species populations to multispecies communities. In parallel, the system can be adopted as a well-defined experimental model for studying the fundamental process of microbial environmental sensing and decision making, and as a possible testbed for evaluating strategies for biofilm prevention and removal. As biofilms are multicellular systems with spatial heterogeneity, the platform can be potentially utilized to interrogate microbial social interactions, spatial organization, and multicellularity development.

### Example 8 Methods

Strains and growth conditions. Lactococcus lactis (L. lactis) NZ9000 was used as the host for expression of biofilm forming proteins. Lactococcal strains were cultured in M17 medium with 0.5% glucose (GM17) at 30° C. Listeria monocytogenes 10403S was grown in TSB medium at 37° C. Antibiotic and chemicals were added as required: chloramphenicol (Cm, 5 μg ml⁻¹), nisin (10 ng ml⁻¹), ZnSO₄ (1 mM) and EDTA (30 μM). A complete description of the strains and plasmids is provided in Table 2.

Plasmid construction. Genomic DNAs of lactic acid bacteria strains were prepared using the CTAB method⁵⁹. Genes of 45 putative surface-binding and aggregation proteins were amplified from genomic DNAs and cloned into the plasmid pleiss-Pcon-gfp¹⁵ to replace the gfp gene. Gibson assembly was used for the construction of all plasmids. The gene fragments dcas9 and mf-lon were amplified from the plasmids pMJ841 and pECGMC3 which were purchased from Addgene. The amylase gene amyE was cloned from Bacillus subtilis 168. Mouse heme-oxygenase 1 gene mHO-1, β-galactosidase gene bga, zinc inducible circuit, zinc repressed circuit, pediocin gene ped and orf29 were all synthesized as Gblock from IDT. Sequences for promoters and genes are listed in Table 3.

TABLE 3
Sequence information for genes, promoters and insertion sequences.

| Gene or | | |
|---|---|---|
| promoter | Sequence | Reference |
| zitR and Zinc | TAATAAAACTTATTGTTTTGATGTTCGGCTTAAGGATGGAAGGATTTTTCAAAT | (5) |
| limitation | AAAAAAGTAAAAAATAATGTTAACTGGTTGACATTATTTTTACTTTGCTATATAA | |
| induced | TTAACCAGTAAACTAATTATGGAGGACGAAATACTATGAGTTTAGCAAATCAAA | |
| promoter | TCGACCAGTTTCTTGGGGCAATTATGCAGTTTGCAGAAAACAAGCATGAAATA | |
| (zitR is | TTACTCGGCGAATGCGAAAGTAATGTTAAGCTAACAAGCACGCAAGAACATAT | |
| underlined) | CTTAATGATTCTAGCTGCAGAGGTTTCGACAAACGCGAGAATTGCCGAGCAAC | |
| | TCAAGATTTCGCCAGCAGCGGTAACTAAAGCTCTCAAAAAATTACAAGAGCAA | |
| | GAACTGATTAAATCAAGTCGGGCAACAAATGACGAACGCGTAGTCCTTTGGA | |
| | GCCTGACAGAAAAAGCAATTCCAGTTGCTAAAGAACATGCTGCTCATCATGAG | |
| | AAAACTCTAAGTACCTACCAAGAATTAGGAGACAAATTTACTGACGAAGAACA | |
| | AAAAGTGATAAGTCAATTCTTATCAGTACTTACGGAGGAGTTTCGATGAAG | |
| | SEQ ID NO: 46 | |
| sczA and zinc | ATGGTCTTCAAGGGAAAACAGTAACCATTATAGGAGTGCTGTTTTGAGATTTC | (6) |
| induced | GATTAAAACAGATATAGTTGATAATCAAGGATTTATAGTATGAAAAAGAGGATC | |
| promoter | GGCGGGTCCTCTTTTGTTGTTGAAAAGATAAAAAACTCAGTAACCTAGAAATA | |
| (sczA is | AGACAACTGAAGCTTTACTCTATATTCAATTCTTTGGAATTAATAAATCCAAATA | |
| underlined) | AAATTGTACAACTTCTTGATCTGTGAAGTCTTGTCCTTTCTTCAACCACCATGT | |
| | CAAAGTTTCAATAAAATTTGACATAACCAAATGTTGCAAATATGATGTTGGTAA | |
| | ATTTGGATGAGCTTCTTTCAAATTATCAGCTAAAACTGAATAAACATGATGTTC | |
| | TAATTCCTTATGTAATTGTCTTAAGAAATAATCATTCTTTGAGAACAATAATGAT | |
| | GTAATATGATCTTGATTCTTATGGAAATGTAAGAATAAATGAGCCAAATAATCT | |
| | TCTGTTGAAATAGCTTGTTCTCTTTCAAACAAATGATGAAACAAATATCTACATA | |
| | ATTGATCCAATAATAATTCTTTAGATTCATAATGACAATAGAATGTTGATCTTCC | |
| | AACATCAGCCAAATCAATAATATCTTGAACAGTTGTAGCTTCATATCCTTTAGC | |
| | ATTTAATAATTGAATAAATGCTTGATAGATGGCTTTTTTGGTTTTGCTGATACGA | |
| | CGGTCAATGTTAGTCATATGGACACTTAAGGCAAATTGTTCAGAACTGAATAA | |
| | AGCTGACGTTTTGCTTCTATCCTTTCTTTGAGTTTTAGTGGATAATGATAATGA | |
| | ACAAGGTGTTCATAAATCTATTATAACAAAGGAATGAGAAAT SEQ ID NO: 47 | |
| gRNA | GTTTTAGAGCTAGAAATAGCAAGTTAAAATAAGGCTAGTCCGTTATCAACTTGA | This work |
| sequence | AAAAGTGGCACCGAGTCGGTGC SEQ ID NO: 48 | |
| IS sequence | GGA TTA TTT GGT AAA TTA TAT TTT GAA GGA SEQ ID NO:49 | This work |
| | GLFGKLYFEG SEQ ID NO: 50 | |
| P45IS5 (IS is | ATGGATAAGAAAGAAGTGAAAAATAGGTTTAGTTTTAGGAAGTTATCCACAGG | This work |
| underlined) | CTTAGCGACAGTATTTTTAGGATCAATTTTCTTTTGGACAAATGGACAAACGGT | |
| | TCAAGCAGATAGTGTAGAGCCAGCTAGTGAACAGGCTGTACAAAATGTTGACT | |
| | CTCAAGTACAGGCTGATAATACTGTTTCGGAAAATACCGTTAATGAAGAAAAT | |
| | GGCTCTACTTCCGAAACTACTACTGAAGTTAAGACAGAAATGCCGTCTGTTGA | |
| | TACAACATCTCAAGCTAAAGATGCAGTAGAAACTTCAGATAATAAGAAAGTTGA | |
| | GCTCCCTCAAGGAGAAGCAGATAAGCAGGTTCCACAAAAGTTAGAGGTTAATA | |
| | AGAGTAATCAAGCAGCTGAAACAACTGATAAAGATACAAAGCAAAATGCTACT | |
| | TCTGCAACACCAGCACAACTTAATGAAAATACAGCTCCAGTTGTTGTAAAAGC | |
| | TAAGTCGGAAGGAAAAGAAGTAGTTAAGGCTACTGATCCGACTGATTATCCAA | |
| | CTGAAGTTGGTCAAATCATTGATCAAGATAAATATATTTATCAAATTTTGTCGCT | |
| | TAATGATCGTAGTGGCCGACCTTCTGATTCGAAGCTGGTTCTTACCACTAATA | |
| | GAAATGATCATAATGACAAGAATATCTATGCTTACGTAGTTGATAGAAATAATA | |
| | GAAGAGTAAGTCAATCAGTTACAGTTGGTGTAGATCAACATACTATTATTAGTG | |
| | TGAATGGTCGCGGATATCAAATTTCTAATACCGGCGGTAGCAATGTCATTGTA | |
| | GATGGCAAAGAAGTGCCAACGCAGAATACTTCTACTGTTACTTCGGGTAATGG | |
| | TACTACTAGTCCAATCTATGGATTAGGTAATACTACTCGTGGTGATTATTCCGC | |
| | AATTGGTGAAATCCCACCAGTATACACTGAAAATTCAGTAATCAAGTATTACTA | |
| | TCGTGATGAAAATGGTAATTTAAAAGAAGCTGAAAGTTCTGATCAGTATCCTAA | |
| | CGTAAACGTTTCGGGTCTTACTGGTCAAGAATTTGTAATTCCTAATGTGGATCA | |
| | ATATAAGCGGGTTATCAAGGGACGTTATTTAAATTCAGATAATTTGCCTACAGG | |
| | TGATTTCACGGGAACGATTTCTCAATTTGGTGAGGGGAAATATTATAAGAAAG | |
| | TCTACTATGATTATGGTACAGATGATGTGGATTATTACGTAGTATATAACCAAG | |
| | TTTCACCTGACGGCACAATGGATGTTAGTCTCTTTAGAGGTGACAATAATACA | |
| | CCTATTGAATCAAGAAGGGTGGGTCCAGGTAGATCTATTCGTTTTACCAGTCG | |
| | TAACTATACTGCTCGTAATCCATATGTGACCGAAACACCACATGAAGTACAATT | |
| | TATTTACGATAAATTAGGTTCCATTGTTCCAGTCGATGAAGATGGTAACGTAAT | |
| | TGGCGACTTAGTCCAATTCAATAATAGTACTGATCCAACTAAGGCTGCTGTAA | |
| | CCGATTCGCCAGTTATTGCTGGTTATACAATTAAGGATCCTACTCAAAGAGAG | |
| | ATTACCCCACATGATCCTGGCAAAAATATTAAGGTAGTCTATGTTCGCAACCAT | |
| | GTGACAGCAGCTATTAAGTATATCGATGATACTGCTGGCGATGACTTAAGTGC | |
| | GTACAACAAGTCAATTACAGCTAAGCCAGGTGAAGCACTTAACTATACTACTA | |
| | AAGATTCAATTACAGAACTCCAGAATAAAGGGTATGTATTAGTAAGTGATAACT | |
| | TCAATGTAACTACTATGCCTGAAAATGGTGGTAATTACGAAGTTCACGTAAAG | |
| | CATGGCACTAAGACAATCGATCCAGATAACCCAACTGATAAGTACACCAAGAA | |
| | GGATTTACAAAAAACAGCTACTCGTACGATTAATTATGTTGATGATCAAGGCAA | |
| | CAAGATTGCAGAATCTGTGACTTCCACAGTTGTTTTCACAGGGACTGGTACTG | |
| | TAGATGCCGTAACCGGTAACTTAGTGAACTTACATCCCGACGGTTCGATTAAA | |
| | GACCAAAACGGTAAGCTTACTTGGACTTACTCAGTTGATGGCGGTGTTGTACA | |
| | AAAAAGTGATACTTACACATTTAGCGCAACAACTGCTCGACCAACTATTGATCA | |
| | CAATAATTCTACTTACAACTTTACTTCTACTACTCCCGCTGATTACAATGCTGG | |
| | CAATGGTGCTGTATCGAGTTATCGTGTGAATAGTACTGATCCACAAAACTTAAT | |
| | TGTTAATGTTGTTTATACCAAGCAAGCTATCTACCATGCAGGTAAGACTGAAAC | |
| | TAAGAGTGTAACTCGCACCATTAATTATTTAGATGGTAAGACTGGCGAAAAGA | |
| | TACCAACTGATTTAATTGCAACTAACCCAGTTGCACAAACAGTTAATTTGCATC | |
| | GTACTGAAATTATTGATGACAACGGCAAGGTGATCGGCTACGGTACAATCAGT | |
| | AAAGATGGTAAATCATACACTATTAACAATGATTGGGTAGTCGACGGTAAGTG | |
| | GGCAAGTGTAACTTCACCTGATTTATCAGCTAAGGGTTATAAAGCTCCACGTT | |
| | TTGAAAATGGTACTTCAGCTGCTAGAGTTGACGAAGTAATTGTTGGTAGTGGT | |
| | ACCAAAGACGCTACTGTTAATGTTTATTACGATCATAATTTGATCCCAATTGGA | |
| | CCAGATAATTTTGATAAGCATGGCGTAGATCGAAGCCAGATTGAGAAGCAGGT | |
| | TAAAGAAACAGTTCATTATGTAGGTGCTGGCGATAAGACTCCTGCTGATCATG | |
| | TGCAAACTTCGAAGTGGACGCGCACTATTACTATTGATGCGGTAACTAAAGAA | |
| | GTTGTACCTAATGGTCAATATACAACTGATTGGACAATTCCAAAGGGTGAGAA | |
| | GACCGAGTATGCTCAAGTAAATACGCCAGTAGTTAATGGCTACTATGCTGATC | |
| | AAGCTAATGTTCCGGCAACGACTGTAACTCAAAATGATATTGAAAAAACAGTA | |
| | ACTTATAAGCAAATTGGATTATTTGGTAAATTATATTTTGAAGGAGGTAGGATT | |
| | GTTCCAGTTGATCCAAATGGTAAGCCAATTCCAGATGCACCAACTCCACAATA | |
| | TCCTAACGATCCAACGGATCCGACTAAGGTACTTCCTAATGTACCGGTGCCAA | |
| | ATATTCCAGGCTACAAGCCAAGTGTGCCAACAGTTACTCCAACTGACCCTGGC | |
| | AAGGATACACAAGTTCCATATACACCGGTAACTCCAACTAATCCAGATAATCC | |
| | AGTCATTCCAACGCCTCAACCGGAACCAAACCCTGATAATGGTAAGGATAAGC | |
| | CGGTCGATCCATCCAAGCCATCAGATGATCCAGTTCATCCTGAATATCCTGGT | |
| | ATTAAGAGGGGACAGGATAAACCTGATAAGGAAAAGACTGATAAGAAGAGAA | |
| | ATGGCAAGACTAAGGGTAAAGAAAATACACCTACTGGAAGAGATGCTGTTAAG | |
| | CGAGCTGGACGAAGCGATGATGCACTTAAATTAGCTAGTGAAGCTAAAAATCG | |
| | CCGTATGACTATTCAAGGTAAGAATGAAGAATTACCACAAGCTGGTGAAGATC | |
| | ATAATGCTATGGCGTTGATTGGTCTTGCATTTGCCACTCTTGCTGGAAGTGTA | |
| | GTCTTTGCTACTGATAGGAAACGGAGATAA SEQ ID NO: 51 | |
| Mouse HO-1 | ATGGAACGTCCACAACCTGATTCAATGCCACAGGATTTATCAGAAGCTTTGAA | This work |
| | AGAGGCTACAAAGGAAGTTCATATACAAGCTGAGAATGCTGAATTTATGAAGA | |
| | ATTTCCAGAAAGGACAAGTTTCTAGAGAAGGATTTAAGTTAGTTATGGCTTCAT | |
| | TGTACCATATATATACAGCTTTGGAAGAGGAAATTGAGAGAAATAAACAGAAT | |
| | CCAGTTTACGCTCCATTATATTTCCCAGAGGAATTACATAGACGTGCTGCATTA | |
| | GAACAAGACATGGCATTCTGGTATGGTCCACACTGGCAAGAGATTATTCCATG | |
| | TACACCAGCTACACAACACTATGTTAAAAGATTACATGAAGTCGGACGTACAC | |
| | ACCCAGAATTATTGGTTGCACATGCTTACACTAGATACTTAGGAGACTTGTCT | |
| | GGAGGTCAGGTTCTTAAGAAAATTGCTCAGAAAGCTATGGCATTACCATCTTC | |
| | AGGAGAGGGTTTAGCATTTTTCACATTCCCAAATATTGATTCACCTACTAAATT | |
| | CAAGCAGTTATACAGAGCTAGAATGAACACATTAGAAATGACTCCAGAAGTAA | |
| | AGCATCGTGTAACAGAAGAGGCTAAAACTGCTTTCTTGTTAAATATTGAGTTAT | |
| | TCGAAGAGTTGCAGGTTATGTTGACTGAGGAACACAAGGATCAATCTCCATCA | |
| | CAGATGGCATCATTACGTCAGCGTCCAGCTTCATTGGTACAAGACACTGCTCC | |
| | AGCAGAAACTCCAAGAGGTAAGCCACAAATTTCAACAAGTTCATCACAAACAC | |
| | CTTTGTTACAGTGGGTTCTTACATTGTCTTTTCTTTTAGCTACTGTAGCAGTTG | |
| | GAATATATGCAATGTAA SEQ ID NO: 52 | |
| bga | ATGCGCAACTTGACCAAGACATCTCTATTACTGGCCGGCTTATGCACAGCGG | This work |
| | CCCAAATGGTTTTTGTAACACATGCCTCAGCTGAGGAAGTAGCATCTTCTAAC | |
| | ACTCAAACAGGTGAAACAACAGTTCACCAAGCCCAGCCTTTGGATAAACTTCC | |
| | TGACGACGTGGCAGCTGCAATTGCAAAGGOGGATGAGAACGGGGGAAGAGA | |
| | ATTTGTAAAACCGAAAGCTGAATCAGAGGGCGGTAAAGTTACCAAGGACACG | |
| | GAGCCTACAAAACCAGCCAACGAAGGTTCTCATGAGTTGGCAAGTCCAAAAG | |
| | TCGAAACGCCGAATAAGGTTGAAGAAGGTACAAAAGCCGAAGATAAACAAAA | |
| | GTCTGAGGAGGCTAACCCTAAGCCGGTCGAATCTGCAAGTACTTCAGGCACT | |
| | GAGCTTAAAGAAGATTCAAAAAAAACTTCTGAGAAGGATCAGGTGAAAGCAGA | |
| | TACAGAAATAAAGCCAAGCTCTGAGAAGAGCCAGGCCCTTAGCGGCGAATCA | |
| | AATAAAGCAGAAGTCGAGAAAGAAAAACAGCTTTTGTCTGAGAGAAAACAAGA | |
| | CTTTAATAAAGACTGGTATTTTAAATTAAATGCCCAGGGAGATTTCAGTAAAAA | |
| | AGACGTGGATGTGCATGATTGGTCAAAATTAAACTTACCGCATGATTGGTCTA | |
| | TTTACTTTGACTTTGATCACAAGAGCCCGGCACGAAACGAGGGGGGTCAGTT | |
| | AAACGGGGGGACCGCTTGGTATCGAAAGACTTTTACCTTAAATGAAGCGGAC | |
| | AAGAATAAGGACGTGCGTATTAACTTTGACGGAGTATACATGGACAGCAAAGT | |
| | CTATGTGAATGGGAAGTTCGTGGGACACTATCCAAGTGGTTACAATCACTTCT | |
| | CTTATGACATTACTGAGTTTCTTAATAAAGATGGATCAGAAAACAGCATTACCG | |
| | TTCAAGTTACTAACAAGCAACCGAGCTCTCGATGGTATTCTGGATCTGGTATC | |
| | TATCGAGACGTTACTCTTAGTTACCGTGATAAAGTCCACGTGGCTGAAAATGG | |
| | TAACCATATTACCACCCCTAAGCTTGCTGAGCAGAAGGAAGGAAATGTTGAAA | |
| | CTCAGGTTCAGTCAAAGATAAAAAATACTGACAAGAAAGCTGCTAAAGTGTTC | |
| | GTTGAACAGCAAATATTTACCAAGGAGGGGAAGGTCGTGAGTGAGTTAGTGC | |
| | GTAGCGAAACTAAAAACTTAGCTGAAAACGAGGTTGCCGACTTTCGTCAGACA | |
| | ATACTTGTTAATAAGCCAACTTTATGGACGACTAAGTCTTATCACCCTCAGTTG | |
| | TATGTGCTTAAGACCAAAGTATACAAGGAGGGTCAATTAGTGGACGTGACGG | |
| | AGGACACATTTGGATATAGATATTTTAACTGGACTGCCAAAGATGGCTTTTCAT | |
| | TGAATGGAGAAAGAATGAAATTTCATGGAGTGAGTATCCATCACGATAATGGA | |
| | GCCTTAGGAGCAGAGGAAAATTATAAAGCTACATACCGAAAATTAAAATTATTG | |
| | AAGGATATGGGTGTCAACAGTATTCGTACCACGCACAACCCTGCGAGCCCAC | |
| | AGTTACTTGACGCCGCGGCAAGTTTAGGTCTTTTAGTACAGGAGGAGGCATT | |
| | CGACACCTGGTATGGTGGGAAAAAGACTTATGATTATGGCCGTTTCTTCGATC | |
| | AAGATGCCACACATCCTGAGGCCAAAAAGGGTGAAAAATGGAGCGATTTCGA | |
| | TTTAAGAACTATGGTTGAACGAGACAAGAATAACCCTTCAATAGTGATGTGGA | |
| | GTTTGGGTAACGAAGTGGAGGAGGCTAACGGCTCTCCACGTAGCATCGAGAC | |
| | CGCGAAAAGATTAAAAACAATCATTAAAGCCATCGACACTGAGAGATACGTAA | |
| | CTATGGGTGAAAACAAATTTTCACGTGCTGCTACCGGAGATTTCCTTAAGCTT | |
| | GCTGAAATAATGGATGCGGTTGGAATGAATTACGGAGAAAGATTTTATGACGC | |
| | CGTTCGTAGAGCCCATCCAGACTGGTTGATATACGGTTCAGAGACCAGCTCA | |
| | GCCACGCGAACACGAGACTCTTATTACAATCCTGCCCAGATACTTGGTCATGA | |
| | CAATCGTCCTAACAGACATTATGAACAGTCTGACTATGGTAACGATAGAGTAG | |
| | GATGGGGTCGTACCGCAACAGAAAGTTGGACATTCGATCGAGATCGAGCTGG | |
| | ATATGCCGGTCAGTTCATCTGGACAGGCATCGACTACATAGGTGAGCCGACC | |
| | CCATGGCATAACCAGGATAACACCCCGGTTAAAAGTAGTTATTTTGGTATAATT | |
| | GACACCGCAGGGTTGCCGAAAAACGATTTCTACCTTTACCGATCAGAGTGGT | |
| | ATTCAGCAAAGGAAAAACCGACAGTTAGAATATTACCACATTGGAATTGGACA | |
| | GAAGAAACCTTAAAAGACCGAAAGATGCTTGTGGATGGAAAAGTACCTGTTCG | |
| | TACTTTTTCAAATGCCGCAAGTGTCGAGTTGTTTTTGAACGGGCAGTCTCTTG | |
| | GTAAAAAGGAGTACACAAAGAAAAGAACTGAGGACGGACGTCCTTATCACGA | |
| | GGGGGCTAAGCCTTCAGAATTGTACTTAGAGTGGTTAGTAAAGTACCAGCCA | |
| | GCACATTTAGAAGCTATAGCTAGAGATGAATCTGGAAAAGAAATTGCTAGAGA | |
| | TAAAATTACAACTGCTGGTAAGCCAGCTGCAGTTAGATTGATTAAGGAAGATC | |
| | ATGCTATTGCAGCTGATGGAAAGGATTTAACATACATATACTATGAAATTGTAG | |
| | ATTCTCAAGGTAACGTAGTTCCTACAGCTAACAATTTAGTAAGATTCCAGTTGC | |
| | ATGGACAGGGACAATTGGTTGGTGTAGACAATGGAGAGCAAGCTAGTCGTGA | |
| | ACGTTACAAAGCTCAAGCTGATGGATCATGGATTCGTAAAGCATTTAACGGAA | |
| | AGGGAGTTGCAATTGTAAAATCAACTGAACAAGCAGGTAAATTTACTTTAACTG | |
| | CTCATTCAGACTTATTGAAATCATCTCAAGTTACAGTATTCACAGGTAAGAAAG | |
| | AAGGACAAGAAAAGACAGTATTAGGAACTGAAGTTGCAAGAGTTAGAACATTG | |
| | ATAGGAAAAGATCCAAAGATGCCTAAAACTGTAGGATTTGTTTACAGCGATGG | |
| | ATCTCGTGAGAAATTACCTGTTACTTGGTCTCAGGTAGATGTTTCACAGGCAG | |
| | GTGTTGTAACAGTTAAAGGAACTGCTAACGGTAGAGAAGTTGAGGCTAGAGTT | |
| | GAGGTATTAGCTATAGCTAAAGAGTTGCCAACTGTTAAGCGTATTGCTCCTGG | |
| | AGCAGATTTGAATACAGTTGATAAATACGTTAGTATATTAGTAACTGATGGATC | |
| | TGTTCAGGAATATGAGGTTGACAGATGGGAGATTGCAGAAGCAGATAAAGCT | |
| | AAGTTATCTGTTGCAGGATCTAGAATTCAAATGACTGGACAGTTAGCAGGTGA | |
| | GACAATTCATGCAACATTGGTTGTAGAAGAAGGTAACGCTGCTGCACCAGCA | |
| | GTTCCAACTGTTACAGTTGGTGGAGAGGCTGTTACAGGTTTAACTTCACAGCA | |
| | ACCAATGCAGTATAGAACTTTGGCTTACGGAGCTCAATTGCCTGAAGTAACAG | |
| | CTTCTGCTGAAAACGCTGATGTTACAGTTCTTCAAGCTTCAGCTGCAAATGGT | |
| | ATGAGAGCATCAATATTTGTACAACCAAAGGATGGTGGACCATTGCAGACATA | |
| | CGCTATTCAGTTTTTGGAAGAAGCACCTAAGATTGATCACTTGAATCTTCAAGT | |
| | AGAGCAAGCTGACGGATTGAAAGAGGATCAAACTGTTAACTTATCAGTTAGAG | |
| | CTCACTATCAAGATGGTACACAAGCTGTTCTTCCAGCAGATAAGGTTTCATTCT | |
| | CAACATCTGGTGAGGGAGAAGTTGCTGTTCGTAAAGGAATGTTGGAATTACAC | |
| | AAACCAGGTGCATTAACATTGAAAGCTGAGTATGAAGGAGCTACTGGACAAAT | |
| | AAACTTGACAATTCAAGCTAATACAGAGAAGAAAATTGCTCAATCAATTAGACC | |
| | AGTTAATGTTGTAACAGATCTTCATCAGGAACCTACATTACCATCTACAGTTAC | |
| | TGTTGAATACGACAAAGGTTTCCCTAAAGCTCATAAGGTTACATGGCAAGCTA | |
| | TTCCTAAAGAGAAATTAGACCATTACCAATCATTTGAAGTTTTGGGTAAGGTTG | |
| | AAGGAATTGACATGGAGGCTCGTGCTAAAGTTAGTGTTGAAGGAATTGTATCA | |
| | GTTGAAGAGGTTTCAGTTACTACACCTATAGCTGAGGCTCCACAATTGCCAGA | |
| | ATCTGTTAGAACTTACGATTCAAACGGACACGTTTCTTCAGCAAAAGTTGCAT | |
| | GGGATGCTATACGTCCAGAACAATACGCACGTGAGGGTGTATTCACAGTTAA | |
| | CGGACGTTTGGAAGGAACTCAATTAACTACTAAATTACATGTAAGAGTATCAG | |
| | CTCAGACTGAGCAGGGAGCTAACATTTCTGACCAATGGACAGGATCTGAATT | |
| | GCCTTTGGCATTCGCATCAGATTCTAATCCAACTGATCCAGTATCAAACGTAA | |
| | ACGATAAATTGATATCTTTCAATGATAGACCTGCTAATAGATGGACTAATTGGA | |
| | ACAGATCTAACCCTGAGGCTTCAGTTGGAGTTTTATTCGGAGACTCAGGTATA | |
| | TTGTCTAAGAGATCTGTAGATAATTTGTCAGTTGGATTCCACGAAGACCATGG | |
| | TGTAGGAGCTCCAAAGTCTTATGTAATTGAATACTATGTAGGAAAGACTGTTC | |
| | CTACAGCTCCAAAAAACCCATCTTTCGTTGGTAACGAGGAACACGTTTTTAAC | |
| | GACCCAGCTAACTGGAAGGAGGTTTCAAACTTGAAGGCTCCTGCACAATTAAA | |
| | GGCTGGAGAGATGAATCACTTTTCTTTCGATAAGGTTGAGACTTATGCTGTTA | |
| | GAATCAGAATGGTTCGTGCTGATAATAAATTAGGTACATCAATTACAGAAGTTC | |
| | AGATATTTGCTAAGCAGGTTGCTGCAGCTAAGCAAGGTCAAACTCGTATTCAA | |
| | GTTGACGGAAAGGATTTAGCAAACTTCAATCCAGACTTGACAGATTATTACTTA | |
| | GAATCAGTTGATGGTAAAGTTCCAGCTGTAACAGCTAGTGTTTCTAATAATGG | |
| | ATTGGCTACAGTTGTTCCATCAGTAAGAGAGGGTGAACCAGTTAGAGTAATTG | |
| | CTAAAGCTGAAAATGGTGATATTTTGGGAGAGTATAGATTGCATTTCACAAAG | |
| | GATAAAGACTTATTATCTAGAAAGCCAGTTGCAGCTGTAAAGCAGGCTAGATT | |
| | ATTGCAGTTAGGTCAACCATTAGACTTACCAACTAAAGTACCAGTATATTTCAC | |
| | AGGTAAGGATGGATATGAAGCTAAAGATATGACAGTTGAATGGGAGGAGGTA | |
| | CCAGCTGAAAACTTAACTAAAGCTGGTCAATTCACAGTACGTGGACGTGTATT | |
| | AGGATCTAATTTGAATGCTGAGTTTACTGTTAGAGTTACTGACAAGTTGGGTG | |
| | AAGCATTAAGTGATAACCCAAACTATGATGAGAACTCAAATCAAGCTTTCGCTT | |
| | CAGCTACTAATGACATTGATGACTCTTCACACGATAGAGTTGACTATATTAATG | |
| | ATAGAGACCATTCAGAGAATAGACGTTGGACTAATTGGTCTAAGACACCATCT | |
| | TCAAATCCAGAAGTTTCTGCTGGAGTTATTTTTAGAGAGAATGGTAAAATAGTT | |
| | GAACGTACAGTTGCTCAGGCTAAATTACATTTCTTTGCAGATTCTGGAACAGA | |
| | TGCTCCATCTAAATTGGTTTTGGAAAGATATGTAGGTCCAGACTTTGAGGTTC | |
| | CTACTTATTATTCAAACTACCAAGCTTACGAATCAGGACATCCATTCAACAATC | |
| | CAGAAAACTGGGAAGCAGTTCCATACCGTGCTGATAAAGACATTGAAGCTGG | |
| | AGACGAAATAAATGTTACATTTAAGGCTGTAAAAGCTAAGGCTATGCGTTGGC | |
| | GTATGGAACGTAAAGCTGATAAGTCAGGAGTTGCAATGATTGAAATGACATTT | |
| | CTTGCTCCATCTGAATTGCCACAGGAATCTACACAGTCAAAGATATTAGTAGA | |
| | TGGTAAAGAATTGGCTGACTTTGCTGAGAATAGACAAGACTATCAGATAACAT | |
| | ACAAAGGTAAGAGACCAAAAGTTGCAGTTGAGGAAAACAATCAAGTTGCATCA | |
| | ACAGTTGTAGACTCAGGAGAGGACAGATTACCAGTTTTGGTTCGTTTAGTTTC | |
| | AGAGTCAGGAAAGCAAGTTAAAGAATATAGAATTCAATTAATTAAGGAGAAAC | |
| | CAGTTTCAGAAAAGACAGTAGCAGCTTAA SEQ ID NO: 53 | |
| ped | AAGTATTATGGTAATGGAGTTACATGTGGTAAACATTCATGTTCTGTAGATTGG | This work |
| | GGTAAAGCTACAACTTGTATAATTAACAATGGAGCTATGGCATGGGCTACTGG | |
| | TGGACATCAAGGAAATCATAAATGTTAA SEQ ID NO: 54 | |
| IcnA | AGAAAACTTATTTCAATTACTTTTTAGATAAAATAATGGGAAGAGGCAATCAGT | |
| promoter; | AGAGTTATTAACATTTGTTAACGAGTTTTATTTTTATATAATCTATAATAGATTTA | |
| also called | TAAAAATAAGGAGATTATT SEQ ID NO: 55 | |
| P_{con} | ALTERNATIVE: | |
| | TTAACATTTGTTAACGAGTTTTATTTTTATATAATCTATAATAGATTTATAAAAAT | |
| | SEQ ID NO: 56 | |
| NisR/NisK | GTGTATAAAATTTTAATAGTTGATGATGATCAGGAAATTTTAAAATTAATGAA | |
| NisR is | GACAGCATTAGAAATGAGAAACTATGAAGTTGCGACGCATCAAAACATTTC | |
| bolded; NisK | ACTTCCCTTGGATATTACTGATTTTCAGGGATTTGATTTGATTTTGTTAGATAT | |
| is underlined | CATGATGTCAAATATTGAAGGGACAGAAATTTGTAAAAGGATTCGCAGAGA | |
| (there is | AATATCAACTCCAATTATCTTTGTTAGTGCGAAAGATACAGAAGAGGATATT | |
| overlap) | ATAAACGGCTTAGGTATTGGTGGGGATGACTATATTACTAAGCCTTTTAGCC | |
| | TTAAACAGTTGGTTGCAAAAGTGGAAGCAAATATAAAGCGAGAGGAACGCA | |
| | ATAAACATGCAGTTCATGTTTTTTCAGAGATTCGTAGAGATTTAGGACCAATT | |
| | ACATTTTATTTAGAAGAAAGGCGAGTCTGTGTCAATGGTCAAACAATTCCAC | |
| | TGACTTGTCGTGAATACGATATTCTTGAATTACTATCACAACGAACTTCTAAA | |
| | GTTTATACGAGAGAGGATATTTATGATGACGTATATGATGAATATTCTAATG | |
| | CACTTTTTCGGTCAATCTCGGAGTATATTTATCAGATTAGGAGTAAGTTTGCA | |
| | CCATACGATATTAATCCGATAAAAACGGTTCGGGGACTTGGGTATCAGTGG | |
| | CATGGGTAAAAAATATTCAATGCGTCGACGGATATGGCAAGCTGTCATTGAAA | |
| | TTATCATAGGTACTTGTCTACTTATCCTGTTGTTACTGGGCTTGACTTTCTTTCT | |
| | ACGACAAATTGGACAAATCAGTGGTTCAGAAACTATTCGTTTATCTTTAGATTC | |
| | AGATAATTTAACTATTTCTGATATCGAACGTGATATGAAACACTACCCATATGA | |
| | TTATATTATGTTTGACAATGATACAAGTAAAATTTTGGGAGGACATTATGTCAA | |
| | GTCGGATGTACCTAGTTTTGTAGCTTCAAAACAGTCTTCACATAATATTACAGA | |
| | AGGAGAAATTACTTATACTTATTCAAGCAATAAGCATTTTTCAGTTGTTTTAAGA | |
| | CAAAACAGTATGCCAGAATTTACAAATCATACGCTTCGTTCAATTTCTTATAAT | |
| | CAATTTACTTACCTTTTCTTTTTTCTTGGTGAAATAATACTCATTATTTTTTCTGT | |
| | CTATCATCTCATTAGAGAATTTTCTAAGAATTTTCAAGCCGTTCAAAAGATTGC | |
| | ATTGAAGATGGGGGAAATAACTACTTTTCCTGAACAAGAGGAATCAAAAATTAT | |
| | TGAATTTGATCAGGTTCTGAATAACTTATATTCGAAAAGTAAGGAGTTAGCTTT | |
| | CCTTATTGAAGCGGAGCGTCATGAAAAGCATGATTTATCCTTCCAGGTTGCTG | |
| | CACTTTCACATGATGTTAAGACACCTTTAACAGTATTAAAAGGAAATATTGAAC | |
| | TGCTAGAGATGACTGAAGTAAATGAACAACAAGCTGATTTTATTGAGTCAATG | |
| | AAAAATAGTTTAACTGTTTTTGACAAGTATTTTAACACAATGATTAGTTATACAA | |
| | AACTTTTGAATGATGAAAATGATTACAAAGCGAGAATCTCCCTGGAGGATTTTT | |
| | TGATAGATTTATCAGTTGAGTTGGAAGAGTTGTCAACAACTTATCAAGTGGATT | |
| | ATCAGCTAGTTAAAAAAACAGATTTAACCACTTTTTACGGAAATACATTAGCTT | |
| | TAAGTCGAGCACTTATCAATATCTTTGTTAATGCCTGTCAGTATGCTAAAGAGG | |
| | GTGAAAAAATAGTTAGTTTGAGTATTTATGATGATGAAAAATATCTCTATTTTGA | |
| | AATCTGGAATAATGGTCATCCTTTTTCTGAACAAGCAAAAAAAAATGCTGGAAA | |
| | ACTATTTTTCACAGAAGATACTGGACGTAGTGGGAAACACTATGGGATTGGAC | |
| | TATCTTTTGCTCAAGGTGTAGCTTTAAAACATCAAGGAAACTTAATTCTCAGTA | |
| | ATCCTCAAAAAGGTGGGGCAGAAGTTATCCTAAAAATAAAAAAGTAA | |
| | SEQ ID NO: 57 | |
| PnisA | GCGAGCATAATAAACGGCTCTGATTAAATTCTGAAGTTTGTTAGATACAATGAT | |
| | TTCGTTCGAAGGAACTACAAAATAAATTATAAGGAGGCACTCAAA SEQ ID | |
| | NO: 58 | |
| PnisF | GGCAGAAGTTATCCTAAAAATAAAAAAGTAATTTAGTAATCTCTAAGGATTACT | |
| | TTTTTTGTTTCTGAATAGATTCTGAAAATTGTTTTATATACTTTTTTTAAACATAA | |
| | AATAAAGTGAGGAAATATA SEQ ID NO: 59 | |
| SCZA | ATGACTAACATTGACCGTCGTATCAGCAAAACCAAAAAAGCCATCTATCAAGC | |
| | ATTTATTCAATTATTAAATGCTAAAGGATATGAAGCTACAACTGTTCAAGATATT | |
| | ATTGATTTGGCTGATGTTGGAAGATCAACATTCTATTGTCATTATGAATCTAAA | |
| | GAATTATTATTGGATCAATTATGTAGATATTTGTTTCATCATTTGTTTGAAAGAG | |
| | AACAAGCTATTTCAACAGAAGATTATTTGGCTCATTTATTCTTACATTTCCATAA | |
| | GAATCAAGATCATATTACATCATTATTGTTCTCAAAGAATGATTATTTCTTAAGA | |
| | CAATTACATAAGGAATTAGAACATCATGTTTATTCAGTTTTAGCTGATAATTTGA | |
| | AAGAAGCTCATCCAAATTTACCAACATCATATTTGCAACATTTGGTTATGTCAA | |
| | ATTTTATTGAAACTTTGACATGGTGGTTGAAGAAAGGACAAGACTTCACAGAT | |
| | CAAGAAGTTGTACAATTTTATTTGGATTTATTAATTCCAAAGAATTGA SEQ ID | |
| | NO: 60 | |
| PsczA/PsczD | ATGGACACTTAAGGCAAATTGTTCAGAACTGAATAAAGCTGACGTTTTGCTTCT | |
| (bidirectional | ATCCTTTCTTTGAGTTTTAGTGGATAATGATAATGAACAAGGTGTTCATAAATC | |
| promoter) | TATTATAACAAAGGAATGAGAAAT SEQ ID NO: 61 | |
| PZITR | TCCTATAATGGTTACTGTTTTCCCTTGAAGACCATATCGGATATTTGGGAGGTC | |
| | TTTTGCATTGATAGTGGTTGTCGCAGAAACTTTATAAGCATTTCCCTCTTTAAA | |
| | AGCTGTGGGAGCACTATCTATTTGGTTGATTATTCCAGTTATCTAGACTCGATA | |
| | ACTTATAAATTACTGACAGATCTGTCAGCTGGTTCAACTAGCGGTGGTCAAAC | |
| | TGTTAGTAATAAAACTTATTGTTTTGATGTTCGGCTTAAGGATGGAAGGATTTT | |
| | TCAAATAAAAAAGTAAAAAATAATGTTAACTGGTTGACATTATTTTTACTTTGCT | |
| | ATATAATTAACCAGTAAACTAATTATGGAGGACGAAATACT SEQ ID NO: 62 | |
| DCAS FROM | ATGGATAAGAAATACTCAATAGGCTTAGCTATCGGCACAAATAGCGTCGGATG | |
| ADDGENE | GGCGGTGATCACTGATGAATATAAGGTTCCGTCTAAAAAGTTCAAGGTTCTGG | |
| PLASMID | GAAATACAGACCGCCACAGTATCAAAAAAAATCTTATAGGGGCTCTTTTATTTG | |
| PMJ841 | ACAGTGGAGAGACAGCGGAAGCGACTCGTCTCAAACGGACAGCTCGTAGAA | |
| (PLASMID | GGTATACACGTCGGAAGAATCGTATTTGTTATCTACAGGAGATTTTTTCAAATG | |
| #39318) | AGATGGCGAAAGTAGATGATAGTTTCTTTCATCGACTTGAAGAGTCTTTTTTGG | |
| | TGGAAGAAGACAAGAAGCATGAACGTCATCCTATTTTTGGAAATATAGTAGAT | |
| | GAAGTTGCTTATCATGAGAAATATCCAACTATCTATCATCTGCGAAAAAAATTG | |
| | GTAGATTCTACTGATAAAGCGGATTTGCGCTTAATCTATTTGGCCTTAGCGCA | |
| | TATGATTAAGTTTCGTGGTCATTTTTTGATTGAGGGAGATTTAAATCCTGATAA | |
| | TAGTGATGTGGACAAACTATTTATCCAGTTGGTACAAACCTACAATCAATTATT | |
| | TGAAGAAAACCCTATTAACGCAAGTGGAGTAGATGCTAAAGCGATTCTTTCTG | |
| | CACGATTGAGTAAATCAAGACGATTAGAAAATCTCATTGCTCAGCTCCCCGGT | |
| | GAGAAGAAAAATGGCTTATTTGGGAATCTCATTGCTTTGTCATTGGGTTTGAC | |
| | CCCTAATTTTAAATCAAATTTTGATTTGGCAGAAGATGCTAAATTACAGCTTTC | |
| | AAAAGATACTTACGATGATGATTTAGATAATTTATTGGCGCAAATTGGAGATCA | |
| | ATATGCTGATTTGTTTTTGGCAGCTAAGAATTTATCAGATGCTATTTTACTTTCA | |
| | GATATCCTAAGAGTAAATACTGAAATAACTAAGGCTCCCCTATCAGCTTCAATG | |
| | ATTAAACGCTACGATGAACATCATCAAGACTTGACTCTTTTAAAAGCTTTAGTT | |
| | CGACAACAACTTCCAGAAAAGTATAAAGAAATCTTTTTTGATCAATCAAAAAAC | |
| | GGATATGCAGGTTATATTGATGGGGGAGCTAGCCAAGAAGAATTTTATAAATT | |
| | TATCAAACCAATTTTAGAAAAAATGGATGGTACTGAGGAATTATTGGTGAAACT | |
| | AAATCGTGAAGATTTGCTGCGCAAGCAACGGACCTTTGACAACGGCTCTATTC | |
| | CCCATCAAATTCACTTGGGTGAGCTGCATGCTATTTTGAGAAGACAAGAAGAC | |
| | TTTTATCCATTTTTAAAAGACAATCGTGAGAAGATTGAAAAAATCTTGACTTTTC | |
| | GAATTCCTTATTATGTTGGTCCATTGGCGCGTGGCAATAGTCGTTTTGCATGG | |
| | ATGACTCGGAAGTCTGAAGAAACAATTACCCCATGGAATTTTGAAGAAGTTGT | |
| | CGATAAAGGTGCTTCAGCTCAATCATTTATTGAACGCATGACAAACTTTGATAA | |
| | AAATCTTCCAAATGAAAAAGTACTACCAAAACATAGTTTGCTTTATGAGTATTTT | |
| | ACGGTTTATAACGAATTGACAAAGGTCAAATATGTTACTGAAGGAATGCGAAA | |
| | ACCAGCATTTCTTTCAGGTGAACAGAAGAAAGCCATTGTTGATTTACTCTTCAA | |
| | AACAAATCGAAAAGTAACCGTTAAGCAATTAAAAGAAGATTATTTCAAAAAAAT | |
| | AGAATGTTTTGATAGTGTTGAAATTTCAGGAGTTGAAGATAGATTTAATGCTTC | |
| | ATTAGGTACCTACCATGATTTGCTAAAAATTATTAAAGATAAAGATTTTTTGGAT | |
| | AATGAAGAAAATGAAGATATCTTAGAGGATATTGTTTTAACATTGACCTTATTT | |
| | GAAGATAGGGAGATGATTGAGGAAAGACTTAAAACATATGCTCACCTCTTTGA | |
| | TGATAAGGTGATGAAACAGCTTAAACGTCGCCGTTATACTGGTTGGGGACGTT | |
| | TGTCTCGAAAATTGATTAATGGTATTAGGGATAAGCAATCTGGCAAAACAATAT | |
| | TAGATTTTTTGAAATCAGATGGTTTTGCCAATCGCAATTTTATGCAGCTGATCC | |
| | ATGATGATAGTTTGACATTTAAAGAAGACATTCAAAAAGCACAAGTGTCTGGA | |
| | CAAGGCGATAGTTTACATGAACATATTGCAAATTTAGCTGGTAGCCCTGCTAT | |
| | TAAAAAAGGTATTTTACAGACTGTAAAAGTTGTTGATGAATTGGTCAAAGTAAT | |
| | GGGGCGGCATAAGCCAGAAAATATCGTTATTGAAATGGCACGTGAAAATCAG | |
| | ACAACTCAAAAGGGCCAGAAAAATTCGCGAGAGCGTATGAAACGAATCGAAG | |
| | AAGGTATCAAAGAATTAGGAAGTCAGATTCTTAAAGAGCATCCTGTTGAAAATA | |
| | CTCAATTGCAAAATGAAAAGCTCTATCTCTATTATCTCCAAAATGGAAGAGACA | |
| | TGTATGTGGACCAAGAATTAGATATTAATCGTTTAAGTGATTATGATGTCGATG | |
| | CCATTGTTCCACAAAGTTTCCTTAAAGACGATTCAATAGACAATAAGGTCTTAA | |
| | CGCGTTCTGATAAAAATCGTGGTAAATCGGATAACGTTCCAAGTGAAGAAGTA | |
| | GTCAAAAAGATGAAAAACTATTGGAGACAACTTCTAAACGCCAAGTTAATCACT | |
| | CAACGTAAGTTTGATAATTTAACGAAAGCTGAACGTGGAGGTTTGAGTGAACT | |
| | TGATAAAGCTGGTTTTATCAAACGCCAATTGGTTGAAACTCGCCAAATCACTAA | |
| | GCATGTGGCACAAATTTTGGATAGTCGCATGAATACTAAATACGATGAAAATG | |
| | ATAAACTTATTCGAGAGGTTAAAGTGATTACCTTAAAATCTAAATTAGTTTCTGA | |
| | CTTCCGAAAAGATTTCCAATTCTATAAAGTACGTGAGATTAACAATTACCATCA | |
| | TGCCCATGATGCGTATCTAAATGCCGTCGTTGGAACTGCTTTGATTAAGAAAT | |
| | ATCCAAAACTTGAATCGGAGTTTGTCTATGGTGATTATAAAGTTTATGATGTTC | |
| | GTAAAATGATTGCTAAGTCTGAGCAAGAAATAGGCAAAGCAACCGCAAAATAT | |
| | TTCTTTTACTCTAATATCATGAACTTCTTCAAAACAGAAATTACACTTGCAAATG | |
| | GAGAGATTCGCAAACGCCCTCTAATCGAAACTAATGGGGAAACTGGAGAAATT | |
| | GTCTGGGATAAAGGGCGAGATTTTGCCACAGTGCGCAAAGTATTGTCCATGC | |
| | CCCAAGTCAATATTGTCAAGAAAACAGAAGTACAGACAGGCGGATTCTCCAAG | |
| | GAGTCAATTTTACCAAAAAGAAATTCGGACAAGCTTATTGCTCGTAAAAAAGAC | |
| | TGGGATCCAAAAAAATATGGTGGTTTTGATAGTCCAACGGTAGCTTATTCAGT | |
| | CCTAGTGGTTGCTAAGGTGGAAAAAGGGAAATCGAAGAAGTTAAAATCCGTTA | |
| | AAGAGTTACTAGGGATCACAATTATGGAAAGAAGTTCCTTTGAAAAAAATCCG | |
| | ATTGACTTTTTAGAAGCTAAAGGATATAAGGAAGTTAAAAAAGACTTAATCATT | |
| | AAACTACCTAAATATAGTCTTTTTGAGTTAGAAAACGGTCGTAAACGGATGCTG | |
| | GCTAGTGCCGGAGAATTACAAAAAGGAAATGAGCTGGCTCTGCCAAGCAAAT | |
| | ATGTGAATTTTTTATATTTAGCTAGTCATTATGAAAAGTTGAAGGGTAGTCCAG | |
| | AAGATAACGAACAAAAACAATTGTTTGTGGAGCAGCATAAGCATTATTTAGATG | |
| | AGATTATTGAGCAAATCAGTGAATTTTCTAAGCGTGTTATTTTAGCAGATGCCA | |
| | ATTTAGATAAAGTTCTTAGTGCATATAACAAACATAGAGACAAACCAATACGTG | |
| | AACAAGCAGAAAATATTATTCATTTATTTACGTTGACGAATCTTGGAGCTCCCG | |
| | CTGCTTTTAAATATTTTGATACAACAATTGATCGTAAACGATATACGTCTACAAA | |
| | AGAAGTTTTAGATGCCACTCTTATCCATCAATCCATCACTGGTCTTTATGAAAC | |
| | ACGCATTGATTTGAGTCAGCTAGGAGGTGACTAA | |
| | SEQ ID NO: 63 | |
| PROTEASE | TTGCGCAACTTGACCAAGACATCTCTATTACTGGCCGGCTTATGCACAGCGG | |
| A: | CCCAAATGGTTTTTGTAACACATGCCTCAGCTGAAGAAAGCATCGAATACGAC | |
| | CATACGTATCAAACCCCTTCATACATCATCGAAAAGTCACCGCAGAAGCCGGT | |
| | ACAAAACACAACCCAGAAAGAATCGCTATTTTCCTATCTTGACAAGCATCAAAC | |
| | GCAGTTTAAGCTCAAAGGGAATGCGAACAGCCATTTTCGCGTTTCGAAAACCA | |
| | TAAAGGATCCAAAGACAAAACAAACGTTTTTTAAATTAACGGAGGTTTACAAAG | |
| | GAATTCCGATTTACGGCTTTGAACAAGCGGTCGCGATGAAGGAAAACAAACAA | |
| | GTGAAAAGTTTCTTTGGAAAGGTGCATCCGCAAATCAAGGACGTCTCCGTCAC | |
| | ACCGTCTATTTCTGAGAAAAAAGCAATACATACAGCAAGGCGTGAGCTCGAG | |
| | GCTTCCATTGGAAAAATCGAATATCTTGATGGGGAACCAAAAGGCGAATTATA | |
| | TATCTATCCACACGACGGTGAATATGATCTCGCCTACCTTGTGAGACTCTOGA | |
| | CATCTGAACCTGAGCCTGGCTATTGGCATTATTTCATCGATGCCAAAAACGGA | |
| | AAGGTCATCGAGTCCTTTAATGCCATTCATGAAGCGGCAGGTACAGGAATCG | |
| | GCGTGTCAGGTGATGAAAAAAGCTTTGACGTCACAGAACAAAATGGGCGCTT | |
| | TTATTTGGCTGACGAAACAAGGGGAAAAGGGATCAATACATTTGACGCGAAGA | |
| | ACCTGAACGAAACCTTGTTTACGCTTTTGTCTCAACTGATCGGGTATACGGGC | |
| | AAAGAAATAGTCAGCGGCACGTCCGTATTTAATGAACCTGCGGCTGTAGACG | |
| | CACACGCAAATGCGCAAGCCGTTTACGATTATTACAGCAAGACATTTGGCCGT | |
| | GATTCTTTTGATCAAAACGGAGCAAGGATTACGTCTACCGTGCATGTCGGCAA | |
| | ACAATGGAACAATGCTGCGTGGAACGGTGTCCAGATGGTATACGGGGATGGA | |
| | GACGGTTCGAAATTTAAGCCGCTGTCTGGATCGCTCGACATTGTCGCGCATG | |
| | AAATCACACACGCAGTCACACAGTATTCCGCCGGTCTTTTATATCAAGGAGAA | |
| | CCCGGTGCATTAAATGAGTCCATTTCTGACATTATGGGCGCGATGGCTGACC | |
| | GTGATGATTGGGAGATCGGCGAAGATGTCTATACTCCTGGTATTGCAGGAGA | |
| | TTCATTGCGGTCATTGGAGGACCCATCTAAGCAGGGAAATCCAGATCATTACT | |
| | CGAACCGCTACACAGGAACAGAGGATTATGGCGGAGTCCATATCAATTCGTC | |
| | CATTCACAATAAAGCAGCTTATCTTCTTGCAGAAGGAGGCGTGCACCACGGT | |
| | GTACAGGTTGAAGGGATTGGGCGTGAAGCAAGTGAACAAATTTACTATCGGG | |
| | CTTTAACATATTATGTAACGGCATCTACAGATTTCAGCATGATGAAGCAAGCG | |
| | GCGATTGAAGCTGCCAATGATTTATACGGTGAAGGCTCGAAGCAATCAGCTTC | |
| | AGTCGAAAAGGCGTATGAGGCTGTCGGCATTCTATGA SEQ ID NO: 64 | |
| PROTEASE | GTGGGTTTAGGTAAGAAATTGTCTGTTGCTGTCGCTGCTTCGTTTATGAGTTT | |
| B: | ATCAATCAGCCTGCCAGGTGTTCAGGCTGCTGAAGGTCATCAGCTTAAAGAG | |
| | AATCAAACAAATTTCCTCTCCAAAAACGCGATTGCGCAATCAGAACTCTCTGC | |
| | ACCAAATGACAAGGCTGTCAAGCAGTTTTTGAAAAAGAACAGCAACATTTTTAA | |
| | AGGTGACCCTTCCAAAAGGCTGAAGCTTGTTGAAAGCACGACTGATGCCCTT | |
| | GGATACAAGCACTTTCGATATGCGCCTGTCGTTAACGGAGTGCCAATTAAAGA | |
| | TTCGCAAGTGATCGTTCACGTCGATAAATCCGATAATGTCTATGCGGTCAATG | |
| | GTGAATTACACAATCAATCTGCTGCAAAAACAGATAACAGCCAAAAAGTCTCTT | |
| | CTGAAAAAGCGCTGGCACTCGCTTTCAAAGCTATCGGCAAATCACCAGACGC | |
| | TGTTTCTAACGGAGCGGCCAAAAACAGCAATAAAGCCGAATTAAAAGCGATAG | |
| | AAACAAAAGACGGCAGCTATCGTCTTGCTTACGACGTGACGATTCGCTATGTC | |
| | GAGCCTGAACCTGCAAACTGGGAAGTCTTAGTTGACGCCGAAACAGGCAGCA | |
| | TTTTAAAACAGCAAAATAAAGTAGAACATGCCGCCGCCACTGGAAGCGGAACA | |
| | ACGCTAAAGGGCGCAACTGTTCCTTTGAACATCTCTTATGAAGGOGGAAAATA | |
| | TGTTCTAAGAGATCTTTCAAAACCAACAGGCACCCAAATCATCACATATGATTT | |
| | GCAAAACAGACAAAGCCGCCTTCCGGGCACGCTTGTCTCAAGCACAACGAAA | |
| | ACATTTACATCTTCATCACAGCGGGCAGCCGTTGACGCACACTATAACCTCGG | |
| | TAAAGTGTACGATTATTTTTATTCAAACTTTAAACGAAACAGCTATGATAACAAA | |
| | GGCAGTAAAATCGTTTCTTCCGTTCACTACGGCACTCAATACAATAACGCTGC | |
| | ATGGACAGGAGACCAGATGATTTACGGTGATGGCGACGGTTCATTCTTCTCTC | |
| | CGCTTTCCGGCTCATTAGATGTGACAGCGCATGAAATGACACATGGCGTCAC | |
| | CCAAGAAACAGCCAACTTGATTTATGAAAATCAGCCAGGTGCATTAAACGAGT | |
| | CTTTCTCTGACGTATTCGGGTATTTTAACGATACAGAAGACTGGGACATCGGT | |
| | GAAGACATTACGGTCAGCCAGCCTGCTCTTCGCAGCCTGTCCAACCCTACAA | |
| | AATACAACCAGCCTGACAATTACGCCAATTACCGAAACCTTCCAAACACAGAT | |
| | GAAGGCGATTATGGCGGTGTACACACAAACAGCGGAATTCCAAACAAAGCCG | |
| | CTTACAACACCATCACAAAACTTGGTGTATCTAAATCACAGCAAATCTATTACC | |
| | GTGCGTTAACAACGTACCTCACGCCTTCTTCCACGTTCAAAGATGCCAAGGCA | |
| | GCTCTCATTCAGTCTGCCCGTGACCTCTACGGCTCAACTGATGCCGCTAAAGT | |
| | TGAAGCAGCCTGGAATGCTGTTGGATTGTAA | |
| | SEQ ID NO: 65 | |
| PROTEASE | GTGAGAAGCAAAAAATTGTGGATCAGCTTGTTGTTTGCGTTAACGTTAATCTTT | |
| C | ACGATGGCGTTCAGCAACATGTCTGCGCAGGCTGCCGGAAAAAGCAGTACAG | |
| | AAAAGAAATACATTGTCGGATTTAAACAGACAATGAGTGCCATGAGTTCCGCC | |
| | AAGAAAAAGGATGTTATTTCTGAAAAAGGCGGAAAGGTTCAAAAGCAATTTAA | |
| | GTATGTTAACGCGGCCGCAGCAACATTGGATGAAAAAGCTGTAAAAGAATTGA | |
| | AAAAAGATCCGAGCGTTGCATATGTGGAAGAAGATCATATTGCACATGAATAT | |
| | GCGCAATCTGTTCCTTATGGCATTTCTCAAATTAAAGCGCCGGCTCTTCACTC | |
| | TCAAGGCTACACAGGCTCTAACGTAAAAGTAGCTGTTATCGACAGCGGAATTG | |
| | ACTCTTCTCATCCTGACTTAAACGTCAGAGGCGGAGCAAGCTTCGTACCTTCT | |
| | GAAACAAACCCATACCAGGACGGCAGTTCTCACGGTACGCATGTAGCCGGTA | |
| | CGATTGCCGCTCTTAATAACTCAATCGGTGTTCTGGGCGTAGCGCCAAGCGC | |
| | ATCATTATATGCAGTAAAAGTGCTTGATTCAACAGGAAGCGGCCAATATAGCT | |
| | GGATTATTAACGGCATTGAGTGGGCCATTTCCAACAATATGGATGTTATCAAC | |
| | ATGAGCCTTGGCGGACCTACTGGTTCTACAGCGCTGAAAACAGTCGTTGACA | |
| | AAGCCGTTTCCAGCGGTATCGTCGTTGCTGCCGCAGCCGGAAACGAAGGTTC | |
| | ATCCGGAAGCACAAGCACAGTCGGCTACCCTGCAAAATATCCTTCTACTATTG | |
| | CAGTAGGTGCGGTAAACAGCAGCAACCAAAGAGCTTCATTCTCCAGCGCAGG | |
| | TTCTGAGCTTGATGTGATGGCTCCTGGCGTGTCCATCCAAAGCACACTTCCTG | |
| | GAGGCACTTACGGCGCTTATAACGGAACGTCCATGGCGACTCCTCACGTTGC | |
| | CGGAGCAGCAGCGTTAATTCTTTCTAAGCACCCGACTTGGACAAACGCGCAA | |
| | GTCCGTGATCGTTTAGAAAGCACTGCAACATATCTTGGAAACTCTTTCTACTAT | |
| | GGAAAAGGGTTAATCAACGTACAAGCAGCTGCACAATAA | |
| | SEQ ID NO: 66 | |

Characterization of biofilm forming proteins. All biofilm forming proteins and their sources are listed in Table 1. Gene expression and biofilm formation were performed by inoculating 150 μl of 1:50 diluted overnight culture of each sample into 96-well cell culture treated plates (Nunclon Delta surface, Thermo Scientific 167008) and 96-well non-treated plates (Falcon, 351172). In addition, for each sample, 2 ml of 1:50 diluted overnight culture was inoculated into a 12-well plate (Thermo Scientific 150628) containing an 18 mm circle cover glass (VWR 16004-300) at the bottom for testing biofilm formation on glass surface. The culture was grown for 24 hours and the biofilm was quantified by crystal violet method⁴⁵.

Auto-aggregation. Cells from overnight cultures of 45 strains were collected by centrifuge at 3000 g for 5 minutes, re-suspended in PBS buffer, and adjusted to a final OD₆₀₀ of 1.0. Three microliters of cell suspensions were added into a 5 ml test tube (Falcon, 352008) and incubated at room temperature. After incubation for 1, 2, 4, and 6 hours, 1 ml of top supernatant was carefully taken from the tube by pipetting and used for measurement of OD₆₀₀ which is labelled as OD_{600_final}. The aggregation rate was calculated as (1−OD_{600_final})/1×100%.

Induction of biofilm formation. For nisin induced or repressed biofilm formation, 150 μl of 1:50 dilution of overnight cultures in fresh GM17/Cm were added to a 96-well cell culture treated plate and incubated at 30° C. for 2 hours. Then nisin was added at a final concentration of 10 ng m⁻¹ and the plate was incubated at 30° C. for 24 hours for biofilm formation. For zinc induced or repressed induction, overnight cultures were directly diluted at 1:50 in GM17/Cm with zinc or EDTA and 150 μl of cultures were added to a 96-well plate at 30° C. for 24 hours for biofilm formation. The biofilms were quantified using the crystal violet method⁴⁵.

Protease treatment. Biofilms were first grown in a 12-well plate with an 18 mm circle cover glass at the bottom for 24 hours. Then, the supernatants were removed by pipetting and biofilms were washed once by PBS buffer. Proteinase K or Trypsin dissolved in PBS was added to biofilms at a final concentration of 10 μg ml⁻¹. Biofilms were treated at 30° C. for 2 hours and then washed once by PBS. The remaining biofilms were quantified by crystal violet staining. For auto-aggregation assay, cells from overnight cultures were collected by centrifuge at 3000 g for 5 minutes, re-suspended in PBS buffer, and adjusted to OD₆₀₀ of 1.0. Three microliters of cell suspensions were added into 5 ml test tubes (Falcon, 352008) and Proteinase K was added at a final concentration of 10 μg ml⁻¹. The test tubes were incubated at room temperature for 4 hours and images were taken.

Transition between planktonic and biofilm states. Overnight cultures were diluted 1:50 by fresh GM17 medium with zinc and inoculated in 12-well plates with each containing an 18 mm circle cover glass at the bottom. The plate was incubated at 30° C. for biofilm formation. Every 12 hours, the supernatant of each sample was carefully removed and fresh medium with zinc was added. At hour 36, the supernatant of each sample was removed and each well was washed once by fresh M17 medium. Then GM17 medium with EDTA was added to the plate for state transition. Every 12 hours, medium was changed with fresh GM17/EDTA. At hour 72, the wells were washed again with M17 medium and then changed back to GM17/Zinc medium. At hour 36, 62, and 108, supernatants were used to measure enzyme activity and biofilms were quantified by crystal violet staining. For nisin induced expression, the supernatant of each sample was taken after induction by nisin for 5 hours to measure protein production.

Measurement of GFP fluorescence. To prepare samples to measure GFP fluorescence of planktonic cells, supernatants were taken from 12-well plates, centrifuged, and re-suspended with PBS buffer. To measure GFP fluorescence of biofilm cells, biofilms were released from the glass cover slips by adding PBS buffer and violently pipetting up and down for 15 seconds. To ensure all the cells including those in the supernatant and in the biofilm of a sample were collected for fluorescence measurement, the cells growing on the bottom of each 12-well plate were scraped off and thoroughly mixed with the corresponding supernatant by vigorously pipetting up and down. Then, the mixture was transferred into a microcentrifuge tube and centrifuged. The resulting cell pellet was re-suspended with PBS buffer by vortex. The GFP fluorescence was measured by a BioTek Synergy H1M reader and OD₆₀₀ was measured by Nanodrop 2000 Spectrophotometers. The relative GFP unit (RFU) is defined as fluorescent units per OD₆₀₀ per 100 μl. Notably, at each time point, six samples were prepared, of which three were taken to measure GFP as described here and the other three were used to measure biofilm formation.

Measurement of enzyme activity. The activity of amylase was measured using EnzChek™ Ultra Amylase Assay Kit (Thermo Fisher, E33651). The activity of mouse Heme Oxygenase-1 in the culture was quantified by Mouse Heme Oxygenase 1 ELISA Kit (abcam, ab204524). To measure β-glucuronidase activity, 50 μl of 20 mM PNPG (p-Nitrophenyl-β-D-glucuronide) was added to 1 ml of cell culture in the 12-well plate that expresses GusA and incubated at room temperature for 15 minutes. Then, 500 μl of supernatant was taken from the 12-well plate and added to a 1.5 ml microcentrifuge tube containing 500 μl of 1 M NaCO₃ for stopping the reaction. The mixture was centrifuged and 200 μl of the mixture was added to a 96-well plate to measure the absorbance at 420 nm. For standard curve, 100 μl of 0-1000 μM PNP (4-Nitrophenol) and 100 μl of 1 M NaCO₃ were added to the same 96-well plate for measurement of absorbance at 420 nm. The relative unit of β-glucuronidase is defined as the micromole of PNP generated per ml of samples per minute.

To measure β-galactosidase activity, 50 μl of supernatant of the bacterial culture was mixed with 25 μl of 20 mM ONPG (o-nitrophenyl-β-galactoside) and 25 μl of PBS buffer in a 96-well plate. The plate was kept at 37° C. for 30 minutes, then 100 μl of 1 M NaCO₃ was added to terminate the reaction. The resulting samples were measured at 420 nm for absorbance. The standard curve was made by dilution of 10 mM ONP (2-Nitrophenol) to the final concentration of 0-1000 μM. 100 μl of each concentration was added to 96 well plate, incubated the same time as samples, and added with 100 μl NaCO₃ at the end of the experiment. The relative unit is defined as the micromole of ONP generated per ml of samples per minute.

To determine the anti-listeria effect of expressed pediocin, agar diffusion assay was performed as previously described⁸⁰. In brief, 25 ml of melted TSB agar (0.85% agar) was cool down to 48° C. by incubating in water bath and added with 200 μl overnight culture of L. monocytogenes 10403S. The cells were gently mixed and poured into a 90 mm plate. A PCR plate was put on the melted agar mix to make wells on it. After incubation at room temperature for half an hour, the PCR plate was removed and pediocin samples were added into the wells. The plate was first incubated at room temperature for 2 hours to diffuse the pediocin into the agar and then incubated at 30° C. for 24 hours to form the inhibition zone.

Scanning electron microscopy (SEM) analysis. Biofilms were grown on 6 mm round glass coverslips in a 24-well plate for 24 hours. Then biofilms were fixed with 2.0% paraformaldehyde and 2.5% glutaraldehyde in 0.1 M Na-Cacodylate buffer (pH 7.4) at 4° C. for 4 hours. After rinse with 0.1 M Na-Cacodylate buffer, they were dehydrated by washing through a graded ethanol series (37, 67, 95, and 3×100% (v/v)] for 10 minutes each. Dehydrated samples were dried in critical point dryer in 100% ethanol and then coated with gold-palladium. Finally, samples were observed using a FEI Quanta FEG 450 ESEM microscope.

Statistical analysis. All of the experiments were performed for at least three times. Replicate numbers of the experiments (n) are indicated in the figure legends. Sample sizes were chosen based on standard experimental requirement in molecular biology. Data are presented as mean±standard deviation (s.d.). Microscopy images are representatives of the images from multiple experimental replicates.

### REFERENCES

1. Cameron, D. E., Bashor, C. J. & Collins, J. J. A brief history of synthetic biology. Nat. Rev. Microbiol. 12, 381-390 (2014).
2. Endy, D. Foundations for engineering biology. Nature 438, 449-453 (2005).
3. Nandagopal, N. & Elowitz, M. B. Synthetic biology: integrated gene circuits. Science 333, 1244-1248 (2011).
4. Pumick, P. E. & Weiss, R. The second wave of synthetic biology: from modules to systems. Nat. Rev. Mol. Cell Biol. 10, 410-422 (2009).
5. Brophy, J. A. & Voigt, C. A. Principles of genetic circuit design. Nat. Methods 11, 508-520 (2014).
6. You, L., Cox, R. S., Weiss, R. & Arnold, F. H. Programmed population control by cell-cell communication and regulated killing. Nature 428, 868-871 (2004).
7. Win, M. N. & Smolke, C. D. Higher-order cellular information processing with synthetic RNA devices. Science 322, 456-460 (2008).
8. Tigges, M., Marquez-Lago, T. T., Stelling, J. & Fussenegger, M. A tunable synthetic mammalian oscillator. Nature 457, 309-312 (2009).
9. Tabor, J. J. et al. A synthetic genetic edge detection program. Cell 137, 1272-1281 (2009).
10. Danino, T., Mondragón-Palomino, O., Tsimring, L. & Hasty, J. A synchronized quorum of genetic clocks. Nature 463, 326-330 (2010).
11. Delebecque, C. J., Lindner, A. B., Silver, P. A. & Aldaye, F. A. Organization of intracellular reactions with rationally designed RNA assemblies. Science 333, 470-474(2011).
12. Qi, L. S. et al. Repurposing CRISPR as an RNA-guided platform for sequence-specific control of gene expression. Cell 152, 1173-1183 (2013).
13. Mee, M. T., Collins, J. J., Church, G. M. & Wang, H. H. Syntrophic exchange in synthetic microbial communities. Proc. Natl. Acad. Sci. U.S.A. 111, E2149-E2156 (2014).
14. Chen, Y., Kim, J. K., Himing, A. J., Josid, K. & Bennett, M. R. Emergent genetic oscillations in a synthetic microbial consortium. Science 349, 986-989 (2015).
15. Kong, W., Meldgin, D. R., Collins, J. J. & Lu, T. Designing microbial consortia with defined social interactions. Nat. Chem. Biol. 14, 821-829 (2018).
16. Carothers, J. M., Goler, J. A. & Keasling, J. D. Chemical synthesis using synthetic biology. Curr. Opin. Biotechnol. 20, 498-503 (2009).
17. Smanski, M. J. et al. Synthetic biology to access and expand nature's chemical diversity. Nat. Rev. Microbiol. 14, 135-149 (2016).
18. Gilbert, C. et al. Living materials with programmable functionalities grown from engineered microbial co-cultures. Nat. Mater. 20, 691-700 (2021).
19. Tang, T.-C. et al. Materials design by synthetic biology. Nat. Rev. Mater. 6, 332-350(2021).
20. Rylott, E. L. & Bruce, N. C. How synthetic biology can help bioremediation. Curr. Opin. Chem. Biol. 58, 86-95 (2020).
21. de Lorenzo, V. Systems biology approaches to bioremediation. Curr. Opin. Biotechnol. 19, 579-589 (2008).
22. Ruder, W. C., Lu, T. & Collins, J. J. Synthetic biology moving into the clinic. Science 333, 1248-1252 (2011).
23. Weber, W. & Fussenegger, M. Emerging biomedical applications of synthetic biology. Nat. Rev. Genet. 13, 21-35 (2012).
24. Kitada, T., DiAndreth, B., Teague, B. & Weiss, R. Programming gene and engineered-cell therapies with synthetic biology. Science 359(2018).
25. O'Toole, G., Kaplan, H. B. & Kolter, R. Biofilm formation as microbial development. Annu. Rev. Microbiol. 54, 49-79 (2000).
26. Hall-Stoodley, L., Costerton, J. W. & Stoodley, P. Bacterial biofilms: from the natural environment to infectious diseases. Nat. Rev. Microbiol. 2, 95-108 (2004).
27. Flemming, H.-C. et al. Biofilms: an emergent form of bacterial life. Nat. Rev. Microbiol. 14, 563-575 (2016).
28. Prindle, A., Liu, J., Asally, M., Garcia-Ojalvo, J. & Suel, G. M. lon channels enable electrical communication in bacterial communities. Nature 527, 59-63 (2015).
29. Flemming, H.-C. & Wingender, J. The biofilm matrix. Nat. Rev. Microbiol. 8, 623-633 (2010).
30. Alldredge, A. L. & Silver, M. W. Characteristics, dynamics and significance of marine snow. Prog. Oceanogr. 20, 41-82 (1988).
31. Azam, F. & Long, R. A. Sea snow microcosms. Nature 414, 495-498 (2001).
32. Zarrinpar, A., Chaix, A., Yooseph, S. & Panda, S. Diet and feeding pattern affect the diurnal dynamics of the gut microbiome. Cell Metab. 20, 1006-1017 (2014).
33. Marinangeli, C., Harding, S., Zafron, M. & Rideout, T. A systematic review of the effect of dietary pulses on microbial populations inhabiting the human gut. Benef. Microbes 11, 457-468 (2020).
34. Wood, T. K., Hong, S. H. & Ma, Q. Engineering biofilm formation and dispersal. Trends Biotechnol. 29, 87-94 (2011).
35. Lee, J., Jayaraman, A. & Wood, T. K. Indole is an inter-species biofilm signal mediated by SdiA. BMC Microbiol. 7, 1-15 (2007).
36. Hong, S. H. et al. Synthetic quorum-sensing circuit to control consortial biofilm formation and dispersal in a microfluidic device. Nat. Commun. 3, 1-8 (2012).
37. Qureshi, N., Annous, B. A., Ezeji, T. C., Karcher, P. & Maddox, I. S. Biofilm reactors for industrial bioconversion processes: employing potential of enhanced reaction rates. Microb. Cell Factories 4, 1-21 (2005).
38. Singh, R., Paul, D. & Jain, R. K. Biofilms: implications in bioremediation. Trends Microbiol. 14, 389-397 (2006).
39. Nicolella, C., Van Loosdrecht, M. & Heijnen, J. Wastewater treatment with particulate biofilm reactors. J. Biotechnol. 80, 1-33 (2000).
40. Jayaraman, A., Earthman, J. & Wood, T. Corrosion inhibition by aerobic biofilms on SAE 1018 steel. Appl. Microbiol. Biotechnol. 47, 62-68 (1997).
41. Tran, P. & Prindle, A. Synthetic biology in biofilms: Tools, challenges, and opportunities. Biotechnol. Prog., e3123 (2021).
42. Glass, D. S. & Riedel-Kruse, I. H. A synthetic bacterial cell-cell adhesion toolbox for programming multicellular morphologies and patterns. Cell 174, 649-658. e16 (2018).
43. Chen, B. et al. Programmable living assembly of materials by bacterial adhesion. Nat. Chem. Biol. 18, 289-294 (2022).
44. Bairoch, A. et al. The universal protein resource (UniProt). Nucleic Acids Res. 33, D154-D159 (2005).
45. O'Toole, G. A. Microtiter dish biofilm formation assay. J. Vis. Exp. (2011).
46. Trunk, T., Khalil, H. S. & Leo, J. C. Bacterial autoaggregation. AIMS Microbiol. 4, 140 (2018).
47. Kuipers, O. P., de Ruyter, P. G., Kleerebezem, M. & de Vos, W. M. Quorum sensing-controlled gene expression in lactic acid bacteria. J. Biotechnol. 64, 15-21 (1998).
48. Jinek, M. et al. A programmable dual-RNA-guided DNA endonuclease in adaptive bacterial immunity. Science 337, 816-821 (2012).
49. Mu, D., Montalbán-López, M., Masuda, Y. & Kuipers, O. P. Zirex: a novel zinc-regulated expression system for Lactococcus lactis. Appl. Environ. Microbiol. 79, 4503-4508 (2013).
50. Llull, D. & Poquet, I. New expression system tightly controlled by zinc availability in Lactococcus lactis. Appl. Environ. Microbiol. 70, 5398-5406 (2004).
51. Zhang, K., Su, L. & Wu, J. Enhanced extracellular pullulanase production in Bacillus subtilis using protease-deficient strains and optimal feeding. Appl. Microbiol. Biotechnol. 102, 5089-5103 (2018).
52. Brøndsted, L., Pedersen, M. & Hammer, K. An activator of transcription regulates phage TP901-1 late gene expression. Appl. Environ. Microbiol. 67, 5626-5633 (2001).
53. Sumrin, A. et al. Purification and medium optimization of α-amylase from Bacillus subtilis 168. Afr. j. biotechnol. 10, 2119-2129 (2011).
54. Paine, A., Eiz-Vesper, B., Blasczyk, R. & Immenschuh, S. Signaling to heme oxygenase-1 and its anti-inflammatory therapeutic potential. Biochem. Pharmacol. 80, 1895-1903 (2010).
55. Douglas, G. L. & Klaenhammer, T. R. Directed chromosomal integration and expression of the reporter gene gusA3 in Lactobacillus acidophilus NCFM. Appl. Environ. Microbiol. 77, 7365-7371 (2011).
56. Cameron, D. E. & Collins, J. J. Tunable protein degradation in bacteria. Nat. Biotechnol. 32, 1276-1281 (2014).
57. Shukla, T. P. & Wierzbicki, L. E. Beta-galactosidase technology: A solution to the lactose problem. Crit. Rev. Food Sci. Nutr. 5, 325-356 (1975).
58. Marugg, J. D. et al. Cloning, expression, and nucleotide sequence of genes involved in production of pediocin PA-1, and bacteriocin from Pediococcus acidilactici PAC1. 0. Appl. Environ. Microbiol. 58, 2360-2367 (1992).
59. Lu, W., Kong, W., Yang, P. & Kong, J. A one-step PCR-based method for specific identification of 10 common lactic acid bacteria and Bifidobacterium in fermented milk. Int. Dairy J. 41, 7-12 (2015).
60. Kong, W., Kapuganti, V. S. & Lu, T. A gene network engineering platform for lactic acid bacteria. Nucleic Acids Res. 44, e37-e37 (2016).

From Tables 1-3

### SUPPLEMENTARY REFERENCES

1. Kuipers, O. P., de Ruyter, P. G. G. A., Kleerebezem, M. & de Vos, W. M. Quorum sensing-controlled gene expression in lactic acid bacteria. J. Biotechnol. 64, 15-21 (1998).
2. Holle, M. J. & Miller M. J. Lytic characterization and application of listerial endolysins PlyP40 and PlyPSA in queso fresco. JDS Commun. 2, 47-50 (2021).
3. Kong, W., Meldgin, D. R., Collins, J. J. & Lu, T. Designing microbial consortia with defined social interactions. Nat. Chem. Biol. 14, 821-829 (2018).
4. Le Loir, Y., Gruss, A., Ehrlich, S. D. & Langella, P. A nine-residue synthetic propeptide enhances secretion efficiency of heterologous proteins in Lactococcus lactis. J. Bacterol. 180, 1895-1903 (1998).
5. Llull, D. & Poquet, I. New expression system tightly controlled by zinc availability in Lactococcus lactis. Appl. Environ. Microbiol. 70, 5398-5406 (2004).
6. Mu, D., Montalben-López, M., Masuda, Y. & Kuipers, O. P. Zirex: a novel zinc-regulated expression system for Lactococcus lactis. Appl. Environ. Microbiol. 79, 4503-4508 (2013).

1. A method of controlling transition between planktonic growth phase and biofilm growth phase in a bacterial host cell comprising growing a bacterial host cell in a medium, wherein the bacterial host cell comprises:
(i) a recombinant polynucleotide encoding one or more biofilm assembly proteins operably linked to a first repressible promoter; and
(ii) a recombinant polynucleotide encoding a protease capable of breaking down the one or more biofilm assembly proteins operably linked to a second repressible promoter;
wherein addition of a repressor for the first repressible promoter to the medium results in suppression of the expression of the recombinant polynucleotide encoding one or more biofilm assembly proteins and expression of the recombinant polynucleotide encoding a protease such that the bacterial host cell exhibits planktonic growth phase; and wherein the absence of the repressor for the first repressible promoter and the presence of the repressor for the second repressible promoter in the medium results in expression of the recombinant polynucleotide encoding one or more biofilm assembly proteins and suppression of the expression of the recombinant polynucleotide encoding a protease such that the bacterial host cell exhibits biofilm growth phase.

2. The method of claim 1, wherein the bacterial host cell additionally comprises:
a recombinant polynucleotide encoding a protein operably linked to an inducible promoter for orthogonal expression in both biofilm growth phase and planktonic growth phase, wherein when an inducer is added to the medium, the bacterial host cell expresses the protein in both biofilm growth phase and planktonic growth phase.

3. The method of claim 1, wherein the bacterial host cell additionally comprises a recombinant polynucleotide encoding a protein operably linked to the second repressible promoter for protein expression in planktonic growth phase.

4. The method of claim 3, wherein the bacterial host cell additionally comprises a recombinant polynucleotide encoding a protein operably linked to the second repressible promoter for protein expression in planktonic growth phase.

5. The method of claim 1, wherein the second repressible promoter is P_{sczD} and wherein the host cell additionally comprises a polynucleotide encoding a sczA operably linked to a P_{sczA} promoter.

6. The method of claim 1, wherein the first repressible promoter is P_{zitR} and wherein the bacterial host cell additionally comprises a polynucleotide encoding zitR operably linked to the P_{zitR} promoter.

7. (canceled)

8. (canceled)

9. (canceled)

10. (canceled)

11. (canceled)

12. A recombinant bacterial host cell comprising a recombinant polynucleotide encoding one or more biofilm assembly proteins operably linked to a first repressible promoter; and a recombinant polynucleotide encoding a protease capable of breaking down the one or more biofilm assembly proteins operably linked to a second repressible promoter.

13. The recombinant bacterial host cell of claim 12 further comprising a recombinant polynucleotide encoding a protein operably linked to an inducible promoter.

14. The recombinant bacterial host cell of claim 12, additionally comprising a recombinant polynucleotide encoding a protein operably linked to the second repressible promoter.

15. The recombinant bacterial host cell of claim 12, further comprising a recombinant polynucleotide encoding a protein operably linked to an inducible promoter and a recombinant polynucleotide encoding a protein operably linked to the second repressible promoter.

16. An expression cassette comprising a polynucleotide encoding one or more biofilm assembly genes operably linked to an inducible promoter, wherein the inducible promoter is P_{nisA} and the expression cassette further comprises a polynucleotide encoding nisK/nisR operably linked to a constitutive promoter.

17. (canceled)

18. (canceled)

19. A population of host cells comprising the expression cassette of claim 16.

20. A method of expressing one or more biofilm assembly genes in a population of host cells such that the host cells form a biofilm comprising growing the population of host cells of claim 19 in culture, and adding nisin to the population of host cells in culture such that the population of host cells expresses the one or more biofilm assembly genes and forms a biofilm.

21. The expression cassette of claim 16, wherein the repressible promoter is P_{sczD}, and wherein the expression cassette further comprises a polynucleotide encoding sczA operably linked to a P_{sczA} promoter.

22. (canceled)

23. A population of host cells comprising the expression cassette of claim 21.

24. A method of expressing one or more biofilm assembly genes in a population of host cells such that the host cells form a biofilm comprising growing the population of host cells of claim 23 in culture, adding zinc to the population of host cells in culture such that the population of host cells express the one or more biofilm assembly genes and forms a biofilm.

25. The expression cassette of claim 16, wherein the repressible promoter is P_{zitR}, and wherein the expression cassette further comprises a polynucleotide encoding zitR that is also operably linked to the repressible promoter P_{zitR}.

26. (canceled)

27. A population of host cells comprising the expression cassette of claim 25.

28. A method of controlling expression of one or more biofilm assembly genes in a population of host cells comprising growing the population of host cells of claim 27 in culture, adding zinc to the population of host cells in culture such that the population of host cells does not express the one or more biofilm assembly genes, and optionally removing the zinc such that the population of host cells expresses the one or more biofilm assembly genes and forms a biofilm.

29. An expression cassette comprising one or more biofilm assembly genes operably linked to a constitutive promoter, a gRNA having specificity for the constitutive promoter, and a polynucleotide encoding a dCas, wherein the gRNA having specificity for the constitutive promoter and the polynucleotide encoding dCas are operably linked to an inducible promoter.

30.-48. (canceled)


SEQUENCE LISTING (68 sequences)

SEQ ID NO: 1 - protein, 2823 aa, Lactobacillus gasseri
MTDAGLTKIQNAVGDNYSVSLADTTGTLVINKAKASAVFSGDPSYTYTGTPVSANDYLGKYSIKLTEPNNPTYNLVAGDIEFKFNGNWTTQAPVKVGQYEVRLSQQGWNHIKAINSDNVEWSATASAGTGTYTINQAKVTADLSGSNSMTYTGSAVTTNDLYSQDSTIKVVINGTDITNLPQTFELKDGDYVWQTTAGQAPKDVGNYQIKLTAAGISHIQKQINDALGAGNVALTTTADNAGTANFEIKQAVAENVQLYGDEQSTYDGDTVTFDPTNLDVKNNFGFHNVEGLTIPNFTSADFDWYDANGENRIAAPKNAGHYTLKLNDQGKQVLADANKNYTFVDQNGKSTISGQITYVVTPAELVVKVTGKASKVYNNQNAKITQDQINQGDIKLVWGNSTTEPTDLGEFTLTPDDLEVVDASGQPAIHANYVDGQQTGDTYYVRLTADALAKIKQLSGAANYNISQATDTATYQIYAHKAELTLTGNQTTAYGTELPFNESKYTLDFTNWVNTNIPKPVITWQNGEMLINGQQPEDGYSYHTGDLYVEGYSDGGVPTNAGSYKVKISANLTKELQKIFPDYDFSGNIDSSTLNSNKTVNNDPVEASHEPASYVITPAEATITINGAQHVKYGESTAIAGDQYTASVTAPVSGNETNVVTDVALTSDDLTTVPSNAGVGSYTIKLTPAGLAKIQAAIIGHGDVTKNYGWTQAGNATANFFVDQMPVTITVSGGRTVTYGTQAWLRAIKANPAGYTLTVTTENGTNLSYTANDGDLVFNQTPGNVGEYQVELSAQGLTNIGKALGTNYAYPQIAADVTAKGTFTVNRGAVTITLKGSDGKPYNAQQTLPSGLNLSKYGLDYSATVYSADGKAQMLNLTANDLQIIGNATNVGTYQVELSQAGQEKIEQLTGNNGANYKWTFKTNADYVVKAATASAELSGSNQKTFDGSAVTTTEVNSNGQILVHLTYPGSNVQSTYTLQDGDYTWETEDGQTISAPTNAGTYTIKLNKQAILAHLQVALNQQAGLGDNDQPNVTVSADKLSGQASFKINPQALTDVTISSPDQSKTYDAQVADLDVNGITITANGIVANNPLVNPGISASDFIWYDETGNKLESAPADVGTYQARLNASTLAELQNANPNYQFSSVTGLINYTINPAPATATISGSATRDYNAQTTSVSDVMNNIKWDATGLVTDQDLNLTGLTANSYAWYSKDADGNYVAMTGNPVNAGTYYLHLTKSAIEQVKADNSNYDFTSVNGEFTYTINAVNGIATLSGSSSKTYDGQAVTTAEVNSTNGDIIVNFTFPGSSAQSTYVLQTGDYTWENKDGQVITAPTSAGTYTIKLSADGITNLQNAINQYAGQGNVTLDVQDLLGAAVYTIKQKALDVILGNNSTGTDGKTYDGQAGVINTQAVNFGVFTTSGLVNGETLNAANLTSDDYEWVDVSGNAITAPINAGTYYIALTANGLKKLQADNPNYVVSESGQFTYVISPAEENVTVSGSQESTSTSIDSANFTVHAPAGVTVPAGMTYEFATGVPSESGVYVIKLTPESITTLEKANPNYKLDISSDAKFILDAILNIEFEDTQDGNKQVGKTITKTGVANSTINDLKLVVPENYELAPDQELPTSYTFGKTLNQNMYIKLVHKLNELNPTDPSTNPDPTNKNWFRENGLVKDITRTINYKGLSDDQFAQIPEAQKVQTVEFTRTAKYDLITGKIVANSEGSWTAVDGKDTFAGFTPFTFAGYTAAPARVEQVKVTGDDKNSQITVAYTANTQTGKISYVDSDGKEVGQTALTGKTDQSVEVNPEAPTGWQIVSGQDIPKTVIATPTGVPTVVVKVEHSTITVTPGTPEKDIPTGPVPGDPSKNYEKLASLMSTPTRTIVVTDPSGKQTRVTQTVNFTRTATFDEVTGEITYSDWKNSEPAEWQAYAAPEVAGYTATSSVSAKSVTAETKNETVNISYTANTQTGKITYVDSDGKEVGQTAISGKTGETVKVTPEVPSGWRIVLGQDIPETVTMGANGGPTVVVKVTHSTITVTPETPEKDIPTGPVPGDPSKNYEKLGSLTSTPTRTIVVTDPSGKQTKVTQTVNFTRTATFDEVTGEITYSDWTSSEPAEWSEYTAPEVAGYTATSNVSVKPVTAETKNETVNISYTANTQTGKITYVDGDGKEVGQTTISGKTGETVKVTPEVPSGWRIVPGQDIPETITATATGVPTVVVKVERSTITVTPETPEKDIPTGPVPGDPSKNYEKLGSLTSTPTRTIVVTDPSGKQTTVTQTVNFTRTATFDEVTGEITYSDWTSSEPAEWQAYTAPEVAGYTATSNVSAKPVTAETKNETVNISYTANTQTGKITYVDGDGKEVGQTTISGKTGETVKVTPEVPSGWRIVPGQDIPETITATATGVPTVVVKVERSTITVTPETPEKDIPTGSVPGDPSKNYEKLASLTSTPTRTIVVTDPSGKQTRVTQTVNFTRTATFDEVTGGITYSDWKLQKSNAASHVAQWDSYTPQVITHYVPSVAEVPAKVVNAHTANSQVEITYAPASESQVIRYVDQNGKEISTQIVPGKYGVDTTFTPKLPNNWQAANTIPTSIKIGENGGLTTIVVEAKTEKVQQAKTVTETIHYHTANGKQLFADKEMEVNFFRTGVKNLVTGEITWNNWNKDKESFNEVPSPKVSGYMASPTKVAVQTVTPNSEDLVFNVIYTKNSQTHPTIPENKPNKPQEENVSKQETKTQDKLIHEYGYKKRADGRLVDHTGHVYPASSKVKENGAIYSEKGELLSVGSRRKHELPQTGLHDNSLIAAIGSLLAGISIFGLLGGRKKKDDDK

SEQ ID NO: 2 - protein, 341 aa, Lactobacillus plantarum
MSFLDRLKGMLQALNSTEAATSATEAPRSIAAQTAAAPTVNQTEALVLVHHLDQDGNELQAADMIAGTIGEEIHLPAVSITGYHLVHIEGLTRWFTTPQASITLTYERQAGQPVWMYAYDIDRRELIGRPTMYRGKLGTPYEVSAPTVAGFKLLRSVGDVTGEYTTTSKTVLFFYRNQNWQQTDLSTGFVQVNKLTAVYPYPGATTTNYLTKLQPGSTYKTYMRVRLVTHETWYAIGDDQWIPETHLQLTTGDTLLLKLPAGYRVQNKRPVRQTGVVSFVPGKQVHTYIEPYGRYLTTVTHGDTVNLIERMADDNGVVWYRLQDQGYLPGRYLTKLDPPFA

SEQ ID NO: 3 - protein, 924 aa, Lactobacillus plantarum
MSKDNQKMTGDSVYRVKMYKDGKRWVYAGATTLALAAGLVFANVNASADTAASSDATTEQVSSAASSAATSSTATSSAATDASSASSTATSTSSTASSTATTSSSAASSTASSAVTSTTSAASSSAETVSATTPASSDATSTSTATVAATAAKASVVTPASAAATATTTATTTAATTAPTVTAPASEAANQTAAGSVDAGTLTSATQSGGSGNLQDQAQYIQENVDGTNIKVTAGHTYAVAIRLTKSQALDWANASGQVSIAPNGSNSNGTWTAVEYATESGKEYSYAAGASTATVDITKLTDADSYVTVLYTFKANDDATTGSRAAYLEFTGTTSVNKLSTNTNNTDANQQIEAWSYATQVMDTSVAAGTVVVHYVDENGNKIADDTTVQGDVDNTYTVTPATFSNYTLDTTKSSALTGTVAADTTDSDGNVTAAGTELTLVYSQNTEASNLTVNYVDADGNTILPSKTYTEGADGTAAEVGGAYSVNAASIDGYTLTGDATQTGTFVSGGNTVTFTYTKDAAPVEQSTVTVNYVDADGNTIKAATTQTLDNGSTYTVETPTIDGYTYKSADAALTGTVDGNKTITLTYTKNATPVEQSTVTVNYVDADGNTIKAATTQTLDNGSTYTVETPTIDGYTYKSADAALTGTVDGNKTITLTYTKDSTTPVENKANLTINYVDADGNTIKASSVTEYIVGQAYTVGQPEIAGYSYNHSTGDAIAGTIGYNGNTVTLVYTKNGGTTTAPTTAPTVAPTTAPTVAPTTAPTTAPTVAPTTAPTTAPTTAPTVAPTTAPGTGDNVNGGGTGTTTTAPVTTPSDDTVDNGNGSSNNGSSTTTSTAPATTVSDDEVTPTTTATTNNGTSGVVPASASLKPVVTTKTTTSDAKTLPQTDEDENGTALAVLGLSTLLMGSALYFGVSRRKHEA

SEQ ID NO: 4 - protein, 750 aa, Lactobacillus plantarum
MRRICKVLMVIISIILGSGAPLNMAIPPLLALAAPDTSSSSTMSSSAISKVTDTNVMAVSADVTSTTDTSDTSSSDSTSATSTTTGNDTTETADTAVESGTVGTVAWTIDDAGVLTLSGGSFADLTGKRSPWYDYASSITNIKITDEITVTTASNYGYLFASLANVATVTGLNKLSMSGVTSTQSIFYRDSKLTSVDFGQTDFSTVTTMESMFEGCSVLTKVNTTNWNVSHVKSFKRTFYMCGKLTMLDVSNWDVTQVTNLDSTFSGCSSLPELDVSRWNTANVTTLASTFYSCSSVKIINASGWDTARVTDMTATFMNCTLATELNVSGWDTAKVTSMSRMFFYCENVIQLDVSGWITSQVTSLGSMFQNCSKVVTLDVGTWDTSKVTDMSFLFGGCSSLTTLNLEKWDTGSVTTLYSTFYNCSGLTSLLVDTWDTSKVTNCFWTFGGCSSLTTLNLRSWDLQSATASYGNFFNGSKKLQHLTLGPNFTFHNDKTMYLPEPSKQLPYNGTWQRNNDDPTYTSAELMTNYDGATMAGTYNWVKTSGTVLVKYVDGDGVEIADEETSSGTSGDAYQTTAKTIDGYTLHATPTNATGTYDASTITVTYVYDGNLFFNSSPTMLDFGSHTISGTTETYAPTLDKTLAVQNNGQISSTWNLTAELDSSGFVGADTGKMLLATLYYQTDDGKMTLSPGVAVQVYSQTTTDHKSVDISEHWSSNLGLLLEVPNGAAMADTYQGTISWRLNNTVANN

SEQ ID NO: 5 - protein, 388 aa, Lactobacillus rhamnosus
MAVQPATLGQELNLNNQQTINADSPTSSNEVVVKCVDDAGNTLVKDTVLQGEVGKPYTIKPATIANYQYAKLANGSAPINGTFSKGTLTVTLVYTKVPVTQRTVNVKYVDEHGNEIAPATTLTGTVGGSYTAVPANVKNYEYAHLAANSAPEKGSFTANPQTVTFVYTEKPAAQGSVTERFVDEAGKRIAPDKTLTGQVGDLYEARPIEISDYAFSRVAQGSAPAGNTFINGNVIVTFVYKQVPATQGSVTVRYVDENGNELAPNRVLAGQSGSAYTTGPITINGYRYVRLAADSAAASGTFPKDTGLVVSFVYTKPAIPVTPTTPETSTVPSTSSQSATTEVITPSAQRRLPNTNEKHEYGIAAVGLALLSLMGLGSTLLFRKAKRQ

SEQ ID NO: 6 - protein, 1227 aa, Lactobacillus plantarum
MYTENTGKHHRNGLPVWLLPLLVVISFWGVSQNIMVVDASSSVTVLPGNGGTLPLVNQLVIKQNDTALQGITNNAGDRGSLTPKNGAQRVLIHKVKDSDTITSTYGTVGTFHGQEVTAKVTISHIKVHDDSHKAPSGMKQTDGAFQIGPGFSSDTTMSNVAQFNVSYEFYYADTHAAVNIQNAFITLSSLDGPVAGTSTGFEYTAYLGAGKIYTVENSIVKQIANPLGGGQLVMAGQTARDASWPYTSSTAATFGVSGTKLEFIYGTTRVNSGNSWLQPVYNVSTITLGTPAIATPTLSATQSATDKQNRTLTYDLQQKVNVLDQDLMTKYKDWSENITIPANAKYAKGEVVNDAGQALPSTAYQVSYDEKTHQVKWHLTDAGIKSLPFKGETYHFKAQVQFSDDVDDQAKVTATGQTAIDKQIKTSNTVTNTIDNQATITVHHYMTDSTDKVAPDETVKVGYGKAYDVTKQVKTITGYKRNATLDEHTRGTASKTTKEAVMYYDPLPYNIHVNYLLTDGQKLDELDVTGLYGDTYTTEATDFEDLYTVDTDRLPTNAQGTVTEKPTTVNYYYQPTTGQWVDVGNQSSVLVRQDTKHNVRSVSQIYANDSGFTVKYNQDAAQVAIAASDTNGTQDNSLVFDYNSKYTFELSKNETVTFKVDDQGQVTATRVLGAEQTVTTFDKSGQLKTVTTVTNANGTKSQQTNTVDGLKSMVTGEQYDLGLLNGLKVTAQKEINPSQAATTESKTTTDTSQSGSNQSTSTTATDQTGTNESTAGSSTNATNASSSVDASSANSQGDTEATSQSGTSASADSKTDSSVASSTSQTTDGETTNTGDTTTGTTTGSGLGFKSPFTEDQNTSSALGSAQTSSSLNSDTSAAVQALIAEPNSTPVVLDEDASFEEGVPVNDPVFSNDEGVSPNNNPSSAATPLAQATNTRARLTQNGKLLYEGTLKADQGEQNLYVSPDTTVEVDGGADGDGFYLDTYDGDKGMAYTLGSGYAWAAENNDVTAAPASSATTSSESAASEPSVNSSDSSRTASSAVDHSTSSASTSDASQSSHSTSSGESSHPESSSGSSTTSDSADVDKQAAARSSQTQSNSVNGSSQAVSSSTVTSQSSVPTKANTKQASSTPTTKANRATVAAATSSTAPRQSRATTASASVPSVTSASAAAASRDKQRSAFKKQHPILNQILPKTNSAVATWLVWLGVGLLLLTVAITMVIKKRGRD

SEQ ID NO: 7 - protein, 1189 aa, Lactobacillus plantarum
MNKRKIITNNPPKWHLITGIAATILASIILTNQDAFAATDSTTAPTTTAPTVQQTAPTNPLSGSQVTLTSTTGSSATGSTTTSSPVATSTAAMPVKSTATSGSLMSAMASTSATSGHAAEPSSSVTEAASTNNLIPTSAAMASSATTKYPTDTTATPNASSSLTSAESSTPNKAMSTSQQTVSSGVIHSTTPASSASMPVPTSVAETASAAAPSVTNSTAANSTAPTSVMTTDSAAESVPLSTSSETSSEKLAAASTTSTSQISDGSEVIHPMTSAISSSSSAPTSGAKMAASAASAASASVITSAVNSIAASTYSADASAASVESAATPDTSHATVPASTATSAATTFQITSVINSLASSTYSEYAEQANAEAASAATTAEKPATSVGTVVPTAATTPTESIDTWMPNKHLQEAVLRELQALKLPDHQFKSVNDITKDDMQLLTQFYGENTYIDGHTPYSLEGLQYATNLKTIWLNNGLNALGGYYNGDVTDISPLAGLTKLTVLNIQHNRVSDLSPIAHLTNLQELDVAYNHIADLSVFKDLPNLKTTTYLGQTILEPLVYVDQDTTSATLKNRFYLPNGQQAVLKSQAAILKPVQLTPNGQFYYRFYFNGAGKAVNGDLSNVVPDGQGGLTFNQLVPQIPGFTGDANGQFVTNGVSINVVPNDKNFYLVAQGSDGSSPVFHVFQPYVLAAKAAPVTIHHIDRNGAALRDSEELTGLVGEDYQSTPADITNYTHVETQGAPQGTFSAEPQAVTYVYDKTAGAPVTVSYQDEQGKTLQPDTTCNGLAGDPYTTKPLEIAGYDLTKTPDNAAGTFTAEPQHVIYIYTKQVPQPVTASYQDEDGKALQPDITHTGEIGAAYETKALEIPDYDLVKTIGNATGTFTKEPQQVTYIYTKQIPQPVTASYQDEDGKTLQPDITHTGEIGAAYETKALEIPGYQLIKTPTNATGSFTKEPQHVLYVYEKQAVLPVTVSYQDADGKPLHADIVLSGDFGQNYQTEQLSIPGYVFNKVVGPTIGTFGTTAQHVVYTYTPEPSGPEQPTPGPEPEPVPEPQPTPAPQPEPTPQPSPTPQPSPAPQPNPAPQPSPVPQPNPAPQPGSSLLAKAPVSQGTTTSQSSPTTSPQPTPIAPVSALAQPGKQQAPATVATHNSGQLPQTSEQSEHGATLGGILAALFTGLGWLGLAAKFKKRE

SEQ ID NO: 8 - protein, 1356 aa, Lactobacillus plantarum
MRYTRGKWRVTNPKVWLFSSVLILGWRIVPTVAQASEAETVTMSSHSVQLETDNQDQLTEVARISKTAVTRDSHSVTAQSSKSADRTSSEQPATTGTVEAVSPTTSEAQQRSTQQDKTAVDQQASDSTAASAGASTNQASAATSSDQAPAANSTGTHHAIDMASSASALGADSGAHSESLSEAQHSGGQGKTIDSDLSGTVHSQSSVSTVTTATPVNSNSSLESDKFTSTRSRAVAATDQMSSRVEKRALNKTNVTKSINIPVATKQPSKQRTVTASSFLTTAKNLADKNYLDQYAKQHGQAALIALIQDWLSTYRIIALTGITIVNSSFDGSVATISGGLHVINTGATIRSGQDDEWETIINGGLSVTNNTITFTTTNGLVDRPVANQDMDFTKPRPTGNGAIKGLPSVTVDSSLINAQEFSQAQINISDFYDQLVTAGTILSATNGGTLSKMLIGESGTADLGSYQGHHYYAVNIDLNDWHSGIRTTGFNNDDVVIYNVVTAAPALTIGGGFSSSTPNLVWNFNHAMRIQNTTMITGKIVAPHAVFTTNQNVDSAAVLQYGYGDVDSAIRETITSQNEHNYGFGQVVTDDPLDYLIAVIKSDGTSIDTLAGFRHLLATGQLKITITDAAGTRLSGLNAVDTHIAGQHCYLITYQFGDQTATTWLNVQPSHEPIIPISRIPEYSAITRTINYQDERTGAVLAGPVIQNVRVVRFAIFNAKTHELLGYDTNGDGIVDTSDGTIAWLLVPPTDQDWVQVVSPDLSAQGYQAPDIPVVAGQTVIINGGDRTMNTNVIVKYQQQTHIATTQRTVTRTINYIDGGTLQPIASLHAVVQTVKYQLLAVVAHDGTILGYDTNGDGQIETQLADEAWLIVGSGPWFGAVKSPDLSHEGYAAPDLKVVPEQMVAGVDDKDVTINVYYRLATQAVTVYQNKRRVISYIDRQTHQSIATTVQQLVIYQRTAIIEKKTGKCLGYDLNGDGLVDTSQADYAWILVGSGQFAAVTSPTLVVQGYTDPDIRTVAAQTVAITDPDLMTTIVTYDHRIITVTPGNPARPGQPVDPDNPNILFPDEGGDTDLTHTVTRIIHYVYEDGTTAAASVLQTVQFQRNAMIDLVTGEVTYQEWVPVSVTEMAGVISPIVAGATTTLTEVAAQQVSVTTADQVVVVTYKKSAIKPEEPGQPEQPSQPEEPGQPEQPSQPEEPGQPEQPSQPEEPGHPEQPSQPEEPGQPEQPSQPEEPGQSEKPGELQKPSQPADSEQPDGLSDQANLSRNQAEQSRTSQPSQAESDQSVVQTNQQKTAASVSGIGWVSGPAVSKRTTKHHRMTTLPQTDEQNTQLSLLGMIGLALSSILGWLKIKSRD

SEQ ID NO: 9 - protein, 1094 aa, Lactobacillus casei
MTAIGAKAFNANLIPEVAIAGTPTIDQEAFSNNRITVLHAATAVPTTPDALNQNADAYTDSAHVSLRDLFSVAISGVSQDQIVVSNIQGTGVAFNTATKSFTMPAGTEQFSFNWSLKAADGTTYTGLYKVHLNDPVIHAHDINLFTGQVWKPELNFGGAVKKNGTEIIEIPLSDLTWTVTDQNGAVIASKDRDGVVTGSVPSDQVIWYTVTYAYGAESGSAKIFYNQRLAASYSLTGTQTATATGQPITVDLTAFSLSLGDGFNAGALQLSDLNFFDASGNQIAADALTKTGVYRVELSKAAWARIAELTNDAGESAANYNFTGTSTAQLIIGRTATGQLNNSGFTYDGTTLASQAPKLVLNVTLSDGSQQAIDLTSTDISLVEADSPDVGTYRYLLNGSGLTRIQAILGDEVTIDQTDINTHPGVITITPATATATVNGTQFVYDGKTTASQASGLQLTLTAGSGTTVVDLSSTDIVVGSDSVNVGDYQYQLSQNGVAKVEQALNANYQLPSDLLGSLTGTITIAPAQGTAELRDDSFIYDGQTEASQVQGLTGDVTIGNVTVPVILTSVDFVVGNDGVNVGSYQYTLTATGIAKLQQAVGSNYQLTVSELAKLTGNINITPATTTADSNDGSFMYDGQTKASQAQGLTAVVELGDDTTSIKLDASDIVVADDGVNVGSYHYRLSTDAITKLQQVAGPNYQLKADDLAALMGIITITPAEGTATVNDTTFVYDGRTKASEASGLNGVVYLSRGTARLTVALTTQDIVVDGDNTTTGTYHYHLSHSGIAKLKAAAGTNYALNETDLNALTGTITITPLTVVATVNNGHFQYDGVTRASQASGLLVTVQLPTGAQTVALTNADIDVANDSATVGTYTYRLSASGIAKVMVALGPNYQINDTTMNGTITITPAVLSGQLSGMQQKIYDGQPGELNAQHFELIFTDGSHIILEDSDLAFADGIAPIVVGRYAVTLSAGGLKRIQALLPNYLLENVDTQQAVFEIVAKSGPLPDTGTGTDTGTGTNTGTSTGHETGKVPSVTGRPSQSINQQTPVKTTHQLPQTGDRSANDLSIVGLILTSIASLFGLAGVRNKKRSE

SEQ ID NO: 10 - protein, 1281 aa, Lactobacillus plantarum
MTMLPLNCQRHYISILKEWGSLKPNNVNNQNKRHQSRWVITSATAMILTTLTIASQAAAADDTVTTTTNEPTNSQLNTNTQVNATQVNLKADTSTSVSTIKSDQSAVAATSPTTSTGSPSEHSSSVNTNPQQQSANPASQSQATTTSESTPTTDIKHPTQTAPAQTTSASTTEPTTESNTESATDSQAKATTTDNQASKQPSQQAAPAPSNSTTTEVNTQSATSSASTDDKIVTNVNQEKLVLKTNQPVVRAISRTASENINDWMPNTLLQQEVLSQLRKQNPDRTWNSAADITKADMLLLTTYYGKDTYIDGKTSYSLEGLQYATNLTTVWLNNNLNAPSGSYYSDVTDISPLANLQKLQVVNIQQNRITDISPLANLKNLTEVDAAYNHISDFSPLKGFKNLKGTFSNQFITLPPAYISADNNIATLAIDCYLPDGSKVQLKPNNGVGETVFYKNGQLYVRWYFNGAGGGNYDSNGHIYYTNMKPQQPGLTGPTFNGTTVIPMDDYYFMTAASDGNNFVVVRPYVLAATAAPITVKYVDALTGESLVTADLTLNGIVGQPYTTQRIDDELPNYDFTNIVGNASGVFTADAQTVTYYYTRKDAGDITIHMVDANGNLVYEPQILPGKHNLGNAYNLDAPTFDHFKLQQTIGNAAGVFTTDPQSITFVYVRLDAGNITVKYQDKQGKQLKPDKTISGSQSLGQAYTTEPLDIENYTLTTTPTNATGTFTDQEQTVIYVYVRRDAGQIVVKYQDSAGNPLAPDKLLDGKEQLGAAYQTEAISIPNFYLVATPANATGTFSTDAQTVIYQYTRSNAGHITVKYQDANGTTLAPDDVLTGDGQLGRPYQTSAKTIENYRLIQTPANATGQFSDQAQTVIYVYTREDAGDITVQYLDENGQQLAADSVLSGQGQLGRPYETSPLNINGYTVKSTQGNTTGTYTVQPQRVVYIYDRTAGQPVTAKYQDQDGKSIHPDVVHSGYLGDNYSTEQLVIDGYTFKAVQGDVSGTFGTSAKTVTYVYERTAGLPVTAKYLDEHGKSIHPDVVHSGYLGDSYSTEQLVIDGYTFKAVQGDVSGTFGTTAKTVTYVYTVNTPTIPDTQGTVTVHYMTKDGIKLNEPTVLSGKTGTTYQTVPLTFTDHELVGQPENAMGLFTADNVDVTYVYQATDTTGTDDIIDPEEPEQPTKPIKPVEPTTPETPNEPGTTVTQPDRIKPTQPAVAVKPAATVKPTLKPAAAQASLVKTTSPVTEHSAQLPQTNEQTGKLAVILGLLLSIVTFGFYGKHRQS

SEQ ID NO: 11 - protein, 1201 aa, Lactobacillus plantarum
MTKSIIKRSMIILNKRKIITNNPPKWHLITGIAATILASIILTNQDAFAATDSTTAPTTTAPTVQQTAPTNPLSGSQVTLTSTTGSSATGSTTTSSPVATSTAAMPVKSTATSGSLMSAMASTSATSGHAAEPSSSVTEAASTNNLIPTSAAMASSATTKYPTDTTATPNASSSLTSAESSTPNKAMSTSQQTVSSGVIHSTTPASSASMPVPTSVAETASAAAPSVTNSTAANSTAPTSVMTTDSAAESVPLSTSSETSSEKLAAASTTSTSQISDGSEVIHPMTSAISSSSSAPTSGAKMAASAASAASASVITSAVNSIAASTYSADASAASVESAATPDTSHATVPASTATSAATTFQITSVINSLASSTYSEYAEQANAEAASAATTAEKPATSVGTVVPTAATTPTESIDTWMPNKHLQEAVLRELQALKLPDHQFKSVNDITKDDMQLLTQFYGENTYIDGHTPYSLEGLQYATNLKTIWLNNGLNALGGYYNGDVTDISPLAGLTKLTVLNIQHNRVSDLSPIAHLTNLQELDVAYNHIADLSVFKDLPNLKTTTYLGQTILEPLVYVDQDTTSATLKNRFYLPNGQQAVLKSQAAILKPVQLTPNGQFYYRFYFNGAGKAVNGDLSNVVPDGQGGLTFNQLVPQIPGFTGDANGQFVTNGVSINVVPNDKNFYLVAQGSDGSSPVFHVFQPYVLAAKAAPVTIHHIDRNGAALRDSEELTGLVGEDYQSTPADITNYTHVETQGAPQGTFSAEPQAVTYVYDKTAGAPVTVSYQDEQGKTLQPDTTCNGLAGDPYTTKPLEIAGYDLTKTPDNAAGTFTAEPQHVIYIYTKQVPQPVTASYQDEDGKALQPDITHTGEIGAAYETKALEIPDYDLVKTIGNATGTFTKEPQQVTYIYTKQIPQPVTASYQDEDGKTLQPDITHTGEIGAAYETKALEIPGYQLIKTPTNATGSFTKEPQHVLYVYEKQAVLPVTVSYQDADGKPLHADIVLSGDFGQNYQTEQLSIPGYVFNKVVGPTIGTFGTTAQHVVYTYTPEPSGPEQPTPGPEPEPVPEPQPTPAPQPEPTPQPSPTPQPSPAPQPNPAPQPSPVPQPNPAPQPGSSLLAKAPVSQGTTTSQSSPTTSPQPTPIAPVSALAQPGKQQAPATVATHNSGQLPQTSEQSEHGATLGGILAALFTGLGWLGLAAKFKKRE

SEQ ID NO: 12 - protein, 1231 aa, Lactobacillus plantarum
MYTENTGKHHRNGLPVWLLPLLVVISFWGVSQNIMVVDASSSVTVLPGNGGTLPLVNQLVIKQNDTALQGITNNAGDRGSLTPKNGAQRVLIHKVKDSDTITSTYGTVGTFHGQEVTAKVTISHIKVHDDSHKAPSGMKQTDGAFQIGPGFSSDTTMSNVAQFNVSYEFYYADTHAAVNIQNAFITLSSLDGPVAGTSTGFEYTAYLGAGKIYTVENSIVKQIANPLGGGQLVMAGQTARDASWPYTSSTAATFGVSGTKLEFIYGTTRVNSGNSWLQPVYNVSTITLGTPAIATPTLSATQSATDKQNRTLTYDLQQKVNVLDQDLMTKYKDWSENITIPANAKYTKGEVVNDAGQALPSTAYQVSYDEKTHQVKWHLTDAGIKSLPFKGETYHFKAQVQFSDDVDDQTKVTATGQTAIDKQTKTSNTVTNTIDNQATITVHHYMTDSTDKVAPDETVKVGYGKAYDVTKQVKTITGYKRNATLDEHTRGTASKTTKEAVMYYDPLPYNIHVNYLLTDGQKLDELDVTGLYGDTYTTEATDFEDLYTVDTDRLPTNAQGTVTEKPTTVNYYYQPTTGQWVDVGNQSSVLVRQDTKHNVRSVSQIYANDSGFTVKYNQDAAQVAIAASDTNGTQDNSLVFDYNSKYTFELSKNETVTFKVDDQGQVTATRVLGAEQTVTTFDKSGQLKTVTTVTNANGTKSQQTNTVDGLKSMVTGEQYDLGLLNGLKVTAQKEINPSQAATTESKTTTDTSQSGSNQSTSTTATDQTETNESTAGSSTNATNASSSVDASSANSQGDTEATSQSGTSASADSKTDSSVASSTSQTTDGKTDGETTNTGDTTTGTTTDSGLGFKSPFTEDQNTSSALGSAQTSSSLNSDTSAAVQALIAEPNSTPVVLGEDASFEEGVPVNDPVFSNDEGVSPNNNPSSAATPLAQATNTRARLTQNGKLLYEGTLKADQGEQNLYVSPDTTVEVDGGDDGDGFYLDTYDGDKGMAYTLGSGYAWAAENNDVTAAPASSATTSSESAASESNTNSSDSSRTASSAVDHSTSSASTSDASQSSHSTSSGESSHPESSSGSSTTSDSADADKQAAARSSQTQSNSVNGSSQAVSSSTVTSQSSVPTKANTKQASSTPTTKANRATVAAATSSTAPRQSRATTASASVPSVTSASAVAASRDKQQSAFKKQHPILNQILPKTNSAVATWLVWLGVGLLLLTVAITMVIKKRGRD

SEQ ID NO: 13 - protein, 2219 aa, Lactobacillus plantarum
MRNRLNRLGLESKSHYKLYKSGRRWVAASITVFSVGIGLTFSQVEQVKAATGTGVDTADNSASVSSDMAEPSNAVVLKSASTATATKTATQDAKAATDVTAATQDTKATTDSTGATSASSNRQSTAATKPAAEVGTASSSADSSASISSTDGASASAPSVTSKFTNTEATSASATKTATTSADTDVLNTETTSSSVANDLTDATTASQTRTETGKTASIPTAEAPTITTAVTSRALPLTGALASRSANTPVTKSAVQAVSAITSEAETKPTVSLVTTGTVSMDYGEASLADLESHISSPDETPANDVAYYIQDAAGNYLEDVNGNKVNLLYALFLDSADVNDYVDVVYTDEHGQVTKYSGDTDFSTLDQIGSYSVTINAAGKAGMSRVMQDYNAYDTSTSDLDDFVPTFSTGASDYTFTINIVPVKITATTGKNGLIILRPSQLYTGSLTMLPVVTVKNATKQNILQISNGEIGDAKPGVAGKVGQRVLTLADFTYTYQGTETNLTGADTGKYAITLNDAGRKAVQAALGSNYILDDAAVFTTTGAVQAAGLGLTIANDTVTYNGKPQGTTVAITAGTAYDHFDFTTTTDTNVGTYDDLTYALADPTQAAILAKNYTVTTTDGTLVITPADLTVTVKDDNPVYDGRAHGMTATVTSGTNYDQLAFTAVAADGSGATTYTTVGTYAMTGTTAADTSNYKISYVNGTLTIDPAKATITIPNKIYWSDGTQKNLAAVVTGTVNGETLKYRVTNGMSAVGTKTITATPDADDSVNKNYTISVIPGTLTIGDIAVKYLYEHVDANGETQVDASETGTATHATDATATDYLTYTTAAKPKTGYVLAPNTGLAYNGTLTDQGGTVTYRYLAKTETAIVTYFDQTDNKVIKTEPLQGAYGTTDAYRTADTIAAYENAGYDLVSDDYPTAGVVYDQDGSVQEYQVTLVHKFVTRTPDNPGTPGEPIDPDNPNGPTYPVGTDFEDLTEQVSQTIQYLYKDGRTAKPNNVQAVNFGRNVTVDEVNGTVVYTDWLTDDGAVTGRFEAVDSPLITGYTADPTSVAGNPGVVWQDDDTTIPVTYTVNTEYATVTYFDQTDNKVIKTEPLQGAYGTTDAYRTADTIAAYENAGYQLYRDDYPTAGVIYDHDGSVQKYQVTLVHKFVTRTPDNPGTPGEPIDPDNPNGPTYPVGTDFEDLTEQVSQTIQYLYKDGRTAKPNNVQAVNFSRNVTVDEVNGTVVYTDWLTDDGTMTGRFEAVDSPSITGYTADPTSVAGRDTVSGTDLSPDVQVYYQANPEKATVTYEDTTTGAVLTTDPVTGDYQTVSNYRTADRIAQYLNMGYELVSDDYPTSGAVFDKDGSTQAYTVKLQHKLLPLTPENPGTPGEPIDPDNPNGPTYPAGTAVQDLIKQVGQTIHYQYQDKSTAADANTQTITFKRSVTVDEVNNKLTYTDWLTGTATTGHYMPVDSPEIKGYVADSTRIAGNDEVRNADADTNIVVTYQAKPENATVTYVDVTTGKTLAIKSLTGDYQTTSSYRTAETIASYVKNGYQLVRDNYPTSGAVFDVNNFAKTYTVTLKHKLVTVTPENPGTPGQPIDPDNPDGPKYPVGTTAQDLTKQVSQTIKYRYQNGASAGTDNVQLITFNRDATIDEVEPTAVYTDWLNGTSATGRYTTVMSPVITGYTADKTQVAGRDSVANTDSDTQVVVTYAAKPEKATVTYVDVTTGKTLVTANLTGDYRTQSNYRTAETIAGYVKNGYELVRDNYPVSGMLFDVDDFAKTYTVTLKHKLVTVTPGNPGTPGQPIDPDNPDGPKYPVGTTAQDLTKQVSQTIKYRYQNGASAGTDSVQLITFNRDATIDEVEPTVVYTDWLDGTSATGRYTTVTSPVIIGYTADRARVTGNDAVTSAAQPTNIIVTYAINAEKATVTYVDVTTDKTLATVSLTGDYQTSSDYRTANTIADYSNQGYVLVRDSYPVSGAIFNDDGVVHSYLVQLAHVTTATTETKTITQTVHYQSTTGTQLHDDTVRAMTFTRTKRVDQVTGDVTYSNWSTNQADHTFERVAAFSIPGYHAVVTGTQAVMVTPASVDDVQTIRYVTDRLSTGETPKTPVKTVTVNKSDKIKTTDTPDKVATVKTPDKAQTVATTTAKQASVKRSVDLKQAQAVEQPAQTRPANVKTVKLAKTTKSVKPTAAHQSATHKQATLPQTNDDRQASVAAELLGLTAATLLVGVSAILKKRHN

SEQ ID NO: 14 - protein, 2209 aa, Lactobacillus casei
MRELGVKKTGHFMLKVGIYLTVILGMIVQLISPALALAAENPTQAVTGTLTIKNQDEQGSPLNGAKYEIQNESHQVVANSEISKDGQATVPNLPVGNYTVTEKQSVSGYTALEQTKNFSVTASGNVTLLFKSRASATLDSGSSSSTAAKPAAAKTPEAEPSATPDAKADTELPNIFTKVALKDGNDQPLGTEVDQQSAVKMEMTFTLPATSTPFPAGASFTTTLPKDQIAFPESGGGNFSGDVASYYFDATTGQLTIKLLKATSNGSWLVHIAASFKALTANDSLNQTLVFHTKDQDTKFPIMFRSNAKPVVVYAHTTTPQSLNPTGIAGTAKFNLNGNETSKTDPTKWDSDPAKRSKNADMALTLTARGSGTDYLKSLTFSDSDLAKIKVSSAPVNILGGFSEELKPLVAGQDFHAVLSDDKRTVKIYLTGGFKKTTGYQVDYTATIDRSLDDTGKVGSALVEGYRYLTGSQSSDGYDYDSVTMRNSGVAITKSGDITNNFRALNWKINWNYSMDTMKAGATLTDRFGKQTSGKDEHDQPNIETDGNQTLDTKSLKVFQVTFDEWATPIVSKVDIAQYFKLTEKGDGEFTLTYLGGGDLPENASFQIQYQTKLKNTPKNGDNLTNIVNDQKNHYDHATYPVRLPSGITKVGGKIDAYNGQMTWRINANRVFRNMKNGKIFDLFPDGVDKLDNDPTADNINTISGENVSANVDDGANDGILVYAQNPDGARTLLKPGTDYDMSTQDADVQSAVKQYNDKDKTNPINANGQEKGIRGFVVTLKGAYAETDSQIVIYTHTKLDMLKLGQVGHDPDALKKALNNRAFFFFDLPPGDDDVASGDSSSTPTPEEGAFSGALKNSWSDAPDTQYWGVLVNQLGLPYGHMHLTDILPRFDGVNYELIPDSIKFYEVTGPDGVDPSNTGDPASSNDVKEIKTSPYYGTGGWSSTALKAEDAAQQRLLPTNTPNTWLKNNPNLAQQLDFDFPNIGTGRVWVVFKTMRANQWNYNDPNFANNATVTDTEPTTAIPTFNPSASKSAQSYWTPISKTVSADTKLKNVLNWKVNLINIQDKYRPMVNPVIEDTLDPRGTGAEINATSFVVTLKVGIADPDTLEEGKDYSLSLDGKKFTITFNRTFGNLVQTANSPLNNYEVSVAYSTSSKSSGWAYNSSSVEWDGSQTTQKPSDGVPPDARIANANGYLPYWGSGISGETLTQLANLVVEKKDSVSGTPIPGVKFRLSDGTHTFEATTKLDSATNKALATFQGLPIGIDYTLTELSTPAGYKPLAPQTIRLNATSDTGTAIQTEAVENEPYQITLSKYDNRAKGQSETDNKHYLLPGATYDLVDTDTQKTLKSGMKTNADGKITIGTASSFSGQYAGDKFTPDLKDGEYVLEDLKPGNYKLVETQAPDHYRGDAHDQATITSGPDKQVWEDSLKAGSVAAIISNKAPSATVTAYNQKKPGQLDIKKQAETITDDKFSDRQPMTGAEFKLYRYGDDGKVDQSKSWDATIISQDGTFIFDSPDLYEGKYQLVETKAPEGYVIPDDLAKGVDVNITGDETLKLPTITEPVYRRALQVAKTDGNFGNPIAGITYALYQNDGTEIAKDLVTDENGQVNLPFNLPAGKYYIQETKSLPPYRPNSDKHPFEVKQTDQTQTAGNLETENKEHPIKVNVTNYQAKTLNVKKVDRTYATHVLPGAVFRLTNSAGYTRDVTTDENGIASFGDLLLGSYSLTEIKAPAGYRLDNTVYPIALSSAETPTAITVNKEIADDPYQVNLTKYDNRVKKDDPASQKKYLLPNAVYKLVDVAANKTLKADMKTNADGQLTFGAASSFDSPLKDGEYAIEGLKPDTSYRLVETEAPEHYEGDAADQANATSGTQKQAWEDSLAAGSVDFNIKADQTQVKLTATNQKKPGQLDLKKQAETIKDDHFPDRQPMTGAEFKLYRYDEAGKVDRSRSWDATITNNDGTVSFKDSDLYEGKYQLVETKAPDGYVIPDELAKGVDVDITGDQTLTLPTITEPVYRRTVSVAKTDGNFGNPIAGITYALYREDGTELAKDLVTDKNGQVNVPFSLPVGHYYIQETKTLPPYRPNTDKHAFEVKQTDQTQTASSLATENKQQPIRVNVTNYQVKTLNVKKVDRTFAAHVLPGAVFRLTNSAGYSRDITTDENGLASFGDLLLGSYSLTEVRAPAGYRLDKTVHAITLSSAITPTPITIDK

SEQ ID NO: 15 - protein, 2648 aa, Lactobacillus plantarum
MNRFITSKQHYKMYKKGRFWVFAGITVATFTLNPLISRADTETTTAATAATTTAGASSSSNSQVLRTTTTSTTGATTQSSATAINAATTNTSAQKKQAVSGTTTDSKTEQPVTAVGENENATSNLSTSDSASASSQAKTGSGDSLDQTSNSSVSVASSSQKVTTQNSDYQNDQGTGSESGIQSNVTDTVVADESLQTNRSSVASPSTSTMASISDSDSKDSNETEKVVDSETSPIAVTATTNTITTTNDKVQLNRALLARAATPATVVSTGTLGTSAWQYTDDGVLTIHAGDWTGVGDVSDVPGDFGSELTKVVIDGPINAGTDTSYMFRYNPNLASIDGLENLDTSKVTDFSMMFMGTKIADFSGLAHWNVSSGTSFDSMFASDSRVQSYDLSQWQLNTVQPVSLKRMFSFNTALISIVLSTWNVRMVTDIDGLFNGDKSLTTADLHGWNLLNVTALSSMFLNDTNLTDLDITGWQTGSTLTSTKFMFEGTPGLKAINIASLDMSNFAAVTEADMNKEPADHDMFLNQDSSGNPLPMNLNALTVGSKTYLVGSSLPDIPTGTGYTGKWVNQADATQTYTSSELMALYNGVDNPADTITWVWETSPSYADFTSKNVTGLIAGPKTTWRVADSVATLKDVNGTDIYATADTVVKVISVNGDTAVTTVDTQTTGTYQVDLQYTDAYGKVWQQTSTVAVAVNQGKLVGKPLTIKMGAKPTYTINDLIDTDNSRNAAGDKLSADELATATVTGLDTSKAGTQTVTLAYTDDATGMVHTTTTTVTMVATKADLTMRNSTIIKGPKNSSWDYRQYVTSVTDFDGNPVSLDGLNIVVDQQPDLTQIGSQTVTLTYTDTLGNVISVPTQVTVVASRAQVTTKAPLTIWPSEVAQLKVADLVTITAANGNPVDTSTDLTDVTMSSIDTSKGGAQTVTITYTDEAGNLVTAYAKVTVDQSDLKTKLTNPIAGPKAKWDYLAGLEWVKDANGKLLDNLATADIKVVTEPDLSVAMVGHDQTVTLSYTDELGKEHLVTAVVNTVASKAKITAVSDQIIIPDEAKKLTATDLVSELIDAAGNKATNFDDVTMSGFDAKAIGPQTVTLMYTDAYGNQTTDSTTVTVDFATITGQATHPIAGPTATWDYRDSVTQVIDANGKIIDVGDADITAMTPDLTPAKVGKPQTVTLTYTDSLGKVHTTDVIVTTTLSEAKITAVADQIIIPDEAKKLTATDLVSELIDAAGNKITNFDGVTMSGFDAKAIGPQTVTLTYSDAYGNQTTDSTTVTVDSATLTLQNHTQVAGPKATWNYADNIKAITDSKGQSLTLSDAKITVVQRPDLSVAGTYKIVLEYTDDLGQAHTETADVEVTASKAAITAVSKQVILAEKATMVTASSLVSTLYDADGVQIYNFDDVTMSGFNAKAIGPQTVTLAYTDAYGNQTTVSTTVTVDFATLTLQNHTQVAGSKATWNYADNIKAVTDSKGQSLTLSNAKITVVQHPDLSVAGTYPIVIEYTDDLGQVHTKTANVEATASKASITAVSKQVILAENANMVTASSLVSALYDVDGFQIHNFDDVTMSGFDAQAIGPQTVTLTYTDAYGNQMTDSTTVIVDLATITGQATHPIAGPAATWDYRDSVTQVIDANGKTIDVDTADITATTPNLTLAKAGKPQTVMLTYTDSLGKVHTTDVIVTTTLSKAKITAVADQVIWPDQAKQLTATDLVDRLYDAEGHLITNYDNVEMSVLDSKLAGQQRLTLTYTDVAGNQSVAYANVTVDQAKLVTKPSTVIAGPTATWSYEAGISQLTNAAGQLITVQPGTIKVLNRPDLNVDSVGQQQLITLIYTDELGKSQSVTAMVTAEASQATLTAKKAVILQPDAAAKLTANDLVTSLTDASGQAVTDYQIVQMSKLDATRPGVQPVSLTYTDAAGNEVSTVVKVTVDQAKMESQNRTQIWGPSMTWDYRQQLATVTDSQGHQLNPDQAKITVITGPQLTAKMIDKPQTVTLMYTDDLQQTHTVSATLTLTASQAALVPRPAQIVWAKDAGQLTPANFLQTITGADGTQVSSLTNVKMSAVDASQPGAQTVTLTYTDDYGNEVTTTAQVTVDQAALTTQTARPVAGPTAHWDYQTNFKTVTNAAGEVINVGDANLKVLTGPDLSTAMVGRPQVVTFSYTDELGLTQTTTAEVTTVASRAHMTTSADQVIWPAVVGKLTVADLVTGLTDAWGQTSQNYQSVTMTTINAQQAGKQQVTLTYTDEVGNVKTATTTVTVDQAALTTQPQTVIAGPTAKWDYHQGIGTITDGMGQPIAVNNAAITVVAMPDLTVAHIGQPQTVQLVYTDSLGQQQTALVQVTTVATQAKISTRPVTVIAGPKTTWSLNDSVDWSTSLAADGTLLTAAQRQRVTVDGTLNLRRASNYPLTLSYMDRAGNLITVTTSINVLASQAQLQVRDSQLTVGNAWTAQDNFERATDAQGQALTLADIAVDGTVNTQRAGQYTLTYHYTDVAGNQLTKTAVVTVVLPEDDHINTTDPDNNDHGETTNPDNNDHAGIADPSETPKPSERPNDSDGHTVDWGVDDRITTKQQPAAATRAQTKVKTTAEPALPANNEHTSAAKAAATPVTRVTDTTADTLPQTGERDRSAQQGAVVLGLTGLLGLMGLGRRRHTHED

SEQ ID NO: 16 - protein, 1357 aa, Lactobacillus plantarum
MRYTRGKWRVTNPKVWLFSSVLILGWRIVPTVAQASEAETVTMSSHSVQLETDSQDQLTEVARISKTAVTRDRHSVTAQSSKSADRTSSEQSATTGTVEAVSPTTSEAQQRSTQQDKTAVDQQASDSTAASAGASTNQASAATSSDQAPAANSTGTHHAIDMASSASALGADSGAHSESLSEAQHSGGQGKTIDSDLSGTVHSQSSVSTVTTATPVNSNSSRAVAATDQMSSRVEKRALNKTNVTKSINIPVATKQPSKQRTVTASSFLTTAKNLADKNYLDQYAKQHGQAALIALIQDWLSTYRIIALTGITIVNSSFDGSVATISGGLHVINTGATIRSGQDDEWETIINGGLSVTNNTITFTTTNGLVDRPVANQDMDFTKPRPTGNGAIKGLPSVTVDSSLINAQEFSQAQINISDFYDQLVTAGTILSATNGGTLSKMLIGESGTADLGSYQGHHYYAVNIDLNDWHSGIRTTGFNNDDVVIYNVVTAAPALTIGGGFSSSTPNLVWNFNHAMRIQNTTMITGKIVAPHAVFTTNQNVDSAAVLQYGYGDVDSAIRETITSQNEHNYGFGQVVTDDPLDYLIAVIKSDGTSIDTLAGFRHLLATGQLKITITDAAGTRLSGLNAVDTHIAGQHCYLITYQFGDQTATTWLNVQPSHEPIIPISRIPEYSAITRTINYQDERTGAVLAGPVIQNVRVVRFAIFNAKTHELLGYDTNGDGIVDTSDGTIAWLLVPPTDQDWVQVVSPDLSAQGYQAPDIPVVAGQTVIINGGDRTMNTNVIVKYQQQTHIATTQRTVTRTINYIDGGTLQPIASLHAVVQTVKYQLLAVVAHDGTILGYDTNGDGQIETQLADEAWLIVGSGPWFGAVKSPDLSHEGYAAPDLKVVPEQMVAGVDDKDVTINVYYRLATQAVTVYQNKRRVISYIDRQTHQSIATTVQQLVIYQRTAIIEKKTGKCLGYDLNGDGLVDTSQADYAWILVGSGQFAAVTSPTLVVQGYTDPDIRTVAAQTVAITDPDLMTTIVTYDHRIITVTPGNPARPGQPVDPDNPNILFPDEGGDTDLTHTVTRIIHYVYEDGTTAAASVLQTVQFQRNAMIDLVTGEVTYQEWVPVSVTEMAGVISPIVAGATTTLTEVAAQQVSVTTADQVVVVTYKKSAIKPEEPGQPEQPSQPEEPGQPEQPSQPEEPGQPEQPSQPEEPGQPEQPSQPEEPGHPEQPSQPEEPGQPEQPSQPEEPGQSEKPGELQKPSQPADSEQPDGLSDQANLSRNQAEQSRTSQPSQAESDQSVVQTNQQKTAASVSGIGWVSAPAVSKRTTKHHRMTTLPQTDEQNTQLSLLGMIGLALSSILGWLKIKSRD

SEQ ID NO: 17 - protein, 1189 aa, Lactobacillus plantarum
MKPNNVNNQNKRHQSRWVITSATAMILTTLTIASQAAAADDTVTTTTNEPTNSQLNTNTQVNATQVNLKADTSTSVSTIKSDQSAVAATSPTTSTGSPSEHSSSVNTNPQQQSANPASQSQATTTSESTPTTDIKHPTQTAPAQTTSASTTEPTTESNTESATDSQAKATTTDNQASKQPSQQAVPASSNSTTTEVNTQSATSSASTDDKIVTNVNQEKLVLKTNQPVVRAISRTASENINDWMPNTLLQQEVLSQLRKQNPDRTWNSAADITKADMLLLTTYYGKDTYIDGKTSYSLEGLQYATNLTTVWLNNNLNAPSGSYYSDVTDISPLANLQKLQVVNIQQNRITDISPLANLKNLTEVDAAYNHISDFSPLKGFKNLKGTFSNQFITLPPAYISADNNIATLAIDCYLPDGSKVQLKPNNGVGETVFYKNGQLYVRWYFNGAGGGNYDSNGHIYYTNMKPQQPGLTGPTFNGTTVIPMDDYYFMTAASDGNNFVVVRPYVLAATAAPITVKYVDALTGESLVTADLTLNGIVGQPYTTQRIDDELPNYDFTNIVGNASGVFTADAQTVTYYYTRKDAGDITIHMVDTNGNLVYEPQILPGKHNLGNAYNLDAPTFDHFKLHQTIGNAAGVFTTDPQSITFVYVRLDAGNITVKYQDKQGHQLKPDKTVSGSQSLGQTYTTEPLGIENYTLMTTPANATGTFTDQEQTVIYVYVRRDAGQIVVKYQDSAGNPLAPDKLLDGKEQLGVAYQTAAISIPNFYLVATPANATGTFSTDTQTVIYQYARSNAGHITVKYQDANGTTLAPDDVLTGDGQLGRPYQTSAKTIENYRLIQTPANATGQFSDQAQTVIYVYTREDAGDITVQYLDENGQQLAADSVLSGQGQLGQPYETSPLNINGYTVKSTQGNTTGTYTVQPQRVVYIYERTAGQPVTAKYQDQDGKSIHPDVVHSGYLGDNYSTEQLVIDGYTFKAVQGDVSGTFGTSAKTVTYVYTESTPTIPDTQGTVTVHYVTKDGIKLNEPTVLSGKTGTTYQTVPLTFTDHELVGQPENATGLFTADNVDVTYVYQATDTTGTDDIIDPEEPEQPTKPIKPTTPETPNEPGTTVTQPDRIKPTQPAVAVKPAATVKPTLKPAAAQASLVKTTSPVTEHSAQLPQTDEQTGKLAVILGLLLSVVTLGFYGKNRQS

SEQ ID NO: 18 - protein, 2032 aa, Lactobacillus plantarum
MQRRRLQRAQLTEKRTYKMYKKGRLWLIAGLSTFTLGASLLPMTGRADTTSTPAEKQGTRTETTGNQITLASKSVGSSSMANDGEEKTNNSQVETSSEASNVTASTEAKSTESTTQTVVDSTVTSTATETTRANGATNQTSKMSIVDTTSNNTEQNQAVGGTTDSTASTATIEDQAKAANRATTDGKINTATVATKTTTTASYATADISTNTIRSAQKLARATVATVATVNSATKTYDGKIDTPNRYTITLTDGTKAPSDWAVTSTANVYTVTDLTDVDTSKFGSSVGTYTLALSTAGITKLAEANSSADITAANVVTGTLTIKQAPVPTAIITIGSASIDYGDAKPSTYTITVPSQYAVPSTWTLASSATDGTTNTYMIASSSGDVIVPTATQSGTYQLVLSDQGLTALQQANPNAAITADTIIAGSLVIAAHDIITMGATTIVVNKTTSTVPVTVNSRTIVVPTGWTIRYDDIQTDAIVYDVPVSDTTYSEAVNTAVVDKYTITLTDDTIETLANLNSSTTFNSTTVGKGVVLVKASAAVAISPANYGAQASAETPVTGLTISHARTKGIDLAYGQALYLILPLINMNPSGMTVANLTDYVIIPSGFKVATNSEGAINIATDPSSVLTSAIEAMMTKNDVTYQGLKVTQLTDYRGRQTFKIHFDKTTVYDGGAFATLKYALLPVIAVQNTGVTSGLIGNQVSSPDSAVVYVTDDSNENNGSYSLNLQNYTNIDSVADALGIADAVTIGSGFTSYLYHYTLSAKTITDTYSLVGNDGTSLGEVTFTGDSGKTYVPMTKLPMTITQNGVTYYLNTSAVSLTQTYSGDSNSNYTVTYQRYVTTTTDTAAKITIAPASKVYDNNATTDPSRYTVYLPTEYTAPSDWTADSAATAVDGTTAYQVSTDYLNTTAIDQNVGTYAVTLNSAGMAALSAANPDFLIAGDVNVGGTLTITQRPVTITLPDTILWANGQEQNITPVITGVVAVQSLDYTLTSGLTDPDTTTITATLTNAAANSNYKLTNSPSGQLTVGAVTVVYQYGYRDKAGTLHVVTTANGTATHGTDVTAKDYLSYTTSDTTATHAKTGYTLQPESTGYQADGTLADVGGQVVYTYLANTEKIAVVYVDQDKNNVILKQIPLSGSFGTPTNYTTAQDIAAYEKLGYVLASDKVPAPLEFDQDTEQTYYVYLKHGTITATVDQPGNVAVSDLMKTSQRTIHYVYADNTPTDLADVLQTVTYTRTATGDAVDRTVLSYGNWTTNVNSYPAIESPTITGYTADQTTIAAAVPASMGETTETTVRYSVNSETIRVQFVDGTTDNQVLSYIDLNGKYGDAADYTVTADIAKYAKLGYEPVNSDLPDQLIYKQNTQVYTVTLAHRHVTVSVDHPGQPGQAIDADYPAGPKYPAGTGRDSLEQTVTRTITYQYASGESAAETVNQSVTFNRTATFDMATGKQLTYGDWTVAPGQSALLAAVTSPTITGYQASVTEVEAASVTSHDKPHLIAITYTAKSQTATVAFVDVTSGKTLPTTVVTGAYGTTNSYSPVSQIAAYEKLGYRLVSNNVPTTGITFDQNDVIKSYTVKLAHQMTTVTPTKPGQPGQPVDPAHPEGPKYPAGTGLKDLTTSVQRVITYVYNDGQTAAPTVTQTVSFERKATFDQVTKVVTYTDWRTPESALTGAYAVVESPIIAGYTPNATRVASVTVSAKDTESRQTVTYQANLETATVTYVDATTGHRLGTSVTLTGRFGTQADYQPTTMIAQYTQAGYVLMGSDYPATGVTFNQAGVVQKYTVYLAHNKIVITAPDQLTKTITQTVHYQDQAGHTLQADTIRALTFTRSGMKDAVTGVATYRDWAPTGLNFTAVSAPTIAKYHALTATTQAVAITAASADDVQTLTYALDVPTPTKPVKLTKPAKPTKPTTSDDLIKPTTKPITAAKPTQLTKPATVVKDFQATTGNQTPAKSTRTLVSSRIKAVKTAPASAIIKPGSKVTEPAHKAQADTTSRLPQTGETRWSEMAAETLGLTLATLLLGFGGLKRKRHEK

SEQ ID NO: 19 - protein, 2457 aa, Lactobacillus gasseri
MVPQFTWGGVNAQAVRADSVNEDATEQVEKKDEANVKAAEVKTTEQKQENNKTAVSATNENAKQNVAENTSDSKKVASNRDVNVIKNDVTTDEKAAAKSSVQTDKDVNANKLNTNTVSVNKLQRNVNVAGLAESKATSEINSTLSVRESMQQKAVSLKANEIARTVIMNKPAGPDQITQSVKLGTMLGSSNGQIIDGKTTKIYTATVIAVGSSTDMKKYRVTVDSDTGEILAGQDLYDTFMNLQPSDFKVNLDAIDQSQIDVPGYTWKITSATPAGANIGKEDYTFGNPQTITIDYTRDVEGNIKKKVTEITDKLVNNQMTTEPARTVILKKTTTGAANDETIVQKADIRGLARTSSKTVAGITEKKIEVAIAPYVEPDKPSSQYYKQYTITFNPDTGQIISGQNDYDQLMALKRSDFKADLPAIEDSQIDVPGYTAIITSATPAGAGLEAETYTFGHPQTITIDYTKVKHTVTYQFKDPFGNQVGTSVPVTGAVGSNQSVNLTLPDGYQLASGSLPTSVTIPESDKIIPIPVKHQLTITLSGESVFNYADDNWQNLVETNELPASGYYVEFNDANARVQLNDGDVTYNENRNAGTYTVSLTEKGLNDIKDQSHDNFIYPDLKDVKSEAKFIINKGNKTISLMGGDTKVFDNTSTLPDQGTFYSGLGLADNDQGRISVYNSDGNPRTIQLTPADVEFWFNGHKIAKDQAKNVGNYNLRLTDDFINKVKAADGNNGNNYEWAYGTNTPTGSDTYTADYVIYQATGKAKLSGNNSKLYDGNAVTTDDVNKGRKITIDLTLPVYKQADEPGDEPQLLGTVDLGKYTLQDGDYTWANGTAPTKGGSYTINLNKDKILAHLQDRLVALAGKGTDPDDSTKSLSNVTISADDMAGQATFAIETTTTYQFVDDDDNGSKVGTPVSKTGLKGESSNISLTVPTNYVLAAGQTLPTSVTFGDTNTTVDIHLKHATKTVDKNNVPDGYTKDDFAETINRTITAKEPTGDVDLSQTTELTRTGTYDEVTKKVISYGNWTTGNFDEVTAPEVAGYTPSQANVAAVTGVTPDYVDPKVVITYAPNDQTGKISYVDVNTGTEVGNTPLTGKTDEEVTINPVAPTGWKIVDGQSIPRTEKATPTGIPPVTVKVEHKTTVVPPTDPKTPKDKLPDNPDKHYPDGVGEKDLNKIIVRQITVVKPDGTREKHDQSVKLTRNATVDEVTGEVIKYGDWTTSNFGEYDAPTVPGYTPSQAKVEGVKVTADSDFAPVTITYTANPHTLNINYVDKDGNKIGNSYQVPGRTDETVAVDVPGHVPANWELVPKQKYTTSITFGSDDPQDQNYVIQHKTTTTDGRDHKDNQDLYREVTRTILMKVPNATSQGRETETLSFYRIKTHDEVTGKDTYSDWASNVTGDKIAFDEFDVSKTNDGKEIAAGYTPTSNDVVLEDKNGDKFVPSQSALKNGVPADSFTVEVAYTPNAQRTTVTYVDENGKEITNPDGSVIPGSHYDLTGVTDQSNVPTNIQNNVPTNWHITDPEVPATITFGADGHTPIKVHVAHNTKPVDKNDVPDGYKESDFSKTINRTITANEPSKSVDLSQKTELTRTGTYDVVTKKVISYGNWTTGKFDEVKAPEVAGYTPNPASVNAESVTADYVDPKLVINYTPNDQTGKISYVDVNTGTEVGITPLTGKTDSDVTITPSAPAGWKIVDGQNIPTTEKATPTGIATVTVKVEHKTTTVPPTDPKTPKDKLPDNPDKHYPDGVSEKDLNKTVVRQITVVKPDGTKESHDQSIKLTRTATVDEVTGEVTKYSDWTTGNFGEYDAPVIPGYTPSQAKVEGVKVTADSDFTPVEITYTPNAQKTTVTYVDENDKEITNPDGSVIPGSHYDVTGVTNKKVDTNIQKNVPTNWHITDPEVPATITFGADGHTPITVHVAHNTKPVDKNDLPDNYKESDFSKTINRTITAKEPNKDVDLSQEIELTRTGTYDEVTKKVISYSDWTTGKFDEVKAPEVAGYTPSQAKVDGVDKVTVDYVDPNVVITYIEDPVGQDITVKKGDTPDPEDGVKNHGDLDKITDPKHPGTKTTYTWKKTPDTSVAGDVPATVVVHYPDGSDKPVDITVHVVDDTPVVPTKNPDPVGQDITVKKGDTPDPEDGVKNHGDLDKITDPKHPGTKTTYTWKKTPDTSVAGDVPATVVVHYPDGSDKSVDITVHVVDDTPVVPTKNPDPVGQDITVKKGDTPDPEDGVNNHGDLDKITDPKHPGTKTTYTWKKTPDTSVAGDVPATVVVHYPDGSDKSVDITVHVVDDTPVVPTKNPDPTGQDIHTPQGKVPTPESAITNKDKMPDGTKYTWKEIPDVNTLGKHPNVVVVTYPDGTAVEVKVNVFVDGTPEVKKETKAPVVKKQVVEPTKVETRQKLVNNYVAPRAVEVQRAQAKGKRQLPQTGAKENIASEVLGMLSVGLGALTAGFASKRRKKNR

SEQ ID NO: 20 - protein, 1529 aa, Lactobacillus salivarius
MEKLLGEKRRYKLYKAKSKWVVSAIITISGVTFLVTSPVSNAQADTVTGSESVKTEATQASSSSVQNNTTAQTTVTTNSNSSNNVSNVQTDTVKEAATSNVDSVASQNQATTAQQAKTTADTADQTVPPTTYKDHVKGNVQTAWDNGYKGQGMVVAVIDSGADTNHKDFSKAPESPAISKEDADKKISELGYGKYTSEKFPFVYNYASRDNNWVKDDGPDASEHGQHVAGIIGADGQPNGNERYAVGVAPETQLMMMRVFNDQFADENTDDIAQAIYDAVKLGANVIQMSLGQGVAAANLNDVEQKAVEYATQHGVFVSISASNNGNSASVTGEEVPYEPGGADGNFEPFSSSTVANPGASRNAMTVAAENSVVGAGDDMADFSSWGPLQDFTLKPDVSAPGVSVTSTGNDNRYNTMSGTSMAGPFNAGVAALVMQRLKSTTNLSGADLVQATKALIMNTAKPMTQQGYDTPVSPRRQGAGEIDAGAATESPVYVVAADGTSSVSLRKVGDSTQFALTFKNLSDKDQTYTFDDFGGGLTEVRDADTGTFHDVYLAGAHVYGNKTVTVKAGQSATYNFTLSLTGLKENQLVEGWLRFVGNDGQNQLVVPYLAYYGDMTSEDVFDKAANQEGTVYGGNYFVNEDNYPRGIADENSLKALVNLEGNYNWQQVAKLYQDGKVAFSPNADGKSDLLKPYAFVKQNLKDLKVEVLDKNGKVVRVVADEQGLDKSYYESGVNKDVTLSVSMRNNPNTLAWDGKVYDDKTGEMVNAADGEYTYRYVATLYNDGANRVQTADYPVVIDTTAPVLSNVKYDATTHTLSFDYKDTGSGFTDYSYAVVKVNDKTFGYKLNDGKNSKFLNAAKTSGTFKAVLDSDTLAALTAAKNALSVAVSDVADNTSTVTLLVNGNNDATTKVSVWNATNGLELDQSSPDYQAATSTYNLRGNATSDFYYNGALVQGDNSGNFVVPVSTSDTAVVFTSDAAGKNVVYKLNTATPKAVFAWQVNNTVKENFGIVLDTVVSNNKDDVVVQAAVTKGDNVEAYARDYFTGAVYKADVKDGLATFHVKVTNNSGRTVLLGWTEVVGPTFNDVQRTSANGVYLGVDTDTENPTPAPAFTSADQLGTNVVQEKADSATIGNPGDLPGHSLKDLTTRADANPDIHFDYLKDNDYNWVGAQAVKDGVYNPSTQVFTLTGKVDPNVKSLVVLGDSYNEDDPVNKVNLNSDGTFSFQFHTAPTSQRPVAYIYTKDDGSTTRGTMELILDTVLPTLSLNNVANLQLDSNGDYQVYTNNKDFSVSGEATDNLDGYRFFFNGDNDYREFHNSGVNFVTEAHQDGSTVTNPYPAYKFSKTFNLADATGETTHVYTLSVVDLTGNTVTRKFYVHYQPASDTVKTVTTDKDGVTKVLVDYNNNTLQVKDSTGNWVNATGVEAAKDYRVVNEYGNVVLLLNVLADKEQDNNKVQVNEVTNNKVEQTVVTKTVSNKSVAKVGKKAAEPVKVLPQTGENNSKSTSVLGAVLASIAGFLGALGLRRVKKD

SEQ ID NO: 21 - protein, 1136 aa, Lactobacillus plantarum
MMVLLQVIAAGATVSLGADMTAQAATLPQLTFAKSTASDNILTNQHFDVELQVGDTASKINTIDLPNEVNLDGPEEFKQIKRVFDDSQYTTGDNGAFTITAKHLTVAYNPDKRRITVQWSDEYPQTKVPIRLTAVKAEKLALVAVADDQKGPALNVEIKQPQTQADQASTSSASSSAATDTNSSTASSSRQATSSAASLDSSRSAATTLSSQAVNQTSASSSEPSQETAANQSSAVTESAGETTDSSASISSSSTASQVFSSAPTKQATASAKSSPLIPVTRLAQLSSNVVDVSQWSQLVDAWKDASVDEINITADISNPTAASGALDSRLSGNIIVNGNGHSVNIGRAGFHTRNNTATSGTMYTATFMNFASLIGSFGNDAGLIGSSTGGDGAGGALNWTFNVSNITVPSGTSYTNTSRRFVSAQGNQVNITGNCRVTTVRENILCGGLDVAAGQTFTGSKIANGDDNSFIWFVYDYQGTGNRQVNVEEGATLNCIRRPASSTSTAYTTYPVIFDAYESINVGKNATFNASVPGNAYSNKYFYGSQYHRNFYADTGSTVNLTSLARSQSPISFSDNATSTIQSSSGANIYVIAATGAPLISGNYARLATVRFINPNNLDLRNSSTGTTAAASSINQDNVGTFEIQDSNISLWKLASSVTGGADYSYSNVSQLLQQGSAVTATDSNLQSNYLSSKMRRISATNQKPQLAFNNPYDGTTKLTDADQKLRTRVIVAMVPDTNGVQDDGTVNYIPQYASAGQLTVSYSVNGKTITAQTDSNGYATANVGTFLKAGTTVTASTSNTSGTTVTATGTVVDVTPPNPATMVSPDPIRVSTGTVSGQNGEPGAQVTLALNGQIQTNVKTVVNANGTWSLNLTGLSLKIGDKIIIYMADSLGNRNPDPNSYPNGQQYHDATFQPAPIFTVAKDLIVNPIDPDDPSKPGTGGTNNLGPLSLDAVPTHLNFGQHSIPTMDTAYPLLSPSAAEDQLATATDGQKYATVGGQKNGQDSVYTQVTDTRDTPSGWQLTAQLSALTATDGTTMTGSYVTLTSGTAQYLNASTSKWVTATDQNQATLPAVIKLTPGATQQTLIAGTTSQQGVGTNQQIWNVNNVALHVKGGRVMAKNYSGTITWQLNSLPSQ

SEQ ID NO: 22 - protein, 1621 aa, Lactobacillus salivarius
MEKPTPIDVTYHYDRMNPASIEDRTDISYHYNKISVPIPNPTKKADKEGKTLIAGDESTQHISQYTGVNQKLDKFAVGDAIQYTNDGRLPVSFDLSKWTVTTSNGTNVTAQGKFTQYDKTFEGKKYHVVSWSPTNVSSLKDNETYTLNTILKTLNDGITDGEIDRAVGGGDGVTFGEAHGYDEFNPTTDKAWKEGSQTVNGKIEINEDIAHAKVTMTMPDPAKLANKLSNVAITDNYSKFANLVTVTGANVYENERNATSDYTIVNNNKVVTATRKNPATANGGTVSLVVDFKVNPDVPSGTKLVNSGSGTINTQTVPTPDAQIVTFTQTPTKHWVEGSQVVDGKTYINDDIVTTQVDMNLPDPKALAKTLSYVSVGDNYRDFADKTVLQSYKVLENGTDVTSQYTITNQGGILQAVRKNAATAPGGKVSLIATFAINHDVKSGTKLTNRGFGRINNHTVDTNTPQIVTFKQDTSKHWVEGSQVVDDKTYINEDMVHGQVTMTLPNKDSLAKSLTDVALVDDYSDYANKVSYVNAQVFENNTDVTSQYNITNAGNKITATRKNPGATPSGSVRLVANFKLNSNLPSGTKLINRGSGRINNNTVNTNEAKILTYVQSTDKHWVEGSQKVDGKTYIDGDTIHGQVTMTLPDKNTLAKALSTVQVIDDYSKFAKMVDYKSAQVLENGKDVTSEYNISNVYGQVVATRKNATATPSGNVTLNVTWTIHKDVPSGTQLVNSGSGRINSHTVPTPDRNIVTYKQDGLKDWINAQGQIVNGKTVIDNDTVHAKLVMTLPDPKTLATPLTKVQLDDNYSKFAGLVDYVSSQVLENGTDVTSQYNITNANDHVIATRKDASKTPGGKVEFRVNFKIHTDVPSGTTLMNSGEVTLNSETVPTPTPNIVTYKPDTDKHWVLDNNVTDNKIYFSGDKAVAQVSVDLPDASKLATPLSKLVLVDNYSDFADKVKLDSAKVLENGKDVTSEYDLTNKDGKVFATRKDAAKTPSGKAVLVTTFTINNGIENATALHNKGSVTVDSITDEVPDTPIVVFTPKAHKDVELGGDVKGDTENSVDGSLILNGSVVTYPITTSDLPAERAEDITKRVVKDTLDKNAEFVGFKAWIENDKGELEDVTSHYKLDKNGQDLTFTEDSYLLGLYNKDKSKQTHTPIIDLVVKVKGDAQKINNKATVLTNDNVTETNEVSVDTPAKPTPTKVDKNEKGVNIDGKNVLPGSVNNYELTMDLAKFKGIKVTDQDLAKGFYFVDDYPEEALDVDPQTFTYKTVDGKTVKGLSAKVYQSLSEVSENVATALKANGITPNGAFVLISADDPAQFFKDYVETGTNIVVNAPMKVKEGFAGKYQNKAWQLTFGQGEATDIVSNNVPKIDPKKDIVISADNRTSLNNHTIELGQNFDYLLKGGILDKDQGHDIYEYKWVDDYDENHDQYNGQFIAPLTVDVTLKDGTVLKAGTDISNHVSQNIDTKTGSVEFSVDKDFLDKVDFDKSGFAADILMSVKRIKAGEVDNTYTNIINGQKFGSNTVHSTTPEPKEPETPATPKTHETPSVPVAQTQTPATPQPVKMVTSTPAPKAPESPALPQTGEANDTLAEEVVGFAAIVAALGMAGTSLKKRED

SEQ ID NO: 23 - protein, 2217 aa, Lactobacillus plantarum
MRNRLNRLGLESKSHYKLYKSGRRWVAASITVFSVGIGLTFSQVEQVKAATGTGVDTADNSASVSSDMAEPSNAVVLKSASTATATKTATQDAKAATDVTAATQDTKATTDSTGATSASSNRQSTAATKPAAEVGTASSSADSSASISSTDGASASAPSVTSKSTNTEATSASATKTATTSADTDVLNTETTSSSVANDLTDATTASQTRTETGKTASIPTAEAPTITTAVTSRALPLTGALASRSANTPVTKSAVQAVSAITSEAETKPTVSLVTTGTVSMDYGEASLADLESHISSPDETPANDVAYYIQDAAGNYLEDVNGNKVNLLYALFLDSADVNDYVDVVYTDEHGQVTKYSGDTDFSTLDQIGSYSVTINAAGKAGMSRVMQDYNAYDTSTSDLDDFVPTFSTGASDYTFTINIVPVKITATTGKNGLIILRPSQLYTGSLTMLPVVTVKNATKQNILQISNGEIGDAKPGVAGKVGQRVLTLADFTYTYQGTETNLTGADTGKYAITLNDAGRKAVQAALGSNYILDDAAVFTTTGAVQAAGLELKIASGTVTYNGKPQGTSVTTGTVYDHFDFTTTTDTNVGTYDDLTYALADPTQAAILAKNYTVTTTDGTLVITPADLTVTVKDDNAVYDGRSHGTTATVTSGTNYDQLVFTAVAADGSGATTYTTVGTYAMTGTTAADTSNYKISYVNGTLTIDPAKATITIPNKIYWSDGTQKNLAAVVTGTVNGETLKYRVTNGMSAVGTKTITATPDADDSVNKNYTISVIPGTLTIGDIAVKYLYEHVDANGETQVDASETGTATHATDATATDYLTYTTAAKPKTGYVLAPNTGLAYNGTLTDQGGTVTYRYLAKTETAIVTYFDQTDNKVIKTEPLQGAYGTTDAYRTADTIAAYENAGYDLVDDDYPTAGGVYDQDGIVQKYQVTLVHKFVTRTPDNPGTPGEPIDPDNPNGPTYPVGTDFEDLTEQVSRTIQYLYKDGRTAKPDNVQAVNFGRNVTVDEVNGTVVYTDWLTDDGAVTGRFEAVDSPLITGYTADSTSIAGNPAVVWQDDDTTIPVTYTVNKEYATVTYFDQTDNKVIKTEPLQGAYGTTDAYRTADTIAAYENAGYQLYRDDYPTAGVVYDQDGSVQKYQVTLVHKFVTRTPDNPGTPGEPIDPDNPNGPTYPVGTDFEDLTEQVSQTIQYLYKDGRTAKPNNVQAVNFSRNVTVDEVNGTVVYTDWLTDDGTMTGRFEAVDSPSITGYTADPTSVAGRDTVSGTDLSPDVQVYYQANPEKATVTYEDMTTGAVLTTDPITGDYQTVSNYRTADRIAQYLNMGYELVSDDYPTSGAVFDKDGSTQAYTVKLQHKLLPLTPENPGTPGEPIDPDNPNGPTYPAGTAVQDLIKQVDQTIHYQYQDKSTAADANTQTITFKRSVTVDEVNNKLTYTDWLTGTATTGRYMPVDSPEIKGYVADSTRIAGNDEVHNADADTNIVVTYQAKPENATVTYVDVTTGKTLAIKSLTGDYQTTSSYRTAETIASYVKNGYQLVRDNYPTSGAVFDVDNFAKTYTVTLKHKLATVTPENPGTPGQPIDPDNPDGPKYPVGTTAQDLTKQVSQTIKYRYQNGASAGTDNVQLITFNRDATIDEVDPTAVYTDWLNGTSASGRYTTVMSPVITGYTADKTQVAGRDSVANTDSDTQVVVTYAAKPEKATVTYVDVTAGKTLATANLTGDYRTQSNYRTAETIAGYVKNGYELVRDNYPVSGMLFDVDDFAKTYTVTLKHKLVTVTPGNPGTPGQPIDPDNPDGPKYPVGTTAQDLTKQVSQTIKYRYQNGASAGTDSVQLITFNRDATIDEVEPTVVYTDWLDGTSATGRYTTVTSPVIIGYTADRARVTGNDAVTSAAQPTNIIVTYALNAEKATVTYVDVTTDKTLATVSLTGDYQTSSDYRTANTIADYSNQGYVLVRDSYPVSGAIFNDDGVVHSYLVQLAHVTTATTETKTITQTVHYQSTTGTQLHDDTVRAMTFTRTKRVDQVTGDVTYSNWSTNQADHTFERVAAFSIPGYHAVVTGTQAVMVTPASVDDVQTIRYVTDRLSTGETPKTPVKTVTVNKSDKIKTTDTPDKVATVKTPDKAQTVATTTAKQASVKRSVDLKQAQAVEQPAQTRPANVKTVKLAKTTKSVKPTAAHQSATHKQATLPQTNDDRQASVAAELLGLTAATLLVGVSAILKKRHN

SEQ ID NO: 24 - protein, 2023 aa, Lactobacillus plantarum
MQRRRLQRAQLTEKRTYKMYKKGRLWLIAGLSTFTLGASLLPMTGRADTTSTPAEKQGTRTETTGNQITLASKSVGSSSMANDGEEKTNNSQVETSSEASNVTASTEAKSTESTTQTVVDSTVTSTATETTRANGATNQTSKMSIVDTTSNNTEQNQAVGGTTDSTASTATIEDQAKAANRATTDGKINTATVATKTTTTASYATADISTNTIRSAQKLARATVATVATVNSATKTYDGKIDTPNRYTITLTDGTKAPSDWAVTSTANVYTVTDLTDVDTSKFGSSVGTYTLALSTAGITKLAEANSSADITAANVVTGTLTIKQAPVPTAIITIGSASIDYGDAKPSTYTITVPSQYAVPSTWTLASSATDGTTNTYMIASSSGDVIVPTATQSGTYQLVLSDQGLTALQQANPNAAITADTIIAGSLVIAAHDIITMGATTIVVNKTTSTVPVTVNSRTIVVPTGWTIRYDDIQTDAIVYDVPVSDTTYSEAVNTAVVDKYTITLTDDTIETLANLNSSTTFNSTTVGKGVVLVKASAAVAISPANYGAQASAETPVTGLTISHARTKGIDLAYGQALYLILPLINMNPSGMTVANLTDYVIIPSGFKVATNSEGAINIATDPSSVLTSAIEAMMTKNDVTYQGLKVTQLTDYRGRQTFKIHFDKTTVYDGGAFATLKYALLPVIAVQNTGVTSGLIGNQVSSPDSAVVYVTDDSNENNGSYSLNLQNYTNIDSVADALGIADAVTIGSGFTSYLYHYTLSAKTITDTYSLVGNDGTSLGEVTFTGDSGKTYVPMTKLPMTITQNGVTYYLNTSAVSLTQTYSGDSNSNYTVTYQRYVTTTTDTAAKITIAPASKVYDNNATTDPSRYTVYLPTEYTAPSDWTADSAATAVDGTTAYQVSTDYLNTTAIDQNVGTYAVTLNSAGMAALSAANPDFLIAGDVNVGGTLTITQRPVTITLPDTILWANGQEQNITPVITGVVAVQSLDYTLTSGLTDPDTTTITATLTNAAANSNYKLTNSPSGQLTVGAVTVVYQYGYRDKAGTLHVVTTANGTATHGTDVTAKDYLSYTTSDTTATHAKTGYTLQPESTGYQADGTLADVGGQVVYTYLANTEKIAVVYVDQDKNNVILKQIPLSGSFGTPTNYTTAQDIAAYEKLGYVLASDKVPAPLEFDQDTEQTYYVYLKHGTITATVDQPGNVAVSDLMKTSQRTIHYVYADNTPTDLADVLQTVTYTRTATVDAVDRTVLSYGNWTTNVNSYPAIESPTITGYTADQTTIAAAVPASMGETTETTVRYSVNSETIRVQFVDGTTDNQVLSYIDLNGKYGDAADYTVTADIAKYAKLGYEPVNSDLPDQLIYKQNTQVYTVTLAHRHVTVSVDHPGQPGQAIDADYPAGPKYPAGTGRDSLEQTVTRTITYQYASGESAAETVNQSVTFNRTATFDMATGKQLTYGDWTVAPGQSALLAAVTSPTITGYQASVTEVEAASVTSHDKPHLIAITYTAKSQTATVAFVDVTSGKTLPTMVVTGAYGTTNSYSPVSQIAAYEQLGYRLVSNNVPTTGITFDQNDVIKSYTVKLAHQMTTVTPTKPGQPGQPVDSAHPEGPKYPAGTGLKDLTTSVQRVITYVYNDGQTAAPTVTQTVSFERKATFDQVTKVVTYMDWRTPESALTGAYAVVESPIIAGYTPNATRVASVTVSAKDTESRQTVTYQANLETAMVTYVDATTGHRLGTSVTLTGRFGTQADYQPTTMIAQYTQAGYVLMGSDYPATGVTFNQAGVVQKYTVYLAHNKIVITAPDQLTKTITQTVHYQDQARHTLQADTIRTLTFTRSGIEDAVTGVATYRDWAPTGLNFTAISAPTIAKYHALTATTQAVAITAASADDVQTLTYALDVPTSIKPGKPTTSDDLIKPTTKPITAAKPTQLTKPAMVVKAVQATTGNQTPAKSTRTLVSSRIKAVKTAPVSAVIKPGSKVTEPAHKAQADTTSRLPQTGETRWSEMAAETLGLTLATLLLGFGGLKRKRHEK

SEQ ID NO: 25 - protein, 1519 aa, Lactobacillus brevis
MRNRLNKMEPEGKTHYKLYKSGRRWVTAGITVFSVGIGLTLSQVGQAKAATNSDTDETENSATVSSSSPTETKNAVVLKSSSAAATSTAAAAVSASTASDSQSTATPAASTSRAVSGAATGAAASDSAATQPTVSSADSQSTENTRWSAASDTTSNAASDQESQQAAGTTDNANSDAASSATTATNTNAMPMTNRITSRAMNVTAAVSEAEAQPTVSLVTTGTVAMSYGDASLADIGLHISSPDETPANNVAYYIQDAAGNYLEDVNGNKVNLLYAFFLDSVDVNGYFDVMYTDVHGHVTKYSEDTDLSTLNQIGSYAVTINAAGKAAMSQVMQRYNAYDTTTNVFVDFVPTFSTGTSDYTFTINIVPAKITATTGVNGLTMLRPSQAYVGSLTMIPLVTVKDSEKKNVLQISNGEIDYAAEDVVGKAGQSILTPADFTYTYQGTETNLTGADTGKYTITLNNAGRAAVQAALGPNYILDDTAIFTTTGAVKAADLGLTIASDTVTYNGQAQGTSVAVTNGTAYDHLDFTTTTGKDVGTYDDLTYALADPTQAAILAKNYNVTTTDGTLVITPADLTVTVKDDHAVYDGRAHGATATVTSGTNYDQLAFTTVAADGSGATAYTKVGTYAMTGTTVADTSNYQISYVNGTLTIDPAKATITIPSQVYWADGTQKNLTAVVTGTVDGETLKYRVTDGMSAVGTKTITATPDADDLVNKNYTISVIPGTLTIGDIAVKYLYEHVDANGETQVDATETGTATHATDATAADYLTYTTVDKPKTGYALAPNTGLAYNGTLTDQGGTVTYLYLAKTETAIVTYFDQTDNKVIKTETLQGAYGTTDAYRTADTIAAYENAGYDLVIDDYPTAGVVYDQDGSIQKYQVTLDHKFVTRTPDNPGTPGEPIDPDNPNGPTYPVGTDFEDLTEQVSRTIQYLYKDGRTAKPDNVQAVNFSRNVTVDEVNGAVVYTDWLTDDGAVTGCFEAVDSPVITGYTADSTSVAGRDTVSGTDLSPDVQVYYQANPEKATVTYEDTTTGVVLTTDWLTGDYQTVSNYRTAERIAQYIKAGYELDVDGYPAAGVVYDQDGIVQAYTVTLKHKFITVTPDNPGVAGDPINPDNPDGPKYPNGTAAKDLSKKVSRTIRYQFENGELAGMDNVQTISFSRNVTIDVVAGTKVYTDWLNDSSLTGSYKAVDSPMIAGYTADILRVAGNTSVLGTDQDNDIVVTYTASSKEATVTYVDTTTGAVLATVSLSGTPDTPSDYRTATTIAAYVKQGYELVSDDYPTSGAPFSEGGVNYTVRLAHATDTTPETKTITQTVHYQASNGTPLHTDTISTITFTRTKVVDHVTGTVVYSGWVTSKDDNTFVSVPAIAISGYHPSVTGTQAVTVTPDSADDVQTIDYVADTVTIKTPDQPLKVKKSQKKQKKVVQVKQLKKIKQPVQMAGATAAALELGKTIRPIKQAAKNKQAVENKQVTTREQATTQKRATLPQTNDNRQASVTAEILGLIVAALLAGLSAMLKRRHEG

SEQ ID NO: 26 - protein, 1111 aa, Lactobacillus brevis
MRNRLNKMGLEGKTHYKLYKSGRNWIAAGITVFSVGMGLAFSQTDQVQAATNTSADGVENSATVSSSSPTETKNTVVLNASSAAATSTAASKDDAAAATSVATAGDSQSTVTSAASASRAVSGAAMEATASDSAATQPTASSADSQSAQSVYESAASGTTSQTAASQESQQVADNAASDAASSATTATNTSPLPKIKMSRAMNATALASEAEAKPTVSLVTTGTVSMNYGDASLADLESYISSPDETPVNDIAYYIQDAAGNYLEDVNGNKVNLLYALFLDSTEVNDYVDIVYTDEHGQVTKYSGDVDLSTLTQIGSYTVTINDAGKAAMNRVMQDYNAYDTLTSDLNGFIPTFSTGAADYSFTVNIVPIKITATTGMNGLNMLRLSQSYTGSLTMLPVVTIKNSQKRNILQINNGEISDAQLGVAGKVGQRILTLADFTYTYQGTETNFTGADAGQYTITLNDAGRKAVQAALGSNYILDDAATFTTTGTVKAADLGLTVASDTVTYNGQAQGTSVAVTSGTAYDHFDFTTTTGKNVGTYNDLTYALTDSTQAAILAKNYNVTTTDGTLVITPAELTVTVNDDHVVYNGQAQKTTATVTSGTNYDDLAFTAVAADGSGASAYTKVGTYAMTGTTAADTSNYKVSYVNGTLTIDPAKATITIPNQVYWADGTQKSLSAVVTGTVNGETLKYRVTDGMSAVGTKTITATPDANDSVNKNYTISVVPGTLTIGDITVKYLYEHVDADGQTQIDATEIGTAAHATDATATDYLTYTTAAKPKTGYALAPNTGLAYNGTLTDQGGTVTYLYLAKNATATVTYIDTTTGSVLHTKNLTGMLDTQSSYQTADTIANYVKKGYVLVSDDYPTSGAIFSEDSANYTVRLAHATDVTAETKTVTQTVHYQDSTGKPLHADTVNTITFTRTKVADQVTGEVTYSDWSSSKGGNTFDVVSVPNVSGYRPDTTKIQAVMVTPASADDVQTVTYSVAESGTGYDVVNPKVPGDPIAEPEPYVPFAGTKKVKAGDTGKLVNKQKVVKAGAAVQTAGKQTVKLSATKSVKPVKTQVDANRVNLTETKRLPQTGEAQSHTETAGLIGLGLATLLAGLGLGCNRRKED

SEQ ID NO: 27 - protein, 915 aa, Lactobacillus plantarum
MQHRQLWYRGGLGLALALVVVGYRGSRTVIRAVPRAQLSVDQKMPSTSSVFSASKLTLQDEANNSAPQVSPEAQESSGPDKQSDLTSGSSTSSSGISSGNSSGSTILENAKNNQTSETATTKAAEMVNGTVKMTLDTNGTLHLSGGSFGASLGSATGSWIVKTLTANGYQPTQVSKIVIDGKITATTMTNYSYLFANLPNVTAIDGLANLNLTGVTDISWLFLNCSQLGALDLNSWDVSSVIRMEGTFQNCAKLVTLNVANWNTDSLQYLIDTFNGDSSLTSLPVGKWNTSKVATMMRTFTDCSSLTSLDIANWDTRVVTNMSAIFRGMSKVKSLPIDKWQTGRVVNMQLVFSGDTSLESINVANWDTSRATALDGTFAKLPNIKSLPLDNWNTSNVQTIRSTFYGDTNLTQLPIDNWNVGKVFDFNSTFSGCASLTTAPVANWNTQSATNLGYTFEGMTSLTSLPVDNWQTGTVTNMAGTFSGVSQLKSLPISKWNTKNVQNMAGTFSKMSSVTALPVDNWQTGNVTTMRGIFTKVSQVKNLPVGKWNTAKVVDMGQVFYGNPQLTSLPIENWNTSSATDFSQLFAEDSGLQTLSLGAWNTTKVTNFESVFQNTSLDKLDLTGWNTNSAQTYTNAFSSKLPPKRLLLGPSFNFFKSESWHLPNPSSEAPYIGKWRSLNNKKVYTSADLMTKYDGKTIVGEFEWATGNTITVKYVDAAGKYLAPDTKISGATGDAYHIKPIEIQGYVPDQPDGVQGNFTDKDETITLMYNPGGLMFVSAPQTINFGQNPITGKSENYGASYDTGLVIQDGRSIGSTWSLNATLSASGFTSKQSARPLAAVLSYKDQQTGGGSILTPGVARLIVNNHQTVSNQGVNILGQKTALGALSLQVPTDRALTDTYQATVTWTLNQGVPNR

SEQ ID NO: 28 - protein, 341 aa, Lactobacillus plantarum; identical to SEQ ID NO: 2

SEQ ID NO: 29 - protein, 1006 aa, Lactobacillus plantarum
MRLIDFKTWIMGTAAMLTLIVTNQTVSAADTATTATETTQTSGSSTLANQVVLRQTTSSSSSSSSSSSSSSSSSSSSSSSSSTKASATGAATETATSKAVTTSESSTQSSSTTATSQTTSGVTAAQATTDSTDTTATSRATANAKADQRAASAKANNEQATTQNQQQTTNMYSGVVTSQKDSARTATTTDQATASVATLSRMSRASLRSLAQRATVAVQGLDATDATVTDDDGVTYSATDVLSLYANYIAKYHWSIADDVSVTAGSTATVTLPENVVFTNGTQHIDVQKSDGTVVGTFTAETGSQTGTLTFNDYYATSDRYNRQGDLTFYVTGTSATTGSSTTGINKVGWADSNSLDADGNPTKMIWQVVANINSEKWQQVAIVDQLGLYQTHEGTMTLETGHYTDGAFVKDAALGTYGFATQQFTYADGVSTPQVTVTVVGQQMTINIDQLDVAVNIFYEVGLTVGHTYTNNAGVTYAPVIGDATDPNEGSSTGEPKSEQSNVAVRFGGSGTASDDIQSYSLVINKTDGDGQSVAGATYQLEDSTGTVLRTDLVTDSVGQLRIGNLSAGTYMLVETAAPSGYQIDTAKHVFTVSAAQATANVVTGSVVDKRIAKTALTVNKVWADVPAGVQTPTVEVTLQRNGQAYQTLQLTSANGYTGTFSDLDVTDVYGNAYTYTVIETAIAGYISSQTTSGETVTLTNTYQTGKLTVIKTDSSGANRLAGAVFAVKNAAGTLVAQLTTDATGQAQLTGLTQGAYTVSEIQAPDGYLINTQAQVLVLNEQSAYQGQLVFADEVEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPSEPVLPGHADEDSDSDQVVTTKTETAKLVKQTNLVTTTRPTKLLGQPIKLVATSKPVVKVTKATNRKSAQQLPQTSEQSMDWLMILGWFLLGLTVVSRQRRFN

SEQ ID NO: 30 - protein, 705 aa, Lactobacillus plantarum
MRRKLVGYMLSMLTVILALFMLGSTAHAKEISVTGLTAGNAIVLDANGKPVTDTSTLNDKAGYQLTYHWSIPDSEVIKAGDTATVEIPTYVSIDHDVVMPLTDSAGQTLGTFTYTKGASTGTITFTDALGTLNSRAGTLSMNAKGNATATEGSAEIAKSGLVVSSESDGAPTVLGWHITVTPGNNSTVVVTDTLGPNQTFIPDSVAAQAVQIINGIQVPQQPLTPTVATNGNVITFTFNNIHSPFVITYNTKVENFNPADTAKWHNTAALDGLGVDATADITYGGNGTAGMTYTIELTKHDAATKAVLAGAVYELQDSTGKVIQTGLTTDSQGQLIVKNLRAGDYQFVETKAPLGYELNTTPVKFTLGGIKPEVAFQVSQDDVKQPVVPTTGDVTLTKTDATTKAALAGAVYELQDATGKVLKMGLTTDTTGQLTVSGLTAGNYQFVETKAPSGYQLNAAPLSFTIKPNQTAVVTVAATDEPVTEPGTTEPSKPGEPGTTEPSKPGEPGTTEPSKPGEPGTTEPSKPGEPGTTEPSKPGEPGTTEPSQPGEPGTTEPSKPDEPGTTEPSQPGKPGKPGEPGTTEPGNPGTTGPTAPQPERPAVPGPSQPAAPKPGQSGLGQPALPGLIKQPSTGVNGAGGTVGNGVTTGMNGFGTPTGSDQSTSAGYNHGTLPQTSEKQSPIWVIFAGLLGLLIAAVGIGYRRRA

SEQ ID NO: 31 - protein, 1074 aa, Lactobacillus plantarum
MIKPRVLTTLLVCSAILTTTVTPAVAAVTPMATPSEQVAEPVASPAVPTAILSLAIQNQQLVDLIGQTQWQTYGQPAVTKDPEFNDQVLNLDGKSAFYTTFTDQQFAKLQNGMAIEAYFKYDPAADANGEHEIFSSQQGGGLGLGVQNNQVVFFAHDGSGYKTPKGTLHKGQWVHAVGVIDKNKTASLYLDGQLVQQVAMPGDLKLAQGTKDFVLGGDAVPGSHVQSMMTGQIRQARLYDQTLTSQQVSQLNVEAQVGKQPVAPVPVDQTIATKLVGPKRIASGHTYGLNVHARQIKATGAAPITMDVVYDAAKFDYVGAERLLQGGKTQIQLIAPGRIRLTTTANLSKAEFKMYAQTRLAHLNLKAKAAGETQIKFEQLTKDTTIELGPAQTVEIQGKYALDYNGDGIIGVGDVALANAADKVAAAKAAEIKPYKHVVVLTTDGGGNPWDPKGMYYAQGAEQGTKTPVWTTNPEIMKKRRNTYTMDLFNKQFAMSTSARAVSPAISAQNYISMLHGRPWDTLPKEYQGTNATMGQEYFADFNKPQAMFPSVFKMLQADNPTRGAAAFSEWGPIVNSIIEPDAAVTTKQSASLKSFDDVANYIGTPEFQSTGLVYMQSDYMDGQGHGHGWYNDNYWDKYAQYDALFKRVMDKLEATGHIHDTLVIANADHGGSGKNHGGWDEYNRSIFMALGGETVDNGRRLHGGSNADISALILNALQVPQTPQMFDSQVFDSLAFLKQTDLSKKKRSVETLKLSRNDQEAKVQLTHNQNRQLTAFDLQLDLAGREVADVKVPTGVQILRQTVANGQLRLTVSASQPVTDLVTIELVPSKTRAAKTIMLSQAMAATADGTEVLVDLDNDNPLTSTAKPDENGSTTTKPDGNGTAVKPDENGSTTTKPDGNGTAVKPDENGSTTTKPDGNGTAVKPDENGSNTTKPGGNGTTVKPDKNGSSTTKPNGNGTAVKPDKHETSTTGSGTVNTSGADKTSTNDNGTSMTAGTASSHASTVTDRVTSGTVLPETSSSAATNHGSHSTGHHGSGWLPQTGEAVQRWLAVAGGVFLMLTGAIAVWWRKRRA

SEQ ID NO: 32 - protein, 1074 aa, Lactobacillus plantarum; identical to SEQ ID NO: 31

SEQ ID NO: 33 - protein, 1039 aa, Lactobacillus plantarum
MEQVKKRYKMYKSGKMWLFAGITLVTLNMNVVTGRADESTHVEALTEPAVATLSEGNAEQQSPVTDAMDESAMSELVTEAQPIKVQAAEEQYTDEIVNQSDDEHANSDQVSVPVTDQVDSETPVPSDEHTATLDTHPNQSTTDDSEQPVSADEQSQDIDTDSTAKVLSSQHKTETINERGSGDLAGVIRNPERPHLTDGYRNDDMEDDDSMAGIWGAGYNADGIKWHFDADSGVLVLDGGDIYDCYGDSPWQSKSWVLQIVKVVISKPIRIIGDSGGFFENLTNVEHYEGLEKIDVSSATDLRYFFSENTHVKELDLSSWQVGNVTDMSYLFFNSPGTSQLTTINISGWDTRRVSEADYMFGPNEKLTRIIGIENLNFESLKEAGGLFIKTGLSELDLSKWKTDSLDNMAAWFMDMHNLTSVKFGSQFKTDQVTWIHLLFSGCSNLTEVDLSGFNLHRVEQNLDMFAGCERLQKITLGPDTDLTPAKIESVGLMDIEANDQYTGYWINVANPQQRLTSAELMNLYSEKNTPIGTYIWEANQAVIDANDITLEVGDDWNWTDSIESLTDQFGQKVDVQALYVANPQAVKLSGDRVNTSQPGTYQVTFKYAGKTVTALVIVKADQTSLTVHDTELHAGGTWHAQDGFDGATDKDGHAIDFNDVTITGEVNTMVPGDYQITYTYGSQTQTITVTVKENQASLNLYQNHATVHTDGQGTSTWQPQSNFQNATDSDGQTLDWSAIEVVGTPDWTTAGDYRLTYQFTDKTGQLVTATMTVTVVIEEADEQAESQSDLQIHDSTITVGESWQPSDNLVLATDVNGGELSLADLVVTGTVDTNQAGVYQVTYQYTDASGQVFTRVATVTVVAASDGDTNTEQPGATNTNDDVNGGSTGSIDGDDQAEIPTDDADQMEGDAADVDANAVIDDATPAVGTNHGKGADRNSGMQTTANGAKSVVTSWTHRSQMTNTASLQHAQTIVGGHHQESRPTESASVAVQPVTAKLGTSALPQTGEAPSRANVMGTVLLGLTMFGSWLGFRRVKRH

SEQ ID NO: 34 - protein, 1038 aa, Lactobacillus plantarum
MRLIVRSVRLFLKKWGITINYRESEVKCYKMYKSGKMWLLASASLLLLNTQLLTAHADEPTSASTSETSVVATNGVSIQNQGSSNQTLASSVSKTDNVVVANDENASITNQTVIDAQPATNDEPQSAASTAALNGTSGAPNSEVAADSMAAVNGLNTVAPATNSYEASRTDDLFSNAAESTVSEQQPEASEQLLLDTADASERKPAADLQHVEQHQLVDDLKVESQHVDTRAVTRADEDEMSGNFGVDWHFDASTGTLTLNGGTLNNSYGDNPWRRKSWAPMIKCIVIADKIVAGTNMNSLFANLDSVTRYEGLEKIDTSAVTNMQSLFKENTSLERLDLSAWQVGNVTTMVNMFMGNFMGTELKYLNLSGWDTHNVANMQNMFQFNGQLRTIDGLTDWDTRSVTTMANMFARTGVRHLNLTSFDSASLVEIDGAFAQMSDLERIEFGTQFTVAKVTQINSLFNDDAKLKVLDLSHFNMQNIEQNWQMLAGLTSLQTLTLGPGLDFSQHGTQPLVDLPEVPKNSKYTGKWVNVADSSQTFTSAELLAQYSGNHANTATFVWETVSAAVITGKDSTLFLNQKWDWTQNIAQLVDQNGQLVDPGVLFNTDPQAVTVSGEPVDTSQPGSYHVILTYAGRQTTVVVTVVANQSQLNLHAQEVAVEIDLATGSAVWRPRDNFASATDADGRSVEWQNVTVLGEPDLTRPGTYEVVYQFTDLTGQLVTATTTVTVTEQEADVEDLTELVVQDTTVTVGDHWQAADNFVSASDATGRLLTLADLVVIGDVDTTQPGTYEITYQYTNANGLQWTQTATITVVEGAGNGETPLPGEPAEPELPEEPGTPEQPETPETPETPETPETPETPETPETPETPETPETPETPETPETPGEPSAPGTPDQPELPEVPEQSEQPGTTEHPDTSDPNSGLTGANAGSSSQREQADTIVRPEFNGGLEKQVTTVFRDNLKLNTAERNEDGIDAKRYAKADTAKPEVTMAPVSHPASVAGELPQTSEQVNRFGLLGLMMLMVTGLASIVGIKRRQG

SEQ ID NO: 35 - protein, 901 aa, Lactobacillus plantarum
MKRNSQQSTTVDHYKMFKDGKHWVYAGITIAGLGSTLMLTTNALAATATPVSATTTSAANAPASVASQLSQAAGATATESTTTSSMTTGEDSNTTSNTDSSATTDTNQITTSTNATETSATEQATSAASATDQASEVANSASGTVTSQTTSATNSTAANTISGNEQAASSATSDATQVTDMVTATTKSTTDSAIDSTDDTSTNTNSTAAATPTSVATTSAASAATSDSGHGLIYETNDTTGNQKSTVTITQSGPYSVTWKKVTTSDKTDTTTVTLDASDIVAVVNTIKDLANQAATPSGKEQLAAAKAKLTTILDELKELPTDIASTIVGNVLYPIVFTGTGSEALSNLRTEMNQHRYDISNTWTGLDPVAYAADRAAAEEYYPTTVTWWDNVTKETWTLPEYNDPTQSVRAYYIQNGDSTKTVIIGQGWTEHVDWIGYVSKIWYDMGYNVLMPSQRGQFLSDGDNLTFGYQDKYDWLNWVKMVDERNGADSQVVFYGQSLGADTVLEAASVPGLSKSVKAVVSDAGYATLPELGSSLYNKAITAVSNALQSIGLPAITSLPFLSYDKIVAAMNARLIKEQGFSVDDLSATDAASKITIPLLLIHTQDDAFIPYTQSLELAAANHSANQEVWILPGTVGGHAAANNAILQYRQHLLAFLTPLLSVADAEDEAVDVDQVTDNRNQGAADNGTTTDSTAQDNVTDETTADEAISDHQTIVDNTTTDTTNITSDTTPDTTNHAKPNDDSTTSYVDLNDTDNAVDNDSDTAVDATRATTTVNQTSTIDQSSVIKGQVSDSIMVSSNATTNTDWLVNHDDSGSAVTASLLQDYSDQEASVTTPATVSATTTNTDSADLVAVSSPASKATTELPQTDETTQSWLATLGTSLLALATGIWAQVRRRFN

SEQ ID NO: 36 - protein, 833 aa, Lactobacillus plantarum
MERKRTNFKMYKIGRRWAFACAVILTMGTTTLVARADDGTTATGTDTASTSSSTTKSVTAKTQTLKTAATTEADVTNQNQPVLDTDGSNSKTAAGTVAGTKAATDTDTNATTNLDETTSANTETGSDTTAGSKTAKETNATTGSESTKETSTITDSATATAARTTTSSNKGATTDSTTSHDTAATATKTTDASSKIAGTTTSDSVAQQTTTTKDQSTTTATPQTAAVALSQAVTHANDAVADGGNVTDDYPDLHNMLRVSSQFHIFAREAELHAHTNGNVAVQNLVGNVNFGTNIIEELLDKDISYIQNISNIAGSSFVSAGETRSNKVIFGENIEIDISNPNRPMVNGVYIDHLLASEVYQDKDGNVYIDFDKEFAKLEQLSASLSEASANVTYTSDSFEDMNQRVIDVTDMQPDADGHIVINLSADVLNTSTPLTIKGLSADADGNTVIINVDTAGATNYQVNSQIKIIYDDGTERNNKETEDFGDNHLLWNFYDSTASDKLATGVINVDRPFQGSILAPAAEIDANQNIDGNIIANKVNVKAETHRWDLQDNVDNENDPEPVPDYEKPVHPSIDAELPDGGEGEEPEYDKPVHPSIDIEMPDDGEGEEPEYDKPVHPSIDIEMPDDGEEEEPEYDKPVHPSIDIEMPDNGEEEEEYDKPVHPSIDVEMPDFDEIEDEEEAEDAEEEFEDDIEDEIEAGVTPDEVVDQIEEEVDNEITADWVTDETATELETAFEEVQKEAVVGDQIKDEETLINLIDRAIAQAKAHHNTALVAQLQALRTKVASALAVAKGQALPQTDEAPSQMISLAGIALASTLVLGAAAVSRRKRQY

SEQ ID NO: 37 - protein, 806 aa, Lactobacillus plantarum
MNKKLLYTSITTAALFVGTQLGVNNAQADTATDNSDTTNQTSATQGSAQTATNEKLATVKPTSQQQYQANVQTAKGNVATAQNQVNTTQTKVATAQGQVTNQSQLVAIGQSQYDAGKAQVDRAQQTLDANNQVLAEAENKVDAAKSQTAAAETQIPADQQQIAANKVAIANQPATEKKAQTAKDAAVTALTQAKTEQATAQSDADAASAVTAAKQATVDQASAAQQKAATQANQAKVAVASAQDAVNKNTQAINSAKTAIQNTTSQINANNQAVSTAQAKVTAAQAALAAAERPTTTTESQNKYDAAEFPQSQLTGAETVSVAYPSNGKYVPNADKINQYMFEYINQLRALNGQPALKQTSTLQNNAIARAAAQVDGGLDHTGSSYAENLTQVYPQWFMSDQETAYNAVMGWYDESNNVESGSFGHRVNLIYSTGDAGVAINLAKHVAAFEVDNAGMTEAQQDKYVDLFDNAHTNAATGTKALPAVTFNYVQTTPADPKKIAAANATLIAATASLNGLQNTGKTLATTLANQNASLQALQNQTSGLQATVTTKQAQVQVAATSLKAANVALTQAQGQLATAQQQQLSPVRNLKTSIAKTAAAQVTATQAAKNLASTKTLIADLTAENARLAAVLAQGQAQVDTANEQLAAGKAQLDRKKTDLAQFKQVLGAARVDLAVAQGDLTATKAFLARVEANKFTTTTAAAADGIAETTNVDQSTGVTAPHATATKTVANSNGTINATSTSVDVSDGDVTTKLVAGAKQQPVAAQATALPQTDEKQSASLTVVGLLAAGFSLLGLTKLRKRA

SEQ ID NO: 38 - protein, 617 aa, Lactobacillus plantarum
MKLSKRGLFWLLGLVSFAILLLFSQPLGAQAATNYHAKDYTTAASVINGPDFKHADTIQIQYQMSFGDTTFKAGDTVTIDMPANLEPRTVGATFDVTDAETGTVIGTGVVGGDGQVVLTMNSAIEGKTNVKIDVNLGMKYRYDDLGEQDVVFDTQDGQDTSVINMVANEANMSKKGTIDKENGTIKWTLLVDRREITMKNLSIADTIGDHQQMIKGIEVYNGEWSSANTYKRRDKLSDDAYQVNYSDNGFDLKFNDTVSNLVVIDYYTKITDTELIDQNYHFKNKAVMEWGGGTSGGKNSEEANGKVYEKVVNGGSGTGDLSSSSSSNSSSSNNSSDVDSSSDDSNSESSSAVDSSSDDSSSESSSAVDSSSDHSSSESSSAVDSSSDDSSSESSSAVDSSSDHSSSESSSAVDSSSDHSSSESSSVVDSSSDHSSSESSSAVDSSSDHSGSESSSDVNTSSESSDNTTTEPDNGHQTGDIEDPEDNTAVYPDIDEDTGTIDVDGGFDSNYDGSTTSNSTNSSKPLKDSTSSVFTSTPANTTTGQDGVDQTPAADTKKSSAKTTVSESDALTPSTPNQVAKLPQTNEAKMDSQALRSVGILLGVLTLGGGALIRHWF

SEQ ID NO: 39 - protein, 570 aa, Lactobacillus plantarum
MRKKWRWLLLALTGIFFLMFGPPLVSQARNVIEATGNDVNSAVIKDSKGKIMAHDAQLPEDQEYTVNYNWRIPDNLKIKAGDTMAFQVPENVRIPHDEAFPMKGTTAGTIGTFFIAAGAHTGLVTFNQAYQTRPRNRKGFVQLDAFGTVPSHPGNLAPILLEKSAEWADEANPRRINWTIRVLPNNNQLVDPTFVDTLSPNQTYVNGSAVLRDETGNIIPVNTSVNGNQLTFNATGSFTSELALTYQTKTNEPTGDATFENNVTYTDKNGNKGSATATISRPVTEPDVPENPGISEPTDPDEDEEPGVTEPEKPGTTEPEKPGVTEPEKPGTTEPEKPGVTEPEKPGTTEPEKPGVTEPEKPGTTEPEKPGVTEPEKPGTTEPEKPGVTEPEKPGTTEPEKPGVTEPEKPGTTEPEKPGVTEPEKPGTTEPEKPGITEPEKPGTVSPEQPSGPKPTNPGTVTPEKPTAVTPAVPNESSPSTPEPSVSGNLSAPANPATNSTNTTATTVPATNPLPASAATAFAGSAPMNKSLPQTNEHSASWSVAIGLALLIGLLGSAFVLTRRTKHRHS

SEQ ID NO: 40 - protein, 1010 aa, Lactobacillus plantarum
MLKKDNFGEHKTHYKLYKCGKNWAIMGITLVSLGVGTVTMTRAAAADSEVTNDSASQHVTSISTDASKNQHTSSNVILTNDDKSVSASINQDASASVVNKAVSATSQENSSVQNTSQATSTSKQESSSTKNTSQTTSTSNQEANSAKSINQTTRTSKQESSSTKNTSQTTSTSNQEANSAKSINQTTRTSNQESSSAKNTSQTTSTSSRKINSTKSQAQSLTITTTGKAVRATSTSVKKYSTKTKVSYSTLLQQLRTSKALISDEAALTHVDKDNFLKYFSLNGSATYDAKTGIVTITPNQNNQVGNFSLTSKIDMNKSFTLTGQVNLGSNPNGADGIGFAFHSGNTTDVGNAGGNLGIGGLQDAIGFKLDTWFNSYQAPSSDKNGSEISSTNSNGFGWNGDSANAPYGTFVKTSNQEISTANGSKVQRWWAQDTGESQALSKADIDGNFHDFVVNYDGATRTLTVSYTQASGKVLTWKTTVDSSYQAMAMVVSASTGAAKNLQQFKLTSFDFQEAATVNVKYVDTTGHQLAQGTANYPDGAYVNGRYTTKQLIIPNYRFIKMDDGSVTGTKSLDANGTLIQSGDNGTVIYVYVPEYMAIVKTVNETINYVDENGHALTTSYTANPIHILTVTNPVDGTTTTYYSTITTSIELDATTGRPVDSGWVLGNSQDFDAVTNPQIKGYTVTSTDAPNSDLQHVSAQTVTGDSGDLEFTVVYTKNAPIVTTESKTVNETIHYVYTDGTTAHDDYVAQPITFTRTVFTDAVTGEKTYGGWSAAQQFAAVDSPAIKGYTPDQSKISTQTVTGDSSDLEFTIVYTKNAPTVTTESKTVNETIHYVYTDGTIAHDDYVAQPITFTRTVSTDAVTGEKTYGGWSAAQQFAAVDSPAIKGYTPDQSKISTQTVTGDSSDLEFTVVYKADSTSTKPVKPEQPTIPTTPTEPVKPGQLTTPAKPDQPMTSDKSVQTITIKFVGQRLPQTNETDQQHMTLSGLLLLAMSGLLGLLGMAKRQHKE

SEQ ID NO: 41 - protein, 419 aa, Lactobacillus plantarum
MSKALKIVMGITMLTGGIMAQKMTVHAAESNTRTGQAVRMNGTVSLASQVENNPAVKAAHYQVTQAVQALTMATTAVKTAMSDLQAAQTTLDAANKTLAKNQKIQTHMGVLKQAATDRHVKATKALDEQLATKKTSQTAVTTAQAAVTKSQAAVQVAQSNFDKDNSAANKVTLQTTQAKLKTVQETLTAAQANLDKTNEHVMMAEEELANAKIEVSGTSRDFQMAQRDYDIVQPQAAVNQAKAAVTAKLQRVAGTQDQVVTAQRELSQAQAGLTTVRARTLATLTAAAEKPMTEKPVGERPVVSHSTGTSTSTNQSAAPQATPAKPTLNQSSSASVPTAQRVVTTQPRQATTVLRTTTSPAMAKPVTQQTVPTTATKTATLPQTGEQTNRVLTVLGFVLLAATSLFGFSKQQRRHKTTD

SEQ ID NO: 42 - protein, 2139 aa, Lactobacillus plantarum
MNRFITSKQHYKMYKKGRFWVFAGITVATFTLNPLISRADTETTTAATAATTTAGASSSSNSQVLRTTTTSTTGATTQSSATAINAATTNTSAQKKQAVSGTTTDSKAEQPVTAVGENENATSNLSTSDSASASSQAKTGSGNSLDQTSNSSVSVASSSQKVTTQNSDYQNDQGTGSESGIQSNVTDTVVADESLQTNRSSVASPSTSTMASIGDSDSKDSNETEKVVDSETSPIVVTATTNTITTTNDKVQLNRALLARAAIPAIVQSGTLGTSQWTMNSDGVVTIGAGDWSNVDDVSALFYTLGSTVTGVVIDGKVNAGEDLSYLFFKSPNLATITGFQNIDTSKVTDFSYMFCGTSVADFSSISHWDVSDSENFDSMFTSNSKVQSIDLSHWELSQAQSIKMRRMFAADTALISMDLSAWNMSMVTNINGMFAGNDLNTMALKSVDLHGWNLKNVTDMGTMFNFDNSLTSVNMSGWQTSSNLSSVDSMFRGTSSLASLDLSSIDLQGVTRKYMLLSQNKLYDPISSSLSTLTLGTMSVLTDTGLPDIPTGTGYTGKWVNQADATQTYTSSELMALYNGVDSPADTITWVWETSPSYADFTSKNVTGLIAGPKTTWRVADSVATLKDVNGTDIYATADTVVKVISVNGDTAVTTVDTQTAGTYQVDLQYTDAYGKVWQQTSTVAVAVNQGKLVGKPLTIKMGAKPTYTINDLIDTDNSRNAAGDKLSADELATATVTGLDTSKAGAQTVTLAYTDDATGMVHTTTTTVTMVATKADLTMRNSTIIKGPKNSSWDYRQYVTSVTDFDGNPVSLDGLNIVVDQQPDLTQIGSQTVTLTYTDALGNVISVPTQVTVVASRAQVTTKAPLTIWPSEVAQLKVADLVTITAANGNPVDTSTDLTDVTMSSIDTSKGGAQTVTITYTDEAGNLVTAYAKVTVDQSDLKTKLTNPIAGPKAKWDYLAGLEWVKDANGKLLDNLATADIKVVTEPDLSVAMVGHDQTVTLSYMDELGKEHLVTAVVNTVASKAKITAVSDQIIIPDEAKKLTATDLVSELIDAAGNKATNFDDVTMSGFDAKAIGPQTVTLTYSDAYGNQTTDSTTVTVDFATITGQATHPIAGPTATWDYRDSVTQVIDANGKIIDVGDADITATTPDLTPAKVGKPQTVTLTYTDSLGKVHTTDVIVTTTLSKAKITAVADQIIWPDQAKQLTATDLVDRLYDAEGHLITNHDNVKMSVLDSKLAGQQRLTLTYTDVAGNQSVAYANVTVDQAKLVTKPSTVIAGPTATWSYEAGISQLTNAAGQLITVQPGTIKVLNRPDLNVDSVGQQQLITLIYTDELGKSQSVTAMVTAEASQAMLTAKAAVIVQPDASAKLTANDLVTSLTDASGQQVTDYQIVRMSKLDATWPGVQPVSLTYTDAAGNEVSTVVKVTVDQAKIDSQNRTQIWGPSMTWDYRQQLATVTDSQGHQFNPDQAKITVITGPQLTAKMIDKPQTVTLMYTDDLQQTHTVSATLTLTASQAALVPRPAQIVWAKDAGLLTPANFSQTITGADGTQVSSLTNVKMSAVDASQPGAQTVTLTYIDDYGNEVTTTAQVTVDQAALTTQTARPVAGPTAKWDYQTNFKTVTNAAGEVINVGDANIKVLTGPDLSTAMVGRPQVVTFSYTDELGLTQTATAKVTTVASRAHMTTSADQVTWPATVGKLTVADLVTGLTDAWGQTSQNYQNVTMTTINAQQAGKQQVTLTYTDEVGNVKTATTTVTVDQAALTTQPQTVIAGPTAKWDYHQGIGTITDGMGQPIAVNNAAITVVAMPDLTVAHIGQPQTVQLVYTDSLGQQQTALVQVTTVATQAKISTRPVTVIAGPKTTWSLNDSVDWSTSLAADGTLLTAAQRQRVTVDGTLNLRRAGNYPLTLSYMDRAGNLITVTTSIDVLASQAQLQVRDSQLTVGNTWAAQDNFERATDAQGQALTLADIAVDGTVNTQHAGRYTLTYHYTDVAGNQLTKTAVVTVVLPEDDHINTADPDNNDHAGITNPSETPKPSEQPNDSDGHTVDWGVDDRITTKQQPAAATRAQTKVKMTAEPALPANNERTSATKAVTRVTDTTADTLPQTGERDRSAQQGAVVLGLTGLLGLMGLGRRRHTHED

SEQ ID NO: 43 - protein, 4326 aa, Lactobacillus acidophilus
MVSKNNRAKQMENVAERQPHFSIRKLTIGAASVLLSTTLWMSVNTSSVHAENIDNSDNDAHEATESNTETPSINDDTKVVVESNSNITSSNDVNAGNNGAETNDTNNEVTASEDTSKGLTVDNKDASVQSTVKSSDEVKKSESTEQKSAKTAQNSTLNNNTVNTEKAESNVAAKSNADTAKSTQQSSAASSANQVSSNADLTQNQAINSTTQVEANNSTNDKKANNDTADLSNIGLKGIETNKIPETTDLPVSELIKSYNNNSNSNEVNVNQVSGLRAAQLFAASFIATQNTGTGNNGAVNIDTYKPDFNLTENPAYQQYFAAIPADQYAFQSYEVVSTGQKIVVTTDRNNIGNNIRFYNVRNGSAQLVYQMTRDTQTNASGSVVKNRPSLQGTFTTAGVASNSTYKGGTYNWSLNQTDTVNFPGIGNLKIGRIDITAGSSNSPVDNGTGAFVTDNSHRITPTWDQGLPIEGIVSGKTWNSAGSNIPDKVTQNIWYVDAETGKVLSHKTSDEAFNGSSYDSTDNGVKTISKDGKAYQLIDRGSDGLYDPSDFSDILNKQLATNNGLPITIGDVLSTPLKGTLRDGRIGNIKGSITNFQGTRAYMRLQTKTDGTIDLNTYTFDPGSTRGNLNTGLSQADVAPGQTVMGAGDTSGSGAFYNGTRPGNRDIIFLYNAEANKQNANITFVNDDTGASLSPQQNSSGDAGSQITFDNAGTTVTNLISQGYVYNGTTGNGVTNGSAGGSFTSVGFPAYDNDDNTNQAFVVHFKNPVQTTTYRQGTEESKTINRTINYYDKVTGEKIPSNLISQNPVTDSVTFTRTQVLDQDGKVVGYGTISTDGKSFRNQDWHTAAGESSTQFDAKRSSDLSAYNYTAPEFQDGTNASIVAAHEVTPTTQDLVYNVYYGHQTQQVTTNEDVTRRFHYIFTDGTTPESHLTPQADQKVTFTGTATKDLVTGKTGDTVWTPSTGTLAQVAGQTVAGYHITGNVNANADGSANAVTVNPDSGDIDVTVVYTPDAKTPDTPQKAKVTIYDKTENNKQLSNFENNNGTKGSAISFDGEPQTLQAYLNSGYVFDSATDANGNSIGTASNITFGNFDSVDGNVQSFNIYLVHGTDTKTEKATTNAHVHYVVAGNEANKPAAPADSPTQTINWTRTNTTDKVTGATTEGTWTPDKNGFTSVTSPDLTNYTPDQAVANFTTPQPNRDQVVTVVYNPNPEVAQKADLVVYDKTDNNKELNNFDNSGKTGTQISFSGSANYVADLIAKGYKIDSFVNDQNQTSNPTSYDQISFSNFDNNSASDQHFKLYLVHDTENVTDKKTTTSTVHYVVSDGKTNPPSDNTQTITWTRPGTKDKVTGVTTPTGNWTTPDNYTDVPTPNLDGYTPDKTNVPAPTPDPNQNPTTVVTYNPKTPEAPTYTGTTENKTVTRTINYYDKVTGEKIPANLISDNPTTQNVTLSRTHVVSSTGQDMGYGTVSADGKTFTKATTVDGWNTGDWAQVTSPDLSNAGYTAPDLAQADQVTVDANTKDAVVNVYYGHQTEVITPKTPHNPGGSINPNDPRNKPSVYPDGLTKEALTTEVTRHINYVGVNEDGTTTPVNGSPDGKNTYTQTVSFERNAVIDKVTGQILGYSTDGTTNVTITDKDRAWTPTTQNMDSVASKTPSEVGYDKVDISTVGGVTVYPGQKVNDVTVTYTKNKSPEVTQKATLEIIDNNDTNAPKQLASFSNEGKSEDQINFANSNEILQSYLSQGYKVQKTAGNLSGDAQSGYTYPTYGNTTQDFKIYLIHDIADKTETATATAQVHYVVADNGVQAPADSDLQTITYTRTNRVDKVTGATVNEGTWQADKSVFTDVKSPDLSKDGYTPSLENVQFNAPERNVNQRVTVVYNRSAQAADLQIIDDNDPQNQRVLATYSAGGESGKQISFDGSNTQLQTYLNNGYTFEKYEGQGMSGDAQNGFTYPSFDNDSQSNQSFKIYLKHATANKTATATTTAHVHYIMADGTKAPDDSAIQTINWTQTNTVDRVTGATINEGTWSSDKNAFTDVDSPTVTGYTPGTKTVKFATPERGVNQVVNVVYTKDAPTPDRQNALVVYQDVNDPAHPVDLGQSDQLTGQAGYSINYSTANKIDEYEKQGYVLVSNGFDANGTKPSFDNVNGNTQTFYVTFKHGIQPVTPTTPGTPDQPINPDNPDGPKYPSGTDQTSLTKDVTRTVTYEGAGNQTPSPVTDTLHFQGTGYLDKVTGKWTDANGKKLSDQTKGITWTITDGTKDEGSFNLVPTKHIDGYTSKVVTNGADDGNGNVKSYTGITHTSDNINVVVQYNPIVAEQGNLIVKFHDDTDNKDLTGVGTDTGTQDVGTQVTYNPSTDLTNLENKGYVYVSTDGNIPSSIVKGTTTVTIHVKHGTVPVTPDNPGTPDQPINPNDPDPNGPKYPTGTDKASIDKTITRIVHYEGADQYTPNDVKQPVHFTAKGVLDKVTGEWITPLAWSEDQTFNGVNSPKIPGYHVESVDKDTTDNQNVDSAKISHTGADYTVTVKYAKDAAPTPDATTGKVAYIDDTTKNTLRTDSLSGNVDANIDYTTQDKISNYINMGYKLVSNNFTDGKEIFNKDASKNSFEVHLVHDTVPVTPDNPGTPDKPINPNDPRPRSEQPKYPTGTSETDLTKDITRTVHYSGADEYTPNDVKQPVHFTAKGVLDKVTGEWITPLTWSEDQTFNGVNSPKIPGYHVVSVDKDADGTNVASSNVSHTGSDYTVNVVYAKDAVKQAENANLHIIDLSDNNKEIANFNDSGDDNAAINFNGAQTTVDALIKGGYKVNSIVQATSDPNNPTKYGTEYSSAASQWMFDDKPGVDQSFYVYVEHDYAPINPENAYGRTDLTQTVTETVHYIDEATNKPVATDYTNTLTFKGQGRVDKVTGKMLKIKSIENGQITYDYNVANEIDISSAKLSDFAWSTPTTLQKVTSPTIAGYTIDAAKTTPSELADGNDIKEIQNVAYDHGNVEATVYYKANPVETHKAGLTIYANGNQVGTASVTGAKDTAINFSSASDIVAAYISNGYKFDHAQDVTNNKEMTGKSYNELNFGNFATTNNSDQQFAIYLTKDETPAKTQQNAQLTVRDVTPGQEMDLGNYTQPGLEGDTISFSSAQEFVQNLLNKGYVWDGASYNGTNLEATNYAGINFGNYDNTDDKNGISQKWVINLVHGVTPVNPDHPDDKDGFTKDYLDRTITRDVTYVYEDGSQAAAPVHQEAHYQGSGYLDNVTGKWVTVENGKITGLAQGLTWTPDQDSTFDQIGAKNIEGYHVSSVSGNGISGFTVGQDGTVGQQTVTKDTPSSTIRVVYVKTPVTPVPANGSIVYIDDTTGNNLENATFGGTVGAKIDYTTADRISYYQGKGYKLVSNNFTDGSQTFKQGENKFEVHLTHVTETKDATKTITRDVTYVYEDGSQADTPVQQTITFTGKTTSDKVTGSEKTTWNNESQTFGATKAIDTTKYQIVGINERNTTANVDRDTGVVASETITPNSQNSAVVITLANKPETPIPANGSITYYDDTTGTTLESAGFSGSVGQKINYTTADRIINYVNKGYDVVSNNFTDGNETFKQGDNKFEVHLVHATTPITPENPGKPGQEVPNPNDPEHPHTIPANFVPQTLTHTVTRDVTYVYADGSQASAPVHQTFTFNGNGVIDLVTGQLVTVENGKITGAGKITWNADSHNFDAIDAIDHDGYYISNVSENNTTANVDTNTGAVAGETITPNSQNSTIIITLTKKPDVPTPVPEQGSIKVTVHDVKTNQDVPGYDKDSGKQNTGTSFTYDKTTTITDLENKGYKVINPNVDIPTKVSNIDQHIVIYVDHNVIPVTPDKPGNGLSENDLNKTVTETVHYVVNGGATEAPADKTTSLKFTGTAYYDSVTKKWTDANGNELSDQSKNVTWTAENGNKFAVVVTPTLEGYTPSVQSGYDDGNKNVKEINNITPDSGNVEVTVTYNKNNVPTPVKQGTIEIIYHDTTDNVDIPGYGQSRIKEDEGTSFSYNPNAKDLPALESKGYVLDGELPTIPTKFTDGDQRVVINVKHGTTTVTPDKPGKPGDPIDPNNPDGPKYPEGTGENNLKVTGTQTIHYIGAGDKTPKDNTQSFEFTKQITFDNVTGKIINDSGWNVTSHTFGSEATPVIDGYHADKTTAGGTTVTPNDLHKTVTVTYTPNVPAVPTPTPTPSPEPKPENTPVEPNTPTPTPDIPDNVTPTPEPENNNVKPHGESIVQKNNDNPKVVSHGQSGNNWTAPHGQHVDQRGNIVTSDNRVVGYVDQNGKAHYTKLPQTGDDQTNDVAAALLGGAAVSLGLIGLAGVKKRRKEDK

SEQ ID NO: 44 - protein, 2650 aa, Lactobacillus acidophilus
MISKNNRIKRMEATSERKQHHGIRTLSVGAVSVLLGTTLWISIPTSTVHADEINIDDNQPKTNLESNESASTDHVEKVIVEQNQSSSEGAQQDINAANDVSAQNDQKSVNKINDEIIKNENVDADIKTNTDNSHAETSYGQTESQEIIENKQKTDVEKNKTQTTDNITPVEQTGNSSENTSTNVTTQSPVDNSTNNDVNVNNSNLADTQAELIDSNTQFYESSPLIDQIGQQGKTTVNSSNNTSSKLNIDDLSPDLSDEVLKANLTQGNQILLNQSNSSDTMAGKNADPTKQLEAMARTATLVAASPNADNYTTVNNYNDLQRAVSNYSVSGVNIDGDIYVFGNLTINRAFTIKGTNNAKLNLNQNAIINNSTLTLEDITVNGSIMGNGTVNIKGDVISNVNESNGYTLTNSEKATPGVKVNWTQTKGYNIQSSTVNVDDNASLTINRSSVGDGIHLLSNGIVNVGNYSQLTINMNTNNELGTGATARYHDAGIFAESNGSFTTGYKSVVTLNTSIGQGIAMTGLRPNVTDNDRFGGYTRDRANGAGQINLGQYSTLNFTGRDGVILGNNSNFNVGEYANVHFENKGRGVALDLANNSNINIADHAVTYFHSVGKNTTNAIGVVVGPSGSYEGYNYIGVNEAGNITIGEDATFRVIMENRGDNAWDDVISLDSQLATTNAAFTSKKGAIIDIRDDNTNFYAELISFPLGAANSRIDIQDPLLLNLQRYSAGGETTGWMAGVGGVAINSTSEKYTANLIYMGGTKGVLSIGGTNYVVYQQIKSDGAQQIWTDVDSVEFHKNGFASQDIFNNGANSDVSISGNGFTSGIRANQIRDNQTDPTLVNLQNSPAYGISTMRASHQIWIPHETSTQIKGTHTNTISYVYEDGTPVMGADSQPLVVTQNLNLARDLTLDLTSEQIKTIQDYALGHTADETLNYIRSGYSVTQDSGWTYTNDQGQKVTDPYASVTSPVKEGYIITIQSTNAPGVTLGADGQTVKANFVFDAANDVVQNGQLSAGYRNQGITGIPDNYQTIVVYKKAEKGSVQVIFYDDTTNDAIPSVGFNSGTEEAGTPVTYTTAQNISDLEKQGYVYVSTDGVIPTTIPNNATLITVHMKHGTNPVNPDQPTDKYTKEDLQKTVTRTINYIDTAGNIIADSVTSTVVFTGSGTIDTVTGNLVTVDASGNIVDQNGQLTWTYSVDGDSAQSGNSYTFAETAAKPSIDYNGSTYNFVSVTPGNYSAGNGSVTSYEVNTNNSHDLTVDVIYNEGATYHTGKTDTKNVTRIINYLDGKTDEKIPINLILANPVEQTVSMYRTEILDSTGKVIGYGTVSQDGKMYTLNNNWIIDGIWESVNSPDLTTNGYKAPRFEDSSLAAIVAEYIVNADTKNATVNVYYDHQVIPIGPDTPDKHGVDINQVEKVVKETVHYVGAGDKTPADQVQTSKWIRTVTVDVVTNEVVPDGEFTTDWTIPSDEKSTYDQVDTPVVNGYYADQANVPATAVTQNDIEKTITYKQIGKVIPVDPSGNQIPGIDTPHFPNDPNDPTKVIPGEKPYVPGYHPETGKPGDAVDPAPGDPSKDVEVPYTPETPIVDQKAVVNYIDSDEENKVITSSGDLIGKPGEQIDYTTIPTITDLTNKGYVLIYDGFPTRVTFDDDDGITQIFTVVLKHGTQTVTPEKPGIPGDPINPNDPDGPKWSDETGKDSLIKTGTQTIHYEGAGSKTPTDNVQNFEFTRTAVIDKVTGEVISTSGWNVTSYTFGNVDTPIVEGYHADKRNAGGTTITPDDLNKMLVVRYTPNGKIIPVDPAGNPIPNVPTPQYPTDPTDPTKVVPDEPVPAIPGYRPSTPIVTPTDPDKDTPVPYAPIQGSIQVIFHDDTSNQTIPDVGYNSGVQDEGTRIDYTTNKNITDLINKGYVYVGTDGNVPAEIVADQNITITVHMKHGTTTITPDQPGKPGEPINPNDPNGPKWPSDTDTKGLTKQGNQTIHYVYVDGNKAADDNVQNVTFVHTLVFDNVTGQVIDDRGWTPESHKFNNVFSPTIDGHHADKIVVDGVTVTVDNPTSETTVVYAKNGQVIREQQEVKASQIVKYVDDEGNELHKSELQEFTFTYTGDAYDEVTGAKVQTGTWNAISTDFPVVDVPVITGYVAVSGYTNNNGKYMAGGFTTTRESSEDQRNRVFTVLYKKVGNIVPVGPDGTTPIPDAPTPSYKNDPTNPTKVIPDEPVPKVPGYTPNTPTVTPGDPTTDTLVPYTPGNPITDQKAVVNYIDADEGNKVIISSGNLIGKAGDKVDYNTSDTIKNLENKGYVLVHNGFPDGVTFDNDDSTIQTYTVILKHGTTTVIPDKPGKPGEPINPNDPDGPKWPDTTGKDNLSKTGTQTIHYTGAGNNTPKDNVQSFTFTRTAVVDNVTGKVISTGAWNVTSHTFGNVDTPVVEGYHADKRTAGNTTITPEDLNKIVTVNYTANGKIIPVDPNGKPIPNVPTPTYPTDPNDPTKVVPNEPVPTIPGYKPSVPTVTPSDPGKDTPVPYAPQTTPVTPNIPVTPNEPSTPTTPDTSAPTPHGEDVPVTPNEPDTPAPAPHGEKPEEPDRPAPAPHAPKAPTAKGNNTPEKEDKTVPTAAAVVKNEQTPEAELPQTGEKNDSAAAILGATAGMIGLIGLSGVKKKKS

SEQ ID NO: 45 - protein, 1208 aa, Lactobacillus acidophilus
MDKKEVKNRFSFRKLSTGLATVFLGSIFFWTNGQTVQADSVEPASEQAVQNVDSQVQADNTVSENTVNEENGSTSETTTEVKTEMPSVDTTSQAKDAVETSDNKKVELPQGEADKQVPQKLEVNKSNQAAETTDKDTKQNATSATPAQLNENTAPVVVKAKSEGKEVVKATDPTDYPTEVGQIIDQDKYIYQILSLNDRSGRPSDSKLVLTTNRNDHNDKNIYAYVVDRNNRRVSQSVTVGVDQHTIISVNGRGYQISNTGGSNVIVDGKEVPTQNTSTVTSGNGTTSPIYGLGNTTRGDYSAIGEIPPVYTENSVIKYYYRDENGNLKEAESSDQYPNVNVSGLTGQEFVIPNVDQYKRVIKGRYLNSDNLPTGDFTGTISQFGEGKYYKKVYYDYGTDDVDYYVVYNQVSPDGTMDVSLFRGDNNTPIESRRVGPGRSIRFTSRNYTARNPYVTETPHEVQFIYDKLGSIVPVDEDGNVIGDLVQFNNSTDPTKAAVTDSPVIAGYTIKDPTQREITPHDPGKNIKVVYVRNHVTAAIKYIDDTAGDDLSAYNKSITAKPGEALNYTTKDSITELQNKGYVLVSDNFNVTTMPENGGNYEVHVKHGTKTIDPDNPTDKYTKKDLQKTATRTINYVDDQGNKIAESVTSTVVFTGTGTVDAVTGNLVNLHPDGSIKDQNGKLTWTYSVDGGVVQKSDTYTFSATTARPTIDHNNSTYNFTSTTPADYNAGNGAVSSYRVNSTDPQNLIVNVVYTKQAIYHAGKTETKSVTRTINYLDGKTGEKIPTDLIATNPVAQTVNLHRTEIIDDNGKVIGYGTISKDGKSYTINNDWVVDGKWASVTSPDLSAKGYKAPRFENGTSAARVDEVIVGSGTKDATVNVYYDHNLIPIGPDNFDKHGVDRSQIEKQVKETVHYVGAGDKTPADHVQTSKWTRTITIDAVTKEVVPNGQYTTDWTIPKGEKTEYAQVNTPVVNGYYADQANVPATTVTQNDIEKTVTYKQIGRIVPVDPNGKPIPDAPTPQYPNDPTDPTKVLPNVPVPNIPGYKPSVPTVTPTDPGKDTQVPYTPVTPTNPDNPVIPTPQPEPNPDNGKDKPVDPSKPSDDPVHPEYPGIKRGQDKPDKEKTDKKRNGKTKGKENTPTGRDAVKRAGRSDDALKLASEAKNRRMTIQGKNEELPQAGEDHNAMALIGLAFATLAGSVVFATDRKRR

SEQ ID NO: 46 - DNA, 584 bp, Lactococcus lactis
  source 1..584: mol_type = genomic DNA
taataaaacttattgttttgatgttcggcttaaggatggaaggatttttcaaataaaaaagtaaaaaataatgttaactggttgacattatttttactttgctatataattaaccagtaaactaattatggaggacgaaatactatgagtttagcaaatcaaatcgaccagtttcttggggcaattatgcagtttgcagaaaacaagcatgaaatattactcggcgaatgcgaaagtaatgttaagctaacaagcacgcaagaacatatcttaatgattctagctgcagaggtttcgacaaacgcgagaattgccgagcaactcaagatttcgccagcagcggtaactaaagctctcaaaaaattacaagagcaagaactgattaaatcaagtcgggcaacaaatgacgaacgcgtagtcctttggagcctgacagaaaaagcaattccagttgctaaagaacatgctgctcatcatgagaaaactctaagtacctaccaagaattaggagacaaatttactgacgaagaacaaaaagtgataagtcaattcttatcagtacttacggaggagtttcgatgaag

SEQ ID NO: 47 - DNA, 854 bp, Streptococcus pneumoniae
  source 1..854: mol_type = genomic DNA
atggtcttcaagggaaaacagtaaccattataggagtgctgttttgagatttcgattaaaacagatatagttgataatcaaggatttatagtatgaaaaagaggatcggcgggtcctcttttgttgttgaaaagataaaaaactcagtaacctagaaataagacaactgaagctttactctatattcaattctttggaattaataaatccaaataaaattgtacaacttcttgatctgtgaagtcttgtcctttcttcaaccaccatgtcaaagtttcaataaaatttgacataaccaaatgttgcaaatatgatgttggtaaatttggatgagcttctttcaaattatcagctaaaactgaataaacatgatgttctaattccttatgtaattgtcttaagaaataatcattctttgagaacaataatgatgtaatatgatcttgattcttatggaaatgtaagaataaatgagccaaataatcttctgttgaaatagcttgttctctttcaaacaaatgatgaaacaaatatctacataattgatccaataataattctttagattcataatgacaatagaatgttgatcttccaacatcagccaaatcaataatatcttgaacagttgtagcttcatatcctttagcatttaataattgaataaatgcttgatagatggcttttttggttttgctgatacgacggtcaatgttagtcatatggacacttaaggcaaattgttcagaactgaataaagctgacgttttgcttctatcctttctttgagttttagtggataatgataatgaacaaggtgttcataaatctattataacaaaggaatgagaaat

SEQ ID NO: 48 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 49 - DNA, 30 bp, synthetic construct
  source 1..30: mol_type = other DNA
ggattatttggtaaattatattttgaagga

SEQ ID NO: 50 - protein, 10 aa, synthetic construct
GLFGKLYFEG

SEQ ID NO: 51 - DNA, 3657 bp, synthetic construct
  source 1..3657: mol_type = other DNA
atggataagaaagaagtgaaaaataggtttagttttaggaagttatccacaggcttagcgacagtatttttaggatcaattttcttttggacaaatggacaaacggttcaagcagatagtgtagagccagctagtgaacaggctgtacaaaatgttgactctcaagtacaggctgataatactgtttcggaaaataccgttaatgaagaaaatggctctacttccgaaactactactgaagttaagacagaaatgccgtctgttgatacaacatctcaagctaaagatgcagtagaaacttcagataataagaaagttgagctccctcaaggagaagcagataagcaggttccacaaaagttagaggttaataagagtaatcaagcagctgaaacaactgataaagatacaaagcaaaatgctacttctgcaacaccagcacaacttaatgaaaatacagctccagttgttgtaaaagctaagtcggaaggaaaagaagtagttaaggctactgatccgactgattatccaactgaagttggtcaaatcattgatcaagataaatatatttatcaaattttgtcgcttaatgatcgtagtggccgaccttctgattcgaagctggttcttaccactaatagaaatgatcataatgacaagaatatctatgcttacgtagttgatagaaataatagaagagtaagtcaatcagttacagttggtgtagatcaacatactattattagtgtgaatggtcgcggatatcaaatttctaataccggcggtagcaatgtcattgtagatggcaaagaagtgccaacgcagaatacttctactgttacttcgggtaatggtactactagtccaatctatggattaggtaatactactcgtggtgattattccgcaattggtgaaatcccaccagtatacactgaaaattcagtaatcaagtattactatcgtgatgaaaatggtaatttaaaagaagctgaaagttctgatcagtatcctaacgtaaacgtttcgggtcttactggtcaagaatttgtaattcctaatgtggatcaatataagcgggttatcaagggacgttatttaaattcagataatttgcctacaggtgatttcacgggaacgatttctcaatttggtgaggggaaatattataagaaagtctactatgattatggtacagatgatgtggattattacgtagtatataaccaagtttcacctgacggcacaatggatgttagtctctttagaggtgacaataatacacctattgaatcaagaagggtgggtccaggtagatctattcgttttaccagtcgtaactatactgctcgtaatccatatgtgaccgaaacaccacatgaagtacaatttatttacgataaattaggttccattgttccagtcgatgaagatggtaacgtaattggcgacttagtccaattcaataatagtactgatccaactaaggctgctgtaaccgattcgccagttattgctggttatacaattaaggatcctactcaaagagagattaccccacatgatcctggcaaaaatattaaggtagtctatgttcgcaaccatgtgacagcagctattaagtatatcgatgatactgctggcgatgacttaagtgcgtacaacaagtcaattacagctaagccaggtgaagcacttaactatactactaaagattcaattacagaactccagaataaagggtatgtattagtaagtgataacttcaatgtaactactatgcctgaaaatggtggtaattacgaagttcacgtaaagcatggcactaagacaatcgatccagataacccaactgataagtacaccaagaaggatttacaaaaaacagctactcgtacgattaattatgttgatgatcaaggcaacaagattgcagaatctgtgacttccacagttgttttcacagggactggtactgtagatgccgtaaccggtaacttagtgaacttacatcccgacggttcgattaaagaccaaaacggtaagcttacttggacttactcagttgatggcggtgttgtacaaaaaagtgatacttacacatttagcgcaacaactgctcgaccaactattgatcacaataattctacttacaactttacttctactactcccgctgattacaatgctggcaatggtgctgtatcgagttatcgtgtgaatagtactgatccacaaaacttaattgttaatgttgtttataccaagcaagctatctaccatgcaggtaagactgaaactaagagtgtaactcgcaccattaattatttagatggtaagactggcgaaaagataccaactgatttaattgcaactaacccagttgcacaaacagttaatttgcatcgtactgaaattattgatgacaacggcaaggtgatcggctacggtacaatcagtaaagatggtaaatcatacactattaacaatgattgggtagtcgacggtaagtgggcaagtgtaacttcacctgatttatcagctaagggttataaagctccacgttttgaaaatggtacttcagctgctagagttgacgaagtaattgttggtagtggtaccaaagacgctactgttaatgtttattacgatcataatttgatcccaattggaccagataattttgataagcatggcgtagatcgaagccagattgagaagcaggttaaagaaacagttcattatgtaggtgctggcgataagactcctgctgatcatgtgcaaacttcgaagtggacgcgcactattactattgatgcggtaactaaagaagttgtacctaatggtcaatatacaactgattggacaattccaaagggtgagaagaccgagtatgctcaagtaaatacgccagtagttaatggctactatgctgatcaagctaatgttccggcaacgactgtaactcaaaatgatattgaaaaaacagtaacttataagcaaattggattatttggtaaattatattttgaaggaggtaggattgttccagttgatccaaatggtaagccaattccagatgcaccaactccacaatatcctaacgatccaacggatccgactaaggtacttcctaatgtaccggtgccaaatattccaggctacaagccaagtgtgccaacagttactccaactgaccctggcaaggatacacaagttccatatacaccggtaactccaactaatccagataatccagtcattccaacgcctcaaccggaaccaaaccctgataatggtaaggataagccggtcgatccatccaagccatcagatgatccagttcatcctgaatatcctggtattaagaggggacaggataaacctgataaggaaaagactgataagaagagaaatggcaagactaagggtaaagaaaatacacctactggaagagatgctgttaagcgagctggacgaagcgatgatgcacttaaattagctagtgaagctaaaaatcgccgtatgactattcaaggtaagaatgaagaattaccacaagctggtgaagatcataatgctatggcgttgattggtcttgcatttgccactcttgctggaagtgtagtctttgctactgataggaaacggagataa

SEQ ID NO: 52 - DNA, 870 bp, Mus musculus
  source 1..870: mol_type = genomic DNA
atggaacgtccacaacctgattcaatgccacaggatttatcagaagctttgaaagaggctacaaaggaagttcatatacaagctgagaatgctgaatttatgaagaatttccagaaaggacaagtttctagagaaggatttaagttagttatggcttcattgtaccatatatatacagctttggaagaggaaattgagagaaataaacagaatccagtttacgctccattatatttcccagaggaattacatagacgtgctgcattagaacaagacatggcattctggtatggtccacactggcaagagattattccatgtacaccagctacacaacactatgttaaaagattacatgaagtcggacgtacacacccagaattattggttgcacatgcttacactagatacttaggagacttgtctggaggtcaggttcttaagaaaattgctcagaaagctatggcattaccatcttcaggagagggtttagcatttttcacattcccaaatattgattcacctactaaattcaagcagttatacagagctagaatgaacacattagaaatgactccagaagtaaagcatcgtgtaacagaagaggctaaaactgctttcttgttaaatattgagttattcgaagagttgcaggttatgttgactgaggaacacaaggatcaatctccatcacagatggcatcattacgtcagcgtccagcttcattggtacaagacactgctccagcagaaactccaagaggtaagccacaaatttcaacaagttcatcacaaacacctttgttacagtgggttcttacattgtcttttcttttagctactgtagcagttggaatatatgcaatgtaa

SEQ ID NO: 53 - DNA, 6384 bp, Bacillus subtilis
  source 1..6384: mol_type = genomic DNA
atgcgcaacttgaccaagacatctctattactggccggcttatgcacagcggcccaaatggtttttgtaacacatgcctcagctgaggaagtagcatcttctaacactcaaacaggtgaaacaacagttcaccaagcccagcctttggataaacttcctgacgacgtggcagctgcaattgcaaaggcggatgagaacgggggaagagaatttgtaaaaccgaaagctgaatcagagggcggtaaagttaccaaggacacggagcctacaaaaccagccaacgaaggttctcatgagttggcaagtccaaaagtcgaaacgccgaataaggttgaagaaggtacaaaagccgaagataaacaaaagtctgaggaggctaaccctaagccggtcgaatctgcaagtacttcaggcactgagcttaaagaagattcaaaaaaaacttctgagaaggatcaggtgaaagcagatacagaaataaagccaagctctgagaagagccaggcccttagcggcgaatcaaataaagcagaagtcgagaaagaaaaacagcttttgtctgagagaaaacaagactttaataaagactggtattttaaattaaatgcccagggagatttcagtaaaaaagacgtggatgtgcatgattggtcaaaattaaacttaccgcatgattggtctatttactttgactttgatcacaagagcccggcacgaaacgaggggggtcagttaaacggggggaccgcttggtatcgaaagacttttaccttaaatgaagcggacaagaataaggacgtgcgtattaactttgacggagtatacatggacagcaaagtctatgtgaatgggaagttcgtgggacactatccaagtggttacaatcacttctcttatgacattactgagtttcttaataaagatggatcagaaaacagcattaccgttcaagttactaacaagcaaccgagctctcgatggtattctggatctggtatctatcgagacgttactcttagttaccgtgataaagtccacgtggctgaaaatggtaaccatattaccacccctaagcttgctgagcagaaggaaggaaatgttgaaactcaggttcagtcaaagataaaaaatactgacaagaaagctgctaaagtgttcgttgaacagcaaatatttaccaaggaggggaaggtcgtgagtgagttagtgcgtagcgaaactaaaaacttagctgaaaacgaggttgccgactttcgtcagacaatacttgttaataagccaactttatggacgactaagtcttatcaccctcagttgtatgtgcttaagaccaaagtatacaaggagggtcaattagtggacgtgacggaggacacatttggatatagatattttaactggactgccaaagatggcttttcattgaatggagaaagaatgaaatttcatggagtgagtatccatcacgataatggagccttaggagcagaggaaaattataaagctacataccgaaaattaaaattattgaaggatatgggtgtcaacagtattcgtaccacgcacaaccctgcgagcccacagttacttgacgccgcggcaagtttaggtcttttagtacaggaggaggcattcgacacctggtatggtgggaaaaagacttatgattatggccgtttcttcgatcaagatgccacacatcctgaggccaaaaagggtgaaaaatggagcgatttcgatttaagaactatggttgaacgagacaagaataacccttcaatagtgatgtggagtttgggtaacgaagtggaggaggctaacggctctccacgtagcatcgagaccgcgaaaagattaaaaacaatcattaaagccatcgacactgagagatacgtaactatgggtgaaaacaaattttcacgtgctgctaccggagatttccttaagcttgctgaaataatggatgcggttggaatgaattacggagaaagattttatgacgccgttcgtagagcccatccagactggttgatatacggttcagagaccagctcagccacgcgaacacgagactcttattacaatcctgcccagatacttggtcatgacaatcgtcctaacagacattatgaacagtctgactatggtaacgatagagtaggatggggtcgtaccgcaacagaaagttggacattcgatcgagatcgagctggatatgccggtcagttcatctggacaggcatcgactacataggtgagccgaccccatggcataaccaggataacaccccggttaaaagtagttattttggtataattgacaccgcagggttgccgaaaaacgatttctacctttaccgatcagagtggtattcagcaaaggaaaaaccgacagttagaatattaccacattggaattggacagaagaaaccttaaaagaccgaaagatgcttgtggatggaaaagtacctgttcgtactttttcaaatgccgcaagtgtcgagttgtttttgaacgggcagtctcttggtaaaaaggagtacacaaagaaaagaactgaggacggacgtccttatcacgagggggctaagccttcagaattgtacttagagtggttagtaaagtaccagccagcacatttagaagctatagctagagatgaatctggaaaagaaattgctagagataaaattacaactgctggtaagccagctgcagttagattgattaaggaagatcatgctattgcagctgatggaaaggatttaacatacatatactatgaaattgtagattctcaaggtaacgtagttcctacagctaacaatttagtaagattccagttgcatggacagggacaattggttggtgtagacaatggagagcaagctagtcgtgaacgttacaaagctcaagctgatggatcatggattcgtaaagcatttaacggaaagggagttgcaattgtaaaatcaactgaacaagcaggtaaatttactttaactgctcattcagacttattgaaatcatctcaagttacagtattcacaggtaagaaagaaggacaagaaaagacagtattaggaactgaagttgcaagagttagaacattgataggaaaagatccaaagatgcctaaaactgtaggatttgtttacagcgatggatctcgtgagaaattacctgttacttggtctcaggtagatgtttcacaggcaggtgttgtaacagttaaaggaactgctaacggtagagaagttgaggctagagttgaggtattagctatagctaaagagttgccaactgttaagcgtattgctcctggagcagatttgaatacagttgataaatacgttagtatattagtaactgatggatctgttcaggaatatgaggttgacagatgggagattgcagaagcagataaagctaagttatctgttgcaggatctagaattcaaatgactggacagttagcaggtgagacaattcatgcaacattggttgtagaagaaggtaacgctgctgcaccagcagttccaactgttacagttggtggagaggctgttacaggtttaacttcacagcaaccaatgcagtatagaactttggcttacggagctcaattgcctgaagtaacagcttctgctgaaaacgctgatgttacagttcttcaagcttcagctgcaaatggtatgagagcatcaatatttgtacaaccaaaggatggtggaccattgcagacatacgctattcagtttttggaagaagcacctaagattgatcacttgaatcttcaagtagagcaagctgacggattgaaagaggatcaaactgttaacttatcagttagagctcactatcaagatggtacacaagctgttcttccagcagataaggtttcattctcaacatctggtgagggagaagttgctgttcgtaaaggaatgttggaattacacaaaccaggtgcattaacattgaaagctgagtatgaaggagctactggacaaataaacttgacaattcaagctaatacagagaagaaaattgctcaatcaattagaccagttaatgttgtaacagatcttcatcaggaacctacattaccatctacagttactgttgaatacgacaaaggtttccctaaagctcataaggttacatggcaagctattcctaaagagaaattagaccattaccaatcatttgaagttttgggtaaggttgaaggaattgacatggaggctcgtgctaaagttagtgttgaaggaattgtatcagttgaagaggtttcagttactacacctatagctgaggctccacaattgccagaatctgttagaacttacgattcaaacggacacgtttcttcagcaaaagttgcatgggatgctatacgtccagaacaatacgcacgtgagggtgtattcacagttaacggacgtttggaaggaactcaattaactactaaattacatgtaagagtatcagctcagactgagcagggagctaacatttctgaccaatggacaggatctgaattgcctttggcattcgcatcagattctaatccaactgatccagtatcaaacgtaaacgataaattgatatctttcaatgatagacctgctaatagatggactaattggaacagatctaaccctgaggcttcagttggagttttattcggagactcaggtatattgtctaagagatctgtagataatttgtcagttggattccacgaagaccatggtgtaggagctccaaagtcttatgtaattgaatactatgtaggaaagactgttcctacagctccaaaaaacccatctttcgttggtaacgaggaacacgtttttaacgacccagctaactggaaggaggtttcaaacttgaaggctcctgcacaattaaaggctggagagatgaatcacttttctttcgataaggttgagacttatgctgttagaatcagaatggttcgtgctgataataaattaggtacatcaattacagaagttcagatatttgctaagcaggttgctgcagctaagcaaggtcaaactcgtattcaagttgacggaaaggatttagcaaacttcaatccagacttgacagattattacttagaatcagttgatggtaaagttccagctgtaacagctagtgtttctaataatggattggctacagttgttccatcagtaagagagggtgaaccagttagagtaattgctaaagctgaaaatggtgatattttgggagagtatagattgcatttcacaaaggataaagacttattatctagaaagccagttgcagctgtaaagcaggctagattattgcagttaggtcaaccattagacttaccaactaaagtaccagtatatttcacaggtaaggatggatatgaagctaaagatatgacagttgaatgggaggaggtaccagctgaaaacttaactaaagctggtcaattcacagtacgtggacgtgtattaggatctaatttgaatgctgagtttactgttagagttactgacaagttgggtgaagcattaagtgataacccaaactatgatgagaactcaaatcaagctttcgcttcagctactaatgacattgatgactcttcacacgatagagttgactatattaatgatagagaccattcagagaatagacgttggactaattggtctaagacaccatcttcaaatccagaagtttctgctggagttatttttagagagaatggtaaaatagttgaacgtacagttgctcaggctaaattacatttctttgcagattctggaacagatgctccatctaaattggttttggaaagatatgtaggtccagactttgaggttcctacttattattcaaactaccaagcttacgaatcaggacatccattcaacaatccagaaaactgggaagcagttccataccgtgctgataaagacattgaagctggagacgaaataaatgttacatttaaggctgtaaaagctaaggctatgcgttggcgtatggaacgtaaagctgataagtcaggagttgcaatgattgaaatgacatttcttgctccatctgaattgccacaggaatctacacagtcaaagatattagtagatggtaaagaattggctgactttgctgagaatagacaagactatcagataacatacaaaggtaagagaccaaaagttgcagttgaggaaaacaatcaagttgcatcaacagttgtagactcaggagaggacagattaccagttttggttcgtttagtttcagagtcaggaaagcaagttaaagaatatagaattcaattaattaaggagaaaccagtttcagaaaagacagtagcagcttaa

SEQ ID NO: 54 - DNA, 135 bp, Listeria monocytogenes
  source 1..135: mol_type = genomic DNA
aagtattatggtaatggagttacatgtggtaaacattcatgttctgtagattggggtaaagctacaacttgtataattaacaatggagctatggcatgggctactggtggacatcaaggaaatcataaatgttaa

SEQ ID NO: 55 - DNA, 129 bp, Listeria monocytogenes
  source 1..129: mol_type = genomic DNA
agaaaacttatttcaattactttttagataaaataatgggaagaggcaatcagtagagttattaacatttgttaacgagttttatttttatataatctataatagatttataaaaataaggagattatt

SEQ ID NO: 56 - DNA, 56 bp, Listeria monocytogenes
  source 1..56: mol_type = genomic DNA
ttaacatttgttaacgagttttatttttatataatctataatagatttataaaaat

SEQ ID NO: 57 - DNA, 2023 bp, Listeria monocytogenes
  source 1..2023: mol_type = genomic DNA
gtgtataaaattttaatagttgatgatgatcaggaaattttaaaattaatgaagacagcattagaaatgagaaactatgaagttgcgacgcatcaaaacatttcacttcccttggatattactgattttcagggatttgatttgattttgttagatatcatgatgtcaaatattgaagggacagaaatttgtaaaaggattcgcagagaaatatcaactccaattatctttgttagtgcgaaagatacagaagaggatattataaacggcttaggtattggtggggatgactatattactaagccttttagccttaaacagttggttgcaaaagtggaagcaaatataaagcgagaggaacgcaataaacatgcagttcatgttttttcagagattcgtagagatttaggaccaattacattttatttagaagaaaggcgagtctgtgtcaatggtcaaacaattccactgacttgtcgtgaatacgatattcttgaattactatcacaacgaacttctaaagtttatacgagagaggatatttatgatgacgtatatgatgaatattctaatgcactttttcggtcaatctcggagtatatttatcagattaggagtaagtttgcaccatacgatattaatccgataaaaacggttcggggacttgggtatcagtggcatgggtaaaaaatattcaatgcgtcgacggatatggcaagctgtcattgaaattatcataggtacttgtctacttatcctgttgttactgggcttgactttctttctacgacaaattggacaaatcagtggttcagaaactattcgtttatctttagattcagataatttaactatttctgatatcgaacgtgatatgaaacactacccatatgattatattatgtttgacaatgatacaagtaaaattttgggaggacattatgtcaagtcggatgtacctagttttgtagcttcaaaacagtcttcacataatattacagaaggagaaattacttatacttattcaagcaataagcatttttcagttgttttaagacaaaacagtatgccagaatttacaaatcatacgcttcgttcaatttcttataatcaatttacttaccttttcttttttcttggtgaaataatactcattattttttctgtctatcatctcattagagaattttctaagaattttcaagccgttcaaaagattgcattgaagatgggggaaataactacttttcctgaacaagaggaatcaaaaattattgaatttgatcaggttctgaataacttatattcgaaaagtaaggagttagctttccttattgaagcggagcgtcatgaaaagcatgatttatccttccaggttgctgcactttcacatgatgttaagacacctttaacagtattaaaaggaaatattgaactgctagagatgactgaagtaaatgaacaacaagctgattttattgagtcaatgaaaaatagtttaactgtttttgacaagtattttaacacaatgattagttatacaaaacttttgaatgatgaaaatgattacaaagcgagaatctccctggaggattttttgatagatttatcagttgagttggaagagttgtcaacaacttatcaagtggattatcagctagttaaaaaaacagatttaaccactttttacggaaatacattagctttaagtcgagcacttatcaatatctttgttaatgcctgtcagtatgctaaagagggtgaaaaaatagttagtttgagtatttatgatgatgaaaaatatctctattttgaaatctggaataatggtcatcctttttctgaacaagcaaaaaaaaatgctggaaaactatttttcacagaagatactggacgtagtgggaaacactatgggattggactatcttttgctcaaggtgtagctttaaaacatcaaggaaacttaattctcagtaatcctcaaaaaggtggggcagaagttatcctaaaaataaaaaagtaa

SEQ ID NO: 58 - DNA, 99 bp, Listeria monocytogenes
  source 1..99: mol_type = genomic DNA
gcgagcataataaacggctctgattaaattctgaagtttgttagatacaatgatttcgttcgaaggaactacaaaataaattataaggaggcactcaaa

SEQ ID NO: 59 - DNA, 129 bp, Listeria monocytogenes
  source 1..129: mol_type = genomic DNA
ggcagaagttatcctaaaaataaaaaagtaatttagtaatctctaaggattactttttttgtttctgaatagattctgaaaattgttttatatactttttttaaacataaaataaagtgaggaaatata

SEQ ID NO: 60 - DNA, 537 bp, Streptococcus pneumoniae
  source 1..537: mol_type = genomic DNA
atgactaacattgaccgtcgtatcagcaaaaccaaaaaagccatctatcaagcatttattcaattattaaatgctaaaggatatgaagctacaactgttcaagatattattgatttggctgatgttggaagatcaacattctattgtcattatgaatctaaagaattattattggatcaattatgtagatatttgtttcatcatttgtttgaaagagaacaagctatttcaacagaagattatttggctcatttattcttacatttccataagaatcaagatcatattacatcattattgttctcaaagaatgattatttcttaagacaattacataaggaattagaacatcatgtttattcagttttagctgataatttgaaagaagctcatccaaatttaccaacatcatatttgcaacatttggttatgtcaaattttattgaaactttgacatggtggttgaagaaaggacaagacttcacagatcaagaagttgtacaattttatttggatttattaattccaaagaattga

SEQ ID NO: 61 - DNA, 132 bp, Streptococcus pneumoniae
  source 1..132: mol_type = genomic DNA
atggacacttaaggcaaattgttcagaactgaataaagctgacgttttgcttctatcctttctttgagttttagtggataatgataatgaacaaggtgttcataaatctattataacaaaggaatgagaaat

SEQ ID NO: 62 - DNA, 365 bp, Lactococcus lactis
  source 1..365: mol_type = genomic DNA
tcctataatggttactgttttcccttgaagaccatatcggatatttgggaggtcttttgcattgatagtggttgtcgcagaaactttataagcatttccctctttaaaagctgtgggagcactatctatttggttgattattccagttatctagactcgataacttataaattactgacagatctgtcagctggttcaactagcggtggtcaaactgttagtaataaaacttattgttttgatgttcggcttaaggatggaaggatttttcaaataaaaaagtaaaaaataatgttaactggttgacattatttttactttgctatataattaaccagtaaactaattatggaggacgaaatact

SEQ ID NO: 63 - DNA, 4107 bp, synthetic construct
  source 1..4107: mol_type = other DNA
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 64 - DNA, 1617 bp, Bacillus subtilis
  source 1..1617: mol_type = genomic DNA
ttgcgcaacttgaccaagacatctctattactggccggcttatgcacagcggcccaaatggtttttgtaacacatgcctcagctgaagaaagcatcgaatacgaccatacgtatcaaaccccttcatacatcatcgaaaagtcaccgcagaagccggtacaaaacacaacccagaaagaatcgctattttcctatcttgacaagcatcaaacgcagtttaagctcaaagggaatgcgaacagccattttcgcgtttcgaaaaccataaaggatccaaagacaaaacaaacgttttttaaattaacggaggtttacaaaggaattccgatttacggctttgaacaagcggtcgcgatgaaggaaaacaaacaagtgaaaagtttctttggaaaggtgcatccgcaaatcaaggacgtctccgtcacaccgtctatttctgagaaaaaagcaatacatacagcaaggcgtgagctcgaggcttccattggaaaaatcgaatatcttgatggggaaccaaaaggcgaattatatatctatccacacgacggtgaatatgatctcgcctaccttgtgagactctcgacatctgaacctgagcctggctattggcattatttcatcgatgccaaaaacggaaaggtcatcgagtcctttaatgccattcatgaagcggcaggtacaggaatcggcgtgtcaggtgatgaaaaaagctttgacgtcacagaacaaaatgggcgcttttatttggctgacgaaacaaggggaaaagggatcaatacatttgacgcgaagaacctgaacgaaaccttgtttacgcttttgtctcaactgatcgggtatacgggcaaagaaatagtcagcggcacgtccgtatttaatgaacctgcggctgtagacgcacacgcaaatgcgcaagccgtttacgattattacagcaagacatttggccgtgattcttttgatcaaaacggagcaaggattacgtctaccgtgcatgtcggcaaacaatggaacaatgctgcgtggaacggtgtccagatggtatacggggatggagacggttcgaaatttaagccgctgtctggatcgctcgacattgtcgcgcatgaaatcacacacgcagtcacacagtattccgccggtcttttatatcaaggagaacccggtgcattaaatgagtccatttctgacattatgggcgcgatggctgaccgtgatgattgggagatcggcgaagatgtctatactcctggtattgcaggagattcattgcggtcattggaggacccatctaagcagggaaatccagatcattactcgaaccgctacacaggaacagaggattatggcggagtccatatcaattcgtccattcacaataaagcagcttatcttcttgcagaaggaggcgtgcaccacggtgtacaggttgaagggattgggcgtgaagcaagtgaacaaatttactatcgggctttaacatattatgtaacggcatctacagatttcagcatgatgaagcaagcggcgattgaagctgccaatgatttatacggtgaaggctcgaagcaatcagcttcagtcgaaaaggcgtatgaggctgtcggcattctatga

SEQ ID NO: 65 - DNA, 1566 bp, Bacillus subtilis
  source 1..1566: mol_type = genomic DNA
gtgggtttaggtaagaaattgtctgttgctgtcgctgcttcgtttatgagtttatcaatcagcctgccaggtgttcaggctgctgaaggtcatcagcttaaagagaatcaaacaaatttcctctccaaaaacgcgattgcgcaatcagaactctctgcaccaaatgacaaggctgtcaagcagtttttgaaaaagaacagcaacatttttaaaggtgacccttccaaaaggctgaagcttgttgaaagcacgactgatgcccttggatacaagcactttcgatatgcgcctgtcgttaacggagtgccaattaaagattcgcaagtgatcgttcacgtcgataaatccgataatgtctatgcggtcaatggtgaattacacaatcaatctgctgcaaaaacagataacagccaaaaagtctcttctgaaaaagcgctggcactcgctttcaaagctatcggcaaatcaccagacgctgtttctaacggagcggccaaaaacagcaataaagccgaattaaaagcgatagaaacaaaagacggcagctatcgtcttgcttacgacgtgacgattcgctatgtcgagcctgaacctgcaaactgggaagtcttagttgacgccgaaacaggcagcattttaaaacagcaaaataaagtagaacatgccgccgccactggaagcggaacaacgctaaagggcgcaactgttcctttgaacatctcttatgaaggcggaaaatatgttctaagagatctttcaaaaccaacaggcacccaaatcatcacatatgatttgcaaaacagacaaagccgccttccgggcacgcttgtctcaagcacaacgaaaacatttacatcttcatcacagcgggcagccgttgacgcacactataacctcggtaaagtgtacgattatttttattcaaactttaaacgaaacagctatgataacaaaggcagtaaaatcgtttcttccgttcactacggcactcaatacaataacgctgcatggacaggagaccagatgatttacggtgatggcgacggttcattcttctctccgctttccggctcattagatgtgacagcgcatgaaatgacacatggcgtcacccaagaaacagccaacttgatttatgaaaatcagccaggtgcattaaacgagtctttctctgacgtattcgggtattttaacgatacagaagactgggacatcggtgaagacattacggtcagccagcctgctcttcgcagcctgtccaaccctacaaaatacaaccagcctgacaattacgccaattaccgaaaccttccaaacacagatgaaggcgattatggcggtgtacacacaaacagcggaattccaaacaaagccgcttacaacaccatcacaaaacttggtgtatctaaatcacagcaaatctattaccgtgcgttaacaacgtacctcacgccttcttccacgttcaaagatgccaaggcagctctcattcagtctgcccgtgacctctacggctcaactgatgccgctaaagttgaagcagcctggaatgctgttggattgtaa

SEQ ID NO: 66 - DNA, 1146 bp, Bacillus subtilis
  source 1..1146: mol_type = genomic DNA
gtgagaagcaaaaaattgtggatcagcttgttgtttgcgttaacgttaatctttacgatggcgttcagcaacatgtctgcgcaggctgccggaaaaagcagtacagaaaagaaatacattgtcggatttaaacagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgtaccttctgaaacaaacccataccaggacggcagttctcacggtacgcatgtagccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtcgttgacaaagccgtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgcaggttctgagcttgatgtgatggctcctggcgtgtccatccaaagcacacttcctggaggcacttacggcgcttataacggaacgtccatggcgactcctcacgttgccggagcagcagcgttaattctttctaagcacccgacttggacaaacgcgcaagtccgtgatcgtttagaaagcactgcaacatatcttggaaactctttctactatggaaaagggttaatcaacgtacaagcagctgcacaataa

SEQ ID NO: 67 - protein, 5 aa, synthetic construct
  VARIANT 3: note = Any amino acid
LPXTG

SEQ ID NO: 68 - protein, 4 aa, synthetic construct
LPTG